SEQUENCE LISTING (266 sequences)

SEQ ID NO: 1 - skipped

SEQ ID NO: 2 - protein, 7 aa, synthetic construct
  REGION 1..7: note = H1 HCDR1; H6 HCDR1; H10 HCDR1; H12 HCDR1; H17 HCDR1; H18 HCDR1
GFTFSSY

SEQ ID NO: 3 - protein, 7 aa, synthetic construct
  REGION 1..7: note = H4 HCDR1
GFTFSTY

SEQ ID NO: 4 - protein, 7 aa, synthetic construct
  REGION 1..7: note = H8 HCDR1
GGSISGY

SEQ ID NO: 5 - protein, 7 aa, synthetic construct
  REGION 1..7: note = H9 HCDR1
GYTFTGY

SEQ ID NO: 6 - protein, 7 aa, synthetic construct
  REGION 1..7: note = H13 HCDR1
GGTFSSY

SEQ ID NO: 7 - protein, 7 aa, synthetic construct
  REGION 1..7: note = H5 HCDR1
GYSFTSY

SEQ ID NO: 8 - protein, 7 aa, synthetic construct
  REGION 1..7: note = H7 HCDR1
GFTVSSN

SEQ ID NO: 9 - protein, 7 aa, synthetic construct
  REGION 1..7: note = H15 HCDR1
GYTLTEL

SEQ ID NO: 10 - protein, 6 aa, synthetic construct
  REGION 1..6: note = H1 HCDR2; H6 HCDR2; H10 HCDR2; H12 HCDR2; H17 HCDR2; H18 HCDR2
SGSGGS

SEQ ID NO: 11 - protein, 6 aa, synthetic construct
  REGION 1..6: note = H4 HCDR2
NQDGTE

SEQ ID NO: 12 - protein, 5 aa, synthetic construct
  REGION 1..5: note = H8 HCDR2
NHSGS

SEQ ID NO: 13 - protein, 6 aa, synthetic construct
  REGION 1..6: note = H9 HCDR2
NPNSGG

SEQ ID NO: 14 - protein, 6 aa, synthetic construct
  REGION 1..6: note = H13 HCDR2
IPIFGT

SEQ ID NO: 15 - protein, 6 aa, synthetic construct
  REGION 1..6: note = H5 HCDR2
DPSDSY

SEQ ID NO: 16 - protein, 5 aa, synthetic construct
  REGION 1..5: note = H7 HCDR2
YSGGS

SEQ ID NO: 17 - protein, 6 aa, synthetic construct
  REGION 1..6: note = H15 HCDR2
DPEDGE

SEQ ID NO: 18 - protein, 11 aa, synthetic construct
  REGION 1..11: note = H1 HCDR3; H10 HCDR3; H12 HCDR3; H17 HCDR3
GYSGYPAVLDY

SEQ ID NO: 19 - protein, 8 aa, synthetic construct
  REGION 1..8: note = H4 HCDR3
EPGIALDY

SEQ ID NO: 20 - protein, 14 aa, synthetic construct
  REGION 1..14: note = H6 HCDR3
DPDPGYFDWVAGDY

SEQ ID NO: 21 - protein, 11 aa, synthetic construct
  REGION 1..11: note = H8 HCDR3
GGGSSGYYLDY

SEQ ID NO: 22 - protein, 13 aa, synthetic construct
  REGION 1..13: note = H9 HCDR3
GVGLWFGEPYFDY

SEQ ID NO: 23 - protein, 23 aa, synthetic construct
  REGION 1..23: note = H13 HCDR3
DQGVVPAAEIPYYDFWSGPPFDP

SEQ ID NO: 24 - protein, 15 aa, synthetic construct
  REGION 1..15: note = H18 HCDR3
KDPDPGYFDWVAGDY

SEQ ID NO: 25 - protein, 13 aa, synthetic construct
  REGION 1..13: note = H5 HCDR3
HPVTNYYYYGMDV

SEQ ID NO: 26 - protein, 11 aa, synthetic construct
  REGION 1..11: note = H7 HCDR3
DYGELHDAFDI

SEQ ID NO: 27 - protein, 13 aa, synthetic construct
  REGION 1..13: note = H15 HCDR3
ERASGSYEYYFDY

SEQ ID NO: 28 - protein, 25 aa, synthetic construct
  REGION 1..25: note = H1 HFR1
QVQLQESGGGLVQPGGSLRLSCAAS

SEQ ID NO: 29 - protein, 25 aa, synthetic construct
  REGION 1..25: note = H4 HFR1
QVQLVQSGGGLVQPGGSLRLSCAAS

SEQ ID NO: 30 - protein, 25 aa, synthetic construct [sequence omitted]

SEQ ID NO: 31 - protein, 25 aa, synthetic construct
  REGION 1..25: note = H8 HFR1
QVQLQESGAGLLKPSETLSLTCAVY

SEQ ID NO: 32 - protein, 25 aa, synthetic construct
  REGION 1..25: note = H9 HFR1
EVQLVESGAEVKKPGASVKVSCKAS

SEQ ID NO: 33 - protein, 25 aa, synthetic construct
  REGION 1..25: note = H10 HFR1
QLQLQESGGGLVQPGGSLRLSCAAS

SEQ ID NO: 34 - protein, 25 aa, synthetic construct
  REGION 1..25: note = H12 HFR1
EVHLVESGGGLVQPGGSLRLSCAAS

SEQ ID NO: 35 - protein, 25 aa, synthetic construct [sequence omitted]

SEQ ID NO: 36 - protein, 25 aa, synthetic construct
  REGION 1..25: note = H5 HFR1
QVQLVQSGAEVKKPGESLRISCKGS

SEQ ID NO: 37 - protein, 25 aa, synthetic construct
  REGION 1..25: note = H7 HFR1
EVQLVESGGGLIQPGGSLRLSCAAS

SEQ ID NO: 38 - protein, 25 aa, synthetic construct
  REGION 1..25: note = H15 HFR1
EVQLVQSGAEVKKPGASVKVSCKVS

SEQ ID NO: 39 - protein, 19 aa, synthetic construct
  REGION 1..19: note = H1 H1 HFR2; H6 HFR2; H10 HFR2; H12 HFR2; H17 HFR2; HFR2
AMSWVRQAPGKGLEWVSAI

SEQ ID NO: 40 - protein, 19 aa, synthetic construct
  REGION 1..19: note = H4 HFR2
WMSWVRQAPGKGLEWVANI

SEQ ID NO: 41 - protein, 19 aa, synthetic construct
  REGION 1..19: note = H8 HFR2
YWSWIRQPPGKGLEWIGEI

SEQ ID NO: 42 - protein, 19 aa, synthetic construct
  REGION 1..19: note = H9 HFR2
YMHWVRQAPGQGLEWMGWI

SEQ ID NO: 43 - protein, 19 aa, synthetic construct
  REGION 1..19: note = H13 HFR2
AISWVRQAPGQGLEWMGGI

SEQ ID NO: 44 - protein, 19 aa, synthetic construct
  REGION 1..19: note = H18 HFR2
AMSWVRQAPGKGLEWFSAI

SEQ ID NO: 45 - protein, 19 aa, synthetic construct
  REGION 1..19: note = H5 HFR2
WISWVRQMPGKGLEWMGRI

SEQ ID NO: 46 - protein, 19 aa, synthetic construct
  REGION 1..19: note = H7 HFR2
YMSWVRQAPGKGLEWVSVI

SEQ ID NO: 47 - protein, 19 aa, synthetic construct
  REGION 1..19: note = H15 HFR2
SMHWVRQAPGKGLEWMGGF

SEQ ID NO: 48 - protein, 41 aa, synthetic construct
  REGION 1..41: note = H1 HFR3; H6 HFR3; H10 HFR3; H12 HFR3; H17 HFR3;
TYYADSVKGRFTISRDNSKNTLYLQMNSLRAEDTAVYYCAK

SEQ ID NO: 49 - protein, 41 aa, synthetic construct
  REGION 1..41: note = H4 HFR3
KYYVDSVKGRFTISRDNAKNSLYLQMNSLRAEDTAVYYCAR

SEQ ID NO: 50 - protein, 41 aa, synthetic construct
  REGION 1..41: note = H8 HFR3
TNYNPSLKSRVTISVDTSKNQFSLKLSSVTAADTAVYYCAR

SEQ ID NO: 51 - protein, 41 aa, synthetic construct
  REGION 1..41: note = H9 HFR3
TNYAQKFQGRVTMTRDTSISTAYMELSRLRSDDTAVYYCAR

SEQ ID NO: 52 - protein, 41 aa, synthetic construct
  REGION 1..41: note = H13 HFR3
ANYAQKFQGRVTITADESTSTAYMELSSLRSEDTAVYYCAR

SEQ ID NO: 53 - protein, 40 aa, synthetic construct
  REGION 1..40: note = H18 HFR3
TYYADSVKGRFTISRDNSKNTLYLQMNSLRAEDTAVYYCA

SEQ ID NO: 54 - protein, 41 aa, synthetic construct
  REGION 1..41: note = H5 HFR3
TNYSPSFQGHVTISADKSISTAYLQWSSLKASDTAMYYCAR

SEQ ID NO: 55 - protein, 41 aa, synthetic construct
  REGION 1..41: note = H7 HFR3
TYYADSVKGRFTISRDNSKNTLYLQMNSLRAEDTAVYYCAT

SEQ ID NO: 56 - protein, 41 aa, synthetic construct
  REGION 1..41: note = H15 HFR3
TIYAQKFQGRVTMTEDTSTDTAYMELSSLRSEDTAVYYCAT

SEQ ID NO: 57 - protein, 11 aa, synthetic construct
  REGION 1..11: note = H1 HFR4
WGLGTLVTVSS

SEQ ID NO: 58 - protein, 11 aa, synthetic construct
  REGION 1..11: note = H4 HFR4; H6 HFR4; H9 HFR4; H10 HFR4; H12 HFR4; H13 HFR4; H17 HFR4; H18 HFR4; H15 HFR4
WGQGTLVTVSS

SEQ ID NO: 59 - protein, 11 aa, synthetic construct
  REGION 1..11: note = H8 HFR4
WGQGALVTVSS

SEQ ID NO: 60 - protein, 11 aa, synthetic construct
  REGION 1..11: note = H5 HFR4; H7 HFR4
WGQGTTVTVSS

SEQ ID NO: 61 - protein, 120 aa, synthetic construct
  REGION 1..120: note = H1 VH
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGLGTLVTVSS

SEQ ID NO: 62 - protein, 117 aa, synthetic construct
  REGION 1..117: note = H4 VH
QVQLVQSGGGLVQPGGSLRLSCAASGFTFSTYWMSWVRQAPGKGLEWVANINQDGTEKYYVDSVKGRFTISRDNAKNSLYLQMNSLRAEDTAVYYCAREPGIALDYWGQGTLVTVSS

SEQ ID NO: 63 - protein, 123 aa, synthetic construct
  REGION 1..123: note = H6 VH
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDPDPGYFDWVAGDYWGQGTLVTVSS

SEQ ID NO: 64 - protein, 119 aa, synthetic construct
  REGION 1..119: note = H8 VH
QVQLQESGAGLLKPSETLSLTCAVYGGSISGYYWSWIRQPPGKGLEWIGEINHSGSTNYNPSLKSRVTISVDTSKNQFSLKLSSVTAADTAVYYCARGGGSSGYYLDYWGQGALVTVSS

SEQ ID NO: 65 - protein, 122 aa, synthetic construct
  REGION 1..122: note = H9 VH
EVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARGVGLWFGEPYFDYWGQGTLVTVSS

SEQ ID NO: 66 - protein, 120 aa, synthetic construct
  REGION 1..120: note = H10 VH
QLQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 67 - protein, 120 aa, synthetic construct
  REGION 1..120: note = H12 VH
EVHLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 68 - protein, 132 aa, synthetic construct
  REGION 1..132: note = H13 VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDQGVVPAAEIPYYDFWSGPPFDPWGQGTLVTVSS

SEQ ID NO: 69 - protein, 120 aa, synthetic construct
  REGION 1..120: note = H17 VH
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 70 - protein, 123 aa, synthetic construct
  REGION 1..123: note = H18 VH
EVQLVESGGGLVQPGGSLRLSCAASGFTFSSYAMSWVRQAPGKGLEWFSAISGSGGSTYYADSVKGRFTISRDNSKNTLYLQMNSLRAEDTAVYYCAKDPDPGYFDWVAGDYWGQGTLVTVSS

SEQ ID NO: 71 - protein, 122 aa, synthetic construct
  REGION 1..122: note = H5 VH
QVQLVQSGAEVKKPGESLRISCKGSGYSFTSYWISWVRQMPGKGLEWMGRIDPSDSYTNYSPSFQGHVTISADKSISTAYLQWSSLKASDTAMYYCARHPVTNYYYYGMDVWGQGTTVTVSS

SEQ ID NO: 72 - protein, 119 aa, synthetic construct
  REGION 1..119: note = H7 VH
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCATDYGELHDAFDIWGQGTTVTVSS

SEQ ID NO: 73 - protein, 122 aa, synthetic construct
  REGION 1..122: note = H15 VH
EVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATERASGSYEYYFDYWGQGTLVTVSS

SEQ ID NO: 74 - protein, 13 aa, synthetic construct
  REGION 1..13: note = H1 LCDR1
SGSTSNTGNNYFS

SEQ ID NO: 75 - protein, 13 aa, synthetic construct
  REGION 1..13: note = H4 LCDR1
SGSSSNIGNNYVS

SEQ ID NO: 76 - protein, 14 aa, synthetic construct
  REGION 1..14: note = H6 LCDR1
TGTSSDVGGYNYVS

SEQ ID NO: 77 - protein, 13 aa, synthetic construct
  REGION 1..13: note = H8 LCDR1
SGSSSNIGNNAVN

SEQ ID NO: 78 - protein, 13 aa, synthetic construct
  REGION 1..13: note = H9 LCDR1
SGSSSNIGSNYVY

SEQ ID NO: 79 - protein, 14 aa, synthetic construct
  REGION 1..14: note = H10 LCDR1
TGTSSDVGGSKYVS

SEQ ID NO: 80 - protein, 13 aa, synthetic construct
  REGION 1..13: note = H12 LCDR1
SGSGSNIGSNTVT

SEQ ID NO: 81 - protein, 13 aa, synthetic construct
  REGION 1..13: note = H13 LCDR1
SGSSSNIGSNSVN

SEQ ID NO: 82 - protein, 14 aa, synthetic construct
  REGION 1..14: note = H17 LCDR1
TGSSSDIGASSRVS

SEQ ID NO: 83 - protein, 14 aa, synthetic construct
  REGION 1..14: note = H18 LCDR1
TGTSSDVGGYTYVS

SEQ ID NO: 84 - protein, 11 aa, synthetic construct
  REGION 1..11: note = H5 LCDR1
RASQGISNWLT

SEQ ID NO: 85 - protein, 11 aa, synthetic construct
  REGION 1..11: note = H7 LCDR1
RASQSISSYLN

SEQ ID NO: 86 - protein, 11 aa, synthetic construct
  REGION 1..11: note = H15 LCDR1
RASQGISSWLA

SEQ ID NO: 87 - protein, 7 aa, synthetic construct
  REGION 1..7: note = H1 LCDR2
KNNLRPS

SEQ ID NO: 88 - protein, 7 aa, synthetic construct
  REGION 1..7: note = H4 LCDR2
DNNKRPS

SEQ ID NO: 89 - protein, 7 aa, synthetic construct
  REGION 1..7: note = H6 LCDR2
EVRKRPS

SEQ ID NO: 90 - protein, 7 aa, synthetic construct
  REGION 1..7: note = H8 LCDR2
YDDLLPS

SEQ ID NO: 91 - protein, 7 aa, synthetic construct
  REGION 1..7: note = H9 LCDR2
NSNQRPS

SEQ ID NO: 92 - protein, 7 aa, synthetic construct
  REGION 1..7: note = H10 LCDR2
DVSYRPS

SEQ ID NO: 93 - protein, 7 aa, synthetic construct
  REGION 1..7: note = H12 LCDR2
NSKLRPS

SEQ ID NO: 94 - protein, 7 aa, synthetic construct
  REGION 1..7: note = H13 LCDR2
NNNQRPS

SEQ ID NO: 95 - protein, 7 aa, synthetic construct
  REGION 1..7: note = H17 LCDR2
GVNNRPS

SEQ ID NO: 96 - protein, 7 aa, synthetic construct
  REGION 1..7: note = H18 LCDR2
EVSKRPS

SEQ ID NO: 97 - protein, 7 aa, synthetic construct
  REGION 1..7: note = H5 LCDR2
SASTLQS

SEQ ID NO: 98 - protein, 7 aa, synthetic construct
  REGION 1..7: note = H7 LCDR2; H15 LCDR2
AASSLQS

SEQ ID NO: 99 - protein, 10 aa, synthetic construct
  REGION 1..10: note = H1 LCDR3
ATWDDNLTLV

SEQ ID NO: 100 - protein, 13 aa, synthetic construct
  REGION 1..13: note = H4 LCDR3
GTWDSSLSAGDWV

SEQ ID NO: 101 - protein, 10 aa, synthetic construct
  REGION 1..10: note = H6 LCDR3
SSYAGSNNLG

SEQ ID NO: 102 - protein, 11 aa, synthetic construct
  REGION 1..11: note = H8 LCDR3
ASWDDSLNGRV

SEQ ID NO: 103 - protein, 11 aa, synthetic construct
  REGION 1..11: note = H9 LCDR3
AAWDDSLSGPV

SEQ ID NO: 104 - protein, 11 aa, synthetic construct
  REGION 1..11: note = H10 LCDR3
SSYTSNSNLPL

SEQ ID NO: 105 - protein, 11 aa, synthetic construct
  REGION 1..11: note = H12 LCDR3
AAWDDSLNGVV

SEQ ID NO: 106 - protein, 12 aa, synthetic construct
  REGION 1..12: note = H13 LCDR3
ATWDDSLNGNVV

SEQ ID NO: 107 - protein, 10 aa, synthetic construct
  REGION 1..10: note = H17 LCDR3
SSHSSGNTLL

SEQ ID NO: 108 - protein, 10 aa, synthetic construct
  REGION 1..10: note = H18 LCDR3
SSYAGSNNVV

SEQ ID NO: 109 - protein, 10 aa, synthetic construct
  REGION 1..10: note = H5 LCDR3
QQANNLRTLT

SEQ ID NO: 110 - protein, 10 aa, synthetic construct
  REGION 1..10: note = H7 LCDR3
QQSYSTLPYT

SEQ ID NO: 111 - protein, 9 aa, synthetic construct
  REGION 1..9: note = H15 LCDR3
QQANSFPYT

SEQ ID NO: 112 - protein, 22 aa, synthetic construct
  REGION 1..22: note = H1 LFR1
QPVLTQSPSASETPGQRVTISC

SEQ ID NO: 113 - protein, 22 aa, synthetic construct
  REGION 1..22: note = H4 LFR1
QSVVTQPPSVSAAPGQKVTISC

SEQ ID NO: 114 - protein, 22 aa, synthetic construct
  REGION 1..22: note = H6 LFR1; H18 LFR1
QSALTQPPSASGSPGQSVTISC

SEQ ID NO: 115 - protein, 22 aa, synthetic construct
  REGION 1..22: note = H8 LFR1
QSVLTQPPSVSEAPRQRVTISC

SEQ ID NO: 116 - protein, 22 aa, synthetic construct
  REGION 1..22: note = H9 LFR1
QSVLTQPPSASGAPGERVTISC

SEQ ID NO: 117 - protein, 22 aa, synthetic construct
  REGION 1..22: note = H10 LFR1
QSALTQPASVSGSPGQSITISC

SEQ ID NO: 118 - protein, 22 aa, synthetic construct
  REGION 1..22: note = H12 LFR1
QPVLTQPPSASGTPGQRVIISC

SEQ ID NO: 119 - protein, 22 aa, synthetic construct
  REGION 1..22: note = H13 LFR1
QSVVTQPPSASGTPGQRVTISC

SEQ ID NO: 120 - protein, 22 aa, synthetic construct
  REGION 1..22: note = H17 LFR1
QSALTQPPSVSGSPGQSVSISC

SEQ ID NO: 121 - protein, 23 aa, synthetic construct
  REGION 1..23: note = H5 LFR1
DIRLTQSPSTVSASIGDRVTITC

SEQ ID NO: 122 - protein, 23 aa, synthetic construct
  REGION 1..23: note = H7 LFR1
DIVMTQSPSSLSASVGDRVTITC

SEQ ID NO: 123 - protein, 23 aa, synthetic construct
  REGION 1..23: note = H15 LFR1
AIQLTQSPSSVSASVGDRVTITC

SEQ ID NO: 124 - protein, 15 aa, synthetic construct
  REGION 1..15: note = H1 LFR2
WYQQVPGMAPKLLIY

SEQ ID NO: 125 - protein, 15 aa, synthetic construct
  REGION 1..15: note = H4 LFR2; H9 LFR2; H13 LFR2
WYQQLPGTAPKLLIY

SEQ ID NO: 126 - protein, 15 aa, synthetic construct
  REGION 1..15: note = H6 LFR2; H10 LFR2; H18 LFR2
WYQQHPGKAPKLMIY

SEQ ID NO: 127 - protein, 15 aa, synthetic construct
  REGION 1..15: note = H8 LFR2
WYQQLPGKAPKLLIY

SEQ ID NO: 128 - protein, 15 aa, synthetic construct
  REGION 1..15: note = H12 LFR2
WYRQFPGAAPKTLIY

SEQ ID NO: 129 - protein, 15 aa, synthetic construct
  REGION 1..15: note = H17 LFR2
WYLQPPGKAPQLIIF

SEQ ID NO: 130 - protein, 15 aa, synthetic construct
  REGION 1..15: note = H5 LFR2; H7 LFR2; H15 LFR2
WYQQKPGKAPKLLIY

SEQ ID NO: 131 - protein, 32 aa, synthetic construct
  REGION 1..32: note = H1 LFR3
GVPDRFSGSKSGTSASLSISGLRSEDEGDYYC

SEQ ID NO: 132 - protein, 32 aa, synthetic construct
  REGION 1..32: note = H4 LFR3
GIPDRFSGSKSGTSATLGITGLQTGDEADYYC

SEQ ID NO: 133 - protein, 32 aa, synthetic construct
  REGION 1..32: note = H6 LFR3
GVPDRFSGSKSGNTASLTVSGLQADDEADYYC

SEQ ID NO: 134 - protein, 32 aa, synthetic construct
  REGION 1..32: note = H8 LFR3
GVSDRFSGSKSGTSASLTISGLQSEDGADYYC

SEQ ID NO: 135 - protein, 32 aa, synthetic construct
  REGION 1..32: note = H9 LFR3
GVPDRFSGSKSGASASLAISGLRSEDEADYYC

SEQ ID NO: 136 - protein, 32 aa, synthetic construct
  REGION 1..32: note = H10 LFR3
GVSDRFSGSKSGNTASLTISGLQAEDEADYYC

SEQ ID NO: 137 - protein, 32 aa, synthetic construct
  REGION 1..32: note = H12 LFR3
WVPDRFTASKSGTSASLAISGLQSEDEADYYC

SEQ ID NO: 138 - protein, 32 aa, synthetic construct
  REGION 1..32: note = H13 LFR3
GVPDRFSGSKSGTSASLAISGLQSEDEADYYC

SEQ ID NO: 139 - protein, 31 aa, synthetic construct
  REGION 1..31: note = H17 LFR3
VPDRFSGSKSGNTASLTISGLRAEDEADYYC

SEQ ID NO: 140 - protein, 32 aa, synthetic construct
  REGION 1..32: note = H18 LFR3
GVPDRFSGSKSGNTASLTVSGLQAEDEADYYC

SEQ ID NO: 141 - protein, 32 aa, synthetic construct
  REGION 1..32: note = H5 LFR3; H7 LFR3; H15 LFR3
GVPSRFSGSGSGTDFTLTISSLQPEDFATYYC

SEQ ID NO: 142 - protein, 10 aa, synthetic construct
  REGION 1..10: note = H1 LFR4; H4 LFR4; H6 LFR4; H8 LFR4; H12 LFR4; H17 LFR4
FGGGTKLTVL

SEQ ID NO: 143 - protein, 10 aa, synthetic construct
  REGION 1..10: note = H9 LFR4; H10 LFR4; H13 LFR4
FGGGTKVTVL

SEQ ID NO: 144 - protein, 10 aa, synthetic construct
  REGION 1..10: note = H18 LFR4
FGGGTELTVL

SEQ ID NO: 145 - protein, 10 aa, synthetic construct
  REGION 1..10: note = H5 LFR4
FGGGTKVDIK

SEQ ID NO: 146 - protein, 10 aa, synthetic construct
  REGION 1..10: note = H7 LFR4
FGQGTKLEIK

SEQ ID NO: 147 - protein, 10 aa, synthetic construct
  REGION 1..10: note = H15 LFR4
FGQGTKVEIK

SEQ ID NO: 148 - protein, 109 aa, synthetic construct
  REGION 1..109: note = H1 VL
QPVLTQSPSASETPGQRVTISCSGSTSNTGNNYFSWYQQVPGMAPKLLIYKNNLRPSGVPDRFSGSKSGTSASLSISGLRSEDEGDYYCATWDDNLTLVFGGGTKLTVL

SEQ ID NO: 149 - protein, 112 aa, synthetic construct
  REGION 1..112: note = H4 VL
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGDWVFGGGTKLTVL

SEQ ID NO: 150 - protein, 110 aa, synthetic construct
  REGION 1..110: note = H6 VL
QSALTQPPSASGSPGQSVTISCTGTSSDVGGYNYVSWYQQHPGKAPKLMIYEVRKRPSGVPDRFSGSKSGNTASLTVSGLQADDEADYYCSSYAGSNNLGFGGGTKLTVL

SEQ ID NO: 151 - protein, 110 aa, synthetic construct
  REGION 1..110: note = H8 VL
QSVLTQPPSVSEAPRQRVTISCSGSSSNIGNNAVNWYQQLPGKAPKLLIYYDDLLPSGVSDRFSGSKSGTSASLTISGLQSEDGADYYCASWDDSLNGRVFGGGTKLTVL

SEQ ID NO: 152 - protein, 110 aa, synthetic construct
  REGION 1..110: note = H9 VL
QSVLTQPPSASGAPGERVTISCSGSSSNIGSNYVYWYQQLPGTAPKLLIYNSNQRPSGVPDRFSGSKSGASASLAISGLRSEDEADYYCAAWDDSLSGPVFGGGTKVTVL

SEQ ID NO: 153 - protein, 111 aa, synthetic construct
  REGION 1..111: note = H10 VL
QSALTQPASVSGSPGQSITISCTGTSSDVGGSKYVSWYQQHPGKAPKLMIYDVSYRPSGVSDRFSGSKSGNTASLTISGLQAEDEADYYCSSYTSNSNLPLFGGGTKVTVL

SEQ ID NO: 154 - protein, 110 aa, synthetic construct
  REGION 1..110: note = H12 VL
QPVLTQPPSASGTPGQRVIISCSGSGSNIGSNTVTWYRQFPGAAPKTLIYNSKLRPSWVPDRFTASKSGTSASLAISGLQSEDEADYYCAAWDDSLNGVVFGGGTKLTVL

SEQ ID NO: 155 - protein, 111 aa, synthetic construct
  REGION 1..111: note = H13 VL
QSVVTQPPSASGTPGQRVTISCSGSSSNIGSNSVNWYQQLPGTAPKLLIYNNNQRPSGVPDRFSGSKSGTSASLAISGLQSEDEADYYCATWDDSLNGNVVFGGGTKVTVL

SEQ ID NO: 156 - protein, 110 aa, synthetic construct
  REGION 1..110: note = H17 VL
QSALTQPPSVSGSPGQSVSISCTGSSSDIGASSRVSWYLQPPGKAPQLIIFGVNNRPSGVPDRFSGSKSGNTASLTISGLRAEDEADYYCSSHSSGNTLLFGGGTKLTVL

SEQ ID NO: 157 - protein, 110 aa, synthetic construct
  REGION 1..110: note = H18 VL
QSALTQPPSASGSPGQSVTISCTGTSSDVGGYTYVSWYQQHPGKAPKLMIYEVSKRPSGVPDRFSGSKSGNTASLTVSGLQAEDEADYYCSSYAGSNNVVFGGGTELTVL

SEQ ID NO: 158 - protein, 108 aa, synthetic construct
  REGION 1..108: note = H5 VL
DIRLTQSPSTVSASIGDRVTITCRASQGISNWLTWYQQKPGKAPKLLIYSASTLQSGVPSRFSGSGSGTDFTLTISSLQPEDFATYYCQQANNLRTLTFGGGTKVDIK

SEQ ID NO: 159 - protein, 108 aa, synthetic construct
  REGION 1..108: note = H7 VL
DIVMTQSPSSLSASVGDRVTITCRASQSISSYLNWYQQKPGKAPKLLIYAASSLQSGVPSRFSGSGSGTDFTLTISSLQPEDFATYYCQQSYSTLPYTFGQGTKLEIK

SEQ ID NO: 160 - protein, 107 aa, synthetic construct
  REGION 1..107: note = H15 VL
AIQLTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKVEIK

SEQ ID NO: 161 - protein, 15 aa, synthetic construct
  REGION 1..15: note = Linker
GGGGSGGGGSGGGGS

SEQ ID NO: 162 - protein, 244 aa, synthetic construct
  REGION 1..244: note = H1 scFv
QPVLTQSPSASETPGQRVTISCSGSTSNTGNNYFSWYQQVPGMAPKLLIYKNNLRPSGVPDRFSGSKSGTSASLSISGLRSEDEGDYYCATWDDNLTLVFGGGTKLTVLGGGGSGGGGSGGGGSQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGLGTLVTVSS

SEQ ID NO: 163 - protein, 244 aa, synthetic construct
  REGION 1..244: note = H4 scFv
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGDWVFGGGTKLTVLGGGGSGGGGSGGGGSQVQLVQSGGGLVQPGGSLRLSCAASGFTFSTYWMSWVRQAPGKGLEWVANINQDGTEKYYVDSVKGRFTISRDNAKNSLYLQMNSLRAEDTAVYYCAREPGIALDYWGQGTLVTVSS

SEQ ID NO: 164 - protein, 248 aa, synthetic construct
  REGION 1..248: note = H6 scFv
QSALTQPPSASGSPGQSVTISCTGTSSDVGGYNYVSWYQQHPGKAPKLMIYEVRKRPSGVPDRFSGSKSGNTASLTVSGLQADDEADYYCSSYAGSNNLGFGGGTKLTVLGGGGSGGGGSGGGGSEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDPDPGYFDWVAGDYWGQGTLVTVSS

SEQ ID NO: 165 - protein, 244 aa, synthetic construct
  REGION 1..244: note = H8 scFv
QSVLTQPPSVSEAPRQRVTISCSGSSSNIGNNAVNWYQQLPGKAPKLLIYYDDLLPSGVSDRFSGSKSGTSASLTISGLQSEDGADYYCASWDDSLNGRVFGGGTKLTVLGGGGSGGGGSGGGGSQVQLQESGAGLLKPSETLSLTCAVYGGSISGYYWSWIRQPPGKGLEWIGEINHSGSTNYNPSLKSRVTISVDTSKNQFSLKLSSVTAADTAVYYCARGGGSSGYYLDYWGQGALVTVSS

SEQ ID NO: 166 - protein, 247 aa, synthetic construct
  REGION 1..247: note = H9 scFv
QSVLTQPPSASGAPGERVTISCSGSSSNIGSNYVYWYQQLPGTAPKLLIYNSNQRPSGVPDRFSGSKSGASASLAISGLRSEDEADYYCAAWDDSLSGPVFGGGTKVTVLGGGGSGGGGSGGGGSEVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARGVGLWFGEPYFDYWGQGTLVTVSS

SEQ ID NO: 167 - protein, 246 aa, synthetic construct
  REGION 1..246: note = H10 scFv
QSALTQPASVSGSPGQSITISCTGTSSDVGGSKYVSWYQQHPGKAPKLMIYDVSYRPSGVSDRFSGSKSGNTASLTISGLQAEDEADYYCSSYTSNSNLPLFGGGTKVTVLGGGGSGGGGSGGGGSQLQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 168 - protein, 245 aa, synthetic construct
  REGION 1..245: note = H12 scFv
QPVLTQPPSASGTPGQRVIISCSGSGSNIGSNTVTWYRQFPGAAPKTLIYNSKLRPSWVPDRFTASKSGTSASLAISGLQSEDEADYYCAAWDDSLNGVVFGGGTKLTVLGGGGSGGGGSGGGGSEVHLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 169 - protein, 258 aa, synthetic construct
  REGION 1..258: note = H13 scFv
QSVVTQPPSASGTPGQRVTISCSGSSSNIGSNSVNWYQQLPGTAPKLLIYNNNQRPSGVPDRFSGSKSGTSASLAISGLQSEDEADYYCATWDDSLNGNVVFGGGTKVTVLGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDQGVVPAAEIPYYDFWSGPPFDPWGQGTLVTVSS

SEQ ID NO: 170 - protein, 244 aa, synthetic construct
  REGION 1..244: note = H17 scFv
QSALTQPPSVSGSPGQSVSISCTGSSSDIGASSRVSWYLQPPGKAPQLIIFGVNNRPSGVPDRFSGSKSGNTASLTISGLRAEDEADYYCSSHSSGNTLLFGGGTKLTVLGGGSGGGGSGGGGSQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSS

SEQ ID NO: 171 - protein, 248 aa, synthetic construct
  REGION 1..248: note = H18 scFv
QSALTQPPSASGSPGQSVTISCTGTSSDVGGYTYVSWYQQHPGKAPKLMIYEVSKRPSGVPDRFSGSKSGNTASLTVSGLQAEDEADYYCSSYAGSNNVVFGGGTELTVLGGGGSGGGGSGGGGSEVQLVESGGGLVQPGGSLRLSCAASGFTFSSYAMSWVRQAPGKGLEWFSAISGSGGSTYYADSVKGRFTISRDNSKNTLYLQMNSLRAEDTAVYYCAKDPDPGYFDWVAGDYWGQGTLVTVSS

SEQ ID NO: 172 - protein, 245 aa, synthetic construct
  REGION 1..245: note = H5 scFv
DIRLTQSPSTVSASIGDRVTITCRASQGISNWLTWYQQKPGKAPKLLIYSASTLQSGVPSRFSGSGSGTDFTLTISSLQPEDFATYYCQQANNLRTLTFGGGTKVDIKGGGGSGGGGSGGGGSQVQLVQSGAEVKKPGESLRISCKGSGYSFTSYWISWVRQMPGKGLEWMGRIDPSDSYTNYSPSFQGHVTISADKSISTAYLQWSSLKASDTAMYYCARHPVTNYYYYGMDVWGQGTTVTVSS

SEQ ID NO: 173 - protein, 242 aa, synthetic construct
  REGION 1..242: note = H7 scFv
DIVMTQSPSSLSASVGDRVTITCRASQSISSYLNWYQQKPGKAPKLLIYAASSLQSGVPSRFSGSGSGTDFTLTISSLQPEDFATYYCQQSYSTLPYTFGQGTKLEIKGGGGSGGGGSGGGGSEVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCATDYGELHDAFDIWGQGTTVTVSS

SEQ ID NO: 174 - protein, 244 aa, synthetic construct
  REGION 1..244: note = H15 scFv
AIQLTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKVEIKGGGGSGGGGSGGGGSEVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATERASGSYEYYFDYWGQGTLVTVSS

SEQ ID NO: 175 - protein, 21 aa, synthetic construct
  REGION 1..21: note = CD8 signal peptide amino acid
MALPVTALLLPLALLLHAARP

SEQ ID NO: 176 - protein, 45 aa, synthetic construct
  REGION 1..45: note = CD8 Hinge amino acid
TTTPAPRPPTPAPTIASQPLSLRPEACRPAAGGAVHTRGLDFACD

SEQ ID NO: 177 - protein, 24 aa, synthetic construct
  REGION 1..24: note = CD8 transmembrane domain amino acid
IYIWAPLAGTCGVLLLSLVITLYC

SEQ ID NO: 178 - protein, 42 aa, synthetic construct
  REGION 1..42: note = 4-1BB costimulatory domain amino acid
KRGRKKLLYIFKQPFMRPVQTTQEEDGCSCRFPEEEEGGCEL

SEQ ID NO: 179 - protein, 112 aa, synthetic construct
  REGION 1..112: note = CD3-zeta amino acid
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 180 - protein, 488 aa, synthetic construct
  REGION 1..488: note = CAR-H1
MALPVTALLLPLALLLHAARPQPVLTQSPSASETPGQRVTISCSGSTSNTGNNYFSWYQQVPGMAPKLLIYKNNLRPSGVPDRFSGSKSGTSASLSISGLRSEDEGDYYCATWDDNLTLVFGGGTKLTVLGGGGSGGGGSGGGGSQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGLGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 181 - protein, 488 aa, synthetic construct
  REGION 1..488: note = CAR-H4
MALPVTALLLPLALLLHAARPQSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGDWVFGGGTKLTVLGGGGSGGGGSGGGGSQVQLVQSGGGLVQPGGSLRLSCAASGFTFSTYWMSWVRQAPGKGLEWVANINQDGTEKYYVDSVKGRFTISRDNAKNSLYLQMNSLRAEDTAVYYCAREPGIALDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 182 - protein, 492 aa, synthetic construct
  REGION 1..492: note = CAR-H6
MALPVTALLLPLALLLHAARPQSALTQPPSASGSPGQSVTISCTGTSSDVGGYNYVSWYQQHPGKAPKLMIYEVRKRPSGVPDRFSGSKSGNTASLTVSGLQADDEADYYCSSYAGSNNLGFGGGTKLTVLGGGGSGGGGSGGGGSEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDPDPGYFDWVAGDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 183 - protein, 488 aa, synthetic construct
  REGION 1..488: note = CAR-H8
MALPVTALLLPLALLLHAARPQSVLTQPPSVSEAPRQRVTISCSGSSSNIGNNAVNWYQQLPGKAPKLLIYYDDLLPSGVSDRFSGSKSGTSASLTISGLQSEDGADYYCASWDDSLNGRVFGGGTKLTVLGGGGSGGGGSGGGGSQVQLQESGAGLLKPSETLSLTCAVYGGSISGYYWSWIRQPPGKGLEWIGEINHSGSTNYNPSLKSRVTISVDTSKNQFSLKLSSVTAADTAVYYCARGGGSSGYYLDYWGQGALVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 184 - protein, 491 aa, synthetic construct
  REGION 1..491: note = CAR-H9
MALPVTALLLPLALLLHAARPQSVLTQPPSASGAPGERVTISCSGSSSNIGSNYVYWYQQLPGTAPKLLIYNSNQRPSGVPDRFSGSKSGASASLAISGLRSEDEADYYCAAWDDSLSGPVFGGGTKVTVLGGGGSGGGGSGGGGSEVQLVESGAEVKKPGASVKVSCKASGYTFTGYYMHWVRQAPGQGLEWMGWINPNSGGTNYAQKFQGRVTMTRDTSISTAYMELSRLRSDDTAVYYCARGVGLWFGEPYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - protein, 490 aa, synthetic construct
  REGION 1..490: note = CAR-H10
MALPVTALLLPLALLLHAARPQSALTQPASVSGSPGQSITISCTGTSSDVGGSKYVSWYQQHPGKAPKLMIYDVSYRPSGVSDRFSGSKSGNTASLTISGLQAEDEADYYCSSYTSNSNLPLFGGGTKVTVLGGGGSGGGGSGGGGSQLQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 186 - protein, 489 aa, synthetic construct
  REGION 1..489: note = CAR-H12
MALPVTALLLPLALLLHAARPQPVLTQPPSASGTPGQRVIISCSGSGSNIGSNTVTWYRQFPGAAPKTLIYNSKLRPSWVPDRFTASKSGTSASLAISGLQSEDEADYYCAAWDDSLNGVVFGGGTKLTVLGGGGSGGGGSGGGGSEVHLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 502 aa, synthetic construct
  REGION 1..502: note = CAR-H13
MALPVTALLLPLALLLHAARPQSVVTQPPSASGTPGQRVTISCSGSSSNIGSNSVNWYQQLPGTAPKLLIYNNNQRPSGVPDRFSGSKSGTSASLAISGLQSEDEADYYCATWDDSLNGNVVFGGGTKVTVLGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDQGVVPAAEIPYYDFWSGPPFDPWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 188 - protein, 488 aa, synthetic construct
  REGION 1..488: note = CAR-H17
MALPVTALLLPLALLLHAARPQSALTQPPSVSGSPGQSVSISCTGSSSDIGASSRVSWYLQPPGKAPQLIIFGVNNRPSGVPDRFSGSKSGNTASLTISGLRAEDEADYYCSSHSSGNTLLFGGGTKLTVLGGGSGGGGSGGGGSQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSGYPAVLDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 189 - protein, 492 aa, synthetic construct
  REGION 1..492: note = CAR-H18
MALPVTALLLPLALLLHAARPQSALTQPPSASGSPGQSVTISCTGTSSDVGGYTYVSWYQQHPGKAPKLMIYEVSKRPSGVPDRFSGSKSGNTASLTVSGLQAEDEADYYCSSYAGSNNVVFGGGTELTVLGGGGSGGGGSGGGGSEVQLVESGGGLVQPGGSLRLSCAASGFTFSSYAMSWVRQAPGKGLEWFSAISGSGGSTYYADSVKGRFTISRDNSKNTLYLQMNSLRAEDTAVYYCAKDPDPGYFDWVAGDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 190 - protein, 489 aa, synthetic construct
  REGION 1..489: note = CAR-H5
MALPVTALLLPLALLLHAARPDIRLTQSPSTVSASIGDRVTITCRASQGISNWLTWYQQKPGKAPKLLIYSASTLQSGVPSRFSGSGSGTDFTLTISSLQPEDFATYYCQQANNLRTLTFGGGTKVDIKGGGGSGGGGSGGGGSQVQLVQSGAEVKKPGESLRISCKGSGYSFTSYWISWVRQMPGKGLEWMGRIDPSDSYTNYSPSFQGHVTISADKSISTAYLQWSSLKASDTAMYYCARHPVTNYYYYGMDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - protein, 486 aa, synthetic construct
  REGION 1..486: note = CAR-H7
MALPVTALLLPLALLLHAARPDIVMTQSPSSLSASVGDRVTITCRASQSISSYLNWYQQKPGKAPKLLIYAASSLQSGVPSRFSGSGSGTDFTLTISSLQPEDFATYYCQQSYSTLPYTFGQGTKLEIKGGGGSGGGGSGGGGSEVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCATDYGELHDAFDIWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 192 - protein, 488 aa, synthetic construct
  REGION 1..488: note = CAR-H15
MALPVTALLLPLALLLHAARPAIQLTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKVEIKGGGGSGGGGSGGGGSEVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATERASGSYEYYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 193 - DNA, 732 bp, synthetic construct
  misc_feature 1..732: note = H1 scFv nucleotide sequence
  source 1..732: mol_type = other DNA
cagcctgtgctgactcagtcaccgtcagcgtctgagacccccgggcagagggtcaccatctcttgttctgggagcacctccaacaccggaaacaattatttttcttggtaccaacaagtcccaggaatggcccccaaactcctcatttataagaacaatctgcggccctcaggggtccctgaccgattctctggctccaagtctggcacctcagcctccctgtccatcagtggactccggtccgaggatgagggtgattattattgtgcaacatgggatgacaacttgactctggtgttcggcggagggaccaagctgaccgtcctaggtggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggcctgggaaccctggtcactgtctcctca

SEQ ID NO: 194 - DNA, 732 bp, synthetic construct
  misc_feature 1..732: note = H4 scFv nucleotide sequence
  source 1..732: mol_type = other DNA
cagtctgtggtgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctggtgattgggtgttcggcggagggaccaagctgaccgtcctaggtggtggtggttctggcggcggcggctccggaggtggtggatcccaggtgcagctggtgcagtctgggggaggcttggtccagcctggggggtccctgagactctcctgtgcagcctctggattcacctttagtacctattggatgagctgggtccgccaggctccagggaagggactggagtgggtggccaacataaaccaagatggaactgagaaatactatgtggactctgtgaagggtcgattcaccatctccagagacaacgccaagaactcactgtatctgcaaatgaacagcctgagagccgaggacacggctgtatattactgtgcgagagagccgggtatagcacttgattactggggccagggaaccctggtcactgtctcctca

SEQ ID NO: 195 - DNA, 744 bp, synthetic construct
  misc_feature 1..744: note = H6 scFv nucleotide sequence
  source 1..744: mol_type = other DNA
caatctgccctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcaggaagcggccctcaggggtccctgatcgcttctctggctccaagtctggcaacacggcctccctgaccgtctctgggctccaggctgacgatgaggctgattattactgcagctcatatgcaggcagcaacaatttgggattcggcggagggaccaagctgaccgtcctaggtggtggtggttctggcggcggcggctccggaggtggtggatccgaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatcccgatccagggtattttgactgggtagcgggggactactggggccagggaaccctggtcaccgtctcctca

SEQ ID NO: 196 - DNA, 732 bp, synthetic construct
  misc_feature 1..732: note = H8 scFv nucleotide sequence
  source 1..732: mol_type = other DNA
cagtctgtgttgacgcagccaccctcggtgtctgaagcccccaggcagagggtcaccatctcctgttctggaagcagctccaacatcggaaataatgctgtaaactggtaccagcagctcccaggaaaggctcccaaactcctcatctattatgatgatctgctgccctcaggggtctctgaccgattctctggctccaagtctggcacctcagcctccctgaccatcagcggcctccagtctgaggatggggctgattattactgtgcatcatgggatgacagccttaatggtcgggtgttcggcggggggaccaagctgaccgtcctaggtggtggtggttctggcggcggcggctccggaggtggtggatcccaggtgcagctgcaggagtcgggcgcaggactgttgaagccttcggagaccctgtccctcacctgcgctgtctatggtgggtccatcagtggctactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggcggcggtagtagtggttattacttggactactggggccagggagccctggtcactgtctcctca

SEQ ID NO: 197 - DNA, 741 bp, synthetic construct
  misc_feature 1..741: note = H9 scFv nucleotide sequence
  source 1..741: mol_type = other DNA
cagtctgtgttgacgcagccgccctcagcgtctggggcccccggggagagggtcaccatctcttgttctggaagcagctccaacatcggaagtaattatgtatactggtaccagcagctcccaggaacggcccccaaactcctcatctataacagtaatcagcggccctcaggggtccctgaccgattctctggctccaagtctggcgcctcagcctccctggccatcagtgggctccggtccgaggatgaggctgattattactgtgcagcatgggatgacagcctgagtggtccggtattcggcggagggaccaaggtcaccgtcctaggtggtggtggttctggcggcggcggctccggaggtggtggatccgaggtgcagctggtggagtccggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaggggttggtctatggttcggggagccttactttgactactggggccagggaaccctggtcactgtctcctca

SEQ ID NO: 198 - DNA, 738 bp, synthetic construct
  misc_feature 1..738: note = H10 scFv nucleotide sequence
  source 1..738: mol_type = other DNA
cagtctgccctgactcagcctgcctccgtgtctggatctcctggacagtcgatcaccatttcctgcactggaaccagcagtgacgttggtggttctaagtatgtctcctggtaccaacaacacccaggcaaagcccccaaactcatgatttatgatgtcagttatcggccctcaggggtttctgatcgcttctctggctccaagtctggcaacacggcctccctgaccatctctgggctccaggctgaggacgaggctgattattactgcagctcatatacaagcaacagcaatctgccgctgttcggcggagggaccaaggtcaccgtcctaggtggtggtggttctggcggcggcggctccggaggtggtggatcccagctgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggccagggaaccctggtcaccgtctcctca

SEQ ID NO: 199 - DNA, 735 bp, synthetic construct
  misc_feature 1..735: note = H12 scFv nucleotide sequence
  source 1..735: mol_type = other DNA
cagccagtgctgactcagccaccctcagcgtctgggacccccgggcagagggtcatcatctcttgttctggaagcggctccaatatcggaagtaacactgtaacttggtaccggcaattcccaggagcggcccccaaaaccctcatttacaatagtaagctgcggccctcctgggtccctgaccgattcactgcctccaagtctggcacctcagcctccctggccatcagtgggctccagtctgaggatgaggctgattattactgtgcagcatgggatgacagcctgaatggtgtggtattcggcggagggaccaagctgaccgtcctaggtggtggtggttctggcggcggcggctccggaggtggtggatccgaggtgcatctggtggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggccagggaaccctggtcactgtctcctca

SEQ ID NO: 200 - DNA, 774 bp, synthetic construct
  misc_feature 1..774: note = H13 scFv nucleotide sequence
  source 1..774: mol_type = other DNA
cagtctgtggtgactcagccaccctcagcgtctgggacccccgggcagagggtcaccatctcttgttctggaagcagctccaacatcggaagtaattctgtaaactggtaccagcaactcccaggaacggcccccaaactcctcatctataataataatcagcggccctcaggggtccctgaccgattctctggctccaagtctggcacctcagcctccctggccatcagtgggctccagtctgaggatgaggctgattattactgtgcgacatgggatgacagcctgaatggtaatgtggtgttcggcggagggaccaaggtgaccgtcctcggtggtggtggttctggcggcggcggctccggaggtggtggatcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagatcagggcgtagtaccagctgctgaaatcccgtattacgatttttggagtggacccccgttcgacccctggggccagggaaccctggtcactgtctcctca

SEQ ID NO: 201 - DNA, 732 bp, synthetic construct
  misc_feature 1..732: note = H17 scFv nucleotide sequence
  source 1..732: mol_type = other DNA
caatctgccctgactcagcctccctccgtgtccgggtctcctggacagtcagtctccatctcctgcactggatccagcagtgacattggtgcttcttctcgtgtctcctggtacctacagcccccagggaaagccccccaactcataatttttggagtcaataatcgaccctcaggggtccctgatcgcttctctggatccaagtctggcaacacggcctccctgaccatctctgggctccgggctgaggacgaggctgactattactgcagctcacattccagcggaaacactttgttattcggcggagggaccaagctgaccgtcctaggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggccagggaaccctggtcaccgtctcctca

SEQ ID NO: 202 - DNA, 744 bp, synthetic construct
  misc_feature 1..744: note = H18 scFv nucleotide sequence
  source 1..744: mol_type = other DNA
cagtctgccctgactcagcctccctccgcgtccgggtctcctggacagtcagtcaccatctcctgcactggaaccagcagtgacgttggtggttatacttatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaacaatgtggtattcggcggagggaccgagctgaccgtcctaggtggtggtggttctggcggcggcggctccggaggtggtggatccgaggtgcaactggtggagtctgggggaggcttggtacagcctggggggtccctgagactctcctgtgcagcctctggattcacctttagcagctatgccatgagctgggtccgccaggctccagggaaggggctggagtggttctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatcccgatccagggtattttgactgggtagcgggggactactggggccagggaaccctggtcaccgtctcctca

SEQ ID NO: 203 - DNA, 735 bp, synthetic construct
  misc_feature 1..735: note = H5 scFv nucleotide sequence
  source 1..735: mol_type = other DNA
gacatccggttgacccagtctccatctaccgtgtctgcctctataggagacagagtcaccatcacttgtcgggcgagtcaggggattagtaactggttgacctggtatcagcagaaaccagggaaagcccctaagctcctgatctattctgcatccactttgcagagtggggtcccatcaaggttcagcggcagtgggtctgggacagacttcactctcaccatcagcagcctgcagcctgaagattttgcaacttactattgtcaacaggcgaacaatttgcgaacgctcactttcggcggtgggaccaaagtggatatcaaaggtggtggtggttctggcggcggcggctccggaggtggtggatcccaggtccagctggtgcagtctggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgagacatcccgtaacaaactactactactacggtatggacgtctggggccaagggaccacggtcaccgtctcctca

SEQ ID NO: 204 - DNA, 726 bp, synthetic construct
  misc_feature 1..726: note = H7 scFv nucleotide sequence
  source 1..726: mol_type = other DNA
gatattgtgatgactcagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccctcccgtacacttttggccaggggaccaagctggagatcaaaggtggtggtggttctggcggcggcggctccggaggtggtggatccgaagtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgaccgactacggtgagctccatgatgcttttgatatctggggccaagggaccacggtcaccgtctcctca

SEQ ID NO: 205 - DNA, 732 bp, synthetic construct
  misc_feature 1..732: note = H15 scFv nucleotide sequence
  source 1..732: mol_type = other DNA
gccatccagttgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccgtacacttttggccaggggaccaaggtggagatcaaaggtggtggtggttctggcggcggcggctccggaggtggtggatccgaggtgcagctggtgcagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagagagggcgagtgggagctacgaatactactttgactactggggccagggaaccctggtcaccgtctcctca

SEQ ID NO: 206 - DNA, 63 bp, synthetic construct
  misc_feature 1..63: note = CD8 signal peptide nucleotide sequence
  source 1..63: mol_type = other DNA
atggccttaccagtgaccgccttgctcctgccgctggccttgctgctccacgccgccaggccg

SEQ ID NO: 207 - DNA, 135 bp, synthetic construct
  misc_feature 1..135: note = CD8 Hinge nucleotide sequence
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 208 - DNA, 72 bp, synthetic construct
  misc_feature 1..72: note = CD8 transmembrane domain nucleotide sequence
  source 1..72: mol_type = other DNA
atctacatctgggcgcccttggccgggacttgtggggtccttctcctgtcactggttatcaccctttactgc

SEQ ID NO: 209 - DNA, 126 bp, synthetic construct
  misc_feature 1..126: note = 4-1BB costimulatory domain nucleotide sequence
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 210 - DNA, 336 bp, synthetic construct
  misc_feature 1..336: note = CD3-zeta nucleotide sequence
  source 1..336: mol_type = other DNA
agagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 211 - DNA, 20 bp, synthetic construct
  misc_feature 1..20: note = CD7gRNA-R2
  source 1..20: mol_type = other DNA
ggaacatggccgggcctccg

SEQ ID NO: 212 - DNA, 20 bp, synthetic construct
  misc_feature 1..20: note = CD7gRNA-R5
  source 1..20: mol_type = other DNA
gcctgggaagctcttacctt

SEQ ID NO: 213 - DNA, 20 bp, synthetic construct
  misc_feature 1..20: note = CD7gRNA-R6
  source 1..20: mol_type = other DNA
gggatctacctgaggcagct

SEQ ID NO: 214 - DNA, 20 bp, synthetic construct
  misc_feature 1..20: note = CD7gRNA-R8
  source 1..20: mol_type = other DNA
gacatcatttactacgagga

SEQ ID NO: 215 - DNA, 20 bp, synthetic construct
  misc_feature 1..20: note = CD7gRNA-R9
  source 1..20: mol_type = other DNA
gcctgcgtgggatctacctg

SEQ ID NO: 216 - DNA, 20 bp, synthetic construct
  misc_feature 1..20: note = CD7gRNA-R10
  source 1..20: mol_type = other DNA
ggacggggtggtgcccacta

SEQ ID NO: 217 - DNA, 20 bp, synthetic construct
  misc_feature 1..20: note = CD7gRNA-R11
  source 1..20: mol_type = other DNA
gggaccctgagaagtcgatg

SEQ ID NO: 218 - DNA, 20 bp, synthetic construct
  misc_feature 1..20: note = CD7gRNA-R12
  source 1..20: mol_type = other DNA
ggtgatagtcaggttgtcct

SEQ ID NO: 219 - DNA, 22 bp, synthetic construct
  misc_feature 1..22: note = Forward primer sequence 1
  source 1..22: mol_type = other DNA
tgcagctggcacgacaggtttc

SEQ ID NO: 220 - DNA, 19 bp, synthetic construct
  misc_feature 1..19: note = reverse primer sequence 1
  source 1..19: mol_type = other DNA
cgtcagactgtagcacgtt

SEQ ID NO: 221 - DNA, 20 bp, synthetic construct
  misc_feature 1..20: note = Forward primer sequence 2
  source 1..20: mol_type = other DNA
aacaattgaattcaggagga

SEQ ID NO: 222 - DNA, 19 bp, synthetic construct
  misc_feature 1..19: note = reverse primer sequence 2
  source 1..19: mol_type = other DNA
cctcctaagaagcgtagtc

SEQ ID NO: 223 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 224 - protein, 215 aa, synthetic construct
  REGION 1..215: note = human CD40L extracellular domain
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 225 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 226 - protein, 41 aa, synthetic construct
  REGION 1..41: note = CD28 cytoplasmic domain
RSKRSRLLHSDYMNMTPRRPGPTRKHYQPYAPPRDFAAYRS

SEQ ID NO: 227 - protein, 249 aa, synthetic construct
  REGION 1..249: note = F2.103
EVQLVESGGGLVQPGGSLRLSCAVSGFTFSTYWMHWVRQAPGKGLVWVSRINSDGSSTTYADSVKGRFTISRDNAKNTLYLQMNSLRAEDTAVYYCARDRVLWIGELSYYGMDVWGQGTTVTVSSGGGGSGGGGSGGGGSDIQMTQSPSTLSASVGDRVTITCRASQSISNWLAWYQQKPGKAPKLLLYKASGLESGVPSRFSGSGSGTEFTLTINSLQPDDFATYYCQQSNSYSWTFGHGTKVEIKRT

SEQ ID NO: 228 - protein, 261 aa, synthetic construct
  REGION 1..261: note = F5.157
EVQLLESGGGLVQPGGSLRLSCAASGFAFSSYAMSWVRQAPGKGLEWVSAISGSGGSTYYADSVKGRFTISRDNSKNTLYLQMNSLRPRTRPYITVRKMGGTMVRGVMGTLTTGAREPWSPSPQGGGGSGGGGSGGGGSIQMTQSPSSVSASAGDRVTITCRASQGISSWLAWYQQKPGKAPKLLIYAGSSLQSGVPSRFSGSGFGTDFTLTIGSLQPEDFATYYCQQASSFPRTFGQGTKVEIKRTVLHHLSSSSRHLMS

SEQ ID NO: 229 - protein, 248 aa, synthetic construct
  REGION 1..248: note = F5.77
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYGSGSYGYFDYWGQGTLVTVSSGGGGSGGGGSGGGGSDIQMTQSPSSVSGSVGDRVTITCRASQGISSWLAWYQQKPGKAPKLLIYAGSSLQSGVPSRFSGSGFGTDFTLTISSLQPEDFATYYCQQASSFPRTFGQGTKVEIKRT

SEQ ID NO: 230 - protein, 246 aa, synthetic construct
  REGION 1..246: note = 4D11
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASGGGGSGGGGSGGGGSAIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRT

SEQ ID NO: 231 - protein, 241 aa, synthetic construct
  REGION 1..241: note = A40C
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSGGGGSGGGGSGGGGSDIQMTQSPSSLSASVGDRVTITCSASSSVSYMLWFQQKPGKAPKLLIYSTSNLASGVPSRFSGSGSGTDFTLTISSLQPEDFATYYCQQRTFYPYTFGGGTKVEIKRT

SEQ ID NO: 232 - protein, 249 aa, synthetic construct
  REGION 1..249: note = 119
QVQLQQSGPGLVKPSQTLSLTCAISGDSVSSNSATWNWIRQSPSRDLEWLGRTYYRSKWYRDYVGSVKSRIIINPDTSNNQFSLQLNSVTPEDTAIYYCTRAQWLGGDYPYYYSMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNTFGPGTKVDIKRT

SEQ ID NO: 233 - protein, 266 aa, synthetic construct
  REGION 1..266: note = 1412
ALPVTALLLPLALLLHAARPQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSVEGGSGGSGGSGGSGGVMDDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 234 - protein, 302 aa, synthetic construct
  REGION 1..302: note = 40L.28.40L.40L
MIETYNQTSPRSAATGLPISMKRSKRSRLLHSDYMNMTPRRPGPTRKHYQPYAPPRDFAAYRSIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 235 - protein, 574 aa, synthetic construct
  REGION 1..574: note = TriCD40L_8-28
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGGGGSGGGGSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRS

SEQ ID NO: 236 - protein, 571 aa, synthetic construct
  REGION 1..571: note = TriCD40L_28-28
NPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGSNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGGGGSGGGGSGGGGSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 237 - protein, 419 aa, synthetic construct
  REGION 1..419: note = 1412-T4-CD40L
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSVEGGSGGSGGSGGSGGVMDDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGYIPEAPRDGQAYVRKDGEWVLLSTFLGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 238 - protein, 500 aa, synthetic construct
  REGION 1..500: note = 1412-F2.103
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSVEGGSGGSGGSGGSGGVMDDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEVQLVESGGGLVQPGGSLRLSCAVSGFTFSTYWMHWVRQAPGKGLVWVSRINSDGSSTTYADSVKGRFTISRDNAKNTLYLQMNSLRAEDTAVYYCARDRVLWIGELSYYGMDVWGQGTTVTVSSGGGGSGGGGSGGGGSDIQMTQSPSTLSASVGDRVTITCRASQSISNWLAWYQQKPGKAPKLLLYKASGLESGVPSRFSGSGSGTEFTLTINSLQPDDFATYYCQQSNSYSWTFGHGTKVEIKRT

SEQ ID NO: 239 - protein, 512 aa, synthetic construct
  REGION 1..512: note = 1412-F5.157
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSVEGGSGGSGGSGGSGGVMDDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEVQLLESGGGLVQPGGSLRLSCAASGFAFSSYAMSWVRQAPGKGLEWVSAISGSGGSTYYADSVKGRFTISRDNSKNTLYLQMNSLRPRTRPYITVRKMGGTMVRGVMGTLTTGAREPWSPSPQGGGGSGGGGSGGGGSIQMTQSPSSVSASAGDRVTITCRASQGISSWLAWYQQKPGKAPKLLIYAGSSLQSGVPSRFSGSGFGTDFTLTIGSLQPEDFATYYCQQASSFPRTFGQGTKVEIKRTVLHHLSSSSRHLMS

SEQ ID NO: 240 - protein, 499 aa, synthetic construct
  REGION 1..499: note = 1412-F5.77
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSVEGGSGGSGGSGGSGGVMDDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYGSGSYGYFDYWGQGTLVTVSSGGGGSGGGGSGGGGSDIQMTQSPSSVSGSVGDRVTITCRASQGISSWLAWYQQKPGKAPKLLIYAGSSLQSGVPSRFSGSGFGTDFTLTISSLQPEDFATYYCQQASSFPRTFGQGTKVEIKRT

SEQ ID NO: 241 - protein, 498 aa, synthetic construct
  REGION 1..498: note = 1412-4D11
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSVEGGSGGSGGSGGSGGVMDDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKGGGGSPQLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASGGGGSGGGGSGGGGSAIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRT

SEQ ID NO: 242 - protein, 364 aa, synthetic construct
  REGION 1..364: note = F2.103.CD28
EVQLVESGGGLVQPGGSLRLSCAVSGFTFSTYWMHWVRQAPGKGLVWVSRINSDGSSTTYADSVKGRFTISRDNAKNTLYLQMNSLRAEDTAVYYCARDRVLWIGELSYYGMDVWGQGTTVTVSSGGGGSGGGGSGGGGSDIQMTQSPSTLSASVGDRVTITCRASQSISNWLAWYQQKPGKAPKLLLYKASGLESGVPSRFSGSGSGTEFTLTINSLQPDDFATYYCQQSNSYSWTFGHGTKVEIKRTASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRS

SEQ ID NO: 243 - protein, 376 aa, synthetic construct
  REGION 1..376: note = F5.157.CD28
EVQLLESGGGLVQPGGSLRLSCAASGFAFSSYAMSWVRQAPGKGLEWVSAISGSGGSTYYADSVKGRFTISRDNSKNTLYLQMNSLRPRTRPYITVRKMGGTMVRGVMGTLTTGAREPWSPSPQGGGGSGGGGSGGGGSIQMTQSPSSVSASAGDRVTITCRASQGISSWLAWYQQKPGKAPKLLIYAGSSLQSGVPSRFSGSGFGTDFTLTIGSLQPEDFATYYCQQASSFPRTFGQGTKVEIKRTVLHHLSSSSRHLMSASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRS

SEQ ID NO: 244 - protein, 363 aa, synthetic construct
  REGION 1..363: note = F5.77.CD28:
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYGSGSYGYFDYWGQGTLVTVSSGGGGSGGGGSGGGGSDIQMTQSPSSVSGSVGDRVTITCRASQGISSWLAWYQQKPGKAPKLLIYAGSSLQSGVPSRFSGSGFGTDFTLTISSLQPEDFATYYCQQASSFPRTFGQGTKVEIKRTASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRS

SEQ ID NO: 245 - protein, 358 aa, synthetic construct
  REGION 1..358: note = F2.103.BB
EVQLVESGGGLVQPGGSLRLSCAVSGFTFSTYWMHWVRQAPGKGLVWVSRINSDGSSTTYADSVKGRFTISRDNAKNTLYLQMNSLRAEDTAVYYCARDRVLWIGELSYYGMDVWGQGTTVTVSSGGGGSGGGGSGGGGSDIQMTQSPSTLSASVGDRVTITCRASQSISNWLAWYQQKPGKAPKLLLYKASGLESGVPSRFSGSGSGTEFTLTINSLQPDDFATYYCQQSNSYSWTFGHGTKVEIKRTTTTPAPRPPTPAPTIASQPLSLRPEACRPAAGGAVHTRGLDFACDIYIWAPLAGTCGVLLLSLVITLYCRGRKKLLYIFKQPFMRPVQTTQEEDGCSCRFPEEEEGGCE

SEQ ID NO: 246 - protein, 370 aa, synthetic construct
  REGION 1..370: note = F5.157.BB
EVQLLESGGGLVQPGGSLRLSCAASGFAFSSYAMSWVRQAPGKGLEWVSAISGSGGSTYYADSVKGRFTISRDNSKNTLYLQMNSLRPRTRPYITVRKMGGTMVRGVMGTLTTGAREPWSPSPQGGGGSGGGGSGGGGSIQMTQSPSSVSASAGDRVTITCRASQGISSWLAWYQQKPGKAPKLLIYAGSSLQSGVPSRFSGSGFGTDFTLTIGSLQPEDFATYYCQQASSFPRTFGQGTKVEIKRTVLHHLSSSSRHLMSTTTPAPRPPTPAPTIASQPLSLRPEACRPAAGGAVHTRGLDFACDIYIWAPLAGTCGVLLLSLVITLYCRGRKKLLYIFKQPFMRPVQTTQEEDGCSCRFPEEEEGGCE

SEQ ID NO: 247 - protein, 357 aa, synthetic construct
  REGION 1..357: note = F5.77.BB
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYGSGSYGYFDYWGQGTLVTVSSGGGGSGGGGSGGGGSDIQMTQSPSSVSGSVGDRVTITCRASQGISSWLAWYQQKPGKAPKLLIYAGSSLQSGVPSRFSGSGFGTDFTLTISSLQPEDFATYYCQQASSFPRTFGQGTKVEIKRTTTTPAPRPPTPAPTIASQPLSLRPEACRPAAGGAVHTRGLDFACDIYIWAPLAGTCGVLLLSLVITLYCRGRKKLLYIFKQPFMRPVQTTQEEDGCSCRFPEEEEGGCE

SEQ ID NO: 248 - protein, 361 aa, synthetic construct
  REGION 1..361: note = 4D11.CD28
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASGGGGSGGGGSGGGGSAIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRTASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRS

SEQ ID NO: 249 - protein, 356 aa, synthetic construct
  REGION 1..356: note = A40C.CD28
QVQLVQSGAEVKKPGASVKVSCTASGFNIKDYYVHWVKQAPGQGLEWMGRIDPEDGDSKYAPKFQGKATMTADTSTSTVYMELSSLRSEDTAVYYCTTSYYVGTYGYWGQGTLVTVSSGGGGSGGGGSGGGGSDIQMTQSPSSLSASVGDRVTITCSASSSVSYMLWFQQKPGKAPKLLIYSTSNLASGVPSRFSGSGSGTDFTLTISSLQPEDFATYYCQQRTFYPYTFGGGTKVEIKRTASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRS

SEQ ID NO: 250 - protein, 364 aa, synthetic construct
  REGION 1..364: note = 119.CD28
QVQLQQSGPGLVKPSQTLSLTCAISGDSVSSNSATWNWIRQSPSRDLEWLGRTYYRSKWYRDYVGSVKSRIIINPDTSNNQFSLQLNSVTPEDTAIYYCTRAQWLGGDYPYYYSMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNTFGPGTKVDIKRTASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRS

SEQ ID NO: 251 - protein, 1045 aa, synthetic construct
  REGION 1..1045: note = A40C.CD28 nucleotide sequence
CAGGTCCAGCTGGTACAATCTGGTGCTGAAGTGAAGAAACCAGGAGCGTCGGTGAAGGTCTCATGCACAGCCTCAGGCTTCAACATCAAGGACTACTACGTGCACTGGGTCAAGCAAGCTCCCGGACAGGGGCTGGAGTGGATGGGGAGAATTGACCCGGAAGACGGCGACTCGAAATACGCGCCGAAGTTCCAGGGCAAGGCCACCATGACAGCCGATACCAGTACATCGACCGTTTACATGGAGCTGTCCAGCCTCCGCTCAGAGGATACCGCGGTATACTACTGTACGACCTCCTACTACGTCGGAACGTACGGTTACTGGGGCCAGGGTACCCTGGTGACAGTGTCCTCGGGGGGCGGCGGGTCTGGCGGGGGAGGCAGCGGGGGCGGCGGGTCGGACATCCAGATGACTCAATCCCCCTCTTCCCTGAGTGCGTCCGTGGGTGACAGGGTGACGATCACTTGTAGCGCTTCGTCCTCGGTGTCGTATATGCTGTGGTTCCAGCAGAAGCCCGGTAAGGCCCCCAAACTGCTCATTTATTCCACCAGCAACCTCGCCAGTGGCGTACCCAGTCGCTTCTCCGGTAGTGGCTCCGGTACCGATTTCACACTGACCATCTCCAGTCTGCAACCTGAAGACTTCGCAACGTACTATTGTCAGCAGCGCACATTCTATCCCTATACGTTTGGGGGCGGGACCAAGGTCGAGATCAAGCGCACAGCTAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATTTTTGGGTGCTGGTGGTGGTTGGTGGAGTCCTGGCTTGCTATAGCTTGCTAGTAACAGTGGCCTTTATTATTTTCTGGGTGAGGAGTAAGAGGAGCAGGCTCCTGCACAGTGACTACATGAACATGACTCCCCGCCGCCCCGGGCCCACCCGCAAGCATTACCAGCCCTATGCCCCACCAC

SEQ ID NO: 252 - protein, 17 aa, synthetic construct
  REGION 1..17: note = linker 1
GSGGGGSGGGGSGGGGS

SEQ ID NO: 253 - protein, 5 aa, synthetic construct
  REGION 1..5: note = linker 2
GGGGS

SEQ ID NO: 254 - DNA, 1467 bp, synthetic construct
  misc_feature 1..1467: note = CD7 CAR-H1 DNA
  source 1..1467: mol_type = other DNA
atggccttaccagtgaccgccttgctcctgccgctggccttgctgctccacgccgccaggccgcagcctgtgctgactcagtcaccgtcagcgtctgagacccccgggcagagggtcaccatctcttgttctgggagcacctccaacaccggaaacaattatttttcttggtaccaacaagtcccaggaatggcccccaaactcctcatttataagaacaatctgcggccctcaggggtccctgaccgattctctggctccaagtctggcacctcagcctccctgtccatcagtggactccggtccgaggatgagggtgattattattgtgcaacatgggatgacaacttgactctggtgttcggcggagggaccaagctgaccgtcctaggtggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggcctgggaaccctggtcactgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 255 - DNA, 1932 bp, synthetic construct
  misc_feature 1..1932: note = CD7 CAR-H1 linear mRNA
  source 1..1932: mol_type = other DNA
taatacgactcactataggatggccttaccagtgaccgccttgctcctgccgctggccttgctgctccacgccgccaggccgcagcctgtgctgactcagtcaccgtcagcgtctgagacccccgggcagagggtcaccatctcttgttctgggagcacctccaacaccggaaacaattatttttcttggtaccaacaagtcccaggaatggcccccaaactcctcatttataagaacaatctgcggccctcaggggtccctgaccgattctctggctccaagtctggcacctcagcctccctgtccatcagtggactccggtccgaggatgagggtgattattattgtgcaacatgggatgacaacttgactctggtgttcggcggagggaccaagctgaccgtcctaggtggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggcctgggaaccctggtcactgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgacagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcctcgacgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 256 - DNA, 2735 bp, synthetic construct
  misc_feature 1..2735: note = CD7 CAR-H1 circRNA
  source 1..2735: mol_type = other DNA
taatacgactcactataggggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaatctagagccaccatggccttaccagtgaccgccttgctcctgccgctggccttgctgctccacgccgccaggccgcagcctgtgctgactcagtcaccgtcagcgtctgagacccccgggcagagggtcaccatctcttgttctgggagcacctccaacaccggaaacaattatttttcttggtaccaacaagtcccaggaatggcccccaaactcctcatttataagaacaatctgcggccctcaggggtccctgaccgattctctggctccaagtctggcacctcagcctccctgtccatcagtggactccggtccgaggatgagggtgattattattgtgcaacatgggatgacaacttgactctggtgttcggcggagggaccaagctgaccgtcctaggtggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggcctgggaaccctggtcactgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgacaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 257 - DNA, 2799 bp, synthetic construct
  misc_feature 1..2799: note = CD7 CAR-H1 circRNA-70A
  source 1..2799: mol_type = other DNA
taatacgactcactataggggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaatctagagccaccatggccttaccagtgaccgccttgctcctgccgctggccttgctgctccacgccgccaggccgcagcctgtgctgactcagtcaccgtcagcgtctgagacccccgggcagagggtcaccatctcttgttctgggagcacctccaacaccggaaacaattatttttcttggtaccaacaagtcccaggaatggcccccaaactcctcatttataagaacaatctgcggccctcaggggtccctgaccgattctctggctccaagtctggcacctcagcctccctgtccatcagtggactccggtccgaggatgagggtgattattattgtgcaacatgggatgacaacttgactctggtgttcggcggagggaccaagctgaccgtcctaggtggtggtggttctggcggcggcggctccggaggtggtggatcccaggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagggtacagtggctaccccgcagtgcttgactactggggcctgggaaccctggtcactgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgacgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtcgacaaaaaaaaaaaaaaaaaaaaaaaaaaaaaaaaaaaaaaaaaaaaaaaaaaaaaaaaaaaaaaaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 258 - DNA, 18 bp, synthetic construct
  misc_feature 1..18: note = T7 Promotor
  source 1..18: mol_type = other DNA
taatacgactcactatag

SEQ ID NO: 259 - DNA, 33 bp, synthetic construct
  misc_feature 1..33: note = Homology arm-1
  source 1..33: mol_type = other DNA
gggagaccctcgaccgtcgattgtccactggtc

SEQ ID NO: 260 - DNA, 182 bp, synthetic construct
  misc_feature 1..182: note = Anabaena intron-1
  source 1..182: mol_type = other DNA
aacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccaccccca

SEQ ID NO: 261 - DNA, 40 bp, synthetic construct
  misc_feature 1..40: note = Spacer-1
  source 1..40: mol_type = other DNA
cgccggaaacgcaatagccgaaaaacaaaaaacaaaaaaa

SEQ ID NO: 262 - DNA, 741 bp, synthetic construct
  misc_feature 1..741: note = CVB3 IRES
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 263 - DNA, 40 bp, synthetic construct
  misc_feature 1..40: note = Spacer-2
  source 1..40: mol_type = other DNA
aaaaaacaaaaaacaaaacggctattatgcgttaccggcg

SEQ ID NO: 264 - DNA, 131 bp, synthetic construct
  misc_feature 1..131: note = Anabaena intron-2
  source 1..131: mol_type = other DNA
agacgctacggacttaaataattgagccttaaagaagaaattctttaagtggatgctctcaaactcagggaaacctaaatctagttatagacaaggcaatcctgagccaagccgaagtagtaattagtaag

SEQ ID NO: 265 - DNA, 35 bp, synthetic construct
  misc_feature 1..35: note = Homology arm-2
  source 1..35: mol_type = other DNA
accagtggacaatcgacggataacagcatatctag

SEQ ID NO: 266 - DNA, 70 bp, synthetic construct
  misc_feature 1..70: note = 70A
  source 1..70: mol_type = other DNA
aaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0161358 A1
(43) Pub. Date: May 22, 2025

(54) MODIFIED IMMUNE EFFECTOR CELLS AND USES THEREOF

(71) Applicant: UTC THERAPEUTICS (SHANGHAI) CO., LTD., Shanghai (CN)

(72) Inventors: Yangbing Zhao, Shanghai (CN); Gengzhen Zhu, Shanghai (CN); Xiaojun Liu, Shanghai (CN)

(21) Appl. No.: 18/839,058

(22) PCT Filed: Feb. 16, 2023
(86) PCT No.: PCT/CN2023/076567

§ 371 Date: Aug. 16, 2024

(30) Foreign Application Priority Data
Feb. 17, 2022 (WO) PCT/CN2022/076628
Feb. 17, 2022 (WO) PCT/CN2022/076634
Feb. 17, 2022 (WO) PCT/CN2022/076635

(51) Int. Cl. A61K 35/17; A61K 38/00; A61K 40/11; A61K 40/31; C07K 16/28; C12N 5/0783; C12N 9/22; C12N 15/11; C12N 15/85

(52) U.S. Cl. CPC A61K 35/17; A61K 40/11; A61K 40/31; C07K 16/2803; C12N 5/0636; C12N 9/22; C12N 15/111; C12N 15/85; A61K 38/00; A61K 2239/13; A61K 2239/48; C07K 2317/565; C07K 2317/622; C12N 2310/20; C12N 2510/00

(57) ABSTRACT

The present application relates to a modified immune effector cell comprising a nucleic acid molecule encoding a fusion protein, the fusion protein comprising a first domain for activating an antigen presenting cell (APC) and a second domain for activating the immune effector cell, and further comprising an isolated nucleic acid molecule for inhibiting the expression of the CD7 gene within the cell. The present application additionally relates to the use of the nucleic acid molecule in the preparation of an anti-CD7 CAR-T cell. The CD7 expressed by the anti-CD7 CAR-T cells of the nucleic acid molecules introduced in the present application is downregulated without affecting CAR expression and while preventing CAR-T cells from killing each other.

### TECHNICAL FIELD

The present application relates to the field of biomedicine, and specifically to a modified immune effector cell and its use.

### BACKGROUND

T lymphocytes can be engineered to express T cell receptors (TCRs)^{[1-3]} or chimeric antigen receptors (CARs)^{[4, 5]} that recognize tumor antigens in order to kill tumors for the treatment of cancer and other diseases. Although T lymphocytes engineered with CAR specific for B cell markers such as CD19 have shown dramatic clinical responses in hematological malignancies, effective immunotherapy in solid tumors has proven challenging, primarily due to immune escape caused by the complex, dynamic tumor microenvironment (TME), which induces T cell dysfunction and exhaustion and limits antitumor immune responses^{[6]}. Strategies to engineer T cells to circumvent the inhibitory effects of the TME offer the opportunity to circumvent inhibitory pathways without causing systemic toxicity, such as the high adverse reaction rates seen with combination therapy using PD-1 and CTLA-4 checkpoint inhibitors^{[7]}.

Raised T cell activation thresholds and reduced T cell activation states are the main mechanisms of most tumor-associated inhibitory effects on T cells, which can be offset by providing adequate co-stimulatory stimulation^{[8]}. CD28 is considered the most prominent co-stimulatory molecule for optimal T cell clonal expansion, differentiation, and effector function. Engagement of CD28 reduces the activation threshold of T cells and leads to enhanced TCR signaling events, which are necessary for effective cytokine production (through enhanced transcriptional activity and messenger RNA stabilization), cell cycle progression, survival, metabolic regulation, and T cell responses. This is because CD28 is a key role in the organization of the immunological synapse (IS), which strengthens the close contact between T cells and APCs.

Studies have shown that co-stimulatory receptors or their ligands can be genetically introduced into T cells to enhance their effector function, persistence, and antitumor activity^{[8-10 ]}. However, T cells expressing co-stimulatory receptors or ligands are expected to be insufficiently functional in neoplastic lesions due to the poor expression of receptors and ligands and the overexpression of inhibitory ligands (such as PD-L1) in the TME^{[11]}. In previous studies, we and others have found that the PD1-CD28 switch receptor, a fusion protein composed of the extracellular part of PD1 and the intracellular part of CD28, can enhance T cell function in vitro and in tumor animal models^{[12-16]}. Studies have shown that the introduction of a PD1-CD28 chimeric switch receptor can be a more efficient and effective approach to rescue T cells from tumor immunosuppression than the strategy of introducing additional co-stimulatory ligands into CART cells^{[8-10]} or making CART cells secrete checkpoint inhibitors^{[17, 18]}.

Effective systemic treatments for relapsed or refractory T-cell acute lymphoblastic leukemia (T-ALL) are limited. Recent clinical applications of chimeric antigen receptor (CAR) immunotherapy have demonstrated that CAR-T cells successfully control B-cell malignancies; however, designing CARs for T-ALL remains a challenge.

Overexpression of CD7 in T-cell malignancies can be an attractive target for immunotherapy of T-ALL. Studies have found that 30% of AML express CD7. However, its presence on normal T cells means that expression of CD7 CAR on these cells is not feasible because it will prove fratricidal. Therefore, the development of safe and effective autologous CD7-CAR T cells for the treatment of T-ALL and AML is of great urgency.

Effective systemic treatments for relapsed or refractory T-cell acute lymphoblastic leukemia (T-ALL) are limited. Recent clinical applications of chimeric antigen receptor (CAR) immunotherapy have demonstrated that CAR-T cells successfully control B-cell malignancies; however, designing CARs for T-ALL remains a challenge.

Overexpression of CD7 in T cell malignancies can be an attractive target for immunotherapy of T-ALL. Studies have found that 30% of AML express CD7. However, its presence on normal T cells means that expression of CD7 CAR on these cells is not feasible because it will prove to be fratricidal. Whether SgRNA that accurately and specifically targets the CD7 gene can be designed and prepared has become a key technology for CRISPR/cas9 to specifically knock out the CD7 gene. Therefore, it is very urgent to develop safe and effective autologous CD7-CAR T cells for the treatment of T-ALL and AML.

CRISPR (Clustered Regularly Interspaced Short Palindromic Repeats) consists of highly conserved repeat sequences and multiple different spacer sequences arranged in sequence. The length of the repeat sequence is usually 21 to 48 bp, and the repeat sequences are separated by 26 to 72 bp spacer sequences. CRISPR-specific editing of target sequences is achieved through complementary recognition of crRNA and tracrRNA and target sequences. TracrRNA and crRNA are now expressed as a chimeric guide RNA (single guide RNA, sgRNA), simplifying the CRISPR-Cas9 system to two components: Cas9 protein and sgRNA. This simplification offers advantages such as easy construction, high efficiency, and low cost, making CRISPR-Cas9 the most suitable choice for genome editing technology.

Circular RNA (circRNA) is a class of non-coding RNAs with their ends joined together, produced through a special type of alternative splicing. CircRNA is a covalently closed ring structure without 5′ and 3′ polarity and cannot be degraded by ribonucleases. Studies have confirmed that circRNA plays an important role in the occurrence and development of diseases. Some circRNAs can serve as translation templates, and some circRNAs can affect the expression of parental genes through cis or trans effects.

### CONTENT OF THE PRESENT INVENTION

The present application provides a modified immune effector cell, which expresses a group of chimeric protein molecules, named Lymphocytes-APCs Co-stimulators (Lymphocytes-APCs Co-stimulators, LACO-Stim), including: a. A membrane fusion protein comprising extracellular CD40 ligand (CD40L) and intracellular CD28, or an scFv of anti-CD40 fused with intracellular CD28; b. A soluble fusion protein composed of extracellular CD40L and an scFv of anti-CD28; c. A bispecific antibody of anti-CD40 and anti-CD28.

When co-introduced with CARs or TCRs into human T lymphocytes, LACO-stim molecules exhibit a strong effect on increasing T cell anti-tumor function, stimulating and maturing APCs, macrophages, and myeloid-derived cells. T cells expressing LACO-stimulatory molecules have the ability to overcome the inhibitory effects of TEM, such as PD1/PD-L1, Treg, and TGF-β. LACO-stim can coordinate the interaction between T lymphocytes and APCs, promote the epitope spreading ability of APCs, and further enhance the anti-tumor activity.

On the one hand, the present application provides a modified immune effector cell, comprising a nucleic acid molecule encoding a fusion protein, wherein the fusion protein comprises a first domain that activates an antigen-presenting cell (APC) and a second domain that activates an immune effector cell, wherein (i) the first domain includes (a) an activating receptor ligand or a receptor binding fragment that binds to the APC, or (b) an activating receptor antibody or an antigen-binding fragment thereof that binds to the APC; and (ii) the second domain includes (a) a co-stimulatory ligand or a receptor binding fragment of the immune effector cell, (b) an antibody or an antigen-binding fragment that binds to a co-stimulatory receptor of the immune effector cell, or (c) a co-stimulatory receptor or a functional fragment thereof of the immune effector cell.

In some embodiments, the N-terminus of the first domain is connected to the C-terminus of the second domain.

In some embodiments, the C-terminus of the first domain is connected to the N-terminus of the second domain.

In some embodiments, the first domain is directly or indirectly connected to the second domain.

In some embodiments, the first domain and the second domain are connected by a linker.

In some embodiments, the linker includes a peptide linker.

In some embodiments, the antibody or antigen-binding fragment is an scFv.

In some embodiments, the APC is selected from the group consisting of a dendritic cell, a macrophage, a myeloid-derived suppressor cell, a monocyte, a B cell, a T cell, and a Langerhans cell.

In some embodiments, the activating receptor of the APC is selected from CD40, CD80, CD86, CD91, DEC-205 and DC-SIGN.

In some embodiments, the first domain comprises a ligand that binds to CD40, CD80, CD86, CD91, DEC-205, DC-SIGN, or a receptor binding fragment thereof.

In some embodiments, the first domain comprises a receptor binding fragment of CD40 ligand (CD40L).

In some embodiments, the first domain comprises an antibody or antigen-binding fragment thereof that binds to an activating receptor of the APC.

In some embodiments, the first binding domain is an anti-CD40 antibody or an antigen-binding fragment thereof.

In some embodiments, the immune effector cell is selected from the group consisting of a T cell, a NK cell, an NKT cell, a macrophage, a neutrophil and a granulocyte.

In some embodiments, the second domain comprises an intracellular domain of a co-stimulatory receptor.

In some embodiments, the co-stimulatory receptor is selected from CD28, 4-1BB, ICOS, CD27, OX40, DAP10, 2B4, CD30, CD2, LIGHT, GITR, TLR, DR3 and CD43.

In some embodiments, the co-stimulatory receptor is CD28 or 4-1BB.

In some embodiments, the second domain is a co-stimulatory ligand or a receptor binding fragment thereof of an immune effector cell.

In some embodiments, the co-stimulatory ligand is selected from CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R and CD44.

In some embodiments, the second domain is an antibody or antigen-binding fragment thereof that binds a co-stimulatory receptor.

In some embodiments, the co-stimulatory receptor is selected from CD28, 4-1BB, ICOS, CD27, OX40, DAP10, 2B4, CD30, CD2, LIGHT, GITR, TLR, DR3 and CD43.

In some embodiments, the co-stimulatory receptor is CD28 or 4-1BB.

In some embodiments, the first domain and the second domain are selected from the following combinations:
i) the first domain comprises CD40L or a receptor binding fragment thereof, and the second domain comprises a CD28 or 4-1BB intracellular domain;
ii) the first domain comprises CD40L or a receptor binding fragment thereof, and the second domain comprises an anti-CD28 antibody or an antigen-binding fragment thereof;
iii) the first domain comprises an anti-CD40 antibody or an antigen-binding fragment thereof, and the second domain comprises a CD28 intracellular domain;
iv) the first domain comprises an anti-CD40 antibody or an antigen-binding fragment thereof, and the second domain comprises an anti-CD28 antibody or an antigen-binding fragment thereof;
v) the first domain comprises CD40L or a receptor binding fragment thereof, and the second domain comprises a CD28 co-stimulatory ligand or a receptor binding fragment thereof; and
vi) the first domain comprises an anti-CD40 antibody or an antigen-binding fragment thereof, and the second domain comprises a CD28 co-stimulatory ligand or a receptor-binding fragment thereof.

In some embodiments, the fusion protein comprises the amino acid sequence shown in any one of SEQ ID NO: 234 to SEQ ID NO: 250.

In some embodiments, the immune effector cell includes engineered immune effector cell.

In some embodiments, the engineered immune effector cell includes a CAR-T cell, a CAR-NK cell or a TCR-T cell.

In some embodiments, the modified immune effector cell has reduced CD7 surface expression compared with corresponding immune cell and express anti-CD7 CAR.

In some embodiments, the engineered immune effector cell includes anti-CD7 CAR-T cell.

In some embodiments, the CAR comprises an extracellular antigen-binding domain, and the extracellular antigen-binding domain comprises the amino acid sequence shown in any one of SEQ ID NO: 162 to SEQ ID NO: 174.

In some embodiments, the CAR comprises the amino acid sequence shown in any one of SEQ ID NO: 180 to SEQ ID NO: 192.

In some embodiments, the expression of CD7 in the modified immune effector cell is absent or suppressed.

In some embodiments, the immune effector cell does not induce fratricide.

Chimeric antigen receptor T (CAR-T) cell, which expresses anti-CD7 CAR and comprises a nucleic acid molecule encoding a fusion protein, wherein the fusion protein comprises a first domain that activates an antigen-presenting cell (APC) and a second domain that activates an immune effector cell, wherein (i) the first domain includes (a) an activating receptor ligand or a receptor binding fragment thereof that binds to an APC, or (b) an activating receptor antibody or an antigen-binding fragment thereof that binds to an APC; and (ii) the second domain includes (a) a co-stimulatory ligand or a receptor binding fragment thereof of an immune effector cell, (b) an antibody or an antigen-binding fragment thereof that binds to a co-stimulatory receptor of an immune effector cell, or (c) a co-stimulatory receptor or a functional fragment thereof of an immune effector cell.

In some embodiments, the expression of CD7 in the CAR-T cell is absent or suppressed.

In some embodiments, the CAR-T cell does not induce fratricide.

On the other hand, the present application provides a drug combination comprising an immune effector cell and a fusion protein, wherein the fusion protein comprises a first domain that activates an antigen-presenting cell (APC) and a second domain that activates an immune effector cell, wherein (i) the first domain includes (a) an activating receptor ligand or a receptor binding fragment thereof that binds to the APC, or (b) an activating receptor antibody or an antigen-binding fragment thereof that binds to the APC; and (ii) the second domain includes (a) a co-stimulatory ligand or a receptor binding fragment thereof of the immune effector cell, or (b) an antibody or an antigen-binding fragment thereof that binds to a co-stimulatory receptor of the immune effector cell.

In some embodiments, the N-terminus of the first domain is connected to the C-terminus of the second domain.

In some embodiments, the C-terminus of the first domain is connected to the N-terminus of the second domain.

In some embodiments, the first domain is directly or indirectly connected to the second domain.

In some embodiments, the first domain and the second domain are connected by a linker.

In some embodiments, the linker includes a peptide linker.

In some embodiments, the antibody or antigen-binding fragment thereof is an scFv.

In some embodiments, the APC is selected from the group consisting of a dendritic cell, a macrophage, a myeloid-derived suppressor cell, a monocyte, a B cell, a T cell, and a Langerhans cell.

In some embodiments, the activating receptor of the APC is selected from CD40, CD80, CD86, CD91, DEC-205 and DC-SIGN.

In some embodiments, the first domain comprises a ligand that binds to CD40, CD80, CD86, CD91, DEC-205, DC-SIGN, or a receptor binding fragment thereof.

In some embodiments, the first domain comprises a receptor binding fragment of CD40 ligand (CD40L).

In some embodiments, the first domain comprises an antibody or antigen-binding fragment thereof that binds to an activating receptor of the APC.

In some embodiments, the antibody is an anti-CD40 antibody or an antigen-binding fragment thereof.

In some embodiments, the immune effector cell is selected from the group consisting of a T cell, a NK cell, an NKT cell, a macrophage, a neutrophil and a granulocyte.

In some embodiments, the second domain comprises an intracellular domain of a co-stimulatory receptor.

In some embodiments, the co-stimulatory receptor is selected from CD28, 4-1BB, ICOS, CD27, OX40, DAP10, 2B4, CD30, CD2, LIGHT, GITR, TLR, DR3 and CD43.

In some embodiments, the co-stimulatory receptor is CD28.

In some embodiments, the second domain is a co-stimulatory ligand or a receptor binding fragment thereof of an immune effector cell.

In some embodiments, the co-stimulatory ligand is selected from CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R and CD44.

In some embodiments, the second domain is an antibody or antigen-binding fragment thereof that binds a co-stimulatory receptor.

In some embodiments, the co-stimulatory receptor is selected from CD28, 4-1BB, ICOS, CD27, OX40, DAP10, 2B4, CD30, CD2, LIGHT, GITR, TLR, DR3 and CD43.

In some embodiments, the co-stimulatory receptor is CD28 or 4-1BB.

In some embodiments, the first domain and the second domain are selected from the following combinations:
i) the first domain comprises CD40L or a receptor binding fragment thereof, and the second domain comprises CD28 or 4-1n co-stimulatory ligand or a receptor binding fragment thereof;
ii) the first domain comprises CD40L or a receptor binding fragment thereof, and the second domain comprises an anti-CD28 antibody or an antigen-binding fragment thereof;
iii) the first domain comprises an anti-CD40 antibody or an antigen-binding fragment thereof, and the second domain comprises a CD28 co-stimulatory ligand or a receptor-binding fragment thereof; and
iv) the first domain comprises an anti-CD40 antibody or an antigen-binding fragment thereof, and the second domain comprises an anti-CD28 antibody or an antigen-binding fragment thereof.

In some embodiments, the fusion protein comprises the amino acid sequence shown in any one of SEQ ID NO: 237 to SEQ ID NO: 241.

In some embodiments, the immune effector cell includes an engineered immune effector cell.

In some embodiments, the engineered immune effector cell includes a CAR-T cell, a CAR-NK cell or a TCR-T cell.

In some embodiments, the drug combination has reduced CD7 surface expression compared with corresponding immune cells and expresses anti-CD7 CAR.

In some embodiments, the engineered immune effector cell includes anti-CD7 CAR-T cell.

In some embodiments, the CAR comprises an extracellular antigen-binding domain, and the extracellular antigen-binding domain comprises the amino acid sequence shown in any one of SEQ ID NO: 162 to SEQ ID NO: 174.

In some embodiments, the CAR comprises the amino acid sequence shown in any one of SEQ ID NO: 180 to SEQ ID NO: 192.

In some embodiments, the immune effector cell does not induce fratricide.

On the other hand, the present application provides a pharmaceutical composition comprising the modified immune effector cell described herein or the drug combination described herein, and a pharmaceutically acceptable carrier.

On the other hand, the present application provides the use of the modified immune effector cells described in the present application, the drug combination described in the present application, or the pharmaceutical composition described in the present application in the preparation of a drug for treating a tumor.

In some embodiments, the tumor includes a hematological tumor and a solid tumor.

In some embodiments, the tumor includes a tumor expressing CD7.

In some embodiments, the tumor includes a CD7-positive hematological malignancy.

In some embodiments, the tumor includes a T-cell malignancy.

In some embodiments, the T-cell malignancy includes acute T-lymphocytic leukemia (T-ALL), acute myeloid leukemia, or NK/T-cell lymphoma.

On the other hand, the present application provides a method for treating a tumor, including administering the modified immune effector cells described herein, the drug combination described herein, or the pharmaceutical composition described herein to a subject in need thereof.

On the other hand, the present application provides a method for killing malignant T cells, the method including contacting the malignant T cells with the modified immune effector cells of the present application, the drug combination of the present application, or the pharmaceutical composition of the present application.

On the other hand, the present application provides a method for preparing a population of chimeric antigen receptor T (CAR-T) cells, wherein the CAR targets CD7, including the following steps:
a. Modify the CD7 gene in the population of T cells;
b. Activating the population of T cells;
c. Transducing the population of T cells with anti-CD7 CAR and fusion protein; and
d. Expanding the population of chimeric antigen receptor T cells.

In some embodiments, CD7 is absent or suppressed in the population of modified T cells compared with the corresponding unmodified cell.

In some embodiments, the modification includes administering to the population of T cells one or more substances selected from the group consisting of antisense RNA, siRNA, shRNA, transcription activator-like effector nuclease (TALEN), zinc finger nuclease (ZFN) and CRISPR/Cas system.

In some embodiments, the modification includes administering a CRISPR/Cas system to the population of T cells.

In some embodiments, the modification includes administering a CRISPR/Cas9 system to the population of T cells.

In some embodiments, the modification includes administering Cas9 and a gRNA targeting the CD7 gene to the population of T cells.

In some embodiments, the gRNA targeting the CD7 gene comprises a nucleotide sequence shown in any one of SEQ ID NO: 211 to SEQ ID NO: 218.

In some embodiments, the Cas9 is delivered to the cell as mRNA or protein.

In some embodiments, the gRNA is delivered simultaneously with the Cas9.

In some embodiments, wherein the delivering is performed by electroporation.

In some embodiments, the CD7 CAR nucleic acid molecule and the nucleic acid molecule of the fusion protein are mRNA.

In some embodiments, the CD7 CAR nucleic acid molecule and the nucleic acid molecule of the fusion protein are circRNA.

In some embodiments, the cirRNA chimeric antigen receptor comprises elements, in the following order: an internal ribosome entry site (IRES) element, a protein coding sequence targeting CD7, and polyadenylic acid (polyA), and the cirRNA fusion protein comprises elements arranged in the following order: an internal ribosome entry site (IRES) element, a fusion protein coding sequence, and polyadenylic acid (polyA).

In some embodiments, the transduction of the anti-CD7 CAR and the fusion protein into the T cell includes introducing a nucleic acid molecule encoding the anti-CD7 CAR and a nucleic acid molecule encoding the fusion protein into the T cell.

On the other hand, cell population comprising the modified immune effector cells described in the present application or the CAR-T cells described in the present application, wherein the cell population is derived from peripheral blood mononuclear cells (PBMC), peripheral blood leukocytes (PBL), tumor infiltrating lymphocytes (TIL), cytokine-induced killer cells (CIK), lymphokine-activated killer cells (LAK) or bone marrow infiltrating lymphocytes (MIL).

On the one hand, the present application provides an antigen-binding protein that specifically binds to CD7 protein.

On the other hand, the present application provides a chimeric antigen receptor and CAR-T cells targeting CD7.

On the other hand, the present application provides a CAR-T cell targeting CD7 that avoids self-killing and its use.

This application discloses the following technical solutions:

On the one hand, an isolated antigen-binding protein is provided, which specifically binds to CD7 protein, wherein the isolated antigen-binding protein comprises a heavy chain variable region (VH), and the VH comprises HCDR1, HCDR2 and HCDR3, wherein HCDR1 comprises the amino acid sequence shown in SEQ ID NO: 1.

In some embodiments, the HCDR1 comprises the amino acid sequence shown in any one of SEQ ID NO: 2 to SEQ ID NO: 9.

In some embodiments, the HCDR2 comprises the amino acid sequence shown in any one of SEQ ID NO: 10 to SEQ ID NO: 17.

In some embodiments, the HCDR3 comprises the amino acid sequence shown in SEQ ID NO: 18 or SEQ ID NO: 27.

In some embodiments, the HCDR1 comprises the amino acid sequence shown in SEQ ID NO: 2, the HCDR2 comprises the amino acid sequence shown in SEQ ID NO: 10, and the HCDR3 comprises the amino acid sequence shown in SEQ ID NO: 18; or
The HCDR1 comprises the amino acid sequence shown in SEQ ID NO: 3, the HCDR2 comprises the amino acid sequence shown in SEQ ID NO: 11, and the HCDR3 comprises the amino acid sequence shown in SEQ ID NO: 19; or
The HCDR1 comprises the amino acid sequence shown in SEQ ID NO: 2, the HCDR2 comprises the amino acid sequence shown in SEQ ID NO: 10, and the HCDR3 comprises the amino acid sequence shown in SEQ ID NO: 20; or
The HCDR1 comprises the amino acid sequence shown in SEQ ID NO: 4, the HCDR2 comprises the amino acid sequence shown in SEQ ID NO: 12, and the HCDR3 comprises the amino acid sequence shown in SEQ ID NO: 21; or
The HCDR1 comprises the amino acid sequence shown in SEQ ID NO: 5, the HCDR2 comprises the amino acid sequence shown in SEQ ID NO: 13, and the HCDR3 comprises the amino acid sequence shown in SEQ ID NO: 22; or
The HCDR1 comprises the amino acid sequence shown in SEQ ID NO: 6, the HCDR2 comprises the amino acid sequence shown in SEQ ID NO: 14, and the HCDR3 comprises the amino acid sequence shown in SEQ ID NO: 23; or
The HCDR1 comprises the amino acid sequence shown in SEQ ID NO: 2, the HCDR2 comprises the amino acid sequence shown in SEQ ID NO: 10, and the HCDR3 comprises the amino acid sequence shown in SEQ ID NO: 24; or
The HCDR1 comprises the amino acid sequence shown in SEQ ID NO: 7, the HCDR2 comprises the amino acid sequence shown in SEQ ID NO: 15, and the HCDR3 comprises the amino acid sequence shown in SEQ ID NO: 25; or
The HCDR1 comprises the amino acid sequence shown in SEQ ID NO: 8, the HCDR2 comprises the amino acid sequence shown in SEQ ID NO: 16, and the HCDR3 comprises the amino acid sequence shown in SEQ ID NO: 26; or
The HCDR1 comprises the amino acid sequence shown in SEQ ID NO: 9, the HCDR2 comprises the amino acid sequence shown in SEQ ID NO: 17, and the HCDR3 comprises the amino acid sequence shown in SEQ ID NO: 27.

In some embodiments, the isolated antigen-binding protein comprises VH, wherein the VH includes framework regions HFR1, HFR2, HFR3 and HFR4, the C-terminus of the HFR1 is directly or indirectly connected to the N-terminus of the HCDR1, and the HFR1 comprises the amino acid sequence shown in any one of SEQ ID NO: 28 to SEQ ID NO: 38.

In some embodiments, the HFR2 is located between the HCDR1 and the HCDR2, and the HFR2 comprises the amino acid sequence shown in any one of SEQ ID NO: 39 to SEQ ID NO: 47.

In some embodiments, the HFR3 is located between the HCDR2 and the HCDR3, and the HFR3 comprises the amino acid sequence shown in any one of SEQ ID NO: 48 to SEQ ID NO: 56.

In some embodiments, the N-terminus of the HFR4 is directly or indirectly connected to the C-terminus of the HCDR3, and the HFR4 comprises the amino acid sequence shown in any one of SEQ ID NO: 57 to SEQ ID NO: 60.

In some embodiments, the isolated antigen-binding protein comprises VH, wherein the VH includes framework regions HFR1, HFR2, HFR3 and HFR4, the C-terminus of the HFR1 is directly or indirectly connected with the N-terminus of the HCDR1, the HFR2 is located between the HCDR1 and the HCDR2, the HFR3 is located between the HCDR2 and the HCDR3, and the N-terminus of the HFR4 is directly or indirectly connected with the C-terminus of the HCDR3; wherein the HFR1 comprises the amino acid sequence shown in SEQ ID NO: 28, the HFR2 comprises the amino acid sequence shown in SEQ ID NO: 39, the HFR3 comprises the amino acid sequence shown in SEQ ID NO: 48, and the HFR4 comprises the amino acid sequence shown in SEQ ID NO: 57; or
The HFR1 comprises the amino acid sequence shown in SEQ ID NO: 29, the HFR2 comprises the amino acid sequence shown in SEQ ID NO: 40, the HFR3 comprises the amino acid sequence shown in SEQ ID NO: 49, and the HFR4 comprises the amino acid sequence shown in SEQ ID NO: 58; or
The HFR1 comprises the amino acid sequence shown in SEQ ID NO: 30, the HFR2 comprises the amino acid sequence shown in SEQ ID NO: 39, the HFR3 comprises the amino acid sequence shown in SEQ ID NO: 48, and the HFR4 comprises the amino acid sequence shown in SEQ ID NO: 58; or
The amino acid sequence shown in SEQ ID NO: 31, the HFR2 comprises the amino acid sequence shown in SEQ ID NO: 41, the HFR3 comprises the amino acid sequence shown in SEQ ID NO: 50, and the HFR4 comprises the amino acid sequence shown in SEQ ID NO: 59; or
The HFR1 comprises the amino acid sequence shown in SEQ ID NO: 32, the HFR2 comprises the amino acid sequence shown in SEQ ID NO: 42, the HFR3 comprises the amino acid sequence shown in SEQ ID NO: 51, and the HIFR4 comprises the amino acid sequence shown in SEQ ID NO: 58; or
The HIFR1 comprises the amino acid sequence shown in SEQ ID NO: 33, the HIFR2 comprises the amino acid sequence shown in SEQ ID NO: 39, the HIFR3 comprises the amino acid sequence shown in SEQ ID NO: 48, and the HIFR4 comprises the amino acid sequence shown in SEQ ID NO: 58; or
The HIFR1 comprises the amino acid sequence shown in SEQ ID NO: 34, the HIFR2 comprises the amino acid sequence shown in SEQ ID NO: 39, the HIFR3 comprises the amino acid sequence shown in SEQ ID NO: 48, and the HIFR4 comprises the amino acid sequence shown in SEQ ID NO: 58; or
The HIFR1 comprises the amino acid sequence shown in SEQ ID NO: 35, the HIFR2 comprises the amino acid sequence shown in SEQ ID NO: 43, the HIFR3 comprises the amino acid sequence shown in SEQ ID NO: 52, and the HIFR4 comprises the amino acid sequence shown in SEQ ID NO: 58; or
The HIFR1 comprises the amino acid sequence shown in SEQ ID NO: 28, the HIFR2 comprises the amino acid sequence shown in SEQ ID NO: 39, the HIFR3 comprises the amino acid sequence shown in SEQ ID NO: 48, and the HIFR4 comprises the amino acid sequence shown in SEQ ID NO: 58; or
The HIFR1 comprises the amino acid sequence shown in SEQ ID NO: 30, the HIFR2 comprises the amino acid sequence shown in SEQ ID NO: 44, the HIFR3 comprises the amino acid sequence shown in SEQ ID NO: 53, and the HIFR4 comprises the amino acid sequence shown in SEQ ID NO: 58; or
The HIFR1 comprises the amino acid sequence shown in SEQ ID NO: 36, the HFR2 comprises the amino acid sequence shown in SEQ ID NO: 45, the HFR3 comprises the amino acid sequence shown in SEQ ID NO: 54, and the HFR4 comprises the amino acid sequence shown in SEQ ID NO: 60; or
The HIFR1 comprises the amino acid sequence shown in SEQ ID NO: 37, the HIFR2 comprises the amino acid sequence shown in SEQ ID NO: 46, the HIFR3 comprises the amino acid sequence shown in SEQ ID NO: 55, and the HIFR4 comprises the amino acid sequence shown in SEQ ID NO: 60; or
The HFR1 comprises the amino acid sequence shown in SEQ ID NO: 38, the HFR2 comprises the amino acid sequence shown in SEQ ID NO: 47, the HFR3 comprises the amino acid sequence shown in SEQ ID NO: 56, and the HFR4 comprises the amino acid sequence shown in SEQ ID NO: 58.

In some embodiments, the isolated antigen-binding protein comprises a VH, wherein the VH comprises the amino acid sequence shown in SEQ ID NO: 61 to SEQ ID NO: 73.

In some embodiments, the isolated antigen-binding protein comprises a VL, wherein the VL comprises LCDR1, LCDR2, and LCDR3, and the LCDR1 comprises the amino acid sequence shown in any one of SEQ ID NO: 74 to SEQ ID NO: 86.

In some embodiments, the isolated antigen-binding protein comprises a VL, wherein the VL comprises LCDR1, LCDR2, and LCDR3, and the LCDR2 comprises the amino acid sequence shown in any one of SEQ ID NO: 87 to SEQ ID NO: 98.

In some embodiments, the isolated antigen-binding protein comprises a VL, wherein the VL comprises LCDR1, LCDR2, and LCDR3, and the LCDR3 comprises the amino acid sequence shown in any one of SEQ ID NO: 99 to SEQ ID NO: 111.

In some embodiments, the isolated antigen-binding protein comprises a VL, wherein the VL comprises LCDR1, LCDR2 and LCDR3, the LCDR1 comprises the amino acid sequence shown in SEQ ID NO: 74, the LCDR2 comprises the amino acid sequence shown in SEQ ID NO: 87, and the LCDR3 comprises the amino acid sequence shown in SEQ ID NO: 99; or
The LCDR1 comprises the amino acid sequence shown in SEQ ID NO: 75, the LCDR2 comprises the amino acid sequence shown in SEQ ID NO: 88, and the LCDR3 comprises the amino acid sequence shown in SEQ ID NO: 100; or
The LCDR1 comprises the amino acid sequence shown in SEQ ID NO: 76, the LCDR2 comprises the amino acid sequence shown in SEQ ID NO: 89, and the LCDR3 comprises the amino acid sequence shown in SEQ ID NO: 101; or
The LCDR1 comprises the amino acid sequence shown in SEQ ID NO: 77, the LCDR2 comprises the amino acid sequence shown in SEQ ID NO: 90, and the LCDR3 comprises the amino acid sequence shown in SEQ ID NO: 102; or
The LCDR1 comprises the amino acid sequence shown in SEQ ID NO: 78, the LCDR2 comprises the amino acid sequence shown in SEQ ID NO: 91, and the LCDR3 comprises the amino acid sequence shown in SEQ ID NO: 103; or
The LCDR1 comprises the amino acid sequence shown in SEQ ID NO: 79, the LCDR2 comprises the amino acid sequence shown in SEQ ID NO: 92, and the LCDR3 comprises the amino acid sequence shown in SEQ ID NO: 104; or
The LCDR1 comprises the amino acid sequence shown in SEQ ID NO: 80, the LCDR2 comprises the amino acid sequence shown in SEQ ID NO: 93, and the LCDR3 comprises the amino acid sequence shown in SEQ ID NO: 105; or
The LCDR1 comprises the amino acid sequence shown in SEQ ID NO: 81, the LCDR2 comprises the amino acid sequence shown in SEQ ID NO: 94, and the LCDR3 comprises the amino acid sequence shown in SEQ ID NO: 106; or
The LCDR1 comprises the amino acid sequence shown in SEQ ID NO: 82, the LCDR2 comprises the amino acid sequence shown in SEQ ID NO: 95, and the LCDR3 comprises the amino acid sequence shown in SEQ ID NO: 107; or
The LCDR1 comprises the amino acid sequence shown in SEQ ID NO: 84, the LCDR2 comprises the amino acid sequence shown in SEQ ID NO: 97, and the LCDR3 comprises the amino acid sequence shown in SEQ ID NO: 109; or
The LCDR1 comprises the amino acid sequence shown in SEQ ID NO: 85, the LCDR2 comprises the amino acid sequence shown in SEQ ID NO: 98, and the LCDR3 comprises the amino acid sequence shown in SEQ ID NO: 110; or
The LCDR1 comprises the amino acid sequence shown in SEQ ID NO: 86, the LCDR2 comprises the amino acid sequence shown in SEQ ID NO: 98, and the LCDR3 comprises the amino acid sequence shown in SEQ ID NO: 111.

In some embodiments, the isolated antigen-binding protein comprises VH and VL, wherein the VH comprises HCDR1, HCDR2 and HCDR3, and the VL comprises LCDR1, LCDR2 and LCDR3; wherein the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 74, the LCDR2 comprises the amino acid sequence shown in SEQ ID NO: 87, and the LCDR3 comprises the amino acid sequence shown in SEQ ID NO: 99; or
The HCDR1 comprises the amino acid sequence shown in SEQ ID NO: 3, the HCDR2 comprises the amino acid sequence shown in SEQ ID NO: 11, the HCDR3 comprises the amino acid sequence shown in SEQ ID NO: 19, the LCDR1 comprises the amino acid sequence shown in SEQ ID NO: 75, the LCDR2 comprises the amino acid sequence shown in SEQ ID NO: 88, and the LCDR3 comprises the amino acid sequence shown in SEQ ID NO: 100; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 20, the LCDR1 comprises the amino acid sequence shown in SEQ ID NO: 76, the LCDR2 comprises the amino acid sequence shown in SEQ ID NO: 89, and the LCDR3 comprises the amino acid sequence shown in SEQ ID NO: 101; or
The HCDR1 comprises the amino acid sequence shown in SEQ ID NO: 4, the HCDR2 comprises the amino acid sequence shown in SEQ ID NO: 12, the HCDR3 comprises the amino acid sequence shown in SEQ ID NO: 21, the LCDR1 comprises the amino acid sequence shown in SEQ ID NO: 77, the LCDR2 comprises the amino acid sequence shown in SEQ ID NO: 90, and the LCDR3 comprises the amino acid sequence shown in SEQ ID NO: 102; or
The HCDR1 comprises the amino acid sequence shown in SEQ ID NO: 5, the HCDR2 comprises the amino acid sequence shown in SEQ ID NO: 13, the HCDR3 comprises the amino acid sequence shown in SEQ ID NO: 22, the LCDR1 comprises the amino acid sequence shown in SEQ ID NO: 91, the LCDR2 comprises the amino acid sequence shown in SEQ ID NO: 91, and the LCDR3 comprises the amino acid sequence shown in SEQ ID NO: 103; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 79, the LCDR2 comprises the amino acid sequence shown in SEQ ID NO: 92, and the LCDR3 comprises the amino acid sequence shown in SEQ ID NO: 104; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 80, the LCDR2 comprises the amino acid sequence shown in SEQ ID NO: 93, and the LCDR3 comprises the amino acid sequence shown in SEQ ID NO: 105; or
The HCDR1 comprises the amino acid sequence shown in SEQ ID NO: 6, the HCDR2 comprises the amino acid sequence shown in SEQ ID NO: 14, the HCDR3 comprises the amino acid sequence shown in SEQ ID NO: 23, the LCDR1 comprises the amino acid sequence shown in SEQ ID NO: 81, the LCDR2 comprises the amino acid sequence shown in SEQ ID NO: 94, and the LCDR3 comprises the amino acid sequence shown in SEQ ID NO: 106; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 82, the LCDR2 comprises the amino acid sequence shown in SEQ ID NO: 95, and the LCDR3 comprises the amino acid sequence shown in SEQ ID NO: 107; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 24, the LCDR1 comprises the amino acid sequence shown in SEQ ID NO: 83, the LCDR2 comprises the amino acid sequence shown in SEQ ID NO: 96, and the LCDR3 comprises the amino acid sequence shown in SEQ ID NO: 108; or
The HCDR1 comprises the amino acid sequence shown in SEQ ID NO: 7, the HCDR2 comprises the amino acid sequence shown in SEQ ID NO: 15, the HCDR3 comprises the amino acid sequence shown in SEQ ID NO: 25, the LCDR1 comprises the amino acid sequence shown in SEQ ID NO: 84, the LCDR2 comprises the amino acid sequence shown in SEQ ID NO: 97, and the LCDR3 comprises the amino acid sequence shown in SEQ ID NO: 109; or
The HCDR1 comprises the amino acid sequence shown in SEQ ID NO: 8, the HCDR2 comprises the amino acid sequence shown in SEQ ID NO: 16, the HCDR3 comprises the amino acid sequence shown in SEQ ID NO: 26, the LCDR1 comprises the amino acid sequence shown in SEQ ID NO: 85, the LCDR2 comprises the amino acid sequence shown in SEQ ID NO: 98, and the LCDR3 comprises the amino acid sequence shown in SEQ ID NO: 110; or
The HCDR1 comprises the amino acid sequence shown in SEQ ID NO: 9, the HCDR2 comprises the amino acid sequence shown in SEQ ID NO: 17, the HCDR3 comprises the amino acid sequence shown in SEQ ID NO: 27, the LCDR1 comprises the amino acid sequence shown in SEQ ID NO: 86, the LCDR2 comprises the amino acid sequence shown in SEQ ID NO: 98, and the LCDR3 comprises the amino acid sequence shown in SEQ ID NO: 111.

In some embodiments, the isolated antigen-binding protein comprises a VL, wherein the VL includes framework region LFR1, LFR2, LFR3 and LFR4, wherein the C-terminus of the LFR1 is directly or indirectly connected to the N-terminus of the LCDR1, and the LFR1 comprises the amino acid sequence shown in any one of SEQ ID NO: 112 to SEQ ID NO: 123.

In some embodiments, the isolated antigen-binding protein comprises a VL, wherein the VL includes a framework region LFR 2, wherein the LFR2 is located between the LCDR1 and the LCDR2, and the LFR2 comprises the amino acid sequence shown in any one of SEQ ID NO: 124 to SEQ ID NO: 130.

In some embodiments, the isolated antigen-binding protein comprises a VL, wherein the VL includes a framework region LFR3, the LFR3 is located between the LCDR2 and the LCDR3, and the LFR3 comprises the amino acid sequence shown in any one of SEQ ID NO: 131 to SEQ ID NO: 141.

In some embodiments, the isolated antigen-binding protein comprises a VL, wherein the VL includes a framework region LFR 4, the N-terminus of the LFR4 is directly or indirectly connected to the C-terminus of the LCDR3, and the LFR4 comprises the amino acid sequence shown in any one of SEQ ID NO: 142 to SEQ ID NO: 147.

In some embodiments, the isolated antigen-binding protein comprises a VL, wherein the VL includes framework regions LFR1, LFR2, LFR3 and LFR4, wherein the C-terminus of the LFR1 is directly or indirectly connected with the N-terminus of the LCDR1, the LFR2 is located between the LCDR1 and the LCDR2, the LFR3 is located between the LCDR2 and the LCDR3, and the N-terminus of the LFR4 is directly or indirectly connected with the C-terminus of the LCDR3; wherein the LFR1 comprises the amino acid sequence shown in SEQ ID NO: 112, the LFR2 comprises the amino acid sequence shown in SEQ ID NO: 124, the LFR3 comprises the amino acid sequence shown in SEQ ID NO: 131, and the LFR4 comprises the amino acid sequence shown in SEQ ID NO: 142; or
The LFR1 comprises the amino acid sequence shown in SEQ ID NO: 113, the LFR2 comprises the amino acid sequence shown in SEQ ID NO: 125, the LFR3 comprises the amino acid sequence shown in SEQ ID NO: 132, and the LFR4 comprises the amino acid sequence shown in SEQ ID NO: 142; or
The LFR1 comprises the amino acid sequence shown in SEQ ID NO: 114, the LFR2 comprises the amino acid sequence shown in SEQ ID NO: 126, the LFR3 comprises the amino acid sequence shown in SEQ ID NO: 133, and the LFR4 comprises the amino acid sequence shown in SEQ ID NO: 142; or
The LFR1 comprises the amino acid sequence shown in SEQ ID NO: 115, the LFR2 comprises the amino acid sequence shown in SEQ ID NO: 127, the LFR3 comprises the amino acid sequence shown in SEQ ID NO: 134, and the LFR4 comprises the amino acid sequence shown in SEQ ID NO: 142; or
The LFR1 comprises the amino acid sequence shown in SEQ ID NO: 116, the LFR2 comprises the amino acid sequence shown in SEQ ID NO: 125, the LFR3 comprises the amino acid sequence shown in SEQ ID NO: 135, and the LFR4 comprises the amino acid sequence shown in SEQ ID NO: 143; or
The LFR1 comprises the amino acid sequence shown in SEQ ID NO: 117, the LFR2 comprises the amino acid sequence shown in SEQ ID NO: 126, the LFR3 comprises the amino acid sequence shown in SEQ ID NO: 136, and the LFR4 comprises the amino acid sequence shown in SEQ ID NO: 143; or
The LFR1 comprises the amino acid sequence shown in SEQ ID NO: 118, the LFR2 comprises the amino acid sequence shown in SEQ ID NO: 128, the LFR3 comprises the amino acid sequence shown in SEQ ID NO: 137, and the LFR4 comprises the amino acid sequence shown in SEQ ID NO: 142; or
The LFR1 comprises the amino acid sequence shown in SEQ ID NO: 119, the LFR2 comprises the amino acid sequence shown in SEQ ID NO: 125, the LFR3 comprises the amino acid sequence shown in SEQ ID NO: 138, and the LFR4 comprises the amino acid sequence shown in SEQ ID NO: 143; or
The LFR1 comprises the amino acid sequence shown in SEQ ID NO: 120, the LFR2 comprises the amino acid sequence shown in SEQ ID NO: 129, the LFR3 comprises the amino acid sequence shown in SEQ ID NO: 139, and the LFR4 comprises the amino acid sequence shown in SEQ ID NO: 142; or
The LFR1 comprises the amino acid sequence shown in SEQ ID NO: 114, the LFR2 comprises the amino acid sequence shown in SEQ ID NO: 126, the LFR3 comprises the amino acid sequence shown in SEQ ID NO: 140, and the LFR4 comprises the amino acid sequence shown in SEQ ID NO: 144; or
The LFR1 comprises the amino acid sequence shown in SEQ ID NO: 121, the LFR2 comprises the amino acid sequence shown in SEQ ID NO: 130, the LFR3 comprises the amino acid sequence shown in SEQ ID NO: 141, and the LFR4 comprises the amino acid sequence shown in SEQ ID NO: 145; or
The LFR1 comprises the amino acid sequence shown in SEQ ID NO: 122, the LFR2 comprises the amino acid sequence shown in SEQ ID NO: 130, the LFR3 comprises the amino acid sequence shown in SEQ ID NO: 141, and the LFR4 comprises the amino acid sequence shown in SEQ ID NO: 146; or
The LFR1 comprises the amino acid sequence shown in SEQ ID NO: 123, the LFR2 comprises the amino acid sequence shown in SEQ ID NO: 130, the LFR3 comprises the amino acid sequence shown in SEQ ID NO: 141, and the LFR4 comprises the amino acid sequence shown in SEQ ID NO: 147.

In some embodiments, the isolated antigen-binding protein comprises a VL, wherein the VL comprises the amino acid sequence shown in any one of SEQ ID NO: 148 to SEQ ID NO: 160.

In some embodiments, the isolated antigen-binding protein comprises VH and VL, wherein the VH comprises the amino acid sequence shown in SEQ ID NO: 61, and the VL comprises the amino acid sequence shown in SEQ ID NO: 148; or
The VH comprises the amino acid sequence shown in SEQ ID NO: 62, and the VL comprises the amino acid sequence shown in SEQ ID NO: 149; or
The VH comprises the amino acid sequence shown in SEQ ID NO: 63, and the VL comprises the amino acid sequence shown in SEQ ID NO: 150; or
The VH comprises the amino acid sequence shown in SEQ ID NO: 64, and the VL comprises the amino acid sequence shown in SEQ ID NO: 151; or
The VH comprises the amino acid sequence shown in SEQ ID NO: 65, and the VL comprises the amino acid sequence shown in SEQ ID NO: 152; or
The VH comprises the amino acid sequence shown in SEQ ID NO: 66, and the VL comprises the amino acid sequence shown in SEQ ID NO: 153; or
The VH comprises the amino acid sequence shown in SEQ ID NO: 67, and the VL comprises the amino acid sequence shown in SEQ ID NO: 154; or
The VH comprises the amino acid sequence shown in SEQ ID NO: 68, and the VL comprises the amino acid sequence shown in SEQ ID NO: 155; or
The VH comprises the amino acid sequence shown in SEQ ID NO: 69, and the VL comprises the amino acid sequence shown in SEQ ID NO: 156; or
The VH comprises the amino acid sequence shown in SEQ ID NO: 70, and the VL comprises the amino acid sequence shown in SEQ ID NO: 157; or
The VH comprises the amino acid sequence shown in SEQ ID NO: 71, and the VL comprises the amino acid sequence shown in SEQ ID NO: 158; or
The VH comprises the amino acid sequence shown in SEQ ID NO: 72, and the VL comprises the amino acid sequence shown in SEQ ID NO: 159; or
The VH comprises the amino acid sequence shown in SEQ ID NO: 73, and the VL comprises the amino acid sequence shown in SEQ ID NO: 160.

In some embodiments, the isolated antigen-binding protein includes an antibody or an antigen-binding fragment thereof.

In some embodiments, the antibody includes a monoclonal antibody, a polyclonal antibody, a dimer, a multimer, a multispecific antibody, a full-length antibody, an antibody fragment, a human antibody, a humanized antibody, or a chimeric antibody.

In some embodiments, the antigen-binding fragment includes Fab, Fab′, Fv fragment, F(ab′)2, scFv, di-scFv and/or dAb.

In some embodiments, the antigen-binding protein includes an scFv.

In some embodiments, the VL and VH are connected by a linker.

In some embodiments, the linker includes a polypeptide linker.

In some embodiments, the polypeptide linker comprises an amino acid sequence represented by (GGGGS)n, wherein n is any integer from 1 to 5.

In some embodiments, it comprises the amino acid sequence shown in any one of SEQ ID NO: 162 to SEQ ID NO: 174.

On the other hand, the present application provides an isolated polypeptide comprising the isolated antigen-binding protein of the present application.

On the other hand, the present application provides an immunoconjugate comprising the antigen-binding protein of the present application.

In some embodiments, the immunoconjugate comprises:
(a) the antigen-binding protein of the present application;
(b) a conjugated moiety selected from the group consisting of a detectable label, a drug, a toxin, a cytokine, a viral coat protein or a VLP, or a combination thereof.

A chimeric antigen receptor comprising at least one extracellular antigen-binding domain comprising the antigen-binding protein described herein.

In some embodiments, the extracellular antigen-binding domain includes an scFv.

In some embodiments, the chimeric antigen receptor includes a transmembrane domain, comprising a transmembrane domain derived from one or more proteins selected from the following group: CD8, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64 and SLAM.

In some embodiments, the transmembrane domain comprises a transmembrane domain derived from CD8.

In some embodiments, the transmembrane domain comprises the amino acid sequence shown in SEQ ID NO: 177.

In some embodiments, the chimeric antigen receptor includes an intracellular co-stimulatory signaling domain, comprising an intracellular co-stimulatory signaling domain derived from one or more proteins selected from the group consisting of CD28, CD137, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40, and MyD88.

In some embodiments, the intracellular co-stimulatory signaling domain is derived from the co-stimulatory signaling domain of 4-1BB.

In some embodiments, the intracellular co-stimulatory signaling domain comprises the amino acid sequence shown in any one of SEQ ID NO: 178.

In some embodiments, the chimeric antigen receptor includes an intracellular signaling domain, wherein the intracellular signaling domain comprising an intracellular signaling domain derived from one or more proteins selected from the following group: CD3ζ, CD3δ, CD3γ, CD3ε, CD79a, CD79b, FceRIγ, FceRIβ, FcγRIIa, bovine leukemia virus gp30, Epstein-Barr virus (EBV) LMP2A, simian immunodeficiency virus PBj14 Nef, DAP10, DAP-12 and a domain comprising at least one ITAM.

In some embodiments, the intracellular signaling domain comprises a signaling domain derived from CD3ζ.

In some embodiments, the intracellular signaling domain comprises the amino acid sequence shown in SEQ ID NO: 179.

In some embodiments, it includes a hinge region between the extracellular antigen-binding domain and the transmembrane domain, wherein the hinge region comprises a hinge region derived from one or more proteins selected from the group consisting of CD28, IgG1, IgG4, IgD, 4-1BB, CD4, CD27, CD7, CD8, PD-1, ICOS, OX40, NKG2D, NKG2C, FcεRIγ, BTLA, GITR, DAP10, TIM1, SLAM, CD30 and LIGHT.

In some embodiments, the hinge region comprises a hinge region derived from CD8.

In some embodiments, the hinge region comprises the amino acid sequence shown in SEQ ID NO: 176.

In some embodiments, the non-targeting portion of the chimeric antigen receptor comprises a transmembrane domain, a hinge region, an intracellular co-stimulatory signaling domain, and an intracellular signaling domain.

In some embodiments, the non-targeting portion of the chimeric antigen receptor comprises the transmembrane domain of the CD8 molecule, the hinge region of CD8, the intracellular co-stimulatory signaling domain of 4-1BB, and the intracellular signaling domain of CD3ζ.

In some embodiments, it further comprises a signal peptide fragment, wherein the C-terminus of the signal peptide fragment is connected to the N-terminus of the extracellular antigen-binding domain.

In some embodiments, the signal peptide fragment comprises a CD8 signal peptide fragment.

In some embodiments, the signal peptide fragment comprises the amino acid sequence shown in SEQ ID NO: 175.

In some embodiments, it comprises the amino acid sequence shown in any one of SEQ ID NO: 180 to SEQ ID NO: 192.

On the other hand, the present application provides one or more isolated nucleic acid molecules encoding the isolated antigen-binding protein, the polypeptide or the chimeric antigen receptor described in the present application.

In some embodiments, the isolated nucleic acid molecule comprises a nucleotide sequence shown in any one of SEQ ID NO: 193 to SEQ ID NO: 205.

On the other hand, the present application provides an expression vector comprising the nucleic acid molecule described in the present application.

In some embodiments, the vector is selected from a DNA vector, an RNA vector, a plasmid, a lentiviral vector, an adenoviral vector, an adeno-associated viral vector and a retroviral vector.

On the other hand, the present application provides a cell, wherein the cell comprises the nucleic acid molecule of the present application or the expression vector described in the present application, and/or ii) the cell expresses the antigen-binding protein described in the present application, the polypeptide described in the present application or the chimeric antigen receptor described in the present application.

On the other hand, the present application provides a method for preparing the isolated antigen-binding protein described herein, the method including culturing the cell described herein under conditions such that the isolated antigen-binding protein described herein is expressed.

On the other hand, the present application provides an engineered cell, comprising the nucleic acid molecule described in the present application or the vector described in the present application, and/or expressing the chimeric antigen receptor described in the present application.

In some embodiments, the cell includes an immune effector cell.

In some embodiments, the immune effector cell includes a human cell.

In some embodiments, the immune effector cell includes a T cell, a B cell, a natural killer cell (NK cell), a macrophage, a NKT cell, a monocyte, a dendritic cell, a granulocyte, a lymphocyte, a leukocyte and/or a peripheral blood mononuclear cell.

In some embodiments, the immune effector cell includes an autologous or allogeneic immune effector cell.

In some embodiments, the immune effector cell includes an allogeneic T cell or autologous T cell.

In some embodiments, the immune effector cell includes a modified immune effector cell.

In some embodiments, the expression of CD7 of the modified immune effector cell is absent or suppressed.

In some embodiments, the modified immune effector cell has reduced surface expression of CD7 compared with corresponding immune cell and express anti-CD7 CAR.

In some embodiments, the immune effector cell includes a CAR-T cell.

In some embodiments, the CAR-T cell does not induce fratricide.

On the other hand, the present application provides a method for preparing a population of chimeric antigen receptor T (CAR-T) cells, wherein the CAR targets CD7, including the following steps:
a. Modifying the CD7 gene in the population of T cells;
b. Activating the population of T cells;
c. Transducing the T cells with the chimeric antigen receptor of the present application; and
d. Expanding the chimeric antigen receptor T cell.

In some embodiments, CD7 is absent or suppressed in the population of modified T cells compared with the corresponding unmodified cells.

In some embodiments, the modification includes administering to the population of T cells one or more substances selected from the group consisting of antisense RNA, siRNA, shRNA, transcription activator-like effector nuclease (TALEN), zinc finger nuclease (ZFN) and CRISPR/Cas system.

In some embodiments, wherein the modification includes administering a CRISPR/Cas system to the population of T cells.

In some embodiments, wherein the modification includes administering a CRISPR/Cas9 system to the population of T cells.

In some embodiments, the modification includes administering Cas9 and a gRNA targeting the CD7 gene to the population of T cells.

In some embodiments, the gRNA targeting the CD7 gene comprises a nucleotide sequence shown in any one of SEQ ID NO: 211 to SEQ ID NO: 218.

In some embodiments, the Cas9 is delivered to the cell as mRNA or protein.

In some embodiments, the gRNA is delivered simultaneously with the Cas9.

In some embodiments, wherein the delivering is performed by electroporation.

In some embodiments, the transduction chimeric antigen receptor includes transducing a circRNA chimeric antigen receptor.

In some embodiments, the cirRNA chimeric antigen receptor comprises, in the following order, an internal ribosome entry site (IRES) element, a protein coding sequence targeting CD7, and polyadenylic acid (polyA).

On the other hand, the present application provides a pharmaceutical composition, comprising the isolated antigen-binding protein described herein, the polypeptide described herein, the immunoconjugate described herein, the nucleic acid molecule described herein, the expression vector described herein, the cell described herein, the chimeric antigen receptor described herein and/or the engineered cell described herein, and optionally a pharmaceutically acceptable carrier.

On the other hand, the present application provides a kit comprising the isolated antigen-binding protein described herein, the polypeptide described herein, the immunoconjugate described herein, the nucleic acid molecule described herein, the expression vector described herein, the cell described herein, the chimeric antigen receptor described herein, the engineered cell described herein, or the pharmaceutical composition described herein.

Use of the isolated antigen-binding protein described herein, the polypeptide described herein, the immunoconjugate described herein, the nucleic acid molecule described herein, the expression vector described herein, the cell described herein, the chimeric antigen receptor described herein, the engineered cell described herein, or the pharmaceutical composition described herein in the preparation of a medicament for preventing and/or treating a CD7-related disease or condition.

In some embodiments, the CD7-related disease or condition includes a tumor.

In some embodiments, the tumor includes a tumor expressing CD7.

In some embodiments, the tumor includes a hematological tumor.

In some embodiments, the tumor includes a CD7-positive hematological malignancy.

In some embodiments, the tumor includes a T-cell malignancy.

On the other hand, the present application provides a method for treating tumors, the method including administering to a subject in need thereof an effective amount of the isolated antigen-binding protein described herein, the polypeptide described herein, the immunoconjugate described herein, the nucleic acid molecule described herein, the expression vector described herein, the cell described herein, the chimeric antigen receptor described herein, the engineered cell described herein, or the pharmaceutical composition described herein.

In some embodiments, the tumor includes a tumor expressing CD7.

In some embodiments, the tumor includes a hematological tumor.

In some embodiments, the tumor includes a CD7-positive hematological malignancy.

In some embodiments, the tumor includes a T-cell malignancy.

In some embodiments, the T-cell malignancy includes acute T-lymphocytic leukemia (T-ALL), acute myeloid leukemia, or NK/T-cell lymphoma.

On the other hand, the present application provides a method for killing malignant T cells, including contacting the malignant T cell with the engineered cell described in the present application.

This application uses CRISPR/Cas9 technology to delete the expression of CD7 in T cells to prevent the occurrence of fratricide between CAR-T cells; and screens out fully human anti-CD7 scFvs from the antibody phage display library, which can help reduce the rejection reaction of the human immune system and improve the durability and therapeutic effect of CAR-T cells.

The purpose of this application is to use CRISPR/Cas9 technology to delete the expression of CD7 in T cells to prevent the occurrence of fratricide between CAR-T cells.

On the one hand, the present application provides a gRNA targeting the CD7 gene, comprising a nucleotide sequence as described in any one of SEQ ID NO: 212 to SEQ ID NO: 218, or an amino acid sequence having at least about 90%, about 91%, about 92%, about 93%, about 94%, about 95%, about 96%, about 97%, about 98%, about 99%, or about 99.5% identity with the nucleotide sequence as described in any one of SEQ ID NO: 212 to SEQ ID NO: 218.

On the other hand, the present application provides an isolated nucleic acid molecule, comprising the gRNA or a DNA molecule encoding the gRNA described in the present application.

On the other hand, the present application provides an expression vector comprising the gRNA described in the present application or the nucleic acid molecule described in the present application.

On the other hand, the present application provides a gene editing system, comprising the gRNA described in the present application, the nucleic acid molecule described in the present application, or the expression vector described in the present application.

In some embodiments, the gene editing system includes a CRISPR/Cas gene editing system.

In some embodiments, the gene editing system includes a CRISPR/Cas9 gene editing system.

In some embodiments, the gene editing system further comprises DNA encoding Cas9, mRNA encoding Cas9, or a Cas9 protein molecule.

In some embodiments, the gene editing system comprises an expression vector encoding the gRNA and Cas9 targeting the CD7 gene described in the present application.

On the other hand, the present application provides a cell, comprising the gRNA described in the present application, the nucleic acid molecule described in the present application, the expression vector described in the present application, or the gene editing system described in the present application.

In some embodiments, it includes cell expressing CD7.

In some embodiments, it includes an immune effector cell.

In some embodiments, the immune effector cell includes a T cell, a B cell, a natural killer (NK) cell, a mast cell or a phagocyte.

In some embodiments, the engineered immune effector cell targets CD7.

In some embodiments, the immune effector cell includes an engineered immune effector cell.

In some embodiments, the engineered immune effector cell includes a CAR-T cell.

In some embodiments, the engineered immune effector cell includes an anti-CD7 CAR-T cell.

In some embodiments, the CAR comprises an extracellular antigen-binding domain, the extracellular antigen-binding domain comprises a heavy chain variable region (VH) and a light chain variable region (VL), the VH can comprise HCDR1, HCDR2 and HCDR3, wherein the HCDR1 can comprise the amino acid sequence shown in SEQ ID NO: 2, the HCDR2 can comprise the amino acid sequence shown in SEQ ID NO: 10, and the HCDR3 can comprise the amino acid sequence shown in SEQ ID NO: 18; or
The HCDR1 can comprise the amino acid sequence shown in SEQ ID NO: 3, the HCDR2 can comprise the amino acid sequence shown in SEQ ID NO: 11, and the HCDR3 can comprise the amino acid sequence shown in SEQ ID NO: 19; or
The HCDR1 can comprise the amino acid sequence shown in SEQ ID NO: 2, the HCDR2 can comprise the amino acid sequence shown in SEQ ID NO: 10, and the HCDR3 can comprise the amino acid sequence shown in SEQ ID NO: 20; or
The HCDR1 can comprise the amino acid sequence shown in SEQ ID NO: 4, the HCDR2 can comprise the amino acid sequence shown in SEQ ID NO: 12, and the HCDR3 can comprise the amino acid sequence shown in SEQ ID NO: 21; or
The HCDR1 can comprise the amino acid sequence shown in SEQ ID NO: 5, the HCDR2 can comprise the amino acid sequence shown in SEQ ID NO: 13, and the HCDR3 can comprise the amino acid sequence shown in SEQ ID NO: 22; or
The HCDR1 can comprise the amino acid sequence shown in SEQ ID NO: 6, the HCDR2 can comprise the amino acid sequence shown in SEQ ID NO: 14, and the HCDR3 can comprise the amino acid sequence shown in SEQ ID NO: 23; or
The HCDR1 can comprise the amino acid sequence shown in SEQ ID NO: 2, the HCDR2 can comprise the amino acid sequence shown in SEQ ID NO: 10, and the HCDR3 can comprise the amino acid sequence shown in SEQ ID NO: 24; or
The HCDR1 can comprise the amino acid sequence shown in SEQ ID NO: 7, the HCDR2 can comprise the amino acid sequence shown in SEQ ID NO: 15, and the HCDR3 can comprise the amino acid sequence shown in SEQ ID NO: 25; or
The HCDR1 can comprise the amino acid sequence shown in SEQ ID NO: 8, the HCDR2 can comprise the amino acid sequence shown in SEQ ID NO: 16, and the HCDR3 can comprise the amino acid sequence shown in SEQ ID NO: 26; or
The HCDR1 can comprise the amino acid sequence shown in SEQ ID NO: 9, the HCDR2 can comprise the amino acid sequence shown in SEQ ID NO: 17, and the HCDR3 can comprise the amino acid sequence shown in SEQ ID NO: 27.

In some embodiments, the VH can comprise the amino acid sequence shown in SEQ ID NO: 61 to SEQ ID NO: 73.

In some embodiments, wherein the VL can comprise LCDR1, LCDR2 and LCDR3, the LCDR1 can comprise the amino acid sequence shown in SEQ ID NO: 74, the LCDR2 can comprise the amino acid sequence shown in SEQ ID NO: 87, and the LCDR3 can comprise the amino acid sequence shown in SEQ ID NO: 99; or
The LCDR1 can comprise the amino acid sequence shown in SEQ ID NO: 75, the LCDR2 can comprise the amino acid sequence shown in SEQ ID NO: 88, and the LCDR3 can comprise the amino acid sequence shown in SEQ ID NO: 100; or
The LCDR1 can comprise the amino acid sequence shown in SEQ ID NO: 76, the LCDR2 can comprise the amino acid sequence shown in SEQ ID NO: 89, and the LCDR3 can comprise the amino acid sequence shown in SEQ ID NO: 101; or
The LCDR1 can comprise the amino acid sequence shown in SEQ ID NO: 77, the LCDR2 can comprise the amino acid sequence shown in SEQ ID NO: 90, and the LCDR3 can comprise the amino acid sequence shown in SEQ ID NO: 102; or
The LCDR1 can comprise the amino acid sequence shown in SEQ ID NO: 78, the LCDR2 can comprise the amino acid sequence shown in SEQ ID NO: 91, and the LCDR3 can comprise the amino acid sequence shown in SEQ ID NO: 103; or
The LCDR1 can comprise the amino acid sequence shown in SEQ ID NO: 79, the LCDR2 can comprise the amino acid sequence shown in SEQ ID NO: 92, and the LCDR3 can comprise the amino acid sequence shown in SEQ ID NO: 104; or
The LCDR1 can comprise the amino acid sequence shown in SEQ ID NO: 80, the LCDR2 can comprise the amino acid sequence shown in SEQ ID NO: 93, and the LCDR3 can comprise the amino acid sequence shown in SEQ ID NO: 105; or
The LCDR1 can comprise the amino acid sequence shown in SEQ ID NO: 81, the LCDR2 can comprise the amino acid sequence shown in SEQ ID NO: 94, and the LCDR3 can comprise the amino acid sequence shown in SEQ ID NO: 106; or
The LCDR1 can comprise the amino acid sequence shown in SEQ ID NO: 82, the LCDR2 can comprise the amino acid sequence shown in SEQ ID NO: 95, and the LCDR3 can comprise the amino acid sequence shown in SEQ ID NO: 107; or
The LCDR1 can comprise the amino acid sequence shown in SEQ ID NO: 84, the LCDR2 can comprise the amino acid sequence shown in SEQ ID NO: 97, and the LCDR3 can comprise the amino acid sequence shown in SEQ ID NO: 109; or
The LCDR1 can comprise the amino acid sequence shown in SEQ ID NO: 85, the LCDR2 can comprise the amino acid sequence shown in SEQ ID NO: 98, and the LCDR3 can comprise the amino acid sequence shown in SEQ ID NO: 110; or
The LCDR1 can comprise the amino acid sequence shown in SEQ ID NO: 86, the LCDR2 can comprise the amino acid sequence shown in SEQ ID NO: 98, and the LCDR3 can comprise the amino acid sequence shown in SEQ ID NO: 111.

In some embodiments, the VH comprises HCDR1, HCDR2 and HCDR3, and the VL comprises LCDR1, LCDR2 and LCDR3; wherein the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 74, the LCDR2 comprises the amino acid sequence shown in SEQ ID NO: 87, and the LCDR3 comprises the amino acid sequence shown in SEQ ID NO: 99; or
The HCDR1 comprises the amino acid sequence shown in SEQ ID NO: 3, the HCDR2 comprises the amino acid sequence shown in SEQ ID NO: 11, the HCDR3 comprises the amino acid sequence shown in SEQ ID NO: 19, the LCDR1 comprises the amino acid sequence shown in SEQ ID NO: 75, the LCDR2 comprises the amino acid sequence shown in SEQ ID NO: 88, and the LCDR3 comprises the amino acid sequence shown in SEQ ID NO: 100; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 20, the LCDR1 comprises the amino acid sequence shown in SEQ ID NO: 76, the LCDR2 comprises the amino acid sequence shown in SEQ ID NO: 89, and the LCDR3 comprises the amino acid sequence shown in SEQ ID NO: 101; or
The HCDR1 comprises the amino acid sequence shown in SEQ ID NO: 4, the HCDR2 comprises the amino acid sequence shown in SEQ ID NO: 12, the HCDR3 comprises the amino acid sequence shown in SEQ ID NO: 21, the LCDR1 comprises the amino acid sequence shown in SEQ ID NO: 77, the LCDR2 comprises the amino acid sequence shown in SEQ ID NO: 90, and the LCDR3 comprises the amino acid sequence shown in SEQ ID NO: 102; or
The HCDR1 comprises the amino acid sequence shown in SEQ ID NO: 5, the HCDR2 comprises the amino acid sequence shown in SEQ ID NO: 13, the HCDR3 comprises the amino acid sequence shown in SEQ ID NO: 22, the LCDR1 comprises the amino acid sequence shown in SEQ ID NO: 91, the LCDR2 comprises the amino acid sequence shown in SEQ ID NO: 91, and the LCDR3 comprises the amino acid sequence shown in SEQ ID NO: 103; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 79, the LCDR2 comprises the amino acid sequence shown in SEQ ID NO: 92, and the LCDR3 comprises the amino acid sequence shown in SEQ ID NO: 104; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 80, the LCDR2 comprises the amino acid sequence shown in SEQ ID NO: 93, and the LCDR3 comprises the amino acid sequence shown in SEQ ID NO: 105; or
The HCDR1 comprises the amino acid sequence shown in SEQ ID NO: 6, the HCDR2 comprises the amino acid sequence shown in SEQ ID NO: 14, the HCDR3 comprises the amino acid sequence shown in SEQ ID NO: 23, the LCDR1 comprises the amino acid sequence shown in SEQ ID NO: 81, the LCDR2 comprises the amino acid sequence shown in SEQ ID NO: 94, and the LCDR3 comprises the amino acid sequence shown in SEQ ID NO: 106; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 82, the LCDR2 comprises the amino acid sequence shown in SEQ ID NO: 95, and the LCDR3 comprises the amino acid sequence shown in SEQ ID NO: 107; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 24, the LCDR1 comprises the amino acid sequence shown in SEQ ID NO: 83, the LCDR2 comprises the amino acid sequence shown in SEQ ID NO: 96, and the LCDR3 comprises the amino acid sequence shown in SEQ ID NO: 108; or
The HCDR1 comprises the amino acid sequence shown in SEQ ID NO: 7, the HCDR2 comprises the amino acid sequence shown in SEQ ID NO: 15, the HCDR3 comprises the amino acid sequence shown in SEQ ID NO: 25, the LCDR1 comprises the amino acid sequence shown in SEQ ID NO: 84, the LCDR2 comprises the amino acid sequence shown in SEQ ID NO: 97, and the LCDR3 comprises the amino acid sequence shown in SEQ ID NO: 109; or
The HCDR1 comprises the amino acid sequence shown in SEQ ID NO: 8, the HCDR2 comprises the amino acid sequence shown in SEQ ID NO: 16, the HCDR3 comprises the amino acid sequence shown in SEQ ID NO: 26, the LCDR1 comprises the amino acid sequence shown in SEQ ID NO: 85, the LCDR2 comprises the amino acid sequence shown in SEQ ID NO: 98, and the LCDR3 comprises the amino acid sequence shown in SEQ ID NO: 110; or
The HCDR1 comprises the amino acid sequence shown in SEQ ID NO: 9, the HCDR2 comprises the amino acid sequence shown in SEQ ID NO: 17, the HCDR3 comprises the amino acid sequence shown in SEQ ID NO: 27, the LCDR1 comprises the amino acid sequence shown in SEQ ID NO: 86, the LCDR2 comprises the amino acid sequence shown in SEQ ID NO: 98, and the LCDR3 comprises the amino acid sequence shown in SEQ ID NO: 111.

In some embodiments, the VL can comprise an amino acid sequence shown in any one of SEQ ID NO: 148 to SEQ ID NO: 160.

In some embodiments, the VH can comprise the amino acid sequence shown in SEQ ID NO: 61, and the VL can comprise the amino acid sequence shown in SEQ ID NO: 148; or
The VH can comprise the amino acid sequence shown in SEQ ID NO: 62, and the VL can comprise the amino acid sequence shown in SEQ ID NO: 149; or
The VH can comprise the amino acid sequence shown in SEQ ID NO: 63, and the VL can comprise the amino acid sequence shown in SEQ ID NO: 150; or
The VH can comprise the amino acid sequence shown in SEQ ID NO: 64, and the VL can comprise the amino acid sequence shown in SEQ ID NO: 151; or
The VH can comprise the amino acid sequence shown in SEQ ID NO: 65, and the VL can comprise the amino acid sequence shown in SEQ ID NO: 152; or
The VH can comprise the amino acid sequence shown in SEQ ID NO: 66, and the VL can comprise the amino acid sequence shown in SEQ ID NO: 153; or
The VH can comprise the amino acid sequence shown in SEQ ID NO: 67, and the VL can comprise the amino acid sequence shown in SEQ ID NO: 154; or
The VH can comprise the amino acid sequence shown in SEQ ID NO: 68, and the VL can comprise the amino acid sequence shown in SEQ ID NO: 155; or
The VH can comprise the amino acid sequence shown in SEQ ID NO: 69, and the VL can comprise the amino acid sequence shown in SEQ ID NO: 156; or
The VH can comprise the amino acid sequence shown in SEQ ID NO: 70, and the VL can comprise the amino acid sequence shown in SEQ ID NO: 157; or
The VH can comprise the amino acid sequence shown in SEQ ID NO: 71, and the VL can comprise the amino acid sequence shown in SEQ ID NO: 158; or
The VH can comprise the amino acid sequence shown in SEQ ID NO: 72, and the VL can comprise the amino acid sequence shown in SEQ ID NO: 159; or
The VH can comprise the amino acid sequence shown in SEQ ID NO: 73, and the VL can comprise the amino acid sequence shown in SEQ ID NO: 160.

In some embodiments, the extracellular antigen-binding domain comprises an scFv.

For example, the extracellular antigen-binding domain can be an anti-CD7 scFv.

In some embodiments, the VL and VH are connected by a linker.

In some embodiments, the linker includes a polypeptide linker.

In some embodiments, the polypeptide linker comprises an amino acid sequence represented by (GGGGS)n, wherein n is any integer from 1 to 5.

In some embodiments, the CAR-T cell of the present application comprises an extracellular domain of a chimeric antigen receptor that specifically binds to CD7. CD7 is a T cell surface membrane-associated glycoprotein. CD7 can be overexpressed in T cell malignancies including T cell acute lymphoblastic leukemia (T-ALL) and non-Hodgkin's T cell lymphoma (NHL). The CAR-T cell disclosed herein can be used to target malignant T cell that overexpresses CD7.

For example, the antigen-specific extracellular domain of the chimeric antigen receptor of the present application can specifically bind to CD7, and comprises an amino acid sequence as shown in any one of SEQ ID NO: 162 to SEQ ID NO: 174 or an amino acid sequence having at least about 90%, about 91%, about 92%, about 93%, about 94%, about 95%, about 96%, about 97%, about 98%, about 99%, or about 99.5% identity to any one of the amino acid sequences shown in SEQ ID NO: 162 to SEQ ID NO: 174.

In some embodiments, the chimeric antigen receptor includes a transmembrane domain, wherein the transmembrane domain comprising a transmembrane domain derived from one or more proteins selected from the following group: CD8, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64 and SLAM.

For example, the transmembrane domain can comprise a transmembrane domain derived from CD8.

For another example, the transmembrane domain comprises the amino acid sequence shown in SEQ ID NO: 177, or an amino acid sequence that is at least about 90%, about 91%, about 92%, about 93%, about 94%, about 95%, about 96%, about 97%, about 98%, about 99%, or about 99.5% identical to the amino acid sequence shown in SEQ ID NO: 177.

In some embodiments, the chimeric antigen receptor includes an intracellular co-stimulatory signaling domain, wherein the intracellular co-stimulatory signaling domain comprising an intracellular co-stimulatory signaling domain derived from one or more proteins selected from the group consisting of CD28, CD137, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40, and MyD88.

For example, the intracellular co-stimulatory signaling domain can comprise a co-stimulatory signaling domain derived from 4-1BB.

For another example, the intracellular co-stimulatory signaling domain can comprise the amino acid sequence shown in any one of SEQ ID NO: 178.

In some embodiments, the chimeric antigen receptor includes an intracellular signaling transduction domain, wherein the intracellular signaling transduction domain comprising an intracellular signaling transduction domain derived from one or more proteins selected from the following group: CD3ζ, CD3δ, CD3γ, CD3ε, CD79a, CD79b, FceRIγ, FceRIβ, FcγRIIa, bovine leukemia virus gp30, Epstein-Barr virus (EBV) LMP2A, simian immunodeficiency virus PBj14 Nef, DAP10, DAP-12 and a domain comprising at least one ITAM.

For example, the intracellular signaling transduction domain can comprise a signaling domain derived from CD3ζ.

For another example, the intracellular signaling transduction domain can comprise the amino acid sequence shown in SEQ ID NO: 179.

In some embodiments, it includes a hinge region between the extracellular antigen-binding domain and the transmembrane domain, wherein the hinge region comprises a hinge region derived from one or more proteins selected from the following groups: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, TIM1, SLAM, CD30 and LIGHT.

For example, the hinge region can comprise a hinge region derived from CD8.

For example, the hinge region can comprise the amino acid sequence shown in SEQ ID NO: 176.

In some embodiments, the non-targeting portion of the chimeric antigen receptor comprises a transmembrane domain, a hinge region, an intracellular co-stimulatory signaling domain, and an intracellular signaling domain.

For example, non-targeting portion of the chimeric antigen receptor comprises the transmembrane domain of the CD8 molecule, the hinge region of CD8, the intracellular co-stimulatory signaling domain of 4-1BB, and the intracellular signaling domain of CD3ζ.

In some embodiments, it further comprises a signal peptide fragment, wherein the C-terminus of the signal peptide fragment is connected to the N-terminus of the extracellular antigen-binding domain.

For example, the signal peptide fragment can comprise a CD8 signal peptide fragment.

For example, the signal peptide fragment can comprise the amino acid sequence shown in SEQ ID NO: 175.

For another example, the chimeric antigen receptor of the present application can comprise an amino acid sequence as shown in any one of SEQ ID NO: 180 to SEQ ID NO: 192, or an amino acid sequence having at least about 90%, about 91%, about 92%, about 93%, about 94%, about 95%, about 96%, about 97%, about 98%, about 99%, or about 99.5% identity with the amino acid sequence as shown in any one of SEQ ID NO: 180 to SEQ ID NO: 192.

On the other hand, the present application provides the use of the gRNA, the nucleic acid molecule, the expression vector, the gene editing system, or the cell described herein in the preparation of a medicament for treating tumors.

In some embodiments, the tumor includes a solid tumor or a hematological tumor.

In some embodiments, the tumor includes a CD7 positive tumor.

In some embodiments, the drug includes CAR-T cells.

In some embodiments, the drug includes CD7-targeting CAR-T cells.

On the other hand, the present application provides a method for gene editing of the CD7 gene in a cell, including using the gRNA described in the present application to mediate Cas9 to perform gene editing on the CD7 gene.

In some embodiments, the gene editing includes gene knockout.

On the other hand, the present application provides a method for regulating T cell function, including introducing the gRNA described in the present application, the nucleic acid molecule described in the present application, the expression vector described in the present application, or the gene editing system described in the present application into T cells.

In some embodiments, the method further includes administering a Cas enzyme to the cell.

In some embodiments, the Cas enzyme includes a Cas9 protein.

In some embodiments, wherein the Cas9 is delivered to the cell as mRNA or protein.

In some embodiments, the gRNA is delivered simultaneously with the Cas9.

In some embodiments, wherein the delivering is by electroporation.

In some embodiments, CD7 gene expression in the regulated T cells is downregulated or knocked out compared with the unregulated T cells.

In some embodiments, the method further includes modifying the specificity of the T cell by introducing a nucleic acid molecule encoding a CAR into the T cell.

In some embodiments, the nucleic acid molecule encoding CAR includes mRNA.

In some embodiments, the mRNA encodes an anti-CD7 CAR.

In some embodiments, the CAR comprises an extracellular antigen-binding domain, and the extracellular antigen-binding domain comprises the amino acid sequence shown in any one of SEQ ID NO: 162 to SEQ ID NO: 174.

In some embodiments, the anti-CD7 CAR comprises the amino acid sequence shown in any one of SEQ ID NO: 180 to SEQ ID NO: 192.

In some embodiments, the regulated T cell has reduced CD7 surface expression compared with corresponding T cell and expresses anti-CD7 CAR.

In some embodiments, the gRNA described herein, the nucleic acid molecule described herein, the expression vector described herein, the gene editing system described herein and/or the nucleic acid molecule encoding CAR are introduced into T cells by any method selected from the following: ultrasonic treatment, electric pulse, electroporation, osmotic pressure shock, calcium phosphate precipitation, DEAE dextran transfection, lipid-mediated delivery and passive delivery.

On the other hand, the present application also provides a circRNA, comprising, in the following order, an internal ribosome entry site (IRES) element, a protein-coding sequence targeting CD7, and a polyadenylation sequence.

In some embodiments, the polyA is at least 45 nucleotides in length.

In some embodiments, the polyA is at least 70 nucleotides in length.

In some embodiments, the protein targeting CD7 includes an antibody or an antigen-binding fragment thereof, a chimeric antigen receptor (CAR) and/or a T cell receptor (TCR).

In some embodiments, the binding domain of the CAR includes a CD7 scFv.

In some embodiments, the protein targeting CD7 includes an antibody including a light chain variable region comprising LCDR1, LCDR2 and LCDR3 and a heavy chain variable region comprising HCDR1, HCDR2 and HCDR3, wherein:

The HCDR1 comprises the amino acid sequence of SEQ ID NO: 2, the HCDR2 comprises the amino acid sequence of SEQ ID NO: 10, the HCDR3 comprises the amino acid sequence of SEQ ID NO: 18, the LCDR1 comprises the amino acid sequence of SEQ ID NO: 74, the LCDR2 comprises the amino acid sequence of SEQ ID NO: 87, and the LCDR3 comprises the amino acid sequence of SEQ ID NO: 99; or the HCDR1 comprises the amino acid sequence of SEQ ID NO: 3, the HCDR2 comprises the amino acid sequence of SEQ ID NO: 11, the HCDR3 comprises the amino acid sequence of SEQ ID NO: 19, the LCDR1 comprises the amino acid sequence of SEQ ID NO: 75, the LCDR2 comprises the amino acid sequence of SEQ ID NO: 88, and the LCDR3 comprises the amino acid sequence of SEQ ID NO: 100; or the HCDR1 comprises the amino acid sequence of SEQ ID NO: 2, the HCDR2 comprises the amino acid sequence of SEQ ID NO: 10, the HCDR3 comprises the amino acid sequence of SEQ ID NO: 20, and the LCDR1 comprises the amino acid sequence of SEQ ID NO: 76, the LCDR2 comprises the amino acid sequence shown in SEQ ID NO: 89, and the LCDR3 comprises the amino acid sequence shown in SEQ ID NO: 101; Or the HCDR1 comprises the amino acid sequence shown in SEQ ID NO: 4, the HCDR2 comprises the amino acid sequence shown in SEQ ID NO: 12, the HCDR3 comprises the amino acid sequence shown in SEQ ID NO: 21, the LCDR1 comprises the amino acid sequence shown in SEQ ID NO: 77, the LCDR2 comprises the amino acid sequence shown in SEQ ID NO: 90, and the LCDR3 comprises the amino acid sequence shown in SEQ ID NO: 102; or the HCDR1 comprises the amino acid sequence shown in SEQ ID NO: 5, the HCDR2 comprises the amino acid sequence shown in SEQ ID NO: 13, the HCDR3 comprises the amino acid sequence shown in SEQ ID NO: 22, the LCDR1 comprises the amino acid sequence shown in SEQ ID NO: 91, the LCDR2 comprises the amino acid sequence shown in SEQ ID NO: 91, and the LCDR3 comprises the amino acid sequence shown in SEQ ID NO: 103; or the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 79, the LCDR2 comprises the amino acid sequence shown in SEQ ID NO: 92, and the LCDR3 comprises the amino acid sequence shown in SEQ ID NO: 104; or the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 80, the LCDR2 comprises the amino acid sequence shown in SEQ ID NO: 93, and the LCDR3 comprises the amino acid sequence shown in SEQ ID NO: 105; or the HCDR1 comprises the amino acid sequence shown in SEQ ID NO: 6, the HCDR2 comprises the amino acid sequence shown in SEQ ID NO: 14, the HCDR3 comprises the amino acid sequence shown in SEQ ID NO: 23, the LCDR1 comprises the amino acid sequence shown in SEQ ID NO: 81, the LCDR2 comprises the amino acid sequence shown in SEQ ID NO: 94, and the LCDR3 comprises the amino acid sequence shown in SEQ ID NO: 106; Or the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 82, the LCDR2 comprises the amino acid sequence shown in SEQ ID NO: 95, and the LCDR3 comprises the amino acid sequence shown in SEQ ID NO: 107; or the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 24, the LCDR1 comprises the amino acid sequence shown in SEQ ID NO: 83, the LCDR2 comprises the amino acid sequence shown in SEQ ID NO: 96, and the LCDR3 comprises the amino acid sequence shown in SEQ ID NO: 108; or the HCDR1 comprises the amino acid sequence shown in SEQ ID NO: wherein the HCDR1 comprises the amino acid sequence of SEQ ID NO: 8, the HCDR2 comprises the amino acid sequence of SEQ ID NO: 16, the HCDR3 comprises the amino acid sequence of SEQ ID NO: 26, the LCDR1 comprises the amino acid sequence of SEQ ID NO: 85, the LCDR2 comprises the amino acid sequence of SEQ ID NO: 98, and the LCDR3 comprises the amino acid sequence of SEQ ID NO: 110; or the HCDR1 comprises the amino acid sequence of SEQ ID NO: 9, the HCDR2 comprises the amino acid sequence of SEQ ID NO: 17, the HCDR3 comprises the amino acid sequence of SEQ ID NO: 27, and the LCDR1 comprises the amino acid sequence of SEQ ID NO: The LCDR2 comprises the amino acid sequence shown in SEQ ID NO: 98, and the LCDR3 comprises the amino acid sequence shown in SEQ ID NO: 111.

In some embodiments, the protein targeting CD7 includes an antibody, the antibody including VH and VL, wherein the VH comprises the amino acid sequence shown in SEQ ID NO: 61, and the VL comprises the amino acid sequence shown in SEQ ID NO: 148; or the VH comprises the amino acid sequence shown in SEQ ID NO: 62, and the VL comprises the amino acid sequence shown in SEQ ID NO: 149; or the VH comprises the amino acid sequence shown in SEQ ID NO: 63, and the VL comprises the amino acid sequence shown in SEQ ID NO: 150; or the VH comprises the amino acid sequence shown in SEQ ID NO: 64, and the VL comprises the amino acid sequence shown in SEQ ID NO: 151; or the VH comprises the amino acid sequence shown in SEQ ID NO: 65, and the VL comprises the amino acid sequence shown in SEQ ID NO: 152; or the VH comprises the amino acid sequence shown in SEQ ID NO: 66, and the VL comprises the amino acid sequence shown in SEQ ID NO: 153; or the VH comprises the amino acid sequence shown in SEQ ID NO: 67, and the VL comprises the amino acid sequence shown in SEQ ID NO: 154; or the VH comprises SEQ or the VH comprises the amino acid sequence shown in SEQ ID NO: 73, and the VL comprises the amino acid sequence shown in SEQ ID NO: 160.

In some embodiments, the circRNA comprises a nucleotide sequence shown in any one of SEQ ID NOs: 256 to 257.

On the other hand, the present application also provides an agent for regulating the expression level and/or activity of CD7, comprising the circRNA described in the present application.

On the other hand, the present application also provides the application of circRNA of the present application in regulating the expression level and/or activity of CD7.

Those skilled in the art can easily perceive other aspects and advantages of the present application from the detailed description below. In the detailed description below, only exemplary embodiments of the present application are shown and described. As will be appreciated by those skilled in the art, the content of the present application enables those skilled in the art to modify the disclosed specific embodiments without departing from the spirit and scope of the invention to which the present application relates. Accordingly, the description in the drawings and specification of the present application is merely exemplary and not restrictive.

### BRIEF DESCRIPTION OF THE DRAWINGS

The specific features of the invention involved in the present application are shown in the attached claims. The features and advantages of the invention of involved in the present application can be better understood by referring to the exemplary embodiments and drawings described in detail below. The drawings are briefly described as follows:

FIG. 1 shows the readout of the anti-human CD7-Fc monoclonal phage ELISA in three 96-well plates.

FIG. 2 shows a schematic diagram of the pDA-CAR vector used to generate anti-CD7 CAR mRNA.

FIG. 3 shows the results of FACS staining of T cells electroporated with different CD7 gRNAs using anti-CD7 antibodies.

FIG. 4 shows the results of FACS staining of different anti-CD7 scFv CAR-T cells using CD7-Fc protein.

FIG. 5 shows the results of FACS staining of A549 cells electroporated with different amounts of CD7 mRNA using anti-CD7 antibody.

FIGS. 6 to 8 show the killing curves of mRNA-based anti-CD7 CAR-T cells (H1-H18) against A549-GFP tumor cells at an E/T ratio of 3:1.

FIG. 9 shows the killing curves of 5 anti-CD7 CAR-T cells (H1, H7, H9, H10, H17) against A549-GFP tumor cells electroporated with 0 or 2 ug CD7 mRNA at an E/T ratio of 3:1.

FIG. 10 shows the killing curve of mRNA-based anti-CD7 CAR (H1, H7, H9, H10, H17)+A40C28 T cells against A549-GFP tumor cells electroporated with 0 or 2 ug CD7 mRNA at an E/T ratio of 3:1.

FIG. 11 shows the CD107a staining results of anti-CD7 CAR-T cells (A40C28+/−) with different mRNAs in co-culture and killing assays with A549-GFP tumor cells electroporated with 0 or 2 ug CD7 mRNA.

FIG. 12 shows the results of FACS staining of 17 different tumor cell lines using isotype control and anti-CD7 mAb.

FIGS. 13 to 14 show the CD107a staining results of anti-CD7 CAR-T cells (A40C28+/−) with different mRNAs in co-culture and killing assays with different tumor cell lines.

FIG. 15 shows a schematic diagram of the LACO-Stim molecule of the present application.

FIG. 16 shows the results of examining the expression of CAR/LACO-stim by flow cytometry.

FIG. 17 shows tumor growth after co-culture with T cells co-expressing CAR and LACO-Stim as shown in Table 3. The E/T ratio is 30:1. E: effector cells (T cells); T: target cells (A549-ESO cells).

FIG. 18 shows tumor growth after co-culture with T cells co-expressing TCR and LACO-Stim as shown in Table 1. The E/T ratio is 30:1. E: effector cells (T cells); T: target cells (A549-ESO cells).

FIG. 19 shows the structure of the circRNA described in the present application.

FIGS. 20A-20B show the effects of the circRNA described in the present application on target cells

### DETAILED DESCRIPTION OF THE PREFERRED EMBODIMENT

The following is an explanation of the implementation of the present invention of specific embodiments. Those skilled in the art can easily understand other advantages and effects of the present invention from the contents disclosed in this specification.

### Definition of Terms

In the present application, the term “antigen-binding protein” generally refers to a protein comprising a portion that binds an antigen, and optionally a scaffold or framework portion that allows the portion that binds the antigen to adopt a conformation that promotes the binding of the antigen-binding protein to the antigen. Examples of antigen-binding proteins include, but are not limited to, antibodies, antigen-binding fragments (e.g., Fab, Fab′, F(ab)₂, Fv fragments, F(ab′)2, scFv, di-scFv and/or dAb), immunoconjugates, multispecific antibodies (e.g., bispecific antibodies), antibody fragments, antibody derivatives, antibody analogs or fusion proteins, etc., as long as they exhibit the desired antigen-binding activity.

In the present application, the term “antibody” is generally used in the broadest sense, and specifically covers monoclonal antibodies, polyclonal antibodies, dimers, multimers, multispecific antibodies (e.g., bispecific antibodies), and antibody fragments, as long as they show the desired biological activity (Miller et al (2003) Jour. of Immunology 170: 4854-4861). Antibodies can be mouse, human, humanized, chimeric antibodies, or derived from other species. Without limitation, an “antibody” typically can comprise a protein of at least two heavy chains (HC) and two light chains (LC) interconnected by disulfide bonds, or an antigen-binding fragment thereof. Each heavy chain comprises a heavy chain variable region (VH) and a heavy chain constant region. In some naturally occurring IgG, IgD and IgA antibodies, the heavy chain constant region comprises three domains, CH1, CH2 and CH3. In some naturally occurring antibodies, each light chain comprises a light chain variable region (VL) and a light chain constant region. The light chain constant region comprises one domain, CL. The VH and VL regions can be further subdivided into regions of hypervariability, called complementarity determining regions (CDRs), which alternate with more conserved regions called framework regions (FRs). Each VH and VL comprises three CDRs and four framework regions (FRs), arranged in the following order from the amino terminus to the carboxyl terminus: FR1, CDR1, FR2, CDR2, FR3, CDR3, and FR4. The variable domains of natural heavy chains and light chains each comprise four framework regions (HFR1, HFR2, HFR3, HFR4, LFR1, LFR2, LFR3, LFR4), mostly adopting a 3-sheet configuration. These regions are connected by three CDRs, forming loops, and in some cases, they form part of the 3-sheet structure. The CDRs in each chain are closely together through the FR region and together with the CDRs from the other chain form the antigen-binding site of the antibody. The constant region of the antibody can mediate the binding of immunoglobulins to host tissues or factors, including various cells of the immune system (e.g., effector cells) and the first component (Clq) of the classical complement system.

When describing the positional relationship of amino acid fragments in an antibody sequence, the term “located between . . . ” generally means that the C-terminus of a certain amino acid fragment is directly or indirectly connected to the N-terminus of the first amino acid fragment, and its N-terminus is directly or indirectly connected to the C-terminus of the second amino acid fragment. In the light chain, for example, the N-terminus of LFR2 is directly or indirectly connected to the C-terminus of LCDR1, and the C-terminus of LFR2 is directly or indirectly connected to the N-terminus of LCDR2. For another example, the N-terminus of LFR3 is directly or indirectly connected to the C-terminus of LCDR2, and the C-terminus of LFR3 is directly or indirectly connected to the N-terminus of LCDR3. In the heavy chain, for example, the N-terminus of HFR2 is directly or indirectly connected to the C-terminus of HCDR1, and the C-terminus of HFR2 is directly or indirectly connected to the N-terminus of HCDR2. For another example, the N-terminus of HFR3 is directly or indirectly connected to the C-terminus of HCDR2, and the C-terminus of HFR3 is directly or indirectly connected to the N-terminus of HCDR3.

In the present application, the term “antigen-binding fragment” generally refers to a part of an antibody molecule, comprising amino acids responsible for the specific binding between the antibody and the antigen. Antigen-binding fragments can include: Fab, Fab′, F(ab)₂ fragments, Fv fragments, dsFv, F(ab′)2, scFv, di-scFv, dAb fragments. The term “Fab” generally refers to a fragment that contains the Fv region, the constant domain of the light chain, and the first constant domain of the heavy chain (CH1). Fab′ differs from Fab in that several residues are added to the carboxyl terminus of the heavy chain CH1 domain, including one or more cysteines from the hinge region of the antibody. The term “Fv fragment” generally refers to an antibody fragment comprising of the VL and VH domains of a single arm of an antibody, and in some cases, the fragment can consist of a VH and a VL dimer in tight non-covalent binding. The term “(Fab)₂” generally refers to a bivalent fragment containing a hinge region and a variable region of a heavy chain, a light chain, and the first constant region. The term “F(ab′)2” generally refers to an antibody fragment comprising two Fab′ fragments connected by disulfide bonds in the hinge region. The term “scFv fragment” generally comprises the VH and VL domains of an antibody, wherein these domains are present as a single polypeptide chain. Typically, the scFv polypeptide further comprises a polypeptide linker between the VH and VL domains, which enables the scFv to form a desired structure for antigen-binding. The term “dAb fragment” generally refers to an antibody fragment consisting of a VH domain. The term “dsFv” generally refers to a disulfide-stabilized Fv fragment, in which the bond between a single light chain variable region and a single heavy chain variable region is a disulfide bond.

In the present application, the term “variable” generally refers to the fact that some parts of the sequence of the variable domain of an antibody vary strongly, which forms the binding and specificity of various specific antibodies to their specific antigens. However, variability is not evenly distributed throughout the variable region of an antibody. It is concentrated in three segments in the light chain and heavy chain variable regions, referred to as complementary determining regions (CDRs) or hypervariable regions (HVRs). The more highly conserved parts within the variable region are called frameworks (FR). In the field, antibodies' CDRs can be defined by various methods, such as the Kabat definition based on sequence variability (see Kabat et al., Sequences of Proteins of Immunological Interest, Fifth Edition, NIH, Bethesda, MD (1991)), the Chothia definition based on the location of structural loop regions (see Al-Lazikani et al., J Mol Biol 273:927-48, 1997), and the KABAT definition based on IMGT-ONTOLOGY concepts and IMGT Scientific chart rules. The method used herein can utilize CDRs defined according to any of these systems. In some embodiments, CDRs defined by Kabat or Chothia are used, or CDRs of scFv antibodies can be delineated by referring to http://abysis.org/.

In the present application, the term “isolated” antigen-binding protein typically refers to an antigen-binding protein that has been identified, separated, and/or recovered from its production environment (e.g., natural or recombinant) components. Contaminant components from the production environment are generally substances that would interfere with the protein's research, diagnostic, or therapeutic uses and can include enzymes, hormones, and other protein or non-protein solutes. An isolated antigen-binding protein or antibody is typically prepared through at least one purification step.

In the present application, the term “monoclonal antibody” generally refers to an antibody obtained from a group of substantially homogeneous antibodies, meaning that the individual antibodies in the group are identical, except for possible minor naturally occurring mutations. Monoclonal antibodies are usually highly specific for a single antigenic site. Unlike conventional polyclonal antibody preparations, which typically include different antibodies directed against various epitopes, each monoclonal antibody is directed against a single epitope on the antigen. In addition to their specificity, monoclonal antibodies have the advantage of being synthetically produced by hybridoma cultures without contamination by other immunoglobulins. The modifier “monoclonal” denotes the characteristic of antibodies obtained from a substantially homogeneous group and is not construed to require production by any particular method. For instance, monoclonal antibodies used in the present application can be prepared in hybridoma cells or via recombinant DNA methods.

In the present application, the term “humanized antibody” generally refers to an antibody in which some or all of the amino acids outside the CDR regions of a non-human antibody (e.g., a mouse antibody) have been replaced with corresponding human immunoglobulin amino acids. In the CDR regions, small additions, deletions, insertions, substitutions, or modifications of amino acids are also permitted as long as they retain the antibody's ability to bind the specific antigen. Humanized antibodies can optionally comprise at least a portion of the constant region of human immunoglobulin. A “humanized antibody” retains the antigen specificity of the original antibody. The “humanized” form of non-human (e.g., murine) antibodies may minimally comprise a chimeric antibody that includes sequences derived from non-human immunoglobulins. In certain cases, CDR residues from a human immunoglobulin (receptor antibody) can be replaced with CDR residues from a non-human species (donor antibody) such as a mouse, rat, rabbit, or non-human primate, possessing desired properties, affinity, and/or capability. In some cases, framework region (FR) residues of a human immunoglobulin can be replaced with corresponding non-human residues. Additionally, humanized antibodies can comprise amino acid modifications that are not present in either the recipient or donor antibodies. These modifications can be made to further improve the performance of the antibody, such as binding affinity.

In the present application, the term “fully human antibody” generally refers to antibodies expressed by transferring human antibody-encoding genes into genetically engineered antibody gene-deficient animals. All parts of the antibody (including the variable and constant regions of the antibody) are encoded by genes of human origin. Fully human antibodies can greatly reduce the immune side effects caused by heterologous antibodies to the human body. Methods for obtaining fully human antibodies in this field include phage display technology, transgenic mouse technology, ribosome display technology, RNA polypeptide technology, etc.

In the present application, the term “scFv” generally refers to a fusion protein comprising at least one antibody fragment including a variable region of a light chain and at least one antibody fragment including a variable region of a heavy chain, wherein the light chain and heavy chain variable regions are adjacent (e.g., via a synthetic linker such as a short flexible polypeptide linker) and can be expressed in a single-chain polypeptide form, and wherein the scFv retains the specificity of the full-length antibody from which it is derived. Unless otherwise specified, as used herein, scFv can have the VL and VH variable regions in any order (e.g., relative to the N-terminus and C-terminus of the polypeptide), and scFv can include VL-linker-VH or can include VH-linker-VL.

In the present application, the term “specificity” generally refers to the number of different types of antigens or antigenic determinants to which a particular immunoglobulin sequence, antigen-binding molecule or antigen-binding protein (e.g., immunoglobulin single variable domain, or polypeptide of the present invention) can bind. The specificity of an antigen-binding protein can be determined of based on affinity and/or avidity. The terms “specific” and “specifically” can be used interchangeably to indicate that biological molecules other than CD7 do not significantly bind to the antibody.

In the present application, the term “affinity” generally refers to a measure of the binding constant of a single monovalent ligand for its associated binding partner, for example, Fab′ for the binding of an antigen or epitope. Affinity can be measured in several ways, including by, for example, plasmon resonance (BiaCore) measuring the binding and dissociation rates (kon and koff, respectively), and expressed as overall binding (Kass) or dissociation constant (KD), where Kass is kon/koff, and KD is koff/kon. KD can also be measured empirically, for example, by measuring the concentration at which the binding of the ligand to the binding partner is half saturated. Another method of measuring KD is by competitive assay, in which a binder or ligand is labeled or tagged, and maintained at a constant concentration, and a test binder or ligand is added at various concentrations to compete away the labeled substance from its associated binding partner, and the concentration at which the label is reduced by half is determined.

In the present application, the terms “KD” and “K_{D}” are used interchangeably and generally refer to the equilibrium dissociation constant. “KD” is the ratio of the dissociation rate constant (kdis, also known as “dissociation rate (off-rate) (koff)” or “kd”) to the association rate constant (kon, also known as “association rate (kon)” or “ka”). The binding affinity of an antigen-binding protein (e.g., an antibody) for an antigen can be expressed using the association rate constant (kon), the dissociation rate constant (kdis), and the equilibrium dissociation constant (KD). Methods for determining the association and dissociation rate constants are well known in the art and include, but are not limited to, biolayer interferometry (BLI), radioimmunoassay (RIA), equilibrium dialysis, surface plasmon resonance (SPR), fluorescence resonance energy transfer (FRET), co-immunoprecipitation (Co-IP), and protein microarray technology. If measured under different conditions (e.g., salt concentration, pH), the affinity of a particular protein-protein interaction can vary.

In the present application, the term “reference antibody” generally refers to an antibody with which the antigen-binding protein described in the present application competes for antigen-binding.

When used in the context of antigen-binding proteins competing for the same epitope, the term “compete” generally refers to competition between antigen-binding proteins, as determined by assays in which the test antigen-binding protein (e.g., an antibody or its immunologically functional fragment) prevents or inhibits (e.g., reduces) the specific binding of a reference antigen-binding protein (e.g., ligand or reference antibody) to a common antigen (e.g., CD7 or a fragment thereof). Various types of competitive binding assays can be used to determine whether one antigen-binding protein competes with another, such as solid-phase direct or indirect radioimmunoassays (RIAs), solid-phase direct or indirect enzyme immunoassays (EIA), sandwich competition assays (see, e.g., Stahli et al., 1983, Methods in Enzymology 9: 242-253); solid-phase direct biotin-avidin EIA (see, e.g., Kirkland et al., 1986, J. Immunol. 137: 3614-3619); solid-phase direct labeled assays; solid-phase direct labeled sandwich assays (see, e.g., Harlow and Lane, 1988, Antibodies, A Laboratory Manual, Cold Spring Harbor Press); solid-phase direct labeled RIA using I-125 (see, e.g., Morel et al., 1988, Molec. Immunol. 25: 7-15); solid-phase direct biotin-avidin EIA (see, e.g., Cheung et al., 1990, Virology 176: 546-552); and direct labeled RIA (Moldenhauer et al., 1990, Scand. J. Immunol. 32: 77-82). Typically, such assays involve the use of purified antigen or cells bearing the antigen bound to a solid surface, an unlabeled test antigen-binding protein, and a labeled reference antigen-binding protein. Competitive inhibition is measured by determining the amount of label bound to the solid surface or cells in the presence of the test antigen-binding protein. Generally, the test antigen-binding protein is present in excess. Antigen-binding proteins identified through competition assays (competing antigen-binding proteins) include those that bind the same epitope as the reference antigen-binding protein and those that bind an epitope sufficiently close to the reference antigen-binding protein's epitope to cause steric hindrance. Typically, when the competing antigen-binding protein is present in excess, it inhibits (e.g., reduces) the specific binding of the reference antigen-binding protein to the common antigen by at least about 40-45%, about 45-50%, about 50-55%, about 55-60%, about 60-65%, about 65-70%, about 70-75%, or about 75% or more. In some cases, binding is inhibited by at least about 80-85%, about 85-90%, about 90-95%, about 95-97%, or about 97% or more.

The term “Fc” generally refers to a polypeptide comprising at least a portion of the CH3, CH2, and hinge regions of the antibody constant domain. Optionally, the Fc region can comprise the CH4 domain present in some antibody classes. “Fc” can encompass: natural monomers, natural dimers (disulfide-linked), modified dimers (disulfide and/or non-covalently linked), and modified monomers (i.e., derivatives). Exemplary modifications include the addition, deletion, or substitution of one or more amino acids in one or more domains. Such modifications can be included to optimize effector functions, half-life, and other properties.

In the present application, the term “isolated” antigen-binding protein generally refers to an antigen-binding protein that has been identified, separated and/or recovered from components of its production environment (e.g., natural or recombinant). Contaminating components of its production environment are generally substances that interfere with its research, diagnostic or therapeutic use, and can include enzymes, hormones and other protein or non-protein solutes. An isolated antigen-binding protein or antibody will generally be prepared by at least one purification step.

In the present application, the term “percent identity” or “identity” generally refers to the degree to which two or more nucleic acids or polypeptide sequences are identical. In the context of amino acid sequences, the term “percent identity” or “identity” generally describes the matching (“hits”) number of amino acids that are consistent between two or more compared amino acid sequences and the number of amino acid residues that make up the total length of these amino acid sequences. In other words, using comparison, for two or more sequences, when these sequences are compared and aligned against the maximum correspondence (as measured using sequence comparison algorithms known in the art), or when manually aligned and visually inspected, the percentage of identical amino acid residues (e.g., 65%, 70%, 75%, 80%, 85%, 90%, 95% or 99% identity) can be determined. Therefore, the sequences that are compared with determine sequence identity can be distinguished by one or more amino acid substitutions, additions or deletions. Suitable programs for aligning protein sequences are known to those skilled in the art. The percent sequence identity of a protein sequence can be determined, for example, using programs such as CLUSTALW, Clustal Omega, FASTA or BLAST, for example using the NCBI BLAST algorithm (Altschul S F et al. (1997), Nucleic Acids Res. 25:3389-3402).

In the present application, the term “immunoconjugate” or “antibody conjugate” generally refers to the connection of an antibody or its antibody fragment to other active agents, such as chemotherapeutic agents, cytotoxins (cytotoxic agents), immunotherapeutic agents, imaging probes, spectroscopic probes, and the like. The connection can be a covalent bond, or a non-covalent interaction, for example, by electrostatic forces. A variety of linkers known in the art can be used to form immunoconjugates. In addition, the immunoconjugate can be provided in the form of a fusion protein, which can be expressed from a polynucleotide encoding the immunoconjugate. The term “fusion protein” generally refers to a protein produced by connecting two or more genes or gene fragments that originally encode independent proteins (including peptides and polypeptides). Translation of the fusion gene produces a single protein with functional properties from each original protein. The term “cytotoxin” or “cytotoxic agent” can include any agent that is harmful to (e.g., kills) cells.

In the present application, the term “chimeric antigen receptor” or “CAR” generally refers to a group of polypeptides, usually two in the simplest embodiment, which, when in an immune effector cell, provide the cell with specificity for a target cell (usually a cancer cell) and generate an intracellular signal. In some embodiments, CAR comprises at least one extracellular antigen-binding domain (such as VHH, scFv or a portion thereof), a transmembrane domain and a cytoplasmic signaling domain (also referred to herein as an “intracellular signaling domain”), comprising a functional signaling domain derived from a stimulatory molecule and/or a co-stimulatory molecule as defined below. In some embodiments, the group of polypeptides is in the same polypeptide chain (e.g., comprising a chimeric fusion protein). In some embodiments, the group of polypeptides is discontinuous with each other, for example, in different polypeptide chains. In some aspects, the group of polypeptides includes a dimerization switch, which can couple polypeptides to each other in the presence of a dimerization molecule, for example, the antigen-binding domain can be coupled to the intracellular signaling domain. On the one hand, the stimulatory molecule of CAR is a ζ chain associated with a T cell receptor complex. On the one hand, the cytoplasmic signaling domain comprises a primary signaling domain (e.g., a primary signaling domain of CD3-ζ). On the one hand, the cytoplasmic signaling domain also comprises one or more functional signaling domains derived from at least one co-stimulatory molecule defined as follows. On the one hand, the co-stimulatory molecule can be selected from 4-1BB (i.e., CD137), CD27, ICOS and/or CD28. On the one hand, CAR comprises a chimeric fusion protein, which can comprise an extracellular antigen recognition domain, a transmembrane domain, and an intracellular signaling domain comprising a functional signaling domain derived from a stimulatory molecule. On the one hand, CAR comprises a chimeric fusion protein, which can comprise an extracellular antigen recognition domain, a transmembrane domain, and an intracellular signaling domain, and the intracellular signaling domain comprises a functional signaling domain derived from a co-stimulatory molecule and a functional signaling domain derived from a stimulatory molecule. On the one hand, CAR includes a chimeric fusion protein, which can comprise an extracellular antigen recognition domain, a transmembrane domain and an intracellular signaling domain, and the intracellular signaling domain comprises a functional signaling domain derived from one or more co-stimulatory molecules and a functional signaling domain derived from a stimulatory molecule. On the one hand, CAR comprises a chimeric fusion protein, which can comprise an extracellular antigen recognition domain, a transmembrane domain and an intracellular signaling domain, and the intracellular signaling domain comprises at least two functional signaling domains derived from one or more co-stimulatory molecules and a functional signaling domain derived from a stimulatory molecule. On the one hand, CAR comprises an optional leader sequence on the amino terminus (N-ter) of the CAR fusion protein. On the one hand, CAR is further comprised in a leader sequence at the N-terminus of the extracellular antigen recognition domain, wherein the leader sequence is optionally removed from the antigen recognition domain (e.g., VHH) during cell processing, and CAR is positioned on the cell membrane.

In the present application, the term “isolated nucleic acid molecule” or “isolated polynucleotide” generally refers to DNA or RNA of genomic, mRNA, cDNA or synthetic origin, or some combination thereof, which is not associated with all or a portion of a polynucleotide found in nature, or connected to a polynucleotide to which it is not connected in nature.

In the present application, the term “vector” generally refers to a nucleic acid molecule that can self-replicate in a suitable host, which transfers the inserted nucleic acid molecule into and/or between host cells. The vector can include a vector that is mainly used to insert DNA or RNA into a cell, a vector that is mainly used to replicate DNA or RNA, and a vector that is mainly used to express the transcription and/or translation of DNA or RNA. The vector also includes a vector with a variety of the above functions. The vector can be a polynucleotide that can be transcribed and translated into a polypeptide when introduced into a suitable host cell. Typically, the vector can produce a desired expression product by cultivating a suitable host cell that contains the vector.

In the present application, the term “cell” generally refers to an individual cell, cell line or cell culture that can or has contained a plasmid or vector including a nucleic acid molecule described in the present application, or that can express an antibody or antigen-binding fragment thereof, a polypeptide or an immunoconjugate described in the present application antigen-binding. The cell can include the offspring of a single host cell. Due to natural, accidental or deliberate mutations, the offspring cell can not be completely identical to the original parent cell in morphology or genome, but it is sufficient that the antibody or antigen-binding fragment thereof described in the present application can be expressed. The cell can be obtained by transfecting the cell in vitro using the vector described in the present application. The cell can be a prokaryotic cell (e.g., Escherichia coli) or a eukaryotic cell (e.g., a yeast cell, such as a COS cell, a Chinese hamster ovary (CHO) cell, a HeLa cell, a HEK293 cell, a COS-1 cell, a NS0 cell or a myeloma cell). In some cases, the cell can be a mammalian cell. For example, the mammalian cell can be a CHO-K1 cell. In the present application, the term “recombinant cell” generally refers to a cell into which a recombinant expression vector has been introduced. The recombinant host cell includes not only a certain specific cell, but also the offspring of these cells.

In the present application, the term “T cell” or “T lymphocyte” can be any T cell, such as a cultured T cell, such as a primary T cell, or a T cell from a cultured T cell line, such as Jurkat, SupT I, etc., or a T cell obtained from a mammal (preferably a primate, species, including monkeys, dogs or humans). If obtained from a mammal, the T cell can be obtained from many sources, including but not limited to blood, bone marrow, lymph nodes, thymus or other tissues or fluids. T cells can also be enriched or transformed. T cells can be obtained by maturing hematopoietic stem cells into T cells in vitro or in vivo. In an exemplary aspect, the T cell is a human T cell. In an exemplary aspect, the T cell is a T cell isolated from a human. T cells can be any type of T cells, including NKT cells, and can have any developmental stage, including but not limited to CD4+/CD8+ double positive T cells; CDA+helper T cells; such as Th1 and Th2 cells, CD8+ T cells (such as cytotoxic T cells); peripheral blood mononuclear cells (PBMC); peripheral blood leukocytes (PBL); tumor infiltrating cells (TIL); memory T cells; untreated T cells, etc. Preferably, the T cells are CD8+ T cells or CD4+ T cells. In some alternatives, the T cells are allogeneic (from different donors of the same species) to the recipient of the cells or the cells to be received (such as the cells are in the form of a therapeutic composition); in some alternatives, the T cells are autologous (the donor and the recipient are the same); in some alternatives, the T cells are syngeneic (the donor and the recipient are different, but they are identical twins).

In the present application, the term “immune effector cell” generally refers to an immune cell that participates in an immune response and performs an effector function. For example, the effector function can include removing foreign antigens or promoting immune effector responses. Immune effector cells can include plasma cells, T cells, B cells, natural killer (NK) cells, natural killer T (NKT) cells, mast cells, and bone marrow-derived phagocytes.

The immune effector cells of the present application can be autologous/autogeneic (“one's own”) or non-autologous (“non-one's own”, such as allogeneic, isogenic or allogeneic). In the present application, the term “autologous” generally refers to cells from the same subject. “Allogenic” generally refers to cells of the same species but genetically different from the compared cells. “Isogenic” generally refers to cells of different subjects that are genetically identical to the compared cells. “Allogenic” generally refers to cells of a different species from the compared cells. In some embodiments, the cells of the present application are autologous or allogenic.

In the present application, the term “modification” generally refers to changing the state or structure of a cell and/or a change in the state or structure of a cell. The change is usually compared with the state or structure of a corresponding cell without the modification, and the change can include a change in the expression level or function of an endogenous gene, such as downregulating, upregulating or not expressing the expression level of an endogenous gene in a cell by genetic engineering means, and the genetic engineering means can include homologous recombination, CRISPR/Cas9 system gene editing, etc.; the change can also include a change in the expression, structure or function of a cell protein, such as a change in the expression level or function of a corresponding protein achieved by a change in the expression level or function of the endogenous gene, such as a change in the expression, structure or function of a protein achieved by regulating protein translation or post-translational modification; the change can also include the introduction of exogenous genes, the expression of exogenous proteins, etc.

In the present application, the term “CRISPR/Cas system” generally refers to a group of molecules comprising RNA-guided nucleases or other effector molecules and gRNA molecules, which can guide and enable the modification of nucleic acids at target sequences by RNA-guided nucleases or other effector molecules, such as causing target sequence degradation. In some embodiments, the CRISPR system comprises gRNA and Cas proteins, for example, Cas9 proteins. Systems comprising Cas9 or its functional mutants are referred to as “Cas9 systems” or “CRISPR/Cas9 systems” In the present application. In some embodiments, gRNA molecules and Cas molecules can be compounded to form ribonucleoprotein (RNP) complexes.

In the present application, the terms “gRNA molecule”, “guide RNA”, “guide RNA molecule” or “gRNA” are used interchangeably and generally refer to a nucleic acid molecule that can promote a specific guide RNA-guided nuclease or other effector molecule (generally complexed with a gRNA molecule) to a target sequence. In some embodiments, a portion of the gRNA hybridizes with the DNA (e.g., through the gRNA guide domain) and a portion of the gRNA molecule binds to the RNA-guided nuclease or other effector molecule (e.g., at least through the gRNA In some embodiments, the gRNA molecule consists of a single continuous polynucleotide molecule, referred to herein as “single guide RNA” or “sgRNA”, etc. In other embodiments, the gRNA molecule consists of multiple (e.g., two) polynucleotide molecules that can associate (generally by hybridization), referred to herein as “dual guide RNA” or “dgRNA”, etc.

In the present application, the term “Cas protein” generally refers to the enzyme responsible for cutting DNA in the CRISPR/Cas system. It can include enzymes from type I, II, and III CRISPR/Cas systems. For example, Cas3, Cas9, Cas10.

In the present application, the term “Cas9 protein” generally refers to an enzyme from a bacterial type II CRISPR/Cas system that is responsible for cutting DNA. Cas9 can include wild-type proteins and functional mutants thereof.

In the present application, the terms “reduce” and “downregulate” are used interchangeably and generally refer to any change that is less than the original. “Reduce” and “downregulate” are relative terms and require a comparison between before and after the measurement. “Reduce” and “downregulate” include complete depletion.

In the present application, the term “downregulation” can be detected by standard methods known in the art (such as those of the present application), and the expression level/amount of a gene, gene product, such as a protein or a biomarker in a first sample is compared with the expression level/amount of a corresponding gene, gene product, such as a protein or a biomarker in a second sample by about 5%, 10%, 20%, 25%, 30%, 40%, 50%, 60%, 70%, 80%, 85%, 90%, 95%, or 100% overall downregulation. In some embodiments, the term “downregulation” refers to the downregulation in the expression level/amount of a gene or biomarker in a first sample, wherein the downregulation is at least about 0.9 times, 0.8 times, 0.7 times, 0.6 times, 0.5 times, 0.4 times, 0.3 times, 0.2 times, 0.1 times, 0.05 times, or 0.01 times of the expression level/amount of the corresponding gene or biomarker in the second sample. In some embodiments, the first sample is a sample obtained from a subject, and the second sample is a reference sample.

In the present application, the term “pharmaceutically acceptable carrier” generally refers to one or more non-toxic substances that do not interfere with the effectiveness of the biological activity of the active ingredient. Such preparations can generally contain salts, buffers, preservatives, compatible carriers, adjuvants and optional other therapeutic agents. Such pharmaceutically acceptable preparations can also generally contain compatible solid or liquid fillers, diluents or encapsulating materials suitable for administration to humans. Non-limiting, pharmaceutically acceptable carriers can include liquids, such as water, saline, glycerol and ethanol. Auxiliary substances, such as wetting agents or emulsifiers, pH buffer substances, etc., can also be present in these carriers. The term “adjuvant” generally refers to any substance that assists or regulates the action of a drug, including but not limited to immunological adjuvants, which enhance or diversify the immune response to an antigen.

In the present application, the term “prevention and/or treatment” includes not only prevention and/or treatment of diseases, but also generally includes prevention of the onset of diseases, slowing or reversing the progression of diseases, preventing or slowing the onset of one or more symptoms associated with diseases, reducing and/or alleviating one or more symptoms associated with diseases, reducing the severity and/or duration of diseases and/or any symptoms associated therewith, and/or preventing further increase in the severity of diseases and/or any symptoms associated therewith, preventing, reducing or reversing any physiological damage caused by diseases, and any pharmacological effects that are generally beneficial to patients being treated. The compositions of the present application form viable therapeutic agents that do not require complete cure or eradication of any symptoms or manifestations of the disease. As recognized in the relevant art, drugs used as therapeutic agents can reduce the severity of a given disease state, but do not need to eliminate every manifestation of the disease to be considered a useful therapeutic agent. Similarly, prophylactically administered treatments constitute viable preventive agents that do not need to be completely effective in preventing the onset of the disease. It is sufficient to simply reduce the impact of the disease in the subject (e.g., by reducing the number or severity of its symptoms, or by increasing the effectiveness of another treatment, or by producing another beneficial effect), or reduce the likelihood of disease occurrence or exacerbation.

In the present application, the terms “disease” or “condition” are used interchangeably and generally refer to any deviation of a subject from a normal state, such as any change in the state of the body or certain organs that prevents or disrupts the performance of functions, and/or causes symptoms such as discomfort, dysfunction, suffering or even death in those who are afflicted or exposed to it.

In the present application, the term “tumor” generally refers to all neoplastic cell growth and proliferation, whether malignant or benign, and all precancerous and cancerous cells and tissues. The terms “cancer”, “cancerous”, “cell proliferative disorders”, “proliferative disorders” and “tumor” are not mutually exclusive when referred to herein. In the present application, the tumor can include solid tumors and/or hematological tumors.

In the present application, the term “administering” generally refers to delivering a protein (including an immunoglobulin) to a human or animal in need thereof by any route known in the art. Pharmaceutical carriers and formulations or compositions are also well known in the art. Routes of administration can include: intravenous, intramuscular, intradermal, subcutaneous, transdermal, mucosal, intratumoral or mucosal. Alternatively, these terms can indicate that a vector for recombinant protein expression is delivered to a cell or cultured cell and/or a cell or organ of a subject. Such administration or introduction can occur in vivo, in vitro or ex vivo. A vector for recombinant protein or polypeptide expression can be introduced into a cell by transfection, which generally refers to the insertion of heterologous DNA into a cell by physical means (e.g., calcium phosphate transfection, electroporation, microinjection or lipofection); infection, which generally refers to the introduction of an infectious agent (i.e., a virus); or transduction, which generally refers to the stable infection of a cell by a virus, or the transfer of genetic material from one microorganism to another by a viral agent (e.g., a bacteriophage).

In the present application, the term “contact” generally refers to two or more different types of substances being contacted together in any order, in any manner, and for any length of time. When applied to cells, the term “contact” generally refers to a method by which the antigen-binding proteins, polypeptides, immunoconjugates, nucleic acids, carriers, cells, and/or pharmaceutical compositions of the present application are delivered to target cells or placed directly close to target cells, and the delivery can be in vitro or in vivo and can involve the use of a recombinant vector system. For example, “contact” can include placing polynucleotides in a beaker, microtiter plate, cell culture flask, or microarray containing nucleic acid molecules. For another example, contact can include placing an antibody in a beaker, microtiter plate, cell culture flask, or microarray containing a polypeptide, and contact can occur in vivo, ex vivo, or in vitro.

In the present application, the term “effective amount” or “effective dose” generally refers to an amount of an active therapeutic agent sufficient to produce the desired therapeutic response without excessive adverse side effects such as toxicity, irritation or allergic reactions. Obviously, the specific “effective amount” will vary with various factors, such as the specific condition being treated, the physiological condition of the patient, the type of animal being treated, the duration of treatment, the nature of concurrent therapy (if any), and the specific formulation and structure of the compound or its derivative used. In this case, if a certain amount results in (without limitation) one or more of the following, it will be considered therapeutically effective: (a) inhibiting cancer cell growth (e.g., AML cells); and (b) killing cancer cells (e.g., AML cells).

In the present application, the term “subject” generally refers to a human or non-human animal, including but not limited to cats, dogs, horses, pigs, cows, sheep, rabbits, mice, rats or monkeys, etc. In some embodiments, the subject is a human. “Subject in need” can refer to a subject (e.g., patient) who suffers from or is at risk of developing a disease or disease that can be treated (e.g., improved, modified, prevented) by inducing T cells to exert a specific effect on the cytotoxicity of malignant T cells.

In the present application, the term “circRNA” is generally referred to as circular RNA, which is a new type of noncoding RNA (ncRNA) molecule. Of the different RNA compositions, circular RNA can be divided into three categories: exon circular RNA (ecircRNA), intron circular RNA (circular intronic RNAs, ciRNAs) and exon-intron circRNA (exon-intron circRNA, EIciRNA).

In the present application, the terms “include”, “comprise”, “have”, “can”, “contain” and their variations are generally intended to be open transitional phrases, terms or words that do not exclude the possibility of additional actions or structures. The term “consist of . . . ” generally means that no other components (or similarly, features, integers, steps, etc.) can be present. Unless the context clearly dictates otherwise, singular forms such as “a”, “an”, “the” in English, “a”, “an” and “described/the” in Chinese generally include plural forms of the referred things.

In the present application, the term “about” generally means approximately, in the region of, roughly, or around. When the term “about” is used to refer to a numerical range, a cutoff or a specific value is used to indicate that the stated value can vary from the recited value by up to 10%. For example, it can vary within a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below the specified value.

### DETAILED DESCRIPTION

### Modified Immune Effector Cells

On the one hand, the present application provides a modified immune effector cell, comprising a nucleic acid molecule encoding a fusion protein, wherein the fusion protein comprises a first domain that activates an antigen presenting cell (APC) and a second domain that activates an immune effector cell, wherein (i) the first domain includes (a) an activating receptor ligand or a receptor binding fragment thereof that binds to the APC, or (b) an activating receptor antibody or an antigen-binding fragment thereof that binds to the APC; and (ii) the second domain includes (a) a co-stimulatory ligand or a receptor binding fragment thereof of the immune effector cell, (b) an antibody or an antigen-binding fragment thereof that binds to a co-stimulatory receptor of the immune effector cell, or (c) a co-stimulatory receptor or a functional fragment thereof of the immune effector cell.

In some embodiments, the APC is selected from the group consisting of a dendritic cell, a macrophage, a myeloid-derived suppressor cell, a monocyte, a B cell, a T cell, and a Langerhans cell.

In some embodiments, the immune effector cell is selected from the group consisting of a T cell, an NK cell, an NKT cell, a macrophage, a neutrophil, and a granulocyte.

In some embodiments, the fusion protein is a membrane protein. In some embodiments, the fusion protein is a soluble protein. In some embodiments, the fusion protein is a bispecific antibody. In some embodiments, the C-terminus of the first domain is connected to the N-terminus of the second domain. In some embodiments, the N-terminus of the first domain is connected to the C-terminus of the second domain.

In some embodiments, the first domain is directly or indirectly connected to the second domain.

In some embodiments, the first domain and the second domain are connected by a linker.

In some embodiments, the linker includes a peptide linker. The linker can be a flexible linker or a rigid linker.

In some embodiments, the linker has an amino acid sequence of (GGGGS)n, n=1, 2, 3, 4, or 5. In some embodiments, the linker has an amino acid sequence of (EAAAK)n, n=1, 2, 3, 4, or 5. In some embodiments, the linker has an amino acid sequence of (PA) nP, n=1, 2, 3, 4, or 5. In some embodiments, the linker has an amino acid sequence of GSGGGGSGGGGSGGGGS (SEQ ID NO: 252). In some embodiments, the linker has an amino acid sequence of GGGGS (SEQ ID NO: 253). In some embodiments, the linker is a CD8 hinge. In some embodiments, the linker is a CD28 hinge. In some embodiments, the linker is an IgG Fc hinge. In some embodiments, the linker can be a trimerization motif selected from the group consisting of T4 fibrin trimerization motif, isoleucine zipper motif, GCN4II motif, Matrilin-1 motif, and type XV collagen trimerization motif.

In the present application, APC refers to any cell that displays one or more antigens on its surface, for example, in combination with one or more major histocompatibility complex (MHC) proteins. The MHC/antigen complex can be recognized by T cells using their T cell receptors (TCRs) and induce an immune response. In some embodiments, the APC is selected from the group consisting of a dendritic cell, a macrophage, a myeloid-derived suppressor cell, a monocyte, a B cell, a T cell, and a Langerhans cell.

### APCs Activate Receptors

As understood in the art, a molecule can activate APC by promoting maturation of APC, proinflammatory state, cytotoxicity, antigen presentation, epitope spreading, cytokine production, co-stimulation of immune effector cell (e.g., T cell), or any combination thereof. In some embodiments, the first domain of the fusion protein provided in the present invention activates APC by promoting maturation and activation of APC (e.g., DC). In some embodiments, the first domain of the fusion protein provided in the present invention activates APC by promoting epitope spreading between APC and other immune effector cell (e.g., T cell). In some embodiments, the first domain of the fusion protein provided in the present invention activates APC by promoting antigen presentation by APC. In some embodiments, the first domain of the fusion protein provided in the present invention activates APC by promoting cytotoxicity of APC to foreign bodies (e.g., cancer cells).

In some embodiments, the fusion protein provided in the present invention includes a first domain that activates APC, which includes a ligand or a receptor binding fragment thereof that binds to an activation receptor of APC. “Activation receptor” refers to a membrane protein expressed on APC that, after binding to a ligand or antibody, can trigger a signal to promote the flow, differentiation, proliferation and/or activation of APC. APC activation receptors include, for example, CD40, CD80, CD86, CD91, DEC-205 and DC-SIGN.

A “ligand” of a receptor refers to a molecule that can selectively bind to a receptor. In some embodiments, the ligand is a polypeptide. A “receptor binding fragment” of a ligand refers to a fragment of a ligand that retains its receptor binding ability. Various ligands can stimulate the growth, differentiation, migration and/or activation of dendritic cell or other APC by binding to activation receptors on APC. (See Banchereau J et al., Nature (1998) 392:245-52; Young J W et al., Stem Cells (1996) 14:376-387; Cella M et al., Curr Opin Immunol. (1997) 9: 10-16; Curti A et al., J. Biol. Regul. Homeost. Agents (2001) 15: 49-52). Examples of ligand that can regulate differentiation, maturation, expansion and/or activation of dendritic cell or other APCs include, for example, CD40 ligand (CD40L), CD80 ligand, CD86 ligand, CD91 ligand (RAP1), DEC-205 ligand and DC-SIGN ligand. In some embodiments, the fusion protein provided in the present invention includes a first domain, which includes a ligand or a receptor binding fragment thereof that binds to an APC activation receptor disclosed in the present invention.

CD40/CD40L: CD40 is a 48 kD transmembrane glycoprotein surface receptor that is a member of the tumor necrosis factor receptor superfamily (TNFRSF). The typical amino acid sequence of human CD40 has been described (see, Accession No.: ALQ33424.1 GI: 957949089), and CD40 was originally described as a co-stimulatory receptor expressed on APCs that plays a central role in B cell and T cell activation. CD40's ligand CD154 (also known as TRAP, T-BAM, CD40 ligand, or CD40L) is a type II integral membrane protein. CD40L has been reported to promote the induction of dendritic cells and promote the occurrence of immunogenic responses. See, e.g., Elgueta R et al., Immunol Rev. (2009) 229(1):10.1111; Ma D & Clark E A, Semin Immunol. 2009 21(5): 265-272; Borges L et al. J Immunol. (1999) 163:1289-1297; Grewal I, Immunol Res. (1997) 16:59-70. Exemplary polynucleotides encoding CD40 ligands and equivalents are described (see, e.g., Genbank Accession Nos. X65453 and L07414), as well as formulations, compositions and methods of use (U.S. Pat. No. 6,290,972). An exemplary amino acid sequence of human CD40L is provided below. The extracellular domain (SEQ ID NO: 224) is underlined.

(SEQ ID NO: 223)
MIETYNQTSPRSAATGLPISMKIFMYLLTVFLITQMIGSALFAVYLHRL
DKIEDERNLHEDFVFMKTIQRCNTGERSLSLLNCEEIKSQFEGFVKDIM
LNKEETKKENSFEMQKGDQNPQIAAHVISEASSKTTSVLQWAEKGYYTM
SNNLVTLENGKQLTVKRQGLYYIYAQVTFCSNREASSQAPFIASLCLKS
PGRFERILLRAANTHSSAKPCGQQSIHLGGVFELQPGASVFVNVTDPSQ
VSHGTGFTSFGLLKL

In some embodiments, the first domain of the fusion protein provided in the present invention comprises CD40L or a receptor binding fragment of CD40L. In some embodiments, the receptor binding fragment of CD40L includes amino acids 119-261 of CD40L (SEQ ID NO: 223). In some embodiments, the receptor binding fragment of CD40L includes the extracellular domain of CD40L.

In some embodiments, the first domain comprises an antibody or antigen-binding fragment thereof that binds to an activation receptor of the APC. In some embodiments, the first domain of the fusion protein provided in the present invention includes an antibody or antigen-binding fragment that binds to CD40, CD80, CD86, CD91, DEC-205 or DC-SIGN. For example, the first domain can be an anti-CD40 antibody or antigen-binding fragment thereof.

### Co-Stimulatory Receptor on Immune Effector Cell

In some embodiments, the fusion protein provided in the present invention comprises a first domain that activates an antigen presenting cell (e.g., a dendritic cell) and a second domain that activates an immune effector cell (e.g., a T cell), wherein the second domain includes (a) a co-stimulatory receptor of the immune effector cell or a functional fragment thereof, (b) a co-stimulatory ligand of the immune effector cell, or a receptor binding fragment thereof, or (c) an antibody that binds to a co-stimulatory receptor of an immune effector cell, or an antigen-binding fragment thereof.

As used in the present invention and as understood in the art, “immune effector cell” refer to a cell that have hematopoietic origin and play a direct role in the immune response to a target (such as a pathogen, cancer cell, or foreign substance). The immune effector cell includes T cell, a B cell, a an natural killer (NK) cell, an NKT cell, a macrophage, a granulocyte, a neutrophil, a eosinophil, a mast cell, and a basophil. In some embodiments, the second domain of the activated immune effector cell of the fusion protein provided in the present invention includes a co-stimulatory receptor for immune effector cell. In some embodiments, the immune effector cell is a T cell, a NK cell, an NKT cell, a macrophage, a neutrophils, or a granulocyte. In some embodiments, the immune effector cell is a T cell. In some embodiments, the immune effector cell is a NK cell. In some embodiments, the immune effector cell is a macrophage.

The “stimulation” of immune effector cells refers to the primary response induced by mediating signaling events in immune effector cells by binding to stimulatory molecules and their cognate ligands, which can change the expression of certain genes and/or the reorganization of cytoskeleton structures, etc. The “stimulatory molecule” of immune effector cells refers to a molecule on immune effector cells, which, after binding to the cognate ligands usually present on APCs, can mediate signaling to promote the maturation, differentiation, proliferation and/or activation of immune effector cells. For example, the stimulatory molecule of T cells, the TCR/CD3 complex triggers the activation of T cells. The ligand of the stimulatory molecule or “stimulatory ligand” refers to a ligand that is usually present on APCs and can bind to the stimulatory molecules on immune effector cells to mediate the primary response of immune effector cells, and the primary response includes but is not limited to maturation, differentiation, activation, initiation of immune response, proliferation, etc. Stimulatory ligands are well known in the art and include, for example, MHC class I molecules of loaded peptides, anti-CD3 antibodies, -superagonist anti-CD28 antibodies and superagonist anti-CD2 antibodies.

As used in the present invention and as understood in the art, “co-stimulatory signal” refers to a signal from a co-stimulatory receptor (e.g., CD28 or 4-1BB), which combines with a primary signal (e.g., TCR/CD3) to promote optimal clonal expansion, differentiation, and effector function of immune effector cells (e.g., T cells). As used in the present invention and as understood in the art, the “co-stimulatory receptor” of an immune effector cell refers to a molecule on an immune effector cell that specifically binds to a “co-stimulatory ligand” to mediate a co-stimulatory response of an immune effector cell, such as enhancing the activation or proliferation of an immune effector cell. Co-stimulatory receptors for immune effector cell includes, but are not limited to, CD28, 4-1BB, ICOS, CD27, OX40, DAP10, CD30, 2B4, CD2, LIGHT, GITR, TLR, DR3, and CD43. A “functional fragment” of a co-stimulatory receptor is a fragment of a co-stimulatory receptor that retains the function of the co-stimulatory receptor to mediate co-stimulatory signals and stimulate immune effector cells. In some embodiments, the functional fragment of a co-stimulatory receptor retains the co-stimulatory domain of the co-stimulatory receptor. In some embodiments, the co-stimulatory domain is a cytoplasmic domain of a co-stimulatory receptor. In some embodiments, the signal from the co-stimulatory receptor of an immune effector cell (e.g., a T cell) reduces the activation threshold of the immune effector cell. In some embodiments, the signal from the T cell co-stimulatory receptor leads to an enhancement of the TCR signaling event, wherein the TCR signal is necessary for efficient cytokine production (by enhancing transcriptional activity and messenger RNA stability), cell cycle progression, survival, metabolic regulation, and T cell responses.

As used herein and as understood in the art, “co-stimulatory ligand” refers to a molecule that specifically binds to a cognate co-stimulatory receptor on an immune effector cell, thereby providing a signal in addition to the primary signal provided by the stimulatory molecule that mediates a response in the immune effector cell, including but not limited to proliferation, activation, differentiation, etc. The co-stimulatory ligand can be present on an APC (e.g., a dendritic cell). Co-stimulatory ligands include, but are not limited to, CD58, CD70, CD83, CD80, CD86, CD137L (4-1BBL), CD252 (OX40L), CD275 (ICOS-L), CD54 (ICAM-1), CD49a, CD112 (PVRL2), CD150 (SLAM), CD155 (PVR), CD265 (RANK), CD270 (HVEM), TL1A, CD127, IL-4R, GITR-L, TIM-4, CD153 (CD30L), CD48, CD160, CD200R (OX2R), and CD44. A “receptor binding fragment” of a co-stimulatory ligand refers to a fragment in which a ligand retains its ability to bind to a receptor.

Some co-stimulatory receptors and co-stimulatory ligands are exemplified below. It should be understood that any co-stimulatory receptor and/or co-stimulatory ligand provided herein or known in the art can be used as part of the fusion protein provided herein.

CD28: Cluster of differentiation 28 (CD28) is a protein expressed on T cells that provides co-stimulatory signals for T cell activation and survival. CD28 is a receptor for CD80 (B7.1) and CD86 (B7.2) proteins. CD28 is a co-stimulatory receptor for optimal clonal expansion, differentiation, and effector function of T cells. CD28 binding lowers the T cell activation threshold and leads to enhanced TCR signaling events, where the TCR signal is required for efficient cytokine production (by enhancing transcriptional activity and messenger RNA stability), cell cycle progression, survival, metabolic regulation, and T cell responses. CD28 is a key factor in the organization of the immune synapse (IS), where CD28 enhances the close contact between T cells and APCs.

In some embodiments, the fusion protein provided in the present invention comprises a first domain that activates APC and a second domain that activates immune effector cell, wherein the second domain comprises a CD28 polypeptide or a functional fragment thereof. In some embodiments, the second domain includes a cytoplasmic domain of CD28. In some embodiments, the fusion protein provided in the present invention comprises a first domain that activates APC and a second domain that activates immune effector cell, wherein the second domain comprises a ligand or its receptor binding fragment that binds CD28. In some embodiments, the fusion protein provided in the present invention comprises a first domain that activates APC and a second domain that activates immune effector cells, wherein the second domain comprises an antibody or its antigen-binding fragment that binds CD28. In some embodiments, the second domain of the fusion protein provided in the present invention includes a functional fragment of CD28, and the functional fragment of CD28 includes a part of the intracellular/cytoplasmic domain of CD28, which can act as a co-stimulatory signaling domain. CD28 can have an amino acid sequence or a functional fragment thereof: the amino acid sequence corresponds to a sequence of GenBank No. P10747 (P10747.1, GI: 115973) or NP_006130 (NP_006130.1, GI: 5453611). In one embodiment, the fusion protein disclosed in the present invention can have an amino acid sequence or a fragment thereof comprising a CD28 cytoplasmic domain, the CD28 cytoplasmic domain corresponding to amino acids 180 to 122 of CD28 (the underlined portion of the following sequence, SEQ ID NO: 226). In another embodiment, the fusion protein disclosed in the present invention can have an amino acid sequence or a functional fragment thereof further comprising a CD28 transmembrane domain, the CD28 transmembrane domain corresponding to amino acids 153 to 179. It should be understood that, if desired, a CD28 sequence shorter or longer than a specific described domain can be comprised in the fusion protein disclosed in the present invention.

(NP_006130; SEQ ID NO: 225)
MLRLLLALNLFPSIQVTGNKILVKQSPMLVAYDNAVNLSCKYSYNLFSREFRA
SLHKGLDSAVEVCVVYGNYSQQLQVYSKTGFNCDGKLGNESVTFYLQNLYVNQTDI
YFCKIEVMYPPPYLDNEKSNGTIIHVKGKHLCPSPLFPGPSKPFWVLVVVGGVLACYS
LLVTVAFIIFWV R SKRSRLLHSDYMNMTPRRPG PTRKHYQPYA PPRDFAAYRS

In some embodiments, the second domain is an antibody or antigen-binding fragment thereof that binds a co-stimulatory receptor.

In some embodiments, the co-stimulatory receptor is selected from CD28, 4-1BB, ICOS, CD27, OX40, DAP10, 2B4, CD30, CD2, LIGHT, GITR, TLR, DR3 and CD43.

In some embodiments, the co-stimulatory receptor is CD28. For example, the second domain can be an anti-CD28 antibody or an antigen-binding fragment thereof.

In some embodiments, the first domain includes (a) a ligand that binds to an activating receptor of the APC, or a receptor binding fragment thereof, or (b) an antibody that binds to an activating receptor of the APC, or an antigen-binding fragment thereof, wherein the activating receptor of the APC is selected from the group consisting of CD40, CD80, CD86, CD91, DEC-205, and DC-SIGN. In some embodiments, the first domain includes a ligand that binds to an activating receptor of the APC, or a receptor binding fragment thereof. In some embodiments, the first domain includes a ligand that binds to a CD40 ligand, or a receptor binding fragment thereof. In some embodiments, the first domain includes CD40L. In some embodiments, the receptor binding fragment of CD40L includes amino acids 119-261 of CD40L (SEQ ID NO: 223). In some embodiments, the receptor binding fragment of CD40L includes the extracellular domain of CD40L. In some embodiments, the first domain of the fusion protein provided in the present invention includes three copies of CD40L or a receptor binding fragment of CD40L. In some embodiments, the first domain of the fusion protein provided in the present invention includes three copies of amino acids 119-261 of CD40L (SEQ ID NO: 223). In some embodiments, the first domain includes a ligand or a receptor binding fragment thereof that binds CD80. In some embodiments, the first domain includes a ligand or a receptor binding fragment thereof that binds CD86. In some embodiments, the first domain includes the extracellular domain of CD28. In some embodiments, the first domain includes CD28. In some embodiments, the first domain includes the extracellular domain of CTLA-4. In some embodiments, the first domain includes CTLA-4. In some embodiments, the first domain includes a ligand or a receptor binding fragment thereof that binds CD91. In some embodiments, the first domain includes domain 3 of RAP1. In some embodiments, the first domain includes RAP1. In some embodiments, the first domain includes a ligand or a receptor binding fragment thereof that binds DEC-205. In some embodiments, the first domain includes a ligand or a receptor binding fragment thereof that binds DC-SIGN. In some embodiments, the first domain includes ICAM2, ICAM3, CD18 or CEACAM1 or a receptor binding fragment thereof. In some embodiments, the first domain includes ICAM2 or a receptor binding fragment thereof. In some embodiments, the first domain includes ICAM3 or a receptor binding fragment thereof. In some embodiments, the first domain includes CD18, or a receptor binding fragment thereof. In some embodiments, the first domain includes CEACAM1 or a receptor binding fragment thereof.

In some embodiments, the first domain includes an antibody or antigen-binding fragment thereof that binds to an activating receptor of an APC. In some embodiments, the activating receptor of the APC is selected from the group consisting of CD40, CD80, CD86, CD91, DEC-205 and DC-SIGN. In some embodiments, the first domain includes an antibody or antigen-binding fragment thereof that binds to CD40. In some embodiments, the first domain includes an antibody or antigen-binding fragment thereof that binds to CD80. In some embodiments, the first domain includes an antibody or antigen-binding fragment thereof that binds to CD86. In some embodiments, the first domain includes an antibody or antigen-binding fragment thereof that binds to CD91. In some embodiments, the first domain includes an antibody or antigen-binding fragment thereof that binds to DEC-205. In some embodiments, the first domain includes an antibody or antigen-binding fragment thereof that binds to DC-SIGN. In some embodiments, the first domain includes a monoclonal antibody. In some embodiments, the first domain includes a chimeric antibody. In some embodiments, the first domain includes a humanized antibody. In some embodiments, the first domain includes a human antibody. In some embodiments, the first domain includes Fab, Fab′, F(ab′)2, Fv, scFv, (scFv)2, single-chain antibody, dual variable region antibody, bispecific antibody, nanobody or single variable region antibody. In some embodiments, the first domain includes a human antibody. In some embodiments, the first structure includes an scFv.

In some embodiments, the first domain of the fusion protein provided in the present invention includes an anti-CD40 antibody or an antigen-binding fragment thereof. In some embodiments, the first domain of the fusion protein provided in the present invention includes an anti-CD40 scFv. In some embodiments, the anti-CD40 antibody or an antigen-binding fragment thereof includes an antibody labeled F2.103, F5.157, F5.77, 4D11, A40C or 119, as shown in Table 1 below.

TABLE 1
Anti-CD40 Antibodies

| Antibody | scFv |
|---|---|
| F2.103 | EVQLVESGGGLVQPGGSLRLSCAVSGFTFSTYWMHWVRQAPGKGLVW |
| | VSRINSDGSSTTYADSVKGRFTISRDNAKNTLYLQMNSLRAEDTAVYYC |
| | ARDRVLWIGELSYYGMDVWGQGTTVTVSSGGGGSGGGGSGGGGSDIQ |
| | MTQSPSTLSASVGDRVTITCRASQSISNWLAWYQQKPGKAPKLLLYKA |
| | SGLESGVPSRFSGSGSGTEFTLTINSLQPDDFATYYCQQSNSYSWTFGHG |
| | TKVEIKRT SEQ ID NO: 227 |
| F5.157 | EVQLLESGGGLVQPGGSLRLSCAASGFAFSSYAMSWVRQAPGKGLEW |
| | VSAISGSGGSTYYADSVKGRFTISRDNSKNTLYLQMNSLRPRTRPYITVR |
| | KMGGTMVRGVMGTLTTGAREPWSPSPQGGGGSGGGGSGGGGSIQMT |
| | QSPSSVSASAGDRVTITCRASQGISSWLAWYQQKPGKAPKLLIYAGSSL |
| | QSGVPSRFSGSGFGTDFTLTIGSLQPEDFATYYCQQASSFPRTFGQGTKV |
| | EIKRTVLHHLSSSSRHLMS SEQ ID NO: 228 |
| F5.77 | EVQLLESGGGLVQPGGSLRLSCAASGFTFSSYAMSWVRQAPGKGLEW |
| | VSAISGSGGSTYYADSVKGRFTISRDNSKNTLYLQMNSLRAEDTAVYYC |
| | AKDGGYYGSGSYGYFDYWGQGTLVTVSSGGGGSGGGGSGGGGSDIQ |
| | MTQSPSSVSGSVGDRVTITCRASQGISSWLAWYQQKPGKAPKLLIYAGS |
| | SLQSGVPSRFSGSGFGTDFTLTISSLQPEDFATYYCQQASSFPRTFGQGTK |
| | VEIKRT |
| | SEQ ID NO: 229 |
| 4D11 | QLQLQESGPGLLKPSETLSLTCTVSGGSISSPGYYGGWIRQPPGKGLEWI |
| | GSIYKSGSTYHNPSLKSRVTISVDTSKNQFSLKLSSVTAADTAVYYCTRP |
| | VVRYFGWFDPWGQGTLVTVSSASGGGGSGGGGSGGGGSAIQLTQSPSS |
| | LSASVGDRVTITCRASQGISSALAWYQQKPGKAPKLLIYDASNLESGVP |
| | SRFSGSGSGTDFTLTISSLQPEDFATYYCQQFNSYPTFGQGTKVEIKRT |
| | SEQ ID NO: 230 |
| A40C | QVQLVQSGAEVKKPGASVKVSCTASGFNIKDYYVHWVKQAPGQGLE |
| | WMGRIDPEDGDSKYAPKFQGKATMTADTSTSTVYMELSSLRSEDTAVY |
| | YCTTSYYVGTYGYWGQGTLVTVSSGGGGSGGGGSGGGGSDIQMTQSP |
| | SSLSASVGDRVTITCSASSSVSYMLWFQQKPGKAPKLLIYSTSNLASGVP |
| | SRFSGSGSGTDFTLTISSLQPEDFATYYCQQRTFYPYTFGGGTKVEIKRT |
| | SEQ ID NO: 231 |
| 119 | QVQLQQSGPGLVKPSQTLSLTCAISGDSVSSNSATWNWIRQSPSRDLEW |
| | LGRTYYRSKWYRDYVGSVKSRIIINPDTSNNQFSLQLNSVTPEDTAIYY |
| | CTRAQWLGGDYPYYYSMDVWGQGTTVTVSSGGGGSGGGGSGGGGSE |
| | IVLTQSPATLSLSPGERATLSCRASQSVSSYLAWYQQKPGQAPRLLIYDA |
| | SNRATGIPARFSGSGSGTDFTLTISSLEPEDFAVYYCQQRSNTFGPGTKVD |
| | IKRT SEQ ID NO: 232 |

In some embodiments, the first domain of the fusion protein provided in the present invention includes an anti-CD40 scFv. In some embodiments, the first domain of the fusion protein provided in the present invention includes an anti-CD40 scFv, and the anti-CD40 scFv has at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity with the sequence shown in any one of SEQ ID NO: 227 to SEQ ID NO: 232.

In some embodiments, the first domain of the fusion protein provided in the present invention includes an anti-CD40 scFv having an amino acid sequence shown in any one of SEQ ID NO: 227 to SEQ ID NO: 232.

In some embodiments, the second domain of the fusion protein provided in the present invention includes a co-stimulatory receptor or a functional fragment thereof of an immune effector cell, wherein the immune cell is a T cell, a NK cell, a NKT cell, a macrophage, a neutrophil or a granulocyte. In some embodiments, the co-stimulatory receptor of the immune effector cell is selected from the group consisting of CD28, 4-1BB, ICOS, CD27, OX40, DAP10, 2B4, CD30, CD2, LIGHT, GITR, DR3 and CD43. In some embodiments, the second domain of the fusion protein provided in the present invention includes a functional fragment of a co-stimulatory receptor, and the co-stimulatory receptor is selected from the group consisting of CD28, 4-1BB, ICOS, CD27, OX40, DAP10, 2B4, CD30, CD2, LIGHT, GITR, DR3 and CD43.

In some embodiments, the functional fragment includes the cytoplasmic domain of a co-stimulatory receptor. In some embodiments, the second domain of the fusion protein provided in the present invention further includes the transmembrane domain of a co-stimulatory receptor. In some embodiments, the second domain of the fusion protein provided in the present invention includes the functional fragments of CD28, 4-1BB, ICOS, CD27, OX40, DAP10, 2B4, CD30, CD2, LIGHT, GITR, DR3 or CD43. In some embodiments, the second domain of the fusion protein provided in the present invention includes the cytoplasmic domain of CD28, 4-1BB, ICOS, CD27, OX40, DAP10, 2B4, CD30, CD2, LIGHT, GITR, DR3 or CD43.

In some embodiments, the second domain of the fusion protein provided in the present invention includes an antibody or its antigen-binding fragment that binds to a co-stimulatory receptor of an immune effector cell. The immune effector cell can be selected from a group consisting of a T cell, a NK cell, an NKT cell, a macrophage, a neutrophil and a granulocyte. In some embodiments, the co-stimulatory receptor of the immune effector cell is selected from a group consisting of CD28, 4-1BB, ICOS, CD27, OX40, DAP10, 2B4, CD30, CD2, LIGHT, GITR, DR3 and CD43. In some embodiments, the second domain of the fusion protein provided in the present invention includes an antibody or its antigen-binding fragment that binds to CD28, 4-1BB, ICOS, CD27, OX40, DAP10, 2B4, CD30, CD2, LIGHT, GITR, DR3 or CD43.

In some embodiments, the second domain includes a monoclonal antibody. In some embodiments, the second domain includes a chimeric antibody. In some embodiments, the second domain includes a humanized antibody. In some embodiments, the second domain includes a human antibody. In some embodiments, the second domain includes a Fab, Fab′, F(ab′)2, Fv, scFv, (scFv)2, a single-chain antibody, a dual variable region antibody, a bispecific antibody, a nanobody or a single variable region antibody. In some embodiments, the second domain includes a human antibody. In some embodiments, the second domain includes an scFv.

In some embodiments, the second domain of the fusion protein provided in the present invention includes an anti-CD28 antibody or an antigen-binding fragment. In some embodiments, the second domain of the fusion protein provided in the present invention includes an anti-CD28 scFv. In some embodiments, the anti-CD28 antibody or an antigen-binding fragment thereof includes an antibody labeled 1412.

TABLE 2
Exemplary anti-CD28 antibody

| Antibody | ScFv |
|---|---|
| 1412 | ALPVTALLLPLALLLHAARPQVQLVQSGAEVKKPGASVKVSCKASGYTF |
| | TSYYIHWVRQAPGQGLEWIGCIYPGNVNTNYNEKFKDRATLTVDTSISTA |
| | YMELSRLRSDDTAVYFCTRSHYGLDWNFDVWGQGTTVTVSSVEGGSGG |
| | SGGSGGSGGVMDDIQMTQSPSSLSASVGDRVTITCHASQNIYVWLNWYQ |
| | QKPGKAPKLLIYKASNLHTGVPSRFSGSGSGTDFTLTISSLQPEDFATYYC |
| | QQGQTYPYTFGGGTKVEIK (SEQ ID NO: 233) |

In some embodiments, the second domain of the fusion protein provided in the present invention includes an anti-CD28 scFv, and the anti-CD28 scFv has at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity with the sequence shown in SEQ ID NO: 233. In some embodiments, the second domain of the fusion protein provided in the present invention includes an anti-CD28 scFv having the amino acid sequence shown in SEQ ID NO: 233.

The fusion protein described in the present invention (i.e., LACO-Stim molecule) can include any combination of APC activators (ligands or antibodies that bind to activation receptors) disclosed in the present invention or otherwise known in the art and immune effector cell activators (co-stimulatory receptors or antibodies that bind to co-stimulatory receptors). For ease of illustration, various forms of CD40-C28 LACO-Stim fusion proteins are provided below, which activate APCs (e.g., dendritic cells) through CD40/CD40L signaling and activate immune effector cells (e.g., T cells) through CD28 signaling.

### Exemplary LACO-Stim (1): APC Activating Receptor Ligand+Co-Stimulatory Receptor (e.g., CD40L-CD28)

In some embodiments, the fusion protein provided in the present invention includes a first domain that activates antigen presenting cell (APC) and a second domain that activates immune effector cell, wherein the first domain includes a ligand or its receptor binding fragment of an activation receptor that binds to an APC, and the second domain includes a co-stimulatory receptor or its functional fragment of an immune effector cell. In some embodiments, the second domain includes the cytoplasmic domain of a co-stimulatory receptor of an immune effector cell. In some embodiments, the C-terminus of the first domain is connected to the N-terminus of the second domain. In some embodiments, the N-terminus of the first domain is connected to the C-terminus of the second domain. In some embodiments, the fusion protein provided in the present invention is a membrane fusion protein. In some embodiments, the first domain and the second domain are connected by a linker. The connector can be a flexible connector or a rigid connector. In some embodiments, the linker has an amino acid sequence of (GGGGS)n, n=1, 2, 3, 4 or 5. In some embodiments, the linker has an amino acid sequence of (EAAAK)n, n=1, 2, 3, 4 or 5. In some embodiments, the linker has an amino acid sequence of (PA)nP, n=1, 2, 3, 4, or 5. In some embodiments, the linker has an amino acid sequence of GSGGGGSGGGGSGGGGS. In some embodiments, the linker has an amino acid sequence of GGGGS.

In some embodiments, the first domain includes a ligand or a receptor binding fragment thereof that binds to an APC activating receptor, and the APC activating receptor is selected from the group consisting of CD40, CD80, CD86, CD91, DEC-205 and DC-SIGN. In some embodiments, the second domain includes a co-stimulatory receptor or a functional fragment thereof, and the co-stimulatory receptor is selected from the group consisting of CD28, 4-1BB, ICOS, CD27, OX40, DAP10, 2B4, CD30, CD2, LIGHT, GITR, TLR, DR3 and CD43.

In some embodiments, the fusion protein provided in the present invention has a first domain and a second domain, the first domain includes CD40L or its receptor binding fragment, and the second domain includes a CD28 cytoplasmic domain. In some embodiments, the fusion protein provided in the present invention has a first domain and a second domain, the first domain includes CD40L or its receptor binding fragment, and the second domain includes a 4-1BB cytoplasmic domain. In some embodiments, the fusion protein provided in the present invention has a first domain and a second domain, the first domain includes CD40L or its receptor binding fragment, and the second domain includes an ICOS cytoplasmic domain. In some embodiments, the fusion protein provided in the present invention has a first domain and a second domain, the first domain includes CD40L or its receptor binding fragment, and the second domain includes a CD27 cytoplasmic domain. In some embodiments, the fusion protein provided in the present invention has a first domain and a second domain, the first domain includes CD40L or its receptor binding fragment, and the second domain includes an OX40 cytoplasmic domain. In some embodiments, the fusion protein provided in the present invention has a first domain and a second domain, the first domain includes CD40L or its receptor binding fragment, and the second domain includes a DAP10 cytoplasmic domain. In some embodiments, the fusion protein provided in the present invention has a first domain and a second domain, the first domain includes CD40L or its receptor binding fragment, and the second domain includes a 2B4 cytoplasmic domain. In some embodiments, the fusion protein provided in the present invention has a first domain and a second domain, the first domain includes CD40L or its receptor binding fragment, and the second domain includes a CD30 cytoplasmic domain. In some embodiments, the fusion protein provided in the present invention has a first domain and a second domain, the first domain includes CD40L or its receptor binding fragment, and the second domain includes a CD2 cytoplasmic domain. In some embodiments, the fusion protein provided in the present invention has a first domain and a second domain, the first domain includes CD40L or its receptor binding fragment, and the second domain includes a LIGHT cytoplasmic domain. In some embodiments, the fusion protein provided in the present invention has a first domain and a second domain, the first domain includes CD40L or its receptor binding fragment, and the second domain includes a GITR cytoplasmic domain. In some embodiments, the fusion protein provided in the present invention has a first domain and a second domain, the first domain includes CD4ML or its receptor binding fragment, and the second domain includes a TLR cytoplasmic domain. In some embodiments, the fusion protein provided in the present invention has a first domain and a second domain, the first domain includes CD4L or a receptor binding fragment thereof, and the second domain includes a DR3 cytoplasmic domain. In some embodiments, the fusion protein provided in the present invention has a first domain and a second domain, the first domain includes CD4L or a receptor binding fragment thereof, and the second domain includes a CD43 cytoplasmic domain. The receptor binding fragment of CD40L can be amino acids 119-261 of the CD4TL (SEQ ID NO: 223). In some embodiments, the first domain includes a full-length CD40L.

| Exemplary LACO-Stim | |
|---|---|
| Fusion Proteins | sequence |
| 40L.28.40L.40L | MIETYNQTSPRSAATGLPISMKRSKRSRLLHSDYMNMTPRRP |
| (CD40L cytoplasmic | GPTRKHYQPYAPPRDFAAYRSIFMYLLTVFLITQMIGSALFAV |
| domain-CD28 cytoplasmic | YLHRRLDKIEDERNLHEDFVFMKTIQRCNTGERSLSLLNCEE |
| domain- | IKSQFEGFVKDIMLNKEETKKENSFEMQKGDQNPQIAAHVIS |
| CD40L transmembrane | EASSKTTSVLQWAEKGYYTMSNNLVTLENGKQLTVKRQGL |
| domain-CD40L | YYIYAQVTFCSNREASSQAPFIASLCLKSPGRFERILLRAANT |
| extracellular domain) | HSSAKPCGQQSIHLGGVFELQPGASVFVNVTDPSQVSHGTG |
| | FTSFGLLKL SEQ ID NO: 234 |
| TriCD40L_8-28 | QLTVKRQGLYYIYAQVTFCSNREASSQAPFIASLCLKSPGRFE |
| (a trimer of the CD40L | NPQIAAHVISEASSKTTSVLQWAEKGYYTMSNNLVTLENGK |
| extracellular domain, | RILLRAANTHSSAKPCGQQSIHLGGVFELQPGASVFVNVTDP |
| the CD8 transmembrane | SQVSHGTGFTSFGLLKLGGGGSGGGSNPQIAAHVISEASSKT |
| domain, and the CD28 | TSVLQWAEKGYYTMSNNLVTLENGKQLTVKRQGLYYIYAQ |
| cytoplasmic domain) | VTFCSNREASSQAPFIASLCLKSPGRFERILLRAANTHSSAKP |
| | CGQQSIHLGGVFELQPGASVFVNVTDPSQVSHGTGFTSFGLL |
| | KLGGGGSGGGSNPQIAAHVISEASSKTTSVLQWAEKGYYTM |
| | SNNLVTLENGKQLTVKRQGLYYIYAQVTFCSNREASSQAPFI |
| | ASLCLKSPGRFERILLRAANTHSSAKPCGQQSIHLGGVFELQ |
| | PGASVFVNVTDPSQVSHGTGFTSFGLLKLGSGGGGSGGGGS |
| | GGGGSTTTPAPRPPTPAPTIASQPLSLRPEACRPAAGGAVHTR |
| | GLDFACDIYIWAPLAGTCGVLLLSLVITLYCRSKRSRLLHSDY |
| | MNMTPRRPGPTRKHYQPYAPPRDFAAYRS SEQ ID NO: 235 |
| TriCD40L 28-28 | NPQIAAHVISEASSKTTSVLQWAEKGYYTMSNNLVTLENGK |
| (a trimer of the CD40L | QLTVKRQGLYYIYAQVTFCSNREASSQAPFIASLCLKSPGRFE |
| extracellular domain, | RILLRAANTHSSAKPCGQQSIHLGGVFELQPGASVFVNVTDP |
| the CD28 | SQVSHGTGFTSFGLLKLGGGGSGGGSNPQIAAHVISEASSKT |
| transmembrane domain, | TSVLQWAEKGYYTMSNNLVTLENGKQLTVKRQGLYYIYAQ |
| and the CD28 | VTFCSNREASSQAPFIASLCLKSPGRFERILLRAANTHSSAKP |
| cytoplasmic domain) | CGQQSIHLGGVFELQPGASVFVNVTDPSQVSHGTGFTSFGLL |
| | KLGGGGSGGGSNPQIAAHVISEASSKTTSVLQWAEKGYYTM |
| | SNNLVTLENGKQLTVKRQGLYYIYAQVTFCSNREASSQAPFI |
| | ASLCLKSPGRFERILLRAANTHSSAKPCGQQSIHLGGVFELQ |
| | PGASVFVNVTDPSQVSHGTGFTSFGLLKLGSGGGGSGGGGS |
| | GGGGSIEVMYPPPYLDNEKSNGTIIHVKGKHLCPSPLFPGPS |
| | KPFWVLVVVGGVLACYSLLVTVAFIIFWVRSKRSRLLHSDY |
| | MNMTPRRPGPTRKHYQPYAPPRDFAAYRS SEQ ID NO: 236 |

In some embodiments, the fusion protein provided in the present invention has an amino acid sequence that is at least 80%, at least 85%, at least 86%, at least 87%, at least 88%, at least 89%, at least 90%, at least 91%, at least 9200, at least 9300, at least 9400 at least 9500 at least 96%, at least 9700 at least 98% or at least 9900 identical to the sequence of the fusion protein labeled 40L.28.40L.40L, TriCD40L_8-28, or TriCD40L_28-28.

As will be understood by those skilled in the art, the CD40L functional fragment or full-length CD40L in the fusion protein exemplified in the present invention can be replaced with different ligands of APC activating receptors disclosed in the present invention or otherwise known in the art, including, for example, the extracellular domain of a CD80 ligand (e.g., CD28 or CTLA-4) or its full length, a CD86 ligand (e.g., CD28 or CTLA-4), a CD91 ligand (e.g., RAP1), a DEC-205 ligand, or a DC-SIGN ligand (e.g., ICAM2, ICAM3, CD18, or CEACAM1). As will be understood by those of person of ordinary skill in the art, the CD28 cytoplasmic domain in the fusion protein exemplified in the present invention can be replaced with the cytoplasmic domains of different co-stimulatory factors of immune effector cells disclosed in the present invention or otherwise known in the art, including, for example, 4-11B1, ICOS, CD27, OX40, DAP10, 2B34, CD30, CD2, LIGHT, GITR, TLR, DR3 or CD43 cytoplasmic domains; or different functional fragments of 4-11B1, ICOS, CD27, OX40, DA-P10, 2B34, CD30, CD2, LIGHT, GITR, TLR, DR3 or CD43, which retain the function of the full-length protein in activating immune effector cells.

Exemplary LACO-Stim (2): APC Activating Receptor Ligand+Antibody Binding to Co-Stimulatory Receptor (e.g., aCD28-CD40L)

In some embodiments, the fusion protein provided in the present invention includes a first domain that activates APC and a second domain that activates immune effector cell, wherein the first domain includes a ligand or its receptor binding fragment that binds to an activation receptor of APC, and wherein the second domain includes an antibody or its antigen-binding fragment that binds to a co-stimulatory receptor of an immune effector cell. In some embodiments, the C-terminus of the first domain is connected to the N-terminus of the second domain. In some embodiments, the N-terminus of the first domain is connected to the C-terminus of the second domain. In some embodiments, the fusion protein provided in the present invention is an antibody-based soluble protein.

In some embodiments, the two domains of the fusion protein disclosed in the present invention are connected by a trimerization motif. In some embodiments, the linker is a trimerization motif selected from the group consisting of a T4 fibrin trimerization motif, an isoleucine zipper, a GCN4II motif, a Matrilin-1 motif, and a type XV collagen trimerization motif. In some embodiments, the linker is a T4 fibrin trimerization motif.

In some embodiments, the first domain includes a ligand or a receptor binding fragment thereof that binds to an APC activating receptor, and the APC activating receptor is selected from the group consisting of CD40, CD80, CD86, CD91, DEC-205 and DC-SIGN. In some embodiments, the second domain includes an antibody or an antigen-binding fragment thereof that binds to a co-stimulatory receptor of an immune effector cell, wherein the co-stimulatory receptor is selected from the group consisting of CD28, 4-1BB, ICOS, CD27, OX40, DAP10, 2B4, CD30, CD2, LIGHT, GITR, TLR, DR3 and CD43.

In some embodiments, the fusion protein provided in the present invention has a first domain and a second domain, the first domain includes CD40L or its receptor binding fragment, and the second domain includes an anti-CD28 antibody or its antigen-binding fragment. In some embodiments, the fusion protein provided in the present invention has a first domain and a second domain, the first domain includes CD40L or its receptor binding fragment, and the second domain includes an anti-4-1BB antibody or its antigen-binding fragment. In some embodiments, the fusion protein provided in the present invention has a first domain and a second domain, the first domain includes CD40L or its receptor binding fragment, and the second domain includes an anti-ICOS antibody or its antigen-binding fragment. In some embodiments, the fusion protein provided in the present invention has a first domain and a second domain, the first domain includes CD40L or its receptor binding fragment, and the second domain includes an anti-CD27 antibody or its antigen-binding fragment. In some embodiments, the fusion protein provided in the present invention has a first domain and a second domain, the first domain includes CD40L or its receptor binding fragment, and the second domain includes an anti-OX40 antibody or its antigen-binding fragment. In some embodiments, the fusion protein provided in the present invention has a first domain and a second domain, the first domain includes CD40L or its receptor binding fragment, and the second domain includes an anti-DAP10 antibody or its antigen-binding fragment. In some embodiments, the fusion protein provided in the present invention has a first domain and a second domain, the first domain includes CD40L or its receptor binding fragment, and the second domain includes an anti-2B4 antibody or its antigen-binding fragment. In some embodiments, the fusion protein provided in the present invention has a first domain and a second domain, the first domain includes CD40L or its receptor binding fragment, and the second domain includes an anti-CD30 antibody or its antigen-binding fragment. In some embodiments, the fusion protein provided in the present invention has a first domain and a second domain, the first domain includes CD40L or its receptor binding fragment, and the second domain includes an anti-CD2 antibody or its antigen-binding fragment. In some embodiments, the fusion protein provided in the present invention has a first domain and a second domain, the first domain includes CD40L or its receptor binding fragment, and the second domain includes an anti-LIGHT antibody or its antigen-binding fragment. In some embodiments, the fusion protein provided in the present invention has a first domain and a second domain, the first domain includes CD40L or a receptor binding fragment thereof, and the second domain includes an anti-GITR antibody or an antigen-binding fragment thereof. In some embodiments, the fusion protein provided in the present invention has a first domain and a second domain, the first domain includes CD40L or a receptor binding fragment thereof, and the second domain includes an anti-TLR antibody or an antigen-binding fragment thereof. In some embodiments, the fusion protein provided in the present invention has a first domain and a second domain, the first domain includes CD40L or a receptor binding fragment thereof, and the second domain includes an anti-DR3 antibody or an antigen-binding fragment thereof. In some embodiments, the fusion protein provided in the present invention has a first domain and a second domain, the first domain includes CD40L or a receptor binding fragment thereof, and the second domain includes an anti-CD43 antibody or an antigen-binding fragment thereof. In some embodiments, the receptor binding fragment of CD40L can have amino acids 119-261 of CD40L (SEQ ID NO: 223).

In some embodiments, the fusion protein provided in the present invention has a first domain including CD40L or its receptor binding fragment and a second domain including an anti-CD28 antibody or its antigen-binding fragment. The anti-CD28 antibody or antigen-binding fragment can be any anti-CD28 antibody or antigen-binding fragment disclosed in the present invention or otherwise known in the art that can activate CD28 signaling. In some embodiments, the anti-CD28 antibody or antigen-binding fragment is an antibody labeled 1412. In some embodiments, the anti-CD28 antibody or antigen-binding fragment includes an anti-CD28 scFv having an amino acid sequence shown in SEQ ID NO: 233.

| Exemplary LACO-Stim | |
|---|---|
| Fusion Proteins | sequence |
| 1412-T4-CD40L (anti- | QVQLVQSGAEVKKPGASVKVSCKASGYTFTSYYIHWVRQA |
| CD28 scFv (1412)-T4 | PGQGLEWIGCIYPGNVNTNYNEKFKDRATLTVDTSISTAYME |
| Fibrin trimerization | LSRLRSDDTAVYFCTRSHYGLDWNFDVWGQGTTVTVSSVE |
| motif-CD40L | GGSGGSGGSGGSGGVMDDIQMTQSPSSLSASVGDRVTITCH |
| extracellular domain) | ASQNIYVWLNWYQQKPGKAPKLLIYKASNLHTGVPSRFSGS |
| | GSGTDFTLTISSLQPEDFATYYCQQGQTYPYTFGGGTKVEIK |
| | GYIPEAPRDGQAYVRKDGEWVLLSTFLGDQNPQIAAHVISE |
| | ASSKTTSVLQWAEKGYYTMSNNLVTLENGKQLTVKRQGLY |
| | YIYAQVTFCSNREASSQAPFIASLCLKSPGRFERILLRAANTH |
| | SSAKPCGQQSIHLGGVFELQPGASVFVNVTDPSQVSHGTGFT |
| | SFGLLKL (SEQ ID NO: 237) |

In some embodiments, the fusion protein provided herein has an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence of the fusion protein labeled 1412-T4-CD40L.

As would be understood by a person of ordinary skill in the art, the CD40L extracellular domain in the fusion protein exemplified in the present invention can be replaced with the extracellular domain or receptor binding fragment of different ligands of APC activation receptors disclosed in the present invention or otherwise known in the art, including, for example, the extracellular domain or receptor binding domain of CD80 ligand (e.g., CD28 or CTLA-4), CD86 ligand (e.g., CD28 or CTLA-4), CD91 ligand (e.g., RAP1), DEC-205 ligand or DC-SIGN ligand (e.g., ICAM2, ICAM3, CD18 or CEACAM1). As would be understood by a person of ordinary skill in the art, the anti-CD28 antibody or antigen-binding fragment in the fusion protein exemplified in the present invention can be replaced with the antibody or antigen-binding fragment of different co-stimulatory factors that bind and activate immune effector cells disclosed in the present invention or otherwise known in the art, including, for example, antibodies or antigen-binding fragments that bind 4-1BB, ICOS, CD27, OX40, DAP10, 2B4, CD30, CD2, LIGHT, GITR, TLR, DR3 or CD43.

Exemplary LACO-Stim (3): Antibody to APC Activating Receptor+Antibody to Co-Stimulatory Receptor (e.g., aCD40/aCD28 Bispecific Ab)

In some embodiments, the present invention provides bispecific antibodies. As would be understood by a person of ordinary skill in the art, “bispecific antibodies” refer to antibodies that have binding specificity for at least two different antigenic epitopes. The epitopes can be from the same antigen or from two different antigens. In some embodiments, the fusion protein provided in the present invention includes a first domain that activates an APC and a second domain that activates an immune effector cell, wherein the first domain includes an antibody or an antigen-binding fragment thereof that binds to an activation receptor of the APC, and wherein the second domain includes an antibody or an antigen-binding fragment thereof that binds to a co-stimulatory receptor of an immune effector cell. Thus, the bispecific antibodies disclosed in the present invention have binding specificity for (1) an activation receptor of an APC (e.g., a dendritic cell) and (2) a co-stimulatory receptor of an immune effector cell (e.g., a T cell). In some embodiments, the C-terminus of the first domain is connected to the N-terminus of the second domain. In some embodiments, the N-terminus of the first domain is connected to the C-terminus of the second domain.

In some embodiments, the first domain and the second domain are connected by a linker. The linker can be a flexible linker or a rigid linker. In some embodiments, the linker has an amino acid sequence of (GGGGS)n, n=1, 2, 3, 4 or 5. In some embodiments, the linker has an amino acid sequence of (EAAAK)n, n=1, 2, 3, 4 or 5. In some embodiments, the linker has an amino acid sequence of (PA) nP, n=1, 2, 3, 4 or 5. In some embodiments, the linker has an amino acid sequence of GSGGGGSGGGGSGGGGS. In some embodiments, the linker has an amino acid sequence of GGGGS.

In some embodiments, the fusion protein provided in the present invention is a bispecific antibody, which includes a first domain or an antigen-binding fragment thereof that binds to an activation receptor of an APC, and a second domain that binds to a co-stimulatory receptor antibody or an antigen-binding fragment thereof of an immune effector cell. In some embodiments, the first domain includes an antibody or an antigen-binding fragment thereof that binds to CD40, CD80, CD86, CD91, DEC-205, or DC-SIGN. In some embodiments, the second domain includes an antibody or an antigen-binding fragment thereof that binds to CD28, 4-1BB, ICOS, CD27, OX40, DAP10, 2B4, CD30, CD2, LIGHT, GITR, TLR, DR3, or CD43.

In some embodiments, the present invention provides a bispecific antibody including a first domain and a second domain, the first domain being an anti-CD40 antibody or an antigen-binding fragment thereof, and the domain second domain including an anti-CD28 antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-4-1BB antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-ICOS antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-CD27 antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-OX40 antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-DAP10 antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-2B4 antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-CD30 antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-CD2 antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-LIGHT antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-GITR antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-TLR antibody or an antigen-binding fragment domain thereof. In some embodiments, the present invention provides a bispecific antibody including a first domain and a second domain, the first domain being an anti-CD40 antibody or an antigen-binding fragment thereof, and the second domain including an anti-DR3 antibody or an antigen-binding fragment thereof. In some embodiments, the present invention provides a bispecific antibody including a first domain and a second domain, the first domain being an anti-CD40 antibody or an antigen-binding fragment thereof, and the second domain including an anti-CD43 antibody or an antigen-binding fragment thereof.

The method for preparing bispecific antibodies is known in the art. For example, bispecific antibodies can be produced recombinantly by co-expressing two pairs of immunoglobulin heavy/light chain. See, e.g., Milstein et al., (1983) Nature 305: 537-39. Alternatively, chemical linkage can be used to prepare bispecific antibodies. See, e.g., Brennan et al., (1985) Science 229: 81. Bispecific antibodies include bispecific antigen-binding fragments. See, e.g., Holliger et al., (1993) Proc. Natl. Acad. Sci. USA 90: 6444-48; Gruber et al. (1994) J. Immunol. 152: 5368. Techniques for making bispecific antibodies include, but are not limited to, recombinant co-expression of two immunoglobulin heavy chain-light chain pairs with different specificities (see Milstein and Cuello, Nature 305: 537 (1983), WO 93/08829, and Traunecker et al., EMBO J. 10: 3655 (1991)), and “knob-in-hole” engineering (see, e.g., U.S. Pat. No. 5,731,168). Multispecific antibodies can also be prepared by engineering electrostatic steering effects to prepare antibody Fc-heterodimer molecules (WO2009/089004A1); cross-linking two or more antibodies or fragments (see, e.g., U.S. Pat. No. 4,676,980, and Brennan et al., Science 229:81 (1985)); using leucine zippers to produce bispecific antibodies (see, e.g., Kostelny et al., J. Immunol. 148(5):1547-1553 (1992)); using “diabody” technology to prepare bispecific antibody fragments (see, e.g., Hollinger et al., Proc. Natl. Acad. Sci. USA, 90:6444-6448 (1993)); and using single-chain Fv (scFv) dimers (see, e.g., Gruber et al., J. Immunol., 152:5368-5476). (1994)); and bispecific antibodies as described, for example, in Tutt et al. J. Immunol. 147: 60 (1991). Engineered antibodies containing three or more functional antigen-binding sites, including “Octopus antibodies”, are also included herein (see, for example, US2006/0025576A1). Bispecific antibodies can also be constructed by linking two different antibodies or portions thereof. For example, a bispecific antibody can include Fab, F(ab′)2, Fab′, scFv and sdAb from two different antibodies.

In some embodiments, the first domain of the fusion protein provided in the present invention includes an anti-CD40 antibody or an antigen-binding fragment thereof. The anti-CD40 antibody or antigen-binding fragment can be any anti-CD40 antibody or antigen-binding fragment disclosed in the present invention or otherwise known in the art that can activate CD40 signaling. In some embodiments, the anti-CD40 antibody or antigen-binding fragment thereof includes an antibody labeled as F2.103, F5.157, F5.77, 4D11, A40C or 119 as provided in Table 1 above. In some embodiments, the anti-CD40 antibody or antigen-binding fragment thereof includes an anti-CD40 scFv having an amino acid sequence as shown in any one of SEQ ID NO: 227 to SEQ ID NO: 232.

In some embodiments, the anti-CD28 antibody or antigen-binding fragment can be any anti-CD28 antibody or antigen-binding fragment disclosed herein or otherwise known in the art that activates CD28 signaling. In some embodiments, the anti-CD28 antibody or antigen-binding fragment is an antibody labeled 1412. In some embodiments, the anti-CD28 antibody or antigen-binding fragment thereof includes an anti-CD28 scFv having an amino acid sequence as shown in SEQ ID NO: 233.

| Exemplary LACO-Stim | |
|---|---|
| Fusion Proteins | sequence |
| 1412-F2.103 (anti- | QVQLVQSGAEVKKPGASVKVSCKASGYTFTSYYIHWVR |
| CD28/anti-CD40 | QAPGQGLEWIGCIYPGNVNTNYNEKFKDRATLTVDTSIST |
| bispecific antibody) | AYMELSRLRSDDTAVYFCTRSHYGLDWNFDVWGQGTTV |
| | TVSSVEGGSGGSGGSGGSGGVMDDIQMTQSPSSLSASVG |
| | DRVTITCHASQNIYVWLNWYQQKPGKAPKLLIYKASNLH |
| | TGVPSRFSGSGSGTDFTLTISSLQPEDFATYYCQQGQTYPY |
| | TFGGGTKVEIKGGGGSEVQLVESGGGLVQPGGSLRLSCA |
| | VSGFTFSTYWMHWVRQAPGKGLVWVSRINSDGSSTTYA |
| | DSVKGRFTISRDNAKNTLYLQMNSLRAEDTAVYYCARDR |
| | VLWIGELSYYGMDVWGQGTTVTVSSGGGGSGGGGSGG |
| | GGSDIQMTQSPSTLSASVGDRVTITCRASQSISNWLAWYQ |
| | QKPGKAPKLLLYKASGLESGVPSRFSGSGSGTEFTLTINSL |
| | QPDDFATYYCQQSNSYSWTFGHGTKVEIKRT SEQ ID NO: |
| | 238 |
| 1412-F5.157 (anti- | QVQLVQSGAEVKKPGASVKVSCKASGYTFTSYYIHWVR |
| CD28/anti-CD40 | QAPGQGLEWIGCIYPGNVNTNYNEKFKDRATLTVDTSIST |
| bispecific antibody) | AYMELSRLRSDDTAVYFCTRSHYGLDWNFDVWGQGTTV |
| | TVSSVEGGSGGSGGSGGSGGVMDDIQMTQSPSSLSASVG |
| | DRVTITCHASQNIYVWLNWYQQKPGKAPKLLIYKASNLH |
| | TGVPSRFSGSGSGTDFTLTISSLQPEDFATYYCQQGQTYPY |
| | TFGGGTKVEIKGGGGSEVQLLESGGGLVQPGGSLRLSCA |
| | ASGFAFSSYAMSWVRQAPGKGLEWVSAISGSGGSTYYAD |
| | SVKGRFTISRDNSKNTLYLQMNSLRPRTRPYITVRKMGGT |
| | MVRGVMGTLTTGAREPWSPSPQGGGGSGGGGSGGGGSI |
| | QMTQSPSSVSASAGDRVTITCRASQGISSWLAWYQQKPG |
| | KAPKLLIYAGSSLQSGVPSRFSGSGFGTDFTLTIGSLQPEDF |
| | ATYYCQQASSFPRTFGQGTKVEIKRTVLHHLSSSSRHLMS |
| | SEQ ID NO: 239 |
| 1412-F5.77 (anti- | QVQLVQSGAEVKKPGASVKVSCKASGYTFTSYYIHWVR |
| CD28/anti-CD40 | QAPGQGLEWIGCIYPGNVNTNYNEKFKDRATLTVDTSIST |
| bispecific antibody) | AYMELSRLRSDDTAVYFCTRSHYGLDWNFDVWGQGTTV |
| | TVSSVEGGSGGSGGSGGSGGVMDDIQMTQSPSSLSASVG |
| | DRVTITCHASQNIYVWLNWYQQKPGKAPKLLIYKASNLH |
| | TGVPSRFSGSGSGTDFTLTISSLQPEDFATYYCQQGQTYPY |
| | TFGGGTKVEIKGGGGSEVQLLESGGGLVQPGGSLRLSCA |
| | ASGFTFSSYAMSWVRQAPGKGLEWVSAISGSGGSTYYAD |
| | SVKGRFTISRDNSKNTLYLQMNSLRAEDTAVYYCAKDGG |
| | YYGSGSYGYFDYWGQGTLVTVSSGGGGSGGGGSGGGGS |
| | DIQMTQSPSSVSGSVGDRVTITCRASQGISSWLAWYQQKP |
| | GKAPKLLIYAGSSLQSGVPSRFSGSGFGTDFTLTISSLQPED |
| | FATYYCQQASSFPRTFGQGTKVEIKRT SEQ ID NO: 240 |
| 1412-4D11 (anti- | QVQLVQSGAEVKKPGASVKVSCKASGYTFTSYYIHWVR |
| CD28/anti-CD40 | QAPGQGLEWIGCIYPGNVNTNYNEKFKDRATLTVDTSIST |
| bispecific antibody) | AYMELSRLRSDDTAVYFCTRSHYGLDWNFDVWGQGTTV |
| | TVSSVEGGSGGSGGSGGSGGVMDDIQMTQSPSSLSASVG |
| | DRVTITCHASQNIYVWLNWYQQKPGKAPKLLIYKASNLH |
| | TGVPSRFSGSGSGTDFTLTISSLQPEDFATYYCQQGQTYPY |
| | TFGGGTKVEIKGGGGSQLQLQESGPGLLKPSETLSLTCTV |
| | SGGSISSPGYYGGWIRQPPGKGLEWIGSIYKSGSTYHNPSL |
| | KSRVTISVDTSKNQFSLKLSSVTAADTAVYYCTRPVVRYF |
| | GWFDPWGQGTLVTVSSASGGGGSGGGGSGGGGSAIQLT |
| | QSPSSLSASVGDRVTITCRASQGISSALAWYQQKPGKAPK |
| | LLIYDASNLESGVPSRFSGSGSGTDFTLTISSLQPEDFATYY |
| | CQQFNSYPTFGQGTKVEIKRT SEQ ID NO: 241 |

In some embodiments, the fusion protein provided herein has an amino acid sequence that is at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the sequence of the fusion protein labeled 1412-F2.103, 1412-F5.157, 1412-F5.77, or 1412-4D11.

As would be understood by a person of ordinary skill in the art, the anti-CD40 antibody or antigen-binding fragment thereof in the fusion protein exemplified in the present invention can be replaced by antibodies or antigen-binding fragments that bind to different APC activation receptors disclosed in the present invention or other known in the art, including, for example, CD80, CD86, CD91, DEC-205 or DC-SIGN. As would be understood by a person of ordinary skill in the art, the anti-CD28 antibody or antigen-binding fragment in the fusion protein exemplified in the present invention can be replaced by antibodies or antigen-binding fragments that bind to different co-stimulatory factors of immune effector cells disclosed in the present invention or other known in the art, including, for example, antibodies or antigen-binding fragments that bind to 4-1BB, ICOS, CD27, OX40, DAP10, 2B4, CD30, CD2, LIGHT, GITR, TLR, DR3 or CD43.

Exemplary LACO-Stim (4): Antibody to Activate Receptor+Co-Stimulatory Receptor (e.g., aCD40-CD28; aCD40-4-1BB)

In some embodiments, the fusion protein provided in the present invention includes a first domain that activates APC and a second domain that activates immune effector cells, wherein the first domain includes an antibody or its antigen-binding fragment that binds to an activation receptor of APC, and wherein the second domain includes a co-stimulatory receptor or its functional fragment of immune effector cells. In some embodiments, the C-terminus of the first domain is connected to the N-terminus of the second domain. In some embodiments, the N-terminus of the first domain is connected to the C-terminus of the second domain. In some embodiments, the present invention provides a membrane fusion protein based on an antibody.

In some embodiments, the first and second domains are connected by a CD8 hinge, a CD28 hinge, or an IgG Fc region.

In some embodiments, the fusion protein provided in the present invention includes a first domain and a second domain, wherein the first domain includes an antibody or an antigen-binding fragment that binds to an activation receptor of an APC, and the second domain includes an antibody or an antigen-binding fragment that binds to a co-stimulatory receptor of an immune effector cell. In some embodiments, the first domain includes an antibody or an antigen-binding fragment thereof that binds to CD40, CD80, CD86, CD91, DEC-205 or DC-SIGN. In some embodiments, the first domain of the fusion protein provided in the present invention includes an anti-CD40 antibody or an antigen-binding fragment. The anti-CD40 antibody or antigen-binding fragment can be any anti-CD40 antibody or antigen-binding fragment that activates CD40 signaling disclosed in the present invention or otherwise known in the art. In some embodiments, the anti-CD40 antibody or antigen-binding fragment thereof includes antibodies labeled as F2.103, F5.157, F5.77, 4D11, A40C or 119 as provided in Table 1 above.

In some embodiments, the second domain includes a co-stimulatory receptor or a functional fragment thereof, and the co-stimulatory receptor is selected from the group consisting of CD28, 4-1BB, ICOS, CD27, OX40, DAP10, 2B4, CD30, CD2, LIGHT, GITR, TLR, DR3 and CD43. In some embodiments, the second domain includes the cytoplasmic domain of a co-stimulatory receptor, and the co-stimulatory receptor is selected from the group consisting of CD28, 4-1BB, ICOS, CD27, OX40, DAP10, 2B4, CD30, CD2, LIGHT, GITR, TLR, DR3 and CD43.

In some embodiments, the second domain of the fusion protein provided in the present invention includes a CD28 cytoplasmic domain. In some embodiments, the second domain of the fusion protein provided in the present invention can have an amino acid sequence that is at least 85%, at least 88%, at least 90%, at least 95%, at least 98% or 100% identical to the sequence shown in the CD28 cytoplasmic domain. In some embodiments, the second domain of the fusion protein provided in the present invention further includes a CD28 transmembrane domain.

In some embodiments, the second domain of the fusion protein provided in the present invention includes a 4-1BB cytoplasmic domain. In some embodiments, the second domain of the fusion protein provided by the text can have an amino acid sequence with at least 85%, at least 88%, at least 90%, at least 95%, at least 98% or 100% identity with the 4-1BB cytoplasmic domain. In some embodiments, the second domain of the fusion protein provided in the present invention further includes a 4-1BB transmembrane domain.

In some embodiments, the fusion protein provided in the present invention has a first domain and a second domain, the first domain includes an anti-CD40 antibody or an antigen-binding fragment thereof, and the second domain includes a CD28 cytoplasmic domain. In some embodiments, the fusion protein provided in the present invention has a first domain and a second domain, the first domain includes an anti-CD40 antibody or an antigen-binding fragment thereof, and the second domain includes a 4-1BB cytoplasmic domain. In some embodiments, the fusion protein provided in the present invention has a first domain and a second domain, the first domain includes an anti-CD40 antibody or an antigen-binding fragment thereof, and the second domain includes an ICOS cytoplasmic domain. In some embodiments, the fusion protein provided in the present invention has a first domain and a second domain, the first domain includes an anti-CD40 antibody or an antigen-binding fragment thereof, and the second domain includes a CD27 cytoplasmic domain. In some embodiments, the fusion protein provided in the present invention has a first domain and a second domain, the first domain includes an anti-CD40 antibody or an antigen-binding fragment thereof, and the second domain includes an OX40 cytoplasmic domain. In some embodiments, the fusion protein provided in the present invention has a first domain and a second domain, the first domain includes an anti-CD40 antibody or an antigen-binding fragment thereof, and the second domain includes a DAP10 cytoplasmic domain. In some embodiments, the fusion protein provided in the present invention has a first domain and a second domain, the first domain includes an anti-CD40 antibody or an antigen-binding fragment thereof, and the second domain includes a 2B4 cytoplasmic domain. In some embodiments, the fusion protein provided in the present invention has a first domain and a second domain, the first domain includes an anti-CD40 antibody or an antigen-binding fragment thereof, and the second domain includes a CD30 cytoplasmic domain. In some embodiments, the fusion protein provided in the present invention has a first domain and a second domain, the first domain includes an anti-CD40 antibody or an antigen-binding fragment thereof, and the second domain includes a CD2 cytoplasmic domain. In some embodiments, the fusion protein provided in the present invention has a first domain and a second domain, the first domain includes an anti-CD40 antibody or an antigen-binding fragment thereof, and the second domain includes a LIGHT cytoplasmic domain. In some embodiments, the fusion protein provided in the present invention has a first domain and a second domain, the first domain includes an anti-CD40 antibody or an antigen-binding fragment thereof, and the second domain includes a GITR cytoplasmic domain. In some embodiments, the fusion protein provided in the present invention has a first domain and a second domain, the first domain includes an anti-CD40 antibody or an antigen-binding fragment thereof, and the second domain includes a TLR cytoplasmic domain. In some embodiments, the fusion protein provided in the present invention has a first domain and a second domain, the first domain includes an anti-CD40 antibody or an antigen-binding fragment thereof, and the second domain includes a DR3 cytoplasmic domain. In some embodiments, the fusion protein provided in the present invention has a first domain and a second domain, the first domain includes an anti-CD40 antibody or an antigen-binding fragment thereof, and the second domain includes a CD43 cytoplasmic domain. In some embodiments, the first domain includes full-length CD40L.

In some embodiments, the fusion protein provided in the present invention further includes a transmembrane region. In some embodiments, the transmembrane region is derived from the same co-stimulatory receptor. In some embodiments, the transmembrane region is derived from different co-stimulatory receptors. In some embodiments, the fusion protein provided in the present invention has a first domain and a second domain, the first domain includes an anti-CD40 antibody or an antigen-binding fragment thereof, and the second domain includes a CD28 transmembrane region and a CD28 cytoplasmic domain. In some embodiments, the fusion protein provided in the present invention has a first domain and a second domain, the first domain includes an anti-CD40 antibody or an antigen-binding fragment thereof, and the second domain includes a 4-1BB transmembrane region and a 4-1BB cytoplasmic domain.

| Exemplary LACO-Stim | |
|---|---|
| Fusion Proteins | sequence |
| F2.103.CD28: (anti- | EVQLVESGGGLVQPGGSLRLSCAVSGFTFSTYWMHWVR |
| CD40 scFv-CD28 | QAPGKGLVWVSRINSDGSSTTYADSVKGRFTISRDNAKN |
| membrane chimeric | TLYLQMNSLRAEDTAVYYCARDRVLWIGELSYYGMDVW |
| fusion protein) | GQGTTVTVSSGGGGSGGGGSGGGGSDIQMTQSPSTLSAS |
| | VGDRVTITCRASQSISNWLAWYQQKPGKAPKLLLYKASG |
| | LESGVPSRFSGSGSGTEFTLTINSLQPDDFATYYCQQSNSY |
| | SWTFGHGTKVEIKRTASTTTPAPRPPTPAPTIASQPLSLRPE |
| | ACRPAAGGAVHTRGLDFACDFWVLVVVGGVLACYSLLV |
| | TVAFIIFWVRSKRSRLLHSDYMNMTPRRPGPTRKHYQPYA |
| | PPRDFAAYRS SEQ ID NO: 242 |
| F5.157.CD28: (anti- | EVQLLESGGGLVQPGGSLRLSCAASGFAFSSYAMSWVRQ |
| CD40 scFv-CD28 | APGKGLEWVSAISGSGGSTYYADSVKGRFTISRDNSKNTL |
| membrane chimeric | YLQMNSLRPRTRPYITVRKMGGTMVRGVMGTLTTGARE |
| fusion protein) | PWSPSPQGGGGSGGGGSGGGGSIQMTQSPSSVSASAGDR |
| | VTITCRASQGISSWLAWYQQKPGKAPKLLIYAGSSLQSGV |
| | PSRFSGSGFGTDFTLTIGSLQPEDFATYYCQQASSFPRTFG |
| | QGTKVEIKRTVLHHLSSSSRHLMSASTTTPAPRPPTPAPTI |
| | ASQPLSLRPEACRPAAGGAVHTRGLDFACDFWVLVVVGG |
| | VLACYSLLVTVAFIIFWVRSKRSRLLHSDYMNMTPRRPGP |
| | TRKHYQPYAPPRDFAAYRS SEQ ID NO: 243 |
| F5.77.CD28 (anti- | EVQLLESGGGLVQPGGSLRLSCAASGFTFSSYAMSWVRQ |
| CD40 scFv-CD28 | APGKGLEWVSAISGSGGSTYYADSVKGRFTISRDNSKNTL |
| membrane chimeric | YLQMNSLRAEDTAVYYCAKDGGYYGSGSYGYFDYWGQ |
| fusion protein): | GTLVTVSSGGGGSGGGGSGGGGSDIQMTQSPSSVSGSVG |
| | DRVTITCRASQGISSWLAWYQQKPGKAPKLLIYAGSSLQS |
| | GVPSRFSGSGFGTDFTLTISSLQPEDFATYYCQQASSFPRTF |
| | GQGTKVEIKRTASTTTPAPRPPTPAPTIASQPLSLRPEACRP |
| | AAGGAVHTRGLDFACDFWVLVVVGGVLACYSLLVTVAFI |
| | IFWVRSKRSRLLHSDYMNMTPRRPGPTRKHYQPYAPPRD |
| | FAAYRS SEQ ID NO: 244 |
| F2.103.BB (anti-CD40 | EVQLVESGGGLVQPGGSLRLSCAVSGFTFSTYWMHWVR |
| scFv-4-1BB membrane | QAPGKGLVWVSRINSDGSSTTYADSVKGRFTISRDNAKN |
| chimeric fusion | TLYLQMNSLRAEDTAVYYCARDRVLWIGELSYYGMDVW |
| protein): | GQGTTVTVSSGGGGSGGGGSGGGGSDIQMTQSPSTLSAS |
| | VGDRVTITCRASQSISNWLAWYQQKPGKAPKLLLYKASG |
| | LESGVPSRFSGSGSGTEFTLTINSLQPDDFATYYCQQSNSY |
| | SWTFGHGTKVEIKRTTTTPAPRPPTPAPTIASQPLSLRPEAC |
| | RPAAGGAVHTRGLDFACDIYIWAPLAGTCGVLLLSLVITL |
| | YCRGRKKLLYIFKQPFMRPVQTTQEEDGCSCRFPEEEEGG |
| | CE SEQ ID NO: 245 |
| F5.157.BB (anti-CD40 | EVQLLESGGGLVQPGGSLRLSCAASGFAFSSYAMSWVRQ |
| scFv-4-1BB membrane | APGKGLEWVSAISGSGGSTYYADSVKGRFTISRDNSKNTL |
| chimeric fusion | YLQMNSLRPRTRPYITVRKMGGTMVRGVMGTLTTGARE |
| protein): | PWSPSPQGGGGSGGGGSGGGGSIQMTQSPSSVSASAGDR |
| | VTITCRASQGISSWLAWYQQKPGKAPKLLIYAGSSLQSGV |
| | PSRFSGSGFGTDFTLTIGSLQPEDFATYYCQQASSFPRTFG |
| | QGTKVEIKRTVLHHLSSSSRHLMSTTTPAPRPPTPAPTIAS |
| | QPLSLRPEACRPAAGGAVHTRGLDFACDIYIWAPLAGTCG |
| | VLLLSLVITLYCRGRKKLLYIFKQPFMRPVQTTQEEDGCSC |
| | RFPEEEEGGCE SEQ ID NO: 246 |
| F5.77.BB (anti-CD40 | EVQLLESGGGLVQPGGSLRLSCAASGFTFSSYAMSWVRQ |
| scFv-4-1BB membrane | APGKGLEWVSAISGSGGSTYYADSVKGRFTISRDNSKNTL |
| chimeric fusion protein) | YLQMNSLRAEDTAVYYCAKDGGYYGSGSYGYFDYWGQ |
| | GTLVTVSSGGGGSGGGGSGGGGSDIQMTQSPSSVSGSVG |
| | DRVTITCRASQGISSWLAWYQQKPGKAPKLLIYAGSSLQS |
| | GVPSRFSGSGFGTDFTLTISSLQPEDFATYYCQQASSFPRTF |
| | GQGTKVEIKRTTTTPAPRPPTPAPTIASQPLSLRPEACRPAA |
| | GGAVHTRGLDFACDIYIWAPLAGTCGVLLLSLVITLYCRG |
| | RKKLLYIFKQPFMRPVQTTQEEDGCSCRFPEEEEGGCE |
| | SEQ ID NO: 247 |
| 4D11.CD28 (anti-CD40 | QLQLQESGPGLLKPSETLSLTCTVSGGSISSPGYYGGWIRQ |
| scFv-CD28 membrane | PPGKGLEWIGSIYKSGSTYHNPSLKSRVTISVDTSKNQFSL |
| chimeric fusion protein) | KLSSVTAADTAVYYCTRPVVRYFGWFDPWGQGTLVTVSS |
| | ASGGGGSGGGGSGGGGSAIQLTQSPSSLSASVGDRVTITC |
| | RASQGISSALAWYQQKPGKAPKLLIYDASNLESGVPSRFS |
| | GSGSGTDFTLTISSLQPEDFATYYCQQ |
| | FNSYPTFGQGTKVEIKRTASTTTPAPRPPTPAPTIASQPLSL |
| | RPEACRPAAGGAVHTRGLDFACDFWVLVVVGGVLACYS |
| | LLVTVAFIIFWVRSKRSRLLHSDYMNMTPRRPGPTRKHYQ |
| | PYAPPRDFAAYRS SEQ ID NO: 248 |
| A40C.CD28 (anti- | QVQLVQSGAEVKKPGASVKVSCTASGFNIKDYYVHWVK |
| CD40 scFv-CD28 | QAPGQGLEWMGRIDPEDGDSKYAPKFQGKATMTADTSTS |
| membrane chimeric | TVYMELSSLRSEDTAVYYCTTSYYVGTYGYWGQGTLVT |
| fusion protein) | VSSGGGGSGGGGSGGGGSDIQMTQSPSSLSASVGDRVTIT |
| | CSASSSVSYMLWFQQKPGKAPKLLIYSTSNLASGVPSRFS |
| | GSGSGTDFTLTISSLQPEDFATYYCQQRTFYPYTFGGGTKV |
| | EIKRTASTTTPAPRPPTPAPTIASQPLSLRPEACRPAAGGAV |
| | HTRGLDFACDFWVLVVVGGVLACYSLLVTVAFIIFWVRS |
| | KRSRLLHSDYMNMTPRRPGPTRKHYQPYAPPRDFAAYRS |
| | SEQ ID NO: 249 |
| 119.CD28 (anti-CD40 | QVQLQQSGPGLVKPSQTLSLTCAISGDSVSSNSATWNWIR |
| scFv-CD28 membrane | QSPSRDLEWLGRTYYRSKWYRDYVGSVKSRIIINPDTSNN |
| chimeric fusion protein) | QFSLQLNSVTPEDTAIYYCTRAQWLGGDYPYYYSMDVW |
| | GQGTTVTVSSGGGGSGGGGSGGGGSEIVLTQSPATLSLSP |
| | GERATLSCRASQSVSSYLAWYQQKPGQAPRLLIYDASNR |
| | ATGIPARFSGSGSGTDFTLTISSLEPEDFAVYYCQQRSNTFG |
| | PGTKVDIKRTASTTTPAPRPPTPAPTIASQPLSLRPEACRPA |
| | AGGAVHTRGLDFACDFWVLVVVGGVLACYSLLVTVAFIIF |
| | WVRSKRSRLLHSDYMNMTPRRPGPTRKHYQPYAPPRDFA |
| | AYRS SEQ ID NO: 250 |

In some embodiments, the fusion protein provided in the present invention has an amino acid sequence that is at least 80%, at least 85% at least 86%, at least 87%, at least 88%, at least 89%, at least 90%, at least 91%, at least 92%, at least 93%, at least 94%, at least 95%, at least 96%, at least 97%, at least 98% or at least 99% identical to the sequence of the fusion protein labeled F2.103.CD28, F5.157.CD28, F5.77.CD28, F5.157.BB, F5.77.BB, 4D11.CD28, A40C.CD28 or 119.CD28.

As would be understood by a person of ordinary skill in the art, the anti-CD40 antibody or its antigen-binding fragment in the fusion protein exemplified in the present invention can be replaced by antibodies or antigen-binding fragments that bind and activate different activation receptors of APC disclosed in the present invention or other known in the art, including, for example, CD80, CD86, CD91, DEC-205 or DC-SIGN. As would be understood by a person of ordinary skill in the art, the CD28 cytoplasmic domain or 4-1BB cytoplasmic domain in the fusion protein exemplified in the present invention can be replaced by the cytoplasmic domains of different co-stimulatory factors of immune effector cells disclosed in the present invention or other known in the art, including, for example, ICOS, CD27, OX40, DAP10, 2B4, CD30, CD2, LIGHT, GITR, TLR, DR3 or CD43 cytoplasmic domains; or 4-1BB, ICOS, CD27, OX40, DAP10, 2B4, CD30, CD2, LIGHT, GITR, TLR, DR3 or CD43 different functional fragments, the functional fragments retain the function of activating immune effector cells of the full-length protein.

Exemplary LACO-Stim (5): Antibody to APC Activating Receptor+Ligand to Co-Stimulatory Receptor (e.g., aCD40-CD80; aCD40-CD86)

In some embodiments, the fusion protein provided in the present invention includes a first domain that activates APC and a second domain that activates immune effector cells, wherein the first domain includes an antibody or its antigen-binding fragment that binds an activation receptor of an antigen presenting cell, and wherein the second domain includes a co-stimulatory ligand or its receptor binding fragment of an immune effector cell. In some embodiments, the C-terminus of the first domain is connected to the N-terminus of the second domain. In some embodiments, the N-terminus of the first domain is connected to the C-terminus of the second domain. In some embodiments, the present invention provides a soluble fusion protein based on an antibody. In some embodiments, the present invention provides a soluble fusion protein based on an antibody.

In some embodiments, the first domain includes antibodies or antigen-binding fragments thereof that bind to CD40, CD80, CD86, CD91, DEC-205, and DC-SIGN. In some embodiments, the first domain includes antibodies or antigen-binding fragments thereof that bind to CD40. In some embodiments, the first domain includes antibodies or antigen-binding fragments thereof that bind to CD80. In some embodiments, the first domain includes antibodies or antigen-binding fragments thereof that bind to CD86. In some embodiments, the first domain includes antibodies or antigen-binding fragments thereof that bind to CD91. In some embodiments, the first domain includes antibodies or antigen-binding fragments thereof that bind to DEC-205. In some embodiments, the first domain includes antibodies or antigen-binding fragments thereof that bind to DC-SIGN. The antibodies and antigen-binding fragments can be any antibodies or antigen-binding fragments disclosed herein or otherwise known in the art.

In some embodiments, the fusion protein provided in the present invention includes a first domain and a second domain, wherein the first domain includes an antibody or an antigen-binding fragment thereof that binds to an APC activation receptor, and the second domain includes a co-stimulatory ligand or a receptor binding fragment thereof of an immune effector cell. In some embodiments, the first domain includes an antibody or an antigen-binding fragment thereof that binds to CD40, CD80, CD86, CD91, DEC-205 or DC-SIGN. In some embodiments, the second domain includes a ligand selected from a group consisting of CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R and CD44, or a receptor binding fragment thereof.

In some embodiments, the first domain of the fusion protein provided in the present invention includes an anti-CD40 antibody or an antigen-binding fragment thereof. The anti-CD40 antibody or antigen-binding fragment can be any anti-CD40 antibody or antigen-binding fragment disclosed herein or otherwise known in the art that activates CD40 signaling. In some embodiments, the anti-CD40 antibody or antigen-binding fragment thereof includes an antibody labeled as F2.103, F5.157, F5.77, 4D11, A40C or 119 as provided in Table 1 above. In some embodiments, the anti-CD40 antibody or antigen-binding fragment thereof includes an anti-CD40 scFv having an amino acid sequence as shown in any one of SEQ ID NO: 227 to SEQ ID NO: 232.

In some embodiments, the fusion protein includes a first domain and a second domain, the first domain including an antibody or antigen-binding fragment that binds to CD40, and the second domain including a ligand and a receptor binding fragment thereof, the ligand being selected from the group consisting of CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R, and CD44. In some embodiments, the fusion protein includes a first domain and a second domain, the first domain including an antibody or antigen-binding fragment that binds to CD40, and the second domain including CD58 or a receptor binding fragment thereof. In some embodiments, the fusion protein includes a first domain and a second domain, the first domain including an antibody or antigen-binding fragment that binds to CD40, and the second domain including CD70 or a receptor binding fragment thereof. In some embodiments, the fusion protein includes a first domain and a second domain, the first domain including an antibody or antigen-binding fragment that binds to CD40, and the second domain including CD83 or its receptor binding fragment. In some embodiments, the fusion protein includes a first domain and a second domain, the first domain including an antibody or antigen-binding fragment that binds to CD40, and the second domain including CD80 or its receptor binding fragment. In some embodiments, the fusion protein includes a first domain and a second domain, the first domain including an antibody or antigen-binding fragment that binds to CD40, and the second domain including CD86 or its receptor binding fragment. In some embodiments, the fusion protein includes a first domain and a second domain, the first domain including an antibody or antigen-binding fragment that binds to CD40, and the second domain including CD137L or its receptor binding fragment. In some embodiments, the fusion protein includes a first domain and a second domain, the first domain including an antibody or antigen-binding fragment that binds to CD40, and the second domain including CD252 or its receptor binding fragment. In some embodiments, the fusion protein includes a first domain and a second domain, the first domain including an antibody or antigen-binding fragment that binds to CD40, and the second domain including CD275 or its receptor binding fragment. In some embodiments, the fusion protein includes a first domain and a second domain, the first domain includes an antibody or antigen-binding fragment that binds to CD40, and the second domain includes CD54 or its receptor binding fragment. In some embodiments, the fusion protein includes a first domain and a second domain, the first domain includes an antibody or antigen-binding fragment that binds to CD40, and the second domain includes CD49a or its receptor binding fragment. In some embodiments, the fusion protein includes a first domain and a second domain, the first domain includes an antibody or antigen-binding fragment that binds to CD40, and the second domain includes CD112 or its receptor binding fragment. In some embodiments, the fusion protein includes a first domain and a second domain, the first domain includes an antibody or antigen-binding fragment that binds to CD40, and the second domain includes CD150 or its receptor binding fragment. In some embodiments, the fusion protein includes a first domain and a second domain, the first domain includes an antibody or antigen-binding fragment that binds to CD40, and the second domain includes CD155 or its receptor binding fragment. In some embodiments, the fusion protein includes a first domain and a second domain, the first domain includes an antibody or antigen-binding fragment that binds to CD40, and the second domain includes CD265 or its receptor binding fragment. In some embodiments, the fusion protein includes a first domain and a second domain, the first domain includes an antibody or antigen-binding fragment that binds to CD40, and the second domain includes CD270 or its receptor binding fragment. In some embodiments, the fusion protein includes a first domain, which includes an antibody or antigen-binding fragment that binds to CD40, and the second domain includes TL1A or its receptor binding fragment. In some embodiments, the fusion protein includes a first domain and a second domain, the first domain includes an antibody or antigen-binding fragment that binds to CD40, and the second domain includes CD127 or its receptor binding fragment. In some embodiments, the fusion protein includes a first domain and a second domain, the first domain includes an antibody or antigen-binding fragment that binds to CD40, and the second domain includes IL-4R or its receptor binding fragment. In some embodiments, the fusion protein includes a first domain, which includes an antibody or antigen-binding fragment that binds to CD40, and the second domain includes GITR-L or its receptor binding fragment. In some embodiments, the fusion protein includes a first domain and a second domain, the first domain includes an antibody or antigen-binding fragment that binds to CD40, and the second domain includes TIM-4 or its receptor binding fragment. In some embodiments, the fusion protein includes a first domain and a second domain, the first domain includes an antibody or antigen-binding fragment that binds to CD40, and the second domain includes CD153 or its receptor binding fragment. In some embodiments, the fusion protein includes a first domain and a second domain, the first domain includes an antibody or antigen-binding fragment that binds to CD40, and the second domain includes CD48 or its receptor binding fragment. In some embodiments, the fusion protein includes a first domain and a second domain, the first domain includes an antibody or antigen-binding fragment that binds to CD40, and the second domain includes CD160 or its receptor binding fragment. In some embodiments, the fusion protein includes a first domain and a second domain, the first domain includes an antibody or antigen-binding fragment that binds to CD40, and the second domain includes CD200R or its receptor binding fragment. In some embodiments, the fusion protein includes a first domain and a second domain, the first domain includes an antibody or antigen-binding fragment that binds to CD40, and the second domain includes CD44 or its receptor binding fragment. As would be understood by a person of ordinary skill in the art can easily determine a suitable receptor binding fragment of a ligand, which retains its binding affinity to the receptor and has the function of activating the receptor.

As would be understood by a person of ordinary skill in the art, the anti-CD40 antibody or antigen-binding fragment thereof in the fusion protein exemplified in the present invention can be replaced by antibodies or antigen-binding fragments disclosed in the present invention or otherwise known in the art that bind to different activation receptors of APCs, including, for example, CD80, CD86, CD91, DEC-205 or DC-SIGN.

### Exemplary LACO-Stim (6): APC Activating Receptor Ligand+Co-Stimulatory Ligand (e.g., CD40L-CD86; CD40L-CD80)

In some embodiments, the fusion protein provided in the present invention includes a first domain that activates APC and a second domain that activates immune effector cell, wherein the first domain includes a ligand or its receptor binding fragment that binds to an activation receptor of APC, and wherein the second domain includes a co-stimulatory ligand or its receptor binding fragment of immune effector cell. In some embodiments, the C-terminus of the first domain is connected to the N-terminus of the second domain. In some embodiments, the N-terminus of the first domain is connected to the C-terminus of the second domain.

In some embodiments, the fusion protein provided in the present invention includes a first domain and a second domain, wherein the first domain includes a ligand or a functional fragment thereof that binds to an activating receptor, wherein the activating receptor is selected from the group consisting of CD40, CD80, CD86, CD91, DEC-205 and DC-SIGN, and the second domain includes a co-stimulatory ligand or a receptor binding fragment thereof, wherein the co-stimulatory ligand is selected from the group consisting of CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R, and CD44.

In some embodiments, the first domain of the fusion protein provided in the present invention includes the extracellular domain of CD40L. In some embodiments, the first domain of the fusion protein provided in the present invention can have an amino acid sequence that is at least 85%, at least 88%, at least 90%, at least 95%, at least 98% or 100% identical to the extracellular domain of CD40L. In some embodiments, the first domain of the fusion protein provided in the present invention includes full-length CD40L. In some embodiments, the first domain of the fusion protein provided in the present invention can have amino acids 119-261 of CD40L (SEQ ID NO: 223). In some embodiments, the first domain of the fusion protein provided in the present invention includes three copies of CD40L or a functional fragment thereof. In some embodiments, the first domain of the fusion protein provided in the present invention includes three copies of the extracellular domain of CD40L. In some embodiments, the first domain of the fusion protein provided in the present invention includes three copies of amino acids 119-261 of CD40L (SEQ ID NO: 223).

In some embodiments, the fusion protein includes a first domain and a second domain, the first domain includes CD40L or its receptor binding fragment, the second domain includes a ligand or its receptor binding fragment, the ligand is selected from the group consisting of CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R, CD44. In some embodiments, the fusion protein includes a first domain and a second domain, the first domain includes CD40L or its receptor binding fragment, and the second domain includes CD58 or its receptor binding fragment. In some embodiments, the fusion protein includes a first domain and a second domain, the first domain includes CD40L or its receptor binding fragment, and the second domain includes CD70 or its receptor binding fragment. In some embodiments, the fusion protein includes a first domain and a second domain, the first domain includes CD40L or its receptor binding fragment, and the second domain includes CD83 or its receptor binding fragment. In some embodiments, the fusion protein includes a first domain and a second domain, the first domain includes CD40L or its receptor binding fragment, and the second domain includes CD80 or its receptor binding fragment. In some embodiments, the fusion protein includes a first domain and a second domain, the first domain includes CD40L or its receptor binding fragment, and the second domain includes CD86 or its receptor binding fragment. In some embodiments, the fusion protein includes a first domain and a second domain, the first domain includes CD40L or its receptor binding fragment, and the second domain includes CD137L or its receptor binding fragment. In some embodiments, the fusion protein includes a first domain and a second domain, the first domain includes CD40L or its receptor binding fragment, and the second domain includes CD252 or its receptor binding fragment. In some embodiments, the fusion protein includes a first domain and a second domain, the first domain includes CD40L or its receptor binding fragment, and the second domain includes CD275 or its receptor binding fragment. In some embodiments, the fusion protein includes a first domain and a second domain, the first domain includes CD40L or its receptor binding fragment, and the second domain includes CD54 or its receptor binding fragment. In some embodiments, the fusion protein includes a first domain and a second domain, the first domain includes CD40L or its receptor binding fragment, and the second domain includes CD49a or its receptor binding fragment. In some embodiments, the fusion protein includes a first domain and a second domain, the first domain includes CD40L or its receptor binding fragment, and the second domain includes CD112 or its receptor binding fragment. In some embodiments, the fusion protein includes a first domain and a second domain, the first domain includes binding CD40L or its receptor binding fragment, and the second domain includes CD150 or its receptor binding fragment. In some embodiments, the fusion protein includes a first domain and a second domain, the first domain includes CD40L or its receptor binding fragment, and the second domain includes CD155 or its receptor binding fragment. In some embodiments, the fusion protein includes a first domain and a second domain, the first domain includes CD40L or its receptor binding fragment, and the second domain includes CD265 or its receptor binding fragment. In some embodiments, the fusion protein includes a first domain and a second domain, the first domain includes CD40L or its receptor binding fragment, and the second domain includes CD270 or its receptor binding fragment. In some embodiments, the fusion protein includes a first domain and a second domain, the first domain includes CD40L or its receptor binding fragment, and the second domain includes TL1A or its receptor binding fragment. In some embodiments, the fusion protein includes a first domain and a second domain, the first domain includes CD40L or its receptor binding fragment, and the second domain includes CD127 or its receptor binding fragment. In some embodiments, the fusion protein includes a first domain and a second domain, the first domain includes CD40L or its receptor binding fragment, and the second domain includes IL-4R or its receptor binding fragment. In some embodiments, the fusion protein includes a first domain and a second domain, the first domain includes CD40L or its receptor binding fragment, and the second domain includes GITR-L or its receptor binding fragment. In some embodiments, the fusion protein includes a first domain and a second domain, the first domain includes CD40L or its receptor binding fragment, and the second domain includes TIM-4 or its receptor binding fragment. In some embodiments, the fusion protein includes a first domain and a second domain, the first domain includes CD40L or its receptor binding fragment, and the second domain includes CD153 or its receptor binding fragment. In some embodiments, the fusion protein includes a first domain and a second domain, the first domain includes CD40L or its receptor binding fragment, and the second domain includes CD48 or its receptor binding fragment. In some embodiments, the fusion protein includes a first domain and a second domain, the first domain includes CD40L or its receptor binding fragment, and the second domain includes CD160 or its receptor binding fragment. In some embodiments, the fusion protein includes a first domain and a second domain, the first domain includes CD40L or its receptor binding fragment, and the second domain includes CD200R or its receptor binding fragment. In some embodiments, the fusion protein includes a first domain and a second domain, the first domain includes binding to CD40L or its receptor binding fragment, and the second domain includes CD44 or its receptor binding fragment. As would be understood by a person of ordinary skill in the art can easily determine a suitable receptor binding fragment of a ligand, which retains its binding affinity to the receptor and has the function of activating the receptor.

As will be appreciated by those skilled in the art, the CD40L or its receptor binding fragment in the fusion protein exemplified in the present invention can be replaced with different ligands of APC activating receptors disclosed in the present invention or otherwise known in the art, including, for example, the extracellular domain or full-length ligand of CD80 ligand (e.g., CD28 or CTLA-4), CD86 ligand (e.g., CD28 or CTLA-4), CD91 ligand (e.g., RAP1), DEC-205 ligand or DC-SIGN ligand (e.g., ICAM2, ICAM3, CD18 or CEACAM1).

### Anti-CD7 CAR

Extracellular domain of the chimeric antigen receptor recognizes and specifically binds to an antigen, typically an antigen expressed on the surface of a malignant tumor (such as CD7). For example, when the antigen-specific extracellular domain binds to an antigen with an affinity constant or interaction affinity (KD) of about 0.1 pM to about 10 μM, or about 0.1 pM to about 1 μM, or about 0.1 pM to about 100 nM, the antigen-specific extracellular domain specifically binds to the antigen. Methods for determining interaction affinity are known in the art. The antigen-specific extracellular domain suitable for the CAR of the present disclosure can be any antigen-binding polypeptide, and a variety of antigen-binding polypeptides are known in the art. In some cases, the antigen-binding domain is a single-chain Fv (scFv). Other antibody-based recognition domains (cAb VHH (camelid antibody variable domain) and humanized forms thereof, IgNAR VH (shark antibody variable domain) and humanized forms thereof, sdAb VH (single domain antibody variable domain) and “camelized” antibody variable domains are suitable for use. In some cases, T cell receptor (TCR)-based recognition domains such as single chain TCR (scTv) are also suitable for use.

Suitable antigens can include T cell-specific antigens and/or antigens that are not specific for T cells. In some embodiments, the antigens specifically bound by the chimeric antigen receptor of the CAR-T cell and the antigens lacking in the CAR-T cell are antigens expressed on malignant T cells, more preferably antigens overexpressed on malignant T cells compared with non-malignant T cells. “Malignant T cells” are T cells derived from T cell malignancies. The term “T cell malignancy” refers to a wide range of highly heterogeneous groupings of malignant tumors derived from T cell precursors, mature T cells or natural killer cells. Non-limiting examples of T-cell malignancies include T-cell acute lymphoblastic leukemia/lymphoma (T-ALL), T-cell large granular lymphocytic (LGL) leukemia, human T-cell leukemia virus type 1-positive (HTLV-1+) adult T-cell leukemia/lymphoma (ATL), T-cell prolymphocytic leukemia (T-PLL), and various peripheral T-cell lymphomas (PTCL), including but not limited to angioimmunoblastic T-cell lymphoma (AITL), ALK-positive anaplastic large cell lymphoma, and ALK-negative anaplastic large cell lymphoma.

In some embodiments, the CAR-T cells of the present application comprise an extracellular domain of a chimeric antigen receptor that specifically binds to CD7. CD7 is a T cell surface membrane-associated glycoprotein. CD7 can be overexpressed in T cell malignancies including T cell acute lymphoblastic leukemia (T-ALL) and non-Hodgkin's T cell lymphoma (NHL). The CAR-T cell disclosed herein can be used to target malignant T cell that overexpress CD7.

In some embodiments, the CAR comprises an extracellular antigen-binding domain comprising a heavy chain variable region (VH) and a light chain variable region (VL), wherein the VH comprises the amino acid sequence shown in SEQ ID NO: 61 to SEQ ID NO: 73.

In some embodiments, the VL comprises the amino acid sequence shown in any one of SEQ ID NO: 148 to SEQ ID NO: 160.

In some embodiments, the VH comprises the amino acid sequence shown in SEQ ID NO: 61, and the VL comprises the amino acid sequence shown in SEQ ID NO: 148; or
The VH comprises the amino acid sequence shown in SEQ ID NO: 62, and the VL comprises the amino acid sequence shown in SEQ ID NO: 149; or
The VH comprises the amino acid sequence shown in SEQ ID NO: 63, and the VL comprises the amino acid sequence shown in SEQ ID NO: 150; or
The VH comprises the amino acid sequence shown in SEQ ID NO: 64, and the VL comprises the amino acid sequence shown in SEQ ID NO: 151; or
The VH comprises the amino acid sequence shown in SEQ ID NO: 65, and the VL comprises the amino acid sequence shown in SEQ ID NO: 152; or
The VH comprises the amino acid sequence shown in SEQ ID NO: 66, and the VL comprises the amino acid sequence shown in SEQ ID NO: 153; or
The VH comprises the amino acid sequence shown in SEQ ID NO: 67, and the VL comprises the amino acid sequence shown in SEQ ID NO: 154; or
The VH comprises the amino acid sequence shown in SEQ ID NO: 68, and the VL comprises the amino acid sequence shown in SEQ ID NO: 155; or
The VH comprises the amino acid sequence shown in SEQ ID NO: 69, and the VL comprises the amino acid sequence shown in SEQ ID NO: 156; or
The VH comprises the amino acid sequence shown in SEQ ID NO: 70, and the VL comprises the amino acid sequence shown in SEQ ID NO: 157; or
The VH comprises the amino acid sequence shown in SEQ ID NO: 71, and the VL comprises the amino acid sequence shown in SEQ ID NO: 158; or
The VH comprises the amino acid sequence shown in SEQ ID NO: 72, and the VL comprises the amino acid sequence shown in SEQ ID NO: 159; or
The VH comprises the amino acid sequence shown in SEQ ID NO: 73, and the VL comprises the amino acid sequence shown in SEQ ID NO: 160.

In some embodiments, the extracellular antigen-binding domain includes an scFv.

For example, the extracellular antigen-binding domain can be an anti-CD7 scFv.

In some embodiments, the VL and VH are connected by a linker.

In some embodiments, the linker includes a polypeptide linker.

In some embodiments, the polypeptide linker comprises an amino acid sequence represented by (GGGGS)n, wherein n is any integer from 1 to 5.

extracellular domain of the chimeric antigen receptor of the present application can specifically bind to CD7, comprising the amino acid sequence shown in any one of SEQ ID NO: 162 to SEQ ID NO: 174, or an amino acid sequence having at least about 90%, about 91%, about 92%, about 93%, about 94%, about 95%, about 96%, about 97%, about 98%, about 99%, or about 99.5% identity with the amino acid sequence shown in any one of SEQ ID NO: 162 to SEQ ID NO: 174.

The chimeric antigen receptor of the present application also comprises an intracellular domain, which provides an intracellular signal to the T cell after the antigen is bound to the antigen-specific extracellular domain. Non-limiting examples of suitable intracellular domains include any one of the ζ chain of the T cell receptor or its homologues (e.g., η, δ, γ or ε), MB 1 chain, B29, Fc RIII, Fc RI, and signaling molecules (such as CD3.ζ. and CD28, CD27, 4-1BB, DAP-10, OX40 and combinations thereof and other similar molecules and fragments). Intracellular signaling portions of other members of the activator protein family can be used, such as Fc.γ.RIII and Fc.ε.RI. Although the entire intracellular domain is generally used, in many cases, it is not necessary to use the entire intracellular polypeptide. To the extent that the truncated portion of the intracellular signaling domain can be used, this truncated portion can be used instead of the complete chain, as long as the truncated portion still transduces the effector function signal. Thus, the intracellular domain of the present application is intended to include any truncated portion of the intracellular domain that is sufficient to transduce the effector function signal.

Typically, the antigen-specific extracellular domain is connected to the intracellular domain of the chimeric antigen receptor through a transmembrane domain. The transmembrane domain traverses the cell membrane, anchors CAR to the T cell surface, and connects the extracellular domain to the intracellular signaling domain, thereby affecting the expression of CAR on the T cell surface. The chimeric antigen receptor can further comprise one or more co-stimulatory domains and/or one or more spacers. The co-stimulatory domain is derived from the intracellular signaling domain of the enhanced in vivo cytokine production, proliferation, cytotoxicity and/or persistence of the co-stimulatory protein. The spacer (i) connects the antigen-specific extracellular domain to the transmembrane domain, (ii) connects the transmembrane domain to the co-stimulatory domain, (iii) connects the co-stimulatory domain to the intracellular domain, and/or (iv) connects the transmembrane domain to the intracellular domain. For example, the inclusion of a spacer domain between the antigen-specific extracellular domain and the transmembrane domain can affect the flexibility of the antigen-binding domain and thus affect the CAR function. Suitable transmembrane domains, co-stimulatory domains and spacers are known in the art.

In some embodiments, the chimeric antigen receptor includes a transmembrane domain, comprising a transmembrane domain derived from one or more proteins selected from the following group: CD8, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64 and SLAM.

For example, the transmembrane domain can comprise a transmembrane domain derived from CD8.

For another example, the transmembrane domain comprises the amino acid sequence shown in SEQ ID NO: 177, or an amino acid sequence that is at least about 90%, about 91%, about 92%, about 93%, about 94%, about 95%, about 96%, about 97%, about 98%, about 99%, or about 99.5% identical to the amino acid sequence shown in SEQ ID NO: 177.

In some embodiments, the chimeric antigen receptor includes an intracellular co-stimulatory signaling domain, comprising an intracellular co-stimulatory signaling domain derived from one or more proteins selected from the group consisting of CD28, CD137, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40, and MyD88.

For example, the intracellular co-stimulatory signaling domain can comprise a co-stimulatory signaling domain derived from 4-1BB.

For another example, the intracellular co-stimulatory signaling domain can comprise the amino acid sequence shown in SEQ ID NO: 178.

In some embodiments, the chimeric antigen receptor includes an intracellular signaling transduction domain, comprising an intracellular signaling transduction domain derived from one or more proteins selected from the following group: CD3ζ, CD3δ, CD3γ, CD3ε, CD79a, CD79b, FceRIγ, FceRIβ, FcγRIIa, bovine leukemia virus gp30, Epstein-Barr virus (EBV) LMP2A, simian immunodeficiency virus PBj14 Nef, DAP10, DAP-12 and a domain comprising at least one ITAM.

For example, the intracellular signaling domain can comprise a signaling domain derived from CD3ζ.

For another example, the intracellular signaling domain can comprise the amino acid sequence shown in SEQ ID NO: 179.

In some embodiments, it includes a hinge region between the extracellular antigen-binding domain and the transmembrane domain, wherein the hinge region comprises a hinge region derived from one or more proteins selected from the following groups: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, TIM1, SLAM, CD30 and LIGHT.

For example, the hinge region can comprise a hinge region derived from CD8.

For example, the hinge region can comprise the amino acid sequence shown in SEQ ID NO: 176.

In some embodiments, the non-targeting portion of the chimeric antigen receptor comprises a transmembrane domain, a hinge region, an intracellular co-stimulatory signaling domain, and an intracellular signaling domain.

non-targeting portion of the chimeric antigen receptor comprises the transmembrane domain of the CD8 molecule, the hinge region of CD8, the intracellular co-stimulatory signaling domain of 4-1BB, and the intracellular signaling domain of CD3ζ.

In some embodiments, it further comprises a signal peptide fragment, wherein the C-terminus of the signal peptide fragment is connected to the N-terminus of the extracellular antigen-binding domain.

For example, the signal peptide fragment can comprise a CD8 signal peptide fragment.

For example, the signal peptide fragment can comprise the amino acid sequence shown in SEQ ID NO: 175.

For another example, the chimeric antigen receptor of the present application can comprise an amino acid sequence as shown in any one of SEQ ID NO: 180 to SEQ ID NO: 192, or an amino acid sequence having at least about 90%, about 91%, about 92%, about 93%, about 94%, about 95%, about 96%, about 97%, about 98%, about 99%, or about 99.5% identity with the amino acid sequence as shown in any one of SEQ ID NO: 180 to SEQ ID NO: 192.

### Engineered Immune Effector Cell

In the present application, immune effector cell includes an engineered immune effector cell. The term “engineered immune cell” generally refers to an immune cell genetically modified by adding additional genetic material in the form of DNA or RNA to the total genetic material of the cell, also referred to as an immune effector cell. For example, engineered immune cells have been genetically modified to express the CAR targeting CD7 of the present invention. The immune effector cell includes a T cell, a B cell, a natural killer cell (NK cells), a macrophage, an NKT cell, a monocyte, a dendritic cell, a granulocyte, a lymphocyte, a leukocyte and/or a peripheral blood mononuclear cell.

In some embodiments, the immune effector cell includes an autologous or allogeneic immune effector cell. For example, wherein the immune effector cell can include an allogeneic T cell or an autologous T cell.

In some embodiments, the engineered immune effector cell includes a CAR-T cell, a CAR-NK cell or a TCR-T cell.

In some embodiments, the modified immune effector cells have reduced CD7 surface expression compared with corresponding immune cells and express anti-CD7 CAR.

In some embodiments, the engineered immune effector cell includes an anti-CD7 CAR-T cell.

In some embodiments, the CAR comprises an extracellular antigen-binding domain, and the extracellular antigen-binding domain comprises the amino acid sequence shown in any one of SEQ ID NO: 162 to SEQ ID NO: 174.

In some embodiments, the CAR comprises the amino acid sequence shown in any one of SEQ ID NO: 180 to SEQ ID NO: 192.

In some embodiments, the expression of CD7 in the modified immune effector cells is absent or suppressed.

In some embodiments, the CAR-T cell lacks the antigen to which the chimeric antigen receptor specifically binds and therefore does not induce fratricide.

In some embodiments, the antigen of the T cell is modified so that the chimeric antigen receptor no longer specifically binds to the modified antigen (such as CD7). For example, the epitope of the antigen recognized by the chimeric antigen receptor can be modified by one or more amino acid changes (e.g., substitution or deletion), or the epitope can be deleted from the antigen. In other embodiments, the expression of the antigen is reduced by at least 50%, at least 60%, at least 70%, at least 80%, at least 90% or more in T cells. Methods for reducing the expression of proteins are known in the art and include, but are not limited to, modifying or replacing a promoter operably connected to a nucleic acid sequence encoding a protein. In other embodiments, T cells are modified so that, for example, the antigen is not expressed by the deletion or destruction of the gene encoding the antigen. Methods for genetically modifying T cells to lack antigens are well known in the art, and non-limiting examples are provided above. In an exemplary embodiment, CRISPR/cas9 gene editing can be used to modify T cells to lack antigens.

Methods for designing, delivering and expressing CARs in T cells and the manufacture of clinical-grade CAR-T cell populations are known in the art. See, for example, Lee et al., Clin. Cancer Res., 2012, 18(10): 2780-90. For example, engineered CARs can be introduced into T cells using retroviruses, which effectively and stably integrate nucleic acid sequences encoding chimeric antigen receptors into the target cell genome. For another example, mRNA encoding CAR is transferred into T cells. Other methods known in the art include, but are not limited to, lentiviral transduction, transposon-based systems, direct RNA transfection, and CRISPR/Cas systems.

CAR-T cells can be generated from any suitable T cell source known in the art, including but not limited to T cells collected from subjects. The subject can be a patient with a T cell malignancy requiring CAR-T cell therapy or a subject of the same type as a subject with a T cell malignancy requiring CAR-T cell therapy. The collected T cells can be expanded ex vivo using methods generally known in the art, and then transduced with CAR to generate CAR-T cells.

On the other hand, the present application provides a cell population comprising the modified immune effector cells described herein, wherein the cell population is derived from peripheral blood mononuclear cells (PBMC), peripheral blood leukocytes (PBL), tumor infiltrating lymphocytes (TIL), cytokine-induced killer cells (CIK), lymphokine-activated killer cells (LAK) or bone marrow infiltrating lymphocytes (MIL).

### Drug Combination

On the other hand, the present application provides a drug combination comprising an immune effector cell and a fusion protein, wherein the fusion protein comprises a first domain that activates an antigen presenting cell (APC) and a second domain that activates an immune effector cell, wherein (i) the first domain includes (a) an activating receptor ligand or a receptor binding fragment thereof that binds to the APC, or (b) an activating receptor antibody or an antigen-binding fragment thereof that binds to the APC; and (ii) the second domain includes (a) a co-stimulatory ligand or a receptor binding fragment thereof of the immune effector cell, or (b) an antibody or an antigen-binding fragment thereof that binds to a co-stimulatory receptor of the immune effector cell.

In some embodiments, the fusion protein is a soluble protein. In some embodiments, the fusion protein is a bispecific antibody.

For example, the first domain and the second domain of the fusion protein can be selected from the following combinations:
ligand of APC activation receptor+antibody binding to co-stimulatory receptor (e.g., aCD28-CD40L);
Antibody to APC activation receptor+antibody to co-stimulatory receptor (e.g., aCD40/aCD28 bispecific Ab);
Antibodies to APC activation receptor+co-stimulatory ligand (e.g., aCD40-CD80;
aCD40-CD86); and
ligand of APC activation receptor+co-stimulatory ligand (e.g., CD40L-CD86; CD40L-CD80).

### Method and Use

On the other hand, the present application provides the use of the modified immune effector cells described in the present application, the drug combination described in the present application, or the pharmaceutical composition described in the present application in the preparation of a drug for treating a tumor.

In some embodiments, the tumor includes a hematological tumor and a solid tumor.

In some embodiments, the tumor includes a tumor expressing CD7.

In some embodiments, the tumor includes a CD7-positive hematological malignancy.

In some embodiments, the tumor includes a T-cell malignancy.

In some embodiments, the T-cell malignancy includes acute T-lymphocytic leukemia (T-ALL), acute myeloid leukemia, or NK/T-cell lymphoma.

On the other hand, the present application provides a method for treating a tumor, including administering the modified immune effector cells described herein, the drug combination described herein, or the pharmaceutical composition described herein to a subject in need thereof.

CAR-T cells can be administered to a subject by an intravenous route (e.g., by intravenous infusion). CAR-T cells can be administered in a single dose or in multiple doses. CAR-T cells can be injected in a pharmaceutical composition suitable for intravenous administration. Suitable pharmaceutical compositions for IV administration are known in the art. The pharmaceutical composition of the present disclosure can also include additional components. For example, such components can be used to maintain the viability and/or activity of injected CAR-T cells.

CAR-T cells can be administered at an effective dose. An effective dose can be one dose or multiple doses and is sufficient to produce the desired therapeutic effect. A typical dose of CAR-T cells can range from about 1×10⁵ to 5×10⁷ cells/Kg of the body weight of the subject receiving therapy. The effective dose can be calculated based on the stage of the malignant tumor, the health status of the subject, and the type of malignant tumor. In the case of administering multiple doses, this dose and the interval between doses can be determined based on the subject's response to therapy.

On the other hand, the present application provides a method for killing malignant T cells, the method including contacting the malignant T cells with the modified immune effector cells described in the present application, the drug combination described in the present application, or the pharmaceutical composition described in the present application.

On the other hand, the present application provides a method for preparing a chimeric antigen receptor T (CAR-T) cell population, wherein the CAR targets CD7, including the following steps:
a. Modify the CD7 gene in the population of T cells;
b. Activating the population of T cells;
c. Transducing the population of anti-CD7 CAR and fusion protein T cells; and
d. Expanding the population of chimeric antigen receptor T cells.

Methods for designing, delivering and expressing CARs in T cells and the manufacture of clinical-grade CAR-T cell populations are known in the art. See, for example, Lee et al., Clin. Cancer Res., 2012, 18(10): 2780-90. For example, engineered CARs can be introduced into T cells using retroviruses, which effectively and stably integrate nucleic acid sequences encoding chimeric antigen receptors into the target cell genome. For another example, mRNA encoding CAR is transferred into T cells. Other methods known in the art include, but are not limited to, lentiviral transduction, transposon-based systems, direct RNA transfection, and CRISPR/Cas systems.

CAR-T cells can be generated from any suitable T cell source known in the art, including but not limited to T cells collected from subjects. The subject can be a patient with a T cell malignancy requiring CAR-T cell therapy or a subject of the same type as a subject with a T cell malignancy requiring CAR-T cell therapy. The collected T cells can be expanded ex vivo using methods generally known in the art, and then transduced with CAR to generate CAR-T cells.

In an exemplary embodiment, the present disclosure provides a T cell comprising a chimeric antigen receptor that specifically binds to CD7, wherein the T cell lacks CD7 (e.g., CD7 KO CAR-T cells). In a non-limiting example, the lack of CD7 is produced by: (a) modifying CD7 expressed by the T cell so that the chimeric antigen receptor no longer specifically binds to the modified CD7, (b) modifying the T cell so that the expression of the antigen is reduced in the T cell by at least 50%, at least 60%, at least 70%, at least 80%, at least 90% or more, or (c) modifying the T cell so that CD7 is not expressed (e.g., by deletion or destruction of a gene encoding CD7).

In some embodiments, the modification includes administering to the T cell population one or more substances selected from the group consisting of antisense RNA, siRNA, shRNA, transcription activator-like effector nuclease (TALEN), zinc finger nuclease (ZFN) and CRISPR/Cas system.

In some embodiments, wherein the modification includes administering a CRISPR/Cas system to the T cell population.

In some embodiments, the modification includes administering a CRISPR/Cas9 system to the T cell population.

In some embodiments, the modification includes administering Cas9 and a gRNA targeting the CD7 gene to the T cell population.

In some embodiments, the gRNA targeting the CD7 gene comprises a nucleotide sequence shown in any one of SEQ ID NO: 211 to SEQ ID NO: 218.

In some embodiments, the Cas9 is delivered to the cell as mRNA or protein.

In some embodiments, the gRNA is delivered simultaneously with the Cas9.

In some embodiments, wherein the delivering is by electroporation.

In some embodiments, the transduction of the anti-CD7 CAR and the fusion protein into the T cell includes introducing a nucleic acid molecule encoding the anti-CD7 CAR and a nucleic acid molecule encoding the fusion protein into the T cells.

### Antigen-Binding Protein

On the one hand, isolated antigen-binding protein is provided, wherein the isolated antigen-binding protein competes with a reference antibody for binding to CD7, wherein the reference antibody comprises a light chain variable region VL and a heavy chain variable region VH;
Wherein the VH comprises the amino acid sequence shown in SEQ ID NO: 61, and the VL comprises the amino acid sequence shown in SEQ ID NO: 148, and the reference antibody is named anti-CD7 scFv-H1 antibody; or
The VH comprises the amino acid sequence shown in SEQ ID NO: 62, the VL comprises the amino acid sequence shown in SEQ ID NO: 149, and the reference antibody is named anti-CD7 scFv-H4 antibody; or
The VH comprises the amino acid sequence shown in SEQ ID NO: 63, the VL comprises the amino acid sequence shown in SEQ ID NO: 150, and the reference antibody is named anti-CD7 scFv-H6 antibody; or
The VH comprises the amino acid sequence shown in SEQ ID NO: 64, the VL comprises the amino acid sequence shown in SEQ ID NO: 151, and the reference antibody is named anti-CD7 scFv-H8 antibody; or
The VH comprises the amino acid sequence shown in SEQ ID NO: 65, the VL comprises the amino acid sequence shown in SEQ ID NO: 152, and the reference antibody is named anti-CD7 scFv-H9 antibody; or
The VH comprises the amino acid sequence shown in SEQ ID NO: 66, the VL comprises the amino acid sequence shown in SEQ ID NO: 153, and the reference antibody is named anti-CD7 scFv-H10 antibody; or
The VH comprises the amino acid sequence shown in SEQ ID NO: 67, the VL comprises the amino acid sequence shown in SEQ ID NO: 154, and the reference antibody is named anti-CD7 scFv-H12 antibody; or
The VH comprises the amino acid sequence shown in SEQ ID NO: 68, the VL comprises the amino acid sequence shown in SEQ ID NO: 155, and the reference antibody is named anti-CD7 scFv-H13 antibody; or
The VH comprises the amino acid sequence shown in SEQ ID NO: 69, the VL comprises the amino acid sequence shown in SEQ ID NO: 156, and the reference antibody is named anti-CD7 scFv-H17 antibody; or
The VH comprises the amino acid sequence shown in SEQ ID NO: 70, the VL comprises the amino acid sequence shown in SEQ ID NO: 157, and the reference antibody is named anti-CD7 scFv-H18 antibody; or
The VH comprises the amino acid sequence shown in SEQ ID NO: 71, the VL comprises the amino acid sequence shown in SEQ ID NO: 158, and the reference antibody is named anti-CD7 scFv-H5 antibody; or
The VH comprises the amino acid sequence shown in SEQ ID NO: 72, the VL comprises the amino acid sequence shown in SEQ ID NO: 159, and the reference antibody is named anti-CD7 scFv-H7 antibody; or
The VH comprises the amino acid sequence shown in SEQ ID NO: 73, the VL comprises the amino acid sequence shown in SEQ ID NO: 160, and the reference antibody is named anti-CD7 scFv-H15 antibody.

In some embodiments, the isolated antigen-binding protein comprises a heavy chain variable region (VH), wherein the VH comprises HCDR1, HCDR2 and HCDR3, wherein HCDR1 comprises the amino acid sequence shown in SEQ ID NO: 1, and the general sequence formula of SEQ ID NO: 1 is shown as G[FGY][ST][FILV][ST][EGST][LNY], wherein the amino acid in [ ] is an optional amino acid.

For example, the HCDR1 can comprise an amino acid sequence shown in any one of SEQ ID NO: 2 to SEQ ID NO: 9.

In some embodiments, the HCDR2 comprises the amino acid sequence shown in any one of SEQ ID NO: 10 to SEQ ID NO: 17.

In some embodiments, the HCDR3 comprises the amino acid sequence shown in SEQ ID NO: 18 or SEQ ID NO: 27.

For another example, the isolated antigen-binding protein comprises a heavy chain variable region (VH), the VH can comprise HCDR1, HCDR2 and HCDR3, wherein the HCDR1 can comprise the amino acid sequence shown in SEQ ID NO: 2, the HCDR2 can comprise the amino acid sequence shown in SEQ ID NO: 10, and the HCDR3 can comprise the amino acid sequence shown in SEQ ID NO: 18; or
The HCDR1 can comprise the amino acid sequence shown in SEQ ID NO: 3, the HCDR2 can comprise the amino acid sequence shown in SEQ ID NO: 11, and the HCDR3 can comprise the amino acid sequence shown in SEQ ID NO: 19; or
The HCDR1 can comprise the amino acid sequence shown in SEQ ID NO: 2, the HCDR2 can comprise the amino acid sequence shown in SEQ ID NO: 10, and the HCDR3 can comprise the amino acid sequence shown in SEQ ID NO: 20; or
The HCDR1 can comprise the amino acid sequence shown in SEQ ID NO: 4, the HCDR2 can comprise the amino acid sequence shown in SEQ ID NO: 12, and the HCDR3 can comprise the amino acid sequence shown in SEQ ID NO: 21; or
The HCDR1 can comprise the amino acid sequence shown in SEQ ID NO: 5, the HCDR2 can comprise the amino acid sequence shown in SEQ ID NO: 13, and the HCDR3 can comprise the amino acid sequence shown in SEQ ID NO: 22; or
The HCDR1 can comprise the amino acid sequence shown in SEQ ID NO: 6, the HCDR2 can comprise the amino acid sequence shown in SEQ ID NO: 14, and the HCDR3 can comprise the amino acid sequence shown in SEQ ID NO: 23; or
The HCDR1 can comprise the amino acid sequence shown in SEQ ID NO: 2, the HCDR2 can comprise the amino acid sequence shown in SEQ ID NO: 10, and the HCDR3 can comprise the amino acid sequence shown in SEQ ID NO: 24; or
The HCDR1 can comprise the amino acid sequence shown in SEQ ID NO: 7, the HCDR2 can comprise the amino acid sequence shown in SEQ ID NO: 15, and the HCDR3 can comprise the amino acid sequence shown in SEQ ID NO: 25; or
The HCDR1 can comprise the amino acid sequence shown in SEQ ID NO: 8, the HCDR2 can comprise the amino acid sequence shown in SEQ ID NO: 16, and the HCDR3 can comprise the amino acid sequence shown in SEQ ID NO: 26; or
The HCDR1 can comprise the amino acid sequence shown in SEQ ID NO: 9, the HCDR2 can comprise the amino acid sequence shown in SEQ ID NO: 17, and the HCDR3 can comprise the amino acid sequence shown in SEQ ID NO: 27.

In some embodiments, the isolated antigen-binding protein comprises VH, wherein the VH includes framework regions HFR1, HFR2, HFR3 and HFR4, the C-terminus of the HFR1 is directly or indirectly connected to the N-terminus of the HCDR1, and the HFR1 comprises the amino acid sequence shown in any one of SEQ ID NO: 28 to SEQ ID NO: 38.

In some embodiments, the HFR2 is located between the HCDR1 and the HCDR2, and the HFR2 comprises the amino acid sequence shown in any one of SEQ ID NO: 39 to SEQ ID NO: 47.

In some embodiments, the HFR3 is located between the HCDR2 and the HCDR3, and the HFR3 comprises the amino acid sequence shown in any one of SEQ ID NO: 48 to SEQ ID NO: 56.

In some embodiments, the N-terminus of the HFR4 is directly or indirectly connected to the C-terminus of the HCDR3, and the HFR4 comprises the amino acid sequence shown in any one of SEQ ID NO: 57 to SEQ ID NO: 60.

For example, the isolated antigen-binding protein comprises VH, wherein the VH can include framework regions HFR1, HFR2, HFR3 and HFR4, the C-terminus of the HFR1 is directly or indirectly connected to the N-terminus of the HCDR1, the HFR2 is located between the HCDR1 and the HCDR2, the HFR3 is located between the HCDR2 and the HCDR3, and the N-terminus of the HFR4 is directly or indirectly connected to the C-terminus of the HCDR3;
wherein the HFR1 can comprise the amino acid sequence shown in SEQ ID NO: 28, the HFR2 can comprise the amino acid sequence shown in SEQ ID NO: 39, the HFR3 can comprise the amino acid sequence shown in SEQ ID NO: 48, and the HFR4 can comprise the amino acid sequence shown in SEQ ID NO: 57; or
The HFR1 can comprise the amino acid sequence shown in SEQ ID NO: 29, the HFR2 can comprise the amino acid sequence shown in SEQ ID NO: 40, the HFR3 can comprise the amino acid sequence shown in SEQ ID NO: 49, and the HFR4 can comprise the amino acid sequence shown in SEQ ID NO: 58; or
The HFR1 can comprise the amino acid sequence shown in SEQ ID NO: 30, the HFR2 can comprise the amino acid sequence shown in SEQ ID NO: 39, the HFR3 can comprise the amino acid sequence shown in SEQ ID NO: 48, and the HFR4 can comprise the amino acid sequence shown in SEQ ID NO: 58; or
The HFR1 can comprise the amino acid sequence shown in SEQ ID NO: 31, the HFR2 can comprise the amino acid sequence shown in SEQ ID NO: 41, the HFR3 can comprise the amino acid sequence shown in SEQ ID NO: 50, and the HFR4 can comprise the amino acid sequence shown in SEQ ID NO: 59; or
The HFR1 can comprise the amino acid sequence shown in SEQ ID NO: 32, the HFR2 can comprise the amino acid sequence shown in SEQ ID NO: 42, the HFR3 can comprise the amino acid sequence shown in SEQ ID NO: 51, and the HFR4 can comprise the amino acid sequence shown in SEQ ID NO: 58; or
The HFR1 can comprise the amino acid sequence shown in SEQ ID NO: 33, the HFR2 can comprise the amino acid sequence shown in SEQ ID NO: 39, the HFR3 can comprise the amino acid sequence shown in SEQ ID NO: 48, and the HFR4 can comprise the amino acid sequence shown in SEQ ID NO: 58; or
The HFR1 can comprise the amino acid sequence shown in SEQ ID NO: 34, the HFR2 can comprise the amino acid sequence shown in SEQ ID NO: 39, the HFR3 can comprise the amino acid sequence shown in SEQ ID NO: 48, and the HFR4 can comprise the amino acid sequence shown in SEQ ID NO: 58; or
The HFR1 can comprise the amino acid sequence shown in SEQ ID NO: 35, the HFR2 can comprise the amino acid sequence shown in SEQ ID NO: 43, the HFR3 can comprise the amino acid sequence shown in SEQ ID NO: 52, and the HFR4 can comprise the amino acid sequence shown in SEQ ID NO: 58; or
The HFR1 can comprise the amino acid sequence shown in SEQ ID NO: 28, the HFR2 can comprise the amino acid sequence shown in SEQ ID NO: 39, the HFR3 can comprise the amino acid sequence shown in SEQ ID NO: 48, and the HFR4 can comprise the amino acid sequence shown in SEQ ID NO: 58; or
The HFR1 can comprise the amino acid sequence shown in SEQ ID NO: 30, the HFR2 can comprise the amino acid sequence shown in SEQ ID NO: 44, the HFR3 can comprise the amino acid sequence shown in SEQ ID NO: 53, and the HFR4 can comprise the amino acid sequence shown in SEQ ID NO: 58; or
The HFR1 can comprise the amino acid sequence shown in SEQ ID NO: 36, the HFR2 can comprise the amino acid sequence shown in SEQ ID NO: 45, the HFR3 can comprise the amino acid sequence shown in SEQ ID NO: 54, and the HFR4 can comprise the amino acid sequence shown in SEQ ID NO: 60; or
The HFR1 can comprise the amino acid sequence shown in SEQ ID NO: 37, the HFR2 can comprise the amino acid sequence shown in SEQ ID NO: 46, the HFR3 can comprise the amino acid sequence shown in SEQ ID NO: 55, and the HFR4 comprises the amino acid sequence shown in SEQ ID NO: 60; or
The HFR1 can comprise the amino acid sequence shown in SEQ ID NO: 38, the HFR2 can comprise the amino acid sequence shown in SEQ ID NO: 47, the HFR3 can comprise the amino acid sequence shown in SEQ ID NO: 56, and the HFR4 can comprise the amino acid sequence shown in SEQ ID NO: 58.

For another example, the isolated antigen-binding protein comprises VH, wherein the VH can comprise the amino acid sequence shown in SEQ ID NO: 61 to SEQ ID NO: 73.

In some embodiments, the isolated antigen-binding protein comprises a VL, wherein the VL comprises LCDR1, LCDR2, and LCDR3, and the LCDR1 comprises the amino acid sequence shown in any one of SEQ ID NO: 74 to SEQ ID NO: 86.

In some embodiments, the isolated antigen-binding protein comprises a VL, wherein the VL comprises LCDR1, LCDR2, and LCDR3, and the LCDR2 comprises the amino acid sequence shown in any one of SEQ ID NO: 87 to SEQ ID NO: 98.

In some embodiments, the isolated antigen-binding protein comprises a VL, wherein the VL comprises LCDR1, LCDR2, and LCDR3, and the LCDR3 comprises the amino acid sequence shown in any one of SEQ ID NO: 99 to SEQ ID NO: 101.

For example, the isolated antigen-binding protein comprises a VL, wherein the VL can comprise LCDR1, LCDR2 and LCDR3, the LCDR1 can comprise the amino acid sequence shown in SEQ ID NO: 74, the LCDR2 can comprise the amino acid sequence shown in SEQ ID NO: 87, and the LCDR3 can comprise the amino acid sequence shown in SEQ ID NO: 99;
or

The LCDR1 can comprise the amino acid sequence shown in SEQ ID NO: 75, the LCDR2 can comprise the amino acid sequence shown in SEQ ID NO: 88, and the LCDR3 can comprise the amino acid sequence shown in SEQ ID NO: 100; or
The LCDR1 can comprise the amino acid sequence shown in SEQ ID NO: 76, the LCDR2 can comprise the amino acid sequence shown in SEQ ID NO: 89, and the LCDR3 can comprise the amino acid sequence shown in SEQ ID NO: 101; or
The LCDR1 can comprise the amino acid sequence shown in SEQ ID NO: 77, the LCDR2 can comprise the amino acid sequence shown in SEQ ID NO: 90, and the LCDR3 can comprise the amino acid sequence shown in SEQ ID NO: 102; or
The LCDR1 can comprise the amino acid sequence shown in SEQ ID NO: 78, the LCDR2 can comprise the amino acid sequence shown in SEQ ID NO: 91, and the LCDR3 can comprise the amino acid sequence shown in SEQ ID NO: 103; or
The LCDR1 can comprise the amino acid sequence shown in SEQ ID NO: 79, the LCDR2 can comprise the amino acid sequence shown in SEQ ID NO: 92, and the LCDR3 can comprise the amino acid sequence shown in SEQ ID NO: 104; or
The LCDR1 can comprise the amino acid sequence shown in SEQ ID NO: 80, the LCDR2 can comprise the amino acid sequence shown in SEQ ID NO: 93, and the LCDR3 can comprise the amino acid sequence shown in SEQ ID NO: 105; or
The LCDR1 can comprise the amino acid sequence shown in SEQ ID NO: 81, the LCDR2 can comprise the amino acid sequence shown in SEQ ID NO: 94, and the LCDR3 can comprise the amino acid sequence shown in SEQ ID NO: 106; or
The LCDR1 can comprise the amino acid sequence shown in SEQ ID NO: 82, the LCDR2 can comprise the amino acid sequence shown in SEQ ID NO: 95, and the LCDR3 can comprise the amino acid sequence shown in SEQ ID NO: 107; or
The LCDR1 can comprise the amino acid sequence shown in SEQ ID NO: 84, the LCDR2 can comprise the amino acid sequence shown in SEQ ID NO: 97, and the LCDR3 can comprise the amino acid sequence shown in SEQ ID NO: 109; or
The LCDR1 can comprise the amino acid sequence shown in SEQ ID NO: 85, the LCDR2 can comprise the amino acid sequence shown in SEQ ID NO: 98, and the LCDR3 can comprise the amino acid sequence shown in SEQ ID NO: 110; or
The LCDR1 can comprise the amino acid sequence shown in SEQ ID NO: 86, the LCDR2 can comprise the amino acid sequence shown in SEQ ID NO: 98, and the LCDR3 can comprise the amino acid sequence shown in SEQ ID NO: 111.

In some embodiments, the isolated antigen-binding protein comprises VH and VL, wherein the VH comprises HCDR1, HCDR2 and HCDR3, and the VL comprises LCDR1, LCDR2 and LCDR3; wherein the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises III the amino acid sequence shown in SEQ ID NO: 74, the LCDR2 comprises the amino acid sequence shown in SEQ ID NO: 87, and the LCDR3 comprises the amino acid sequence shown in SEQ ID NO: 99; or
The HCDR1 comprises the amino acid sequence shown in SEQ ID NO: 3, the HCDR2 comprises the amino acid sequence shown in SEQ ID NO: 11, the HCDR3 comprises the amino acid sequence shown in SEQ ID NO: 19, the LCDR1 comprises the amino acid sequence shown in SEQ ID NO: 75, the LCDR2 comprises the amino acid sequence shown in SEQ ID NO: 88, and the LCDR3 comprises the amino acid sequence shown in SEQ ID NO: 100; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 20, the LCDR1 comprises the amino acid sequence shown in SEQ ID NO: 76, the LCDR2 comprises the amino acid sequence shown in SEQ ID NO: 89, and the LCDR3 comprises the amino acid sequence shown in SEQ ID NO: 101; or
The HCDR1 comprises the amino acid sequence shown in SEQ ID NO: 4, the HCDR2 comprises the amino acid sequence shown in SEQ ID NO: 12, the HCDR3 comprises the amino acid sequence shown in SEQ ID NO: 21, the LCDR1 comprises the amino acid sequence shown in SEQ ID NO: 77, the LCDR2 comprises the amino acid sequence shown in SEQ ID NO: 90, and the LCDR3 comprises the amino acid sequence shown in SEQ ID NO: 102; or
The HCDR1 comprises the amino acid sequence shown in SEQ ID NO: 5, the HCDR2 comprises the amino acid sequence shown in SEQ ID NO: 13, the HCDR3 comprises the amino acid sequence shown in SEQ ID NO: 22, the LCDR1 comprises the amino acid sequence shown in SEQ ID NO: 91, the LCDR2 comprises the amino acid sequence shown in SEQ ID NO: 91, and the LCDR3 comprises the amino acid sequence shown in SEQ ID NO: 103; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 79, the LCDR2 comprises the amino acid sequence shown in SEQ ID NO: 92, and the LCDR3 comprises the amino acid sequence shown in SEQ ID NO: 104; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 80, the LCDR2 comprises the amino acid sequence shown in SEQ ID NO: 93, and the LCDR3 comprises the amino acid sequence shown in SEQ ID NO: 105; or
The HCDR1 comprises the amino acid sequence shown in SEQ ID NO: 6, the HCDR2 comprises the amino acid sequence shown in SEQ ID NO: 14, the HCDR3 comprises the amino acid sequence shown in SEQ ID NO: 23, the LCDR1 comprises the amino acid sequence shown in SEQ ID NO: 81, the LCDR2 comprises the amino acid sequence shown in SEQ ID NO: 94, and the LCDR3 comprises the amino acid sequence shown in SEQ ID NO: 106; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 82, the LCDR2 comprises the amino acid sequence shown in SEQ ID NO: 95, and the LCDR3 comprises the amino acid sequence shown in SEQ ID NO: 107; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 24, the LCDR1 comprises the amino acid sequence shown in SEQ ID NO: 83, the LCDR2 comprises the amino acid sequence shown in SEQ ID NO: 96, and the LCDR3 comprises the amino acid sequence shown in SEQ ID NO: 108; or
The HCDR1 comprises the amino acid sequence shown in SEQ ID NO: 7, the HCDR2 comprises the amino acid sequence shown in SEQ ID NO: 15, the HCDR3 comprises the amino acid sequence shown in SEQ ID NO: 25, the LCDR1 comprises the amino acid sequence shown in SEQ ID NO: 84, the LCDR2 comprises the amino acid sequence shown in SEQ ID NO: 97, and the LCDR3 comprises the amino acid sequence shown in SEQ ID NO: 109; or
The HCDR1 comprises the amino acid sequence shown in SEQ ID NO: 8, the HCDR2 comprises the amino acid sequence shown in SEQ ID NO: 16, the HCDR3 comprises the amino acid sequence shown in SEQ ID NO: 26, the LCDR1 comprises the amino acid sequence shown in SEQ ID NO: 85, the LCDR2 comprises the amino acid sequence shown in SEQ ID NO: 98, and the LCDR3 comprises the amino acid sequence shown in SEQ ID NO: 110; or
The HCDR1 comprises the amino acid sequence shown in SEQ ID NO: 9, the HCDR2 comprises the amino acid sequence shown in SEQ ID NO: 17, the HCDR3 comprises the amino acid sequence shown in SEQ ID NO: 27, the LCDR1 comprises the amino acid sequence shown in SEQ ID NO: 86, the LCDR2 comprises the amino acid sequence shown in SEQ ID NO: 98, and the LCDR3 comprises the amino acid sequence shown in SEQ ID NO: 111.

In some embodiments, the isolated antigen-binding protein comprises VL, wherein the VL includes framework regions LFR1, LFR2, LFR3 and LFR4, wherein the C-terminus of LFR1 is directly or indirectly connected to the N-terminus of LCDR1, and the LFR1 comprises the amino acid sequence shown in any one of SEQ ID NO: 112 to SEQ ID NO: 123.

In some embodiments, the isolated antigen-binding protein comprises a VL, wherein the VL includes a framework region LFR 2, wherein the LFR2 is located between the LCDR1 and the LCDR2, and the LFR2 comprises the amino acid sequence shown in any one of SEQ ID NO: 124 to SEQ ID NO: 130.

In some embodiments, the isolated antigen-binding protein comprises a VL, wherein the VL includes a framework region LFR3, the LFR3 is located between the LCDR2 and the LCDR3, and the LFR3 comprises the amino acid sequence shown in any one of SEQ ID NO: 131 to SEQ ID NO: 141.

In some embodiments, the isolated antigen-binding protein comprises VL, wherein the VL includes a framework region LFR 4, the N-terminus of the LFR4 is directly or indirectly connected to the C-terminus of the LCDR3, and the LFR4 comprises the amino acid sequence shown in any one of SEQ ID NO: 142 to SEQ ID NO: 147.

For example, the isolated antigen-binding protein comprises VL, wherein the VL can include framework regions LFR1, LFR2, LFR3 and LFR4, wherein the C-terminus of the LFR1 is directly or indirectly connected to the N-terminus of the LCDR1, the LFR2 is located between the LCDR1 and the LCDR2, the LFR3 is located between the LCDR2 and the LCDR3, and the N-terminus of the LFR4 is directly or indirectly connected to the C-terminus of the LCDR3; wherein the LFR1 can comprise the amino acid sequence shown in SEQ ID NO: 112, the LFR2 can comprise the amino acid sequence shown in SEQ ID NO: 124, the LFR3 can comprise the amino acid sequence shown in SEQ ID NO: 131, and the LFR4 can comprise the amino acid sequence shown in SEQ ID NO: 142; or
The LFR1 can comprise the amino acid sequence shown in SEQ ID NO: 113, the LFR2 can comprise the amino acid sequence shown in SEQ ID NO: 125, the LFR3 can comprise the amino acid sequence shown in SEQ ID NO: 132, and the LFR4 can comprise the amino acid sequence shown in SEQ ID NO: 142; or
The LFR1 can comprise the amino acid sequence shown in SEQ ID NO: 114, the LFR2 can comprise the amino acid sequence shown in SEQ ID NO: 126, the LFR3 can comprise the amino acid sequence shown in SEQ ID NO: 133, and the LFR4 can comprise the amino acid sequence shown in SEQ ID NO: 142; or
The LFR1 can comprise the amino acid sequence shown in SEQ ID NO: 115, the LFR2 can comprise the amino acid sequence shown in SEQ ID NO: 127, the LFR3 can comprise the amino acid sequence shown in SEQ ID NO: 134, and the LFR4 can comprise the amino acid sequence shown in SEQ ID NO: 142; or
The LFR1 can comprise the amino acid sequence shown in SEQ ID NO: 116, the LFR2 can comprise the amino acid sequence shown in SEQ ID NO: 125, the LFR3 can comprise the amino acid sequence shown in SEQ ID NO: 135, and the LFR4 can comprise the amino acid sequence shown in SEQ ID NO: 143; or
The LFR1 can comprise the amino acid sequence shown in SEQ ID NO: 117, the LFR2 can comprise the amino acid sequence shown in SEQ ID NO: 126, the LFR3 can comprise the amino acid sequence shown in SEQ ID NO: 136, and the LFR4 can comprise the amino acid sequence shown in SEQ ID NO: 143; or
The LFR1 can comprise the amino acid sequence shown in SEQ ID NO: 118, the LFR2 can comprise the amino acid sequence shown in SEQ ID NO: 128, the LFR3 can comprise the amino acid sequence shown in SEQ ID NO: 137, and the LFR4 can comprise the amino acid sequence shown in SEQ ID NO: 142; or
The LFR1 can comprise the amino acid sequence shown in SEQ ID NO: 119, the LFR2 can comprise the amino acid sequence shown in SEQ ID NO: 125, the LFR3 can comprise the amino acid sequence shown in SEQ ID NO: 138, and the LFR4 can comprise the amino acid sequence shown in SEQ ID NO: 143; or
The LFR1 can comprise the amino acid sequence shown in SEQ ID NO: 120, the LFR2 can comprise the amino acid sequence shown in SEQ ID NO: 129, the LFR3 can comprise the amino acid sequence shown in SEQ ID NO: 139, and the LFR4 can comprise the amino acid sequence shown in SEQ ID NO: 142; or
The LFR1 can comprise the amino acid sequence shown in SEQ ID NO: 114, the LFR2 can comprise the amino acid sequence shown in SEQ ID NO: 126, the LFR3 can comprise the amino acid sequence shown in SEQ ID NO: 140, and the LFR4 can comprise the amino acid sequence shown in SEQ ID NO: 144; or
The LFR1 can comprise the amino acid sequence shown in SEQ ID NO: 121, the LFR2 can comprise the amino acid sequence shown in SEQ ID NO: 130, the LFR3 can comprise the amino acid sequence shown in SEQ ID NO: 141, and the LFR4 can comprise the amino acid sequence shown in SEQ ID NO: 145; or
The LFR1 can comprise the amino acid sequence shown in SEQ ID NO: 122, the LFR2 can comprise the amino acid sequence shown in SEQ ID NO: 130, the LFR3 can comprise the amino acid sequence shown in SEQ ID NO: 141, and the LFR4 can comprise the amino acid sequence shown in SEQ ID NO: 146; or
The LFR1 can comprise the amino acid sequence shown in SEQ ID NO: 123, the LFR2 can comprise the amino acid sequence shown in SEQ ID NO: 130, the LFR3 can comprise the amino acid sequence shown in SEQ ID NO: 141, and the LFR4 can comprise the amino acid sequence shown in SEQ ID NO: 147.

For example, the isolated antigen-binding protein comprises VL, wherein the VL can comprise the amino acid sequence shown in any one of SEQ ID NO: 148 to SEQ ID NO: 160.

For another example, the isolated antigen-binding protein comprises VH and VL, wherein the VH can comprise the amino acid sequence shown in SEQ ID NO: 61, and the VL can comprise the amino acid sequence shown in SEQ ID NO: 148; or
The VH can comprise the amino acid sequence shown in SEQ ID NO: 62, and the VL can comprise the amino acid sequence shown in SEQ ID NO: 149; or
The VH can comprise the amino acid sequence shown in SEQ ID NO: 63, and the VL can comprise the amino acid sequence shown in SEQ ID NO: 150; or
The VH can comprise the amino acid sequence shown in SEQ ID NO: 64, and the VL can comprise the amino acid sequence shown in SEQ ID NO: 151; or
The VH can comprise the amino acid sequence shown in SEQ ID NO: 65, and the VL can comprise the amino acid sequence shown in SEQ ID NO: 152; or
The VH can comprise the amino acid sequence shown in SEQ ID NO: 66, and the VL can comprise the amino acid sequence shown in SEQ ID NO: 153; or
The VH can comprise the amino acid sequence shown in SEQ ID NO: 67, and the VL can comprise the amino acid sequence shown in SEQ ID NO: 154; or
The VH can comprise the amino acid sequence shown in SEQ ID NO: 68, and the VL can comprise the amino acid sequence shown in SEQ ID NO: 155; or
The VH can comprise the amino acid sequence shown in SEQ ID NO: 69, and the VL can comprise the amino acid sequence shown in SEQ ID NO: 156; or
The VH can comprise the amino acid sequence shown in SEQ ID NO: 70, and the VL can comprise the amino acid sequence shown in SEQ ID NO: 157; or
The VH can comprise the amino acid sequence shown in SEQ ID NO: 71, and the VL can comprise the amino acid sequence shown in SEQ ID NO: 158; or
The VH can comprise the amino acid sequence shown in SEQ ID NO: 72, and the VL can comprise the amino acid sequence shown in SEQ ID NO: 159; or
The VH can comprise the amino acid sequence shown in SEQ ID NO: 73, and the VL can comprise the amino acid sequence shown in SEQ ID NO: 160.

In some embodiments, the isolated antigen-binding protein includes an antibody or an antigen-binding fragment thereof.

In some embodiments, the antibody includes a monoclonal antibody, a polyclonal antibody, a dimer, a multimer, a multispecific antibody, a full-length antibody, an antibody fragment, a human antibody, a humanized antibody, or a chimeric antibody.

In some embodiments, the antigen-binding fragment includes Fab, Fab′, Fv fragment, F(ab′)2, scFv, di-scFv and/or dAb.

In some embodiments, the antigen-binding protein includes an scFv.

For example, the antigen-binding protein can be an anti-CD7 scFv antibody.

In some embodiments, the VL and VH are connected by a linker.

In some embodiments, the linker includes a polypeptide linker.

In some embodiments, the polypeptide linker comprises an amino acid sequence represented by (GGGGS)n, wherein n is any integer from 1 to 5.

For example, the antigen-binding protein comprises the amino acid sequence shown in any one of SEQ ID NO: 162 to SEQ ID NO: 174.

### Immunoconjugate

On the other hand, the present application provides an immunoconjugate comprising the antigen-binding protein described in the present application.

In the present application, the term “immunoconjugate” or “antibody conjugate” generally refers to the connection of an antibody or its antibody fragment to other active agents, such as chemotherapeutic agents, toxins, immunotherapeutic agents, imaging probes, spectroscopic probes, and the like. The connection can be a covalent bond, or a non-covalent interaction, for example, by electrostatic forces. A variety of linkers known in the art can be used to form immunoconjugates. In addition, the immunoconjugate can be provided in the form of a fusion protein, which can be expressed from a polynucleotide encoding the immunoconjugate. As used herein, “fusion protein” refers to a protein produced by connecting two or more genes or gene fragments that originally encoded independent proteins (including peptides and polypeptides). Translation of the fusion gene produces a single protein with functional properties from each original protein.

In some embodiments, the immunoconjugate comprises:
(a) the antigen-binding protein described in the present application;
(b) a conjugated moiety selected from the group consisting of a detectable label, a drug, a toxin, a cytokine, a viral coat protein or a VLP, or a combination thereof.

Cytotoxins or cytotoxic agents include any agent that is detrimental to (e.g., kills) cells. Examples include paclitaxel, cytochalasin B, gramicidin D, ethidium bromide, emetine, mitomycin, etoposide, teniposide, vincristine, vinblastine, colchicine, doxorubicin, daunorubicin, dihydroxyanthraquinone, mitoxantrone, plicamycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., nitrogen mustard, thiotepa chlorambucil, melphalan, carmustine (BSNU) and lomustine (CCNU), cyclophosphamide, busulfan, dibromomannitol, streptozotocin, mitomycin C and cis-dichlorodiamine platinum (II) (DDP) cisplatin), anthracyclines (e.g., daunorubicin (formerly known as erythromycin) and doxorubicin), antibiotics (e.g., dactinomycin (formerly actinomycin)), bleomycin, plicamycin and anthramycin (AMC)), and antimitotic agents (e.g., vincristine and vinblastine). The antibodies used in the present application can be conjugated with a radioactive isotope (e.g., radioiodine) to generate a cytotoxic radiopharmaceutical for the treatment of cancer.

### Chimeric Antigen Receptor

Extracellular domain of the chimeric antigen receptor recognizes and specifically binds to an antigen, typically an antigen expressed on the surface of a malignant tumor (such as CD7). For example, when the antigen-specific extracellular domain binds to an antigen with an affinity constant or interaction affinity (KD) of about 0.1 pM to about 10 μM, or about 0.1 pM to about 1 μM, or about 0.1 pM to about 100 nM, the antigen-specific extracellular domain specifically binds to the antigen. Methods for determining interaction affinity are known in the art. The antigen-specific extracellular domain suitable for the CAR of the present disclosure can be any antigen-binding polypeptide, and a variety of antigen-binding polypeptides are known in the art. In some cases, the antigen-binding domain is a single-chain Fv (scFv). Other antibody-based recognition domains (cAb VHH (camelid antibody variable domain) and humanized forms thereof, IgNAR VH (shark antibody variable domain) and humanized forms thereof, sdAb VH (single domain antibody variable domain) and “camelized” antibody variable domains are suitable for use. In some cases, T cell receptor (TCR)-based recognition domains such as single chain TCR (scTv) are also suitable for use.

Suitable antigen can include T cell-specific antigen and/or antigen that are not specific for T cell. In some embodiments, the antigen specifically bounds by the chimeric antigen receptor of the CAR-T cell and the antigen lacking in the CAR-T cell are antigen expressed on malignant T cell, more preferably antigen overexpressed on malignant T cell compared with non-malignant T cell. “Malignant T cell” are T cell derived from T cell malignancies. The term “T cell malignancy” refers to a wide range of highly heterogeneous groupings of malignant tumors derived from T cell precursor, mature T cell or natural killer cell. Non-limiting examples of T-cell malignancies include T-cell acute lymphoblastic leukemia/lymphoma (T-ALL), T-cell large granular lymphocytic (LGL) leukemia, human T-cell leukemia virus type 1-positive (HTLV-1+) adult T-cell leukemia/lymphoma (ATL), T-cell prolymphocytic leukemia (T-PLL), and various peripheral T-cell lymphoma (PTCL), including but not limited to angioimmunoblastic T-cell lymphoma (AITL), ALK-positive anaplastic large cell lymphoma, and ALK-negative anaplastic large cell lymphoma.

In some embodiments, the CAR-T cells of the present application comprise an extracellular domain of a chimeric antigen receptor that specifically binds to CD7. CD7 is a T cell surface membrane-associated glycoprotein. CD7 can be overexpressed in T cell malignancies including T cell acute lymphoblastic leukemia (T-ALL) and non-Hodgkin's T cell lymphoma (NHL). The CAR-T cell disclosed herein can be used to target malignant T cell that overexpress CD7.

For example, extracellular domain of the chimeric antigen receptor of the present application can specifically bind to CD7, comprising the amino acid sequence shown in any one of SEQ ID NO: 162 to SEQ ID NO: 174, or an amino acid sequence having at least about 90%, about 91%, about 92%, about 93%, about 94%, about 95%, about 96%, about 97%, about 98%, about 99%, or about 99.5% identity with the amino acid sequence shown in any one of SEQ ID NO: 162 to SEQ ID NO: 174.

The chimeric antigen receptor of the present application further comprises an intracellular domain, which provides an intracellular signal to the T cell after the antigen is bound to the antigen-specific extracellular domain. Non-limiting examples of suitable intracellular domains include any one of the ζ chain of the T cell receptor or its homologues (e.g., η, δ, γ or ε), MB 1 chain, B29, Fc RIII, Fc RI, and signaling molecules (such as CD3.ζ. and CD28, CD27, 4-1BB, DAP-10, OX40 and combinations thereof and other similar molecules and fragments). Intracellular signaling portions of other members of the activating protein family can be used, such as Fc.γ.RIII and Fc.ε.RI. Although the entire intracellular domain is generally used, in many cases, it is not necessary to use the entire intracellular polypeptide. To the extent that the truncated portion of the intracellular signaling domain can be used, this truncated portion can be used instead of the complete chain, as long as the truncated portion still transduces the effector function signal. Thus, the intracellular domain of the present application is intended to include any truncated portion of the intracellular domain that is sufficient to transduce the effector function signal.

Typically, the antigen-specific extracellular domain is connected to the intracellular domain of the chimeric antigen receptor through a transmembrane domain. The transmembrane domain traverses the cell membrane, anchors CAR to the T cell surface, and connects the extracellular domain to the intracellular signaling domain, thereby affecting the expression of CAR on the T cell surface. The chimeric antigen receptor can further comprise one or more co-stimulatory domains and/or one or more spacers. The co-stimulatory domain is derived from the intracellular signaling domain of the enhanced in vivo cytokine production, proliferation, cytotoxicity and/or persistence of the co-stimulatory protein. The spacer (i) connects the antigen-specific extracellular domain to the transmembrane domain, (ii) connects the transmembrane domain to the co-stimulatory domain, (iii) connects the co-stimulatory domain to the intracellular domain, and/or (iv) connects the transmembrane domain to the intracellular domain. For example, the inclusion of a spacer domain between the antigen-specific extracellular domain and the transmembrane domain can affect the flexibility of the antigen-binding domain and thus affect the CAR function. Suitable transmembrane domains, co-stimulatory domains and spacers are known in the art.

In some embodiments, the chimeric antigen receptor includes a transmembrane domain, comprising a transmembrane domain derived from one or more proteins selected from the following group: CD8, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64 and SLAM.

For example, the transmembrane domain can comprise a transmembrane domain derived from CD8.

For another example, the transmembrane domain comprises the amino acid sequence shown in SEQ ID NO: 177, or an amino acid sequence that is at least about 90%, about 91%, about 92%, about 93%, about 94%, about 95%, about 96%, about 97%, about 98%, about 99%, or about 99.5% identical to the amino acid sequence shown in SEQ ID NO: 177.

In some embodiments, the chimeric antigen receptor includes an intracellular co-stimulatory signaling domain, comprising an intracellular co-stimulatory signaling domain derived from one or more proteins selected from the following group: CD28, CD137, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40 and MyD88.

For example, the intracellular co-stimulatory signaling domain can comprise a co-stimulatory signaling domain derived from 4-1BB.

For another example, the intracellular co-stimulatory signaling domain can comprise the amino acid sequence shown in any one of SEQ ID NO: 178.

In some embodiments, the chimeric antigen receptor includes an intracellular signaling transduction domain, comprising an intracellular signaling transduction domain derived from one or more proteins selected from the following group: CD3ζ, CD3δ, CD3γ, CD3ε, CD79a, CD79b, FeRIγ, FceRIβ, FcγRIIa, bovine leukemia virus gp30, Epstein-Barr virus (EBV) LMP2A, simian immunodeficiency virus PBj14 Nef, DAP10, DAP-12 and a domain comprising at least one ITAM.

For example, the intracellular signaling domain can comprise a signaling domain derived from CD3ζ.

For another example, the intracellular signaling domain can comprise the amino acid sequence shown in SEQ ID NO: 179.

In some embodiments, it includes a hinge region between the extracellular antigen-binding domain and the transmembrane domain, wherein the hinge region comprises a hinge region derived from one or more proteins selected from the following groups: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, TIM1, SLAM, CD30 and LIGHT.

For example, the hinge region can comprise a hinge region derived from CD8.

For example, the hinge region can comprise the amino acid sequence shown in SEQ ID NO: 176.

In some embodiments, the non-targeting portion of the chimeric antigen receptor comprises a transmembrane domain, a hinge region, an intracellular co-stimulatory signaling domain, and an intracellular signaling domain.

For example, the non-targeting portion of the chimeric antigen receptor comprises the transmembrane domain of the CD8 molecule, the hinge region of CD8, the intracellular co-stimulatory signaling domain of 4-1BB, and the intracellular signaling domain of CD3ζ.

In some embodiments, it further comprises a signal peptide fragment, wherein the C-terminus of the signal peptide fragment is connected to the N-terminus of the extracellular antigen-binding domain.

For example, the signal peptide fragment can comprise a CD8 signal peptide fragment.

For example, the signal peptide fragment can comprise the amino acid sequence shown in SEQ ID NO: 175.

For another example, the chimeric antigen receptor of the present application can comprise an amino acid sequence shown in any one of SEQ ID NO: 180 to SEQ ID NO: 192, or an amino acid sequence having at least about 90%, about 91%, about 92%, about 93%, about 94%, about 95%, about 96%, about 97%, about 98%, about 99%, or about 99.5% identity with the amino acid sequence shown in any one of SEQ ID NO: 180 to SEQ ID NO: 192.

### Chimeric Antigen Receptor Cell

On the other hand, the present application provides an engineered cell, comprising the nucleic acid molecule of the present application or the vector of the present application, and/or expressing the chimeric antigen receptor of the present application.

For example, the chimeric antigen receptor can comprise an amino acid sequence shown in any one of SEQ ID NO: 180 to SEQ ID NO: 192, or an amino acid sequence that is at least about 90%, about 91%, about 92%, about 93%, about 94%, about 95%, about 96%, about 97%, about 98%, about 99%, or about 99.5% identical to the amino acid sequence shown in any one of SEQ ID NO: 180 to SEQ ID NO: 192.

In some embodiments, the cell includes an immune effector cell.

In some embodiments, the immune effector cell includes a human cell.

In some embodiments, the immune effector cell includes a T cell, a B cell, a natural killer cell (NK cell), a macrophage, an NKT cell, a monocyte, a dendritic cell, a granulocyte, a lymphocyte, a leukocyte and/or a peripheral blood mononuclear cell.

In some embodiments, the immune effector cell includes an autologous or allogeneic immune effector cell.

In some embodiments, the immune effector cell includes an allogeneic T cell or an autologous T cell.

In some embodiments, the immune effector cell includes a modified immune effector cell.

In some embodiments, the expression of CD7 in the modified immune effector cell is absent or suppressed.

In some embodiments, the modified immune effector cell has reduced CD7 surface expression compared with corresponding immune cell and expresses anti-CD7 CAR.

In some embodiments, the immune effector cell includes a CAR-T cell.

In some embodiments, the CAR-T cell lacks the antigen to which the chimeric antigen receptor specifically binds and therefore does not induce fratricide.

In some embodiments, the antigen of the T cell is modified so that the chimeric antigen receptor no longer specifically binds to the modified antigen (such as CD7). For example, the epitope of the antigen recognized by the chimeric antigen receptor can be modified by one or more amino acid changes (e.g., substitution or deletion), or the epitope can be deleted from the antigen. In other embodiments, the expression of the antigen is reduced by at least 50%, at least 60%, at least 70%, at least 80%, at least 90% or more in the T cell. Methods for reducing the expression of proteins are known in the art and include, but are not limited to, modifying or replacing a promoter operably connected to a nucleic acid sequence encoding a protein. In other embodiments, the T cell is modified so that the antigen is not expressed, for example, by the deletion or destruction of the gene encoding the antigen. Methods for genetically modifying T cell to lack antigens are well known in the art, and non-limiting examples are provided above. In an exemplary embodiment, CRISPR/cas9 gene editing can be used to modify T cell to lack antigens.

The CAR-T cells covered by the present disclosure can also lack endogenous T cell receptor (TCR) signaling. In various embodiments, it can be desirable to reduce or eliminate endogenous TCR signaling in the CAR-T cells disclosed herein. For example, when allogeneic T cells are used to produce CAR-T cells, reducing or eliminating endogenous TCR signaling in CAR-T cells can prevent or reduce graft-versus-host disease (GvHD). Methods for reducing or eliminating endogenous TCR signaling are known in the art, and include but are not limited to modifying a portion of the TCR receptor (e.g., TCR receptor alpha chain (TRAC) etc.). TRAC modification can block TCR-mediated signaling. Therefore, TRAC modification can allow the safe use of allogeneic T cells as a source of CAR-T cells without inducing life-threatening GvHD.

Alternatively or in addition, the CAR-T cells covered by the present disclosure can also comprise one or more suicide genes. As used herein, “suicide gene” refers to a nucleic acid sequence introduced into a CAR-T cell by a standard method known in the art, which, when activated, causes the death of the CAR-T cell. If necessary, the suicide gene can promote the effective tracking and elimination of CAR-T cells in vivo. Promoting killing by activating the suicide gene can occur by methods known in the art.

In an exemplary embodiment, the present disclosure provides a T cell comprising a chimeric antigen receptor that specifically binds to CD7, wherein the T cell lacks CD7 (e.g., CD7ΔCART7 cell). In a non-limiting example, the lack of CD7 is produced by: (a) modifying CD7 expressed by T cell so that the chimeric antigen receptor no longer specifically binds to the modified CD7, (b) modifying T cell so that the expression of the antigen is reduced in T cell by at least 50%, at least 60%, at least 70%, at least 80%, at least 90% or more, or (c) modifying T cell so that CD7 is not expressed (e.g., by deletion or destruction of a gene encoding CD7).

Methods for designing, delivering and expressing CARs in T cell and the manufacture of clinical-grade CAR-T cell populations are known in the art. See, for example, Lee et al., Clin. Cancer Res., 2012, 18(10): 2780-90. For example, engineered CARs can be introduced into T cells using retroviruses, which effectively and stably integrate nucleic acid sequences encoding chimeric antigen receptors into the target cell genome. For another example, mRNA encoding CARs is transferred into T cells. Other methods known in the art include, but are not limited to, lentiviral transduction, transposon-based systems, direct RNA transfection, and CRISPR/Cas system.

CAR-T cells can be generated from any suitable T cell source known in the art, including but not limited to T cells collected from subjects. The subject can be a patient with a T cell malignancy requiring CAR-T cell therapy or a subject of the same type as a subject with a T cell malignancy requiring CAR-T cell therapy. The collected T cells can be expanded ex vivo using methods generally known in the art, and then transduced with CAR to generate CAR-T cells.

### Use

On the other hand, the present application provides a pharmaceutical composition, comprising the isolated antigen-binding protein described herein, the polypeptide described herein, the immunoconjugate described herein, the nucleic acid molecule described herein, the expression vector described herein, the cell described herein, the chimeric antigen receptor described herein and/or the engineered cell described herein, and optionally a pharmaceutically acceptable carrier.

On the other hand, the present application provides a kit comprising the isolated antigen-binding protein described herein, the polypeptide described herein, the immunoconjugate described herein, the nucleic acid molecule described herein, the expression vector described herein, the cell described herein, the chimeric antigen receptor described herein, the engineered cell described herein, or the pharmaceutical composition described herein.

Use of the isolated antigen-binding protein described herein, the polypeptide described herein, the immunoconjugate described herein, the nucleic acid molecule described herein, the expression vector described herein, the cell described herein, the chimeric antigen receptor described herein, the engineered cell described herein, or the pharmaceutical composition described herein in the preparation of a medicament for preventing and/or treating a CD7-related disease or condition.

In some embodiments, the CD7-related disease or condition includes a tumor.

In some embodiments, the tumor includes a tumor expressing CD7.

In some embodiments, the tumor includes a hematological tumor.

In some embodiments, the tumor includes a CD7-positive hematological malignancy.

In some embodiments, the tumor includes a T-cell malignancy.

On the other hand, the present application provides a method for treating tumors, the method including administering to a subject in need thereof an effective amount of the isolated antigen-binding protein described herein, the polypeptide described herein, the immunoconjugate described herein, the nucleic acid molecule described herein, the expression vector described herein, the cell described herein, the chimeric antigen receptor described herein, the engineered cell described herein, or the pharmaceutical composition described herein.

In some embodiments, the T-cell malignancy includes a CD7-positive hematological malignancy.

In some embodiments, the T-cell malignancy includes acute T-lymphocytic leukemia (T-ALL), acute myeloid leukemia, or NK/T-cell lymphoma.

On the other hand, the present application provides a method for treating a tumor, the method including administering an effective amount of CAR-T cells described in the present application to a subject in need thereof. Wherein the CAR-T cells target CD7, and CD7 is absent or suppressed. CAR-T cell therapy can also be accompanied by other therapies, including but not limited to immunotherapy, chemotherapy or radiotherapy.

CAR-T cells can be administered to a subject by an intravenous route (e.g., by intravenous infusion). CAR-T cells can be administered in a single dose or in multiple doses. CAR-T cells can be injected in a pharmaceutical composition suitable for intravenous administration. Suitable pharmaceutical compositions for IV administration are known in the art. The pharmaceutical composition of the present disclosure can also comprise additional components. For example, such components can be used to maintain the viability and/or activity of injected CAR-T cells.

CAR-T cells can be administered at an effective dose. An effective dose can be one dose or multiple doses and is sufficient to produce the desired therapeutic effect. A typical dose of CAR-T cells can range from about 1×10⁵ to 5×10⁷ cells/Kg of the body weight of the subject receiving therapy. The effective dose can be calculated based on the stage of the malignant tumor, the health status of the subject, and the type of malignant tumor. In the case of administering multiple doses, this dose and the interval between doses can be determined based on the subject's response to therapy.

On the other hand, the present application provides a method for killing malignant T cells, comprising contacting the malignant T cells with the engineered cells of the present application.

### Isolated Nucleic Acid Molecule and Vector

On the one hand, the present application provides a gRNA targeting the CD7 gene, comprising a nucleotide sequence shown in any one of SEQ ID NO: 212 to SEQ ID NO: 218, or an amino acid sequence having at least about 90%, about 91%, about 92%, about 93%, about 94%, about 95%, about 96%, about 97%, about 98%, about 99%, or about 99.5% identity with the nucleotide sequence shown in any one of SEQ ID NO: 212 to SEQ ID NO: 218.

On the other hand, the present application provides an isolated nucleic acid molecule, comprising the gRNA described in the present application or a DNA molecule encoding the gRNA.

On the other hand, the present application provides a vector comprising the nucleic acid molecule described in the present application.

In the present application, the “vector” generally refers to a nucleic acid molecule capable of self-replication in a suitable host, for transferring the inserted nucleic acid molecule into a host cell and/or between host cells. The vector can include a vector mainly used to insert DNA or RNA into a cell, a vector mainly used to replicate DNA or RNA, and a vector mainly used for expression of transcription and/or translation of DNA or RNA. The vector also includes vectors with a variety of the above functions. The vector can be a polynucleotide that can be transcribed and translated into a polypeptide when introduced into a suitable host cell. Typically, the vector can produce a desired expression product by culturing a suitable host cell containing the vector. The vector can include additional features other than the transgenic insertion sequence and the backbone: promoter, genetic marker, antibiotic resistance, reporter gene, targeting sequence, protein purification tag. The vector referred to as an expression vector (expression construct) is specifically used to express a transgene in a target cell and typically has a control sequence. The vector described in the present application can be an expression vector, which can include a viral vector (lentiviral vector and/or retroviral vector), a phage vector, a phagemid, a cosmid, an artificial chromosome such as a yeast artificial chromosome (YAC), a bacterial artificial chromosome (BAC) or a P1-derived artificial chromosome (PAC) and/or a plasmid.

In some embodiments, the vector includes a viral vector, and a viral vector can be used to introduce the nucleic acid molecules of the present application into cells. Such viral vectors include, for example, recombinant retroviruses, adenoviruses, adeno-associated viruses and herpes simplex virus-1. Retroviral vectors and adeno-associated virus vectors are usually understood as recombinant gene delivery systems selected for transferring exogenous genes in vivo, particularly entering the human body. Alternatively, they can be used to introduce exogenous genes into T cells in vitro. These vectors effectively deliver genes into T cells, and the transferred nucleic acids are stably integrated into the chromosomal DNA of the host cell.

### Cell

On the other hand, the present application provides a cell, which includes the nucleic acid molecule described in the present application or the vector described in the present application.

In some embodiments, they include immune effector cell.

In some embodiments, the immune effector cell includes a T cell, a B cell, a natural killer cell (NK cell), a macrophage, a NKT cell, a monocyte, a dendritic cell, a granulocyte, a lymphocyte, a leukocyte and/or a peripheral blood mononuclear cell.

In some embodiments, the immune effector cell includes an autologous or an allogeneic immune effector cell.

In some embodiments, the immune effector cell includes an allogeneic T cell or an autologous T cell.

In some embodiments, the immune effector cell includes an engineered immune effector cell.

In some embodiments, the engineered immune effector cell includes a CAR-T cell.

In some embodiments, the expression of antigen CD7 is reduced in T cells by at least 50%, at least 60%, at least 70%, at least 80%, at least 90% or more. In an exemplary embodiment, CRISPR/cas9 gene editing can be used to modify T cells to lack antigen CD7.

gRNA of the present invention can be introduced into T cells by transfection methods well known in the art. These methods include ultrasonic treatment, electric pulse, electroporation, osmotic pressure shock, calcium phosphate precipitation and DEAE dextran transfection, lipid-mediated delivery, passive delivery, etc. The term “transfection” includes a variety of techniques that can be used to introduce nucleic acids into mammalian cells, including electroporation, calcium phosphate precipitation, DEAE-dextran treatment, lipofection, microinjection and viral infection. Suitable methods for transfecting mammalian cells can be found in Sambrook et al. (Molecular Cloning: A Laboratory Manual, 2nd Edition, Cold Spring Harbor Laboratory Press (1989)) and other laboratory textbooks.

In some embodiments, the present invention comprises a method of delivering a CRISPR enzyme, the method comprising delivering a nucleic acid molecule, such as a plasmid or RNA or mRNA, encoding the CRISPR enzyme to a cell. In some methods of the present invention, the CRISPR enzyme is Cas9. In other embodiments, the CRISPR enzyme can be delivered directly to the cell.

### Chimeric Antigen Receptor

In the present application, the term “chimeric antigen receptor” or “CAR” generally refers to a group of polypeptides, usually two in the simplest embodiment, which, when in an immune effector cell, provide the cell with specificity for a target cell (usually a cancer cell) and generate an intracellular signal. In some embodiments, CAR comprises at least one extracellular antigen-binding domain, a transmembrane domain, and a cytoplasmic signaling domain (also referred to herein as an “intracellular signaling domain”), comprising a functional signaling domain derived from a stimulatory molecule and/or a co-stimulatory molecule as defined below. In some embodiments, the group of polypeptides is in the same polypeptide chain (e.g., comprising a chimeric fusion protein). In some embodiments, the group of polypeptides is discontinuous with each other, for example, in different polypeptide chains. In some aspects, the group of polypeptides includes a dimerization switch, which can couple polypeptides to each other in the presence of a dimerization molecule, for example, the antigen-binding domain can be coupled to the intracellular signaling domain. On the one hand, the stimulatory molecule of CAR is a ζ chain associated with a T cell receptor complex. On the one hand, the cytoplasmic signaling domain comprises a primary signaling domain (e.g., a primary signaling domain of CD3-ζ). On the one hand, the cytoplasmic signaling domain further comprises one or more functional signaling domains derived from at least one co-stimulatory molecule as defined below. On the one hand, the co-stimulatory molecule is selected from 4-1BB (i.e., CD137), CD27, ICOS and/or CD28. On the one hand, CAR comprises a chimeric fusion protein comprising an extracellular antigen recognition domain, a transmembrane domain, and an intracellular signaling domain comprising a functional signaling domain derived from a stimulatory molecule. On the one hand, CAR comprises a chimeric fusion protein comprising an extracellular antigen recognition domain, a transmembrane domain, and an intracellular signaling domain, and the intracellular signaling domain comprises a functional signaling domain derived from a co-stimulatory molecule and a functional signaling domain derived from a stimulatory molecule. On the one hand, CAR comprises a chimeric fusion protein comprising an extracellular antigen recognition domain, a transmembrane domain and an intracellular signaling domain, and the intracellular signaling domain comprises a functional signaling domain derived from one or more co-stimulatory molecules and a functional signaling domain derived from a stimulatory molecule. On the one hand, CAR includes a chimeric fusion protein comprising an extracellular antigen recognition domain, a transmembrane domain and an intracellular signaling domain, and the intracellular signaling domain comprises at least two functional signaling domains derived from one or more co-stimulatory molecules and a functional signaling domain derived from a stimulatory molecule. On the one hand, CAR comprises an optional leader sequence on the amino terminus (N-ter) of the CAR fusion protein. On the one hand, CAR further comprises a leader sequence at the N-terminus of the extracellular antigen recognition domain, wherein the leader sequence is optionally removed from the antigen recognition domain (e.g., scFv) during cell processing, and CAR is positioned on the cell membrane.

On the other hand, the present application provides a CAR-T cell, which is a CAR-T cell modified by the nucleic acid molecule described in the present application.

The term “CAR-T” or “CAR-T cell” generally refers to a T cell that can express CAR (also known as a “chimeric antigen receptor”). The CAR generally refers to a fusion protein comprising an extracellular domain capable of binding to an antigen and at least one intracellular domain. CAR is the core component of a chimeric antigen receptor T cell (CAR-T), which can include a targeting portion (e.g., a portion that binds to a tumor-associated antigen (TAA)), a hinge region, a transmembrane region, and an intracellular domain.

In some embodiments, the CAR comprises an extracellular antigen-binding domain, and the extracellular antigen-binding domain comprises the amino acid sequence shown in any one of SEQ ID NO: 162 to SEQ ID NO: 174.

In some embodiments, the anti-CD7 CAR comprises the amino acid sequence shown in any one of SEQ ID NO: 180 to SEQ ID NO: 192.

### Combination

On the other hand, the present application provides a composition comprising:
a. the nucleic acid molecule described in the present application, the vector described in the present application, the cell described in the present application, or the CAR-T cell described in the present application; and
b. Pharmaceutically acceptable carriers.

A pharmaceutically acceptable carrier generally refers to a substance suitable for administration to a subject, wherein the carrier is biologically harmless or does not cause other adverse effects. Such carriers are generally inert components of a drug. Typically, the carrier is administered to a subject together with the active ingredient without causing any undesirable biological effects or interacting in a harmful manner with any other components of the pharmaceutical composition contained therein. Suitable pharmaceutical carriers are described in Martin, Remington's Pharmaceutical Sciences, 18th ed., Mark Publishers, Easton, Pennsylvania, (1990), the contents of which are incorporated herein by reference.

The more specific form of the present application provides a pharmaceutical composition, comprising a therapeutically effective amount of nucleic acid molecules, vectors or cells and a pharmaceutically acceptable diluent, preservative, solubilizer, emulsifier, adjuvant and/or carrier. Such compositions include various buffer contents (e.g., phosphate, Tris-HCl, acetate), pH and ionic strength agents and additives, such as detergents and solubilizers (e.g., Tween 80, polysorbate 80), antioxidants (e.g., ascorbic acid, sodium metabisulfite), preservatives (e.g., thimerosal, benzyl alcohol) and fillers (e.g., lactose, mannitol). These substances can be incorporated into a granular formulation of a polymeric compound, such as, but not limited to, polylactic acid or polyglycolic acid, or incorporated into a liposome. Hyaluronic acid can also be used. Such compositions can affect the physical state, stability, in vivo release rate and in vivo clearance rate of the disclosed composition. The composition can be prepared in liquid form, or can be a dry powder, such as a lyophilized form. It should be understood that the pharmaceutical composition provided by the present disclosure can be administered by any means known in the art. For example, pharmaceutical compositions for administration can be administered by injection, orally, or via the pulmonary or nasal routes.

### Method and Use

On the other hand, the present application provides a method for regulating T cell function, including introducing the gRNA of the present application, the nucleic acid molecule of the present application, the expression vector of the present application, or the gene editing system of the present application into T cells.

In some embodiments, the method further includes administering a Cas enzyme to the cell.

It should be understood that the terms Cas enzyme and CRISPR enzyme are generally used interchangeably herein unless otherwise specified. In some embodiments, wherein the Cas enzyme includes a Cas9 protein. It should be understood that the present invention includes more Cas9s from other microbial species, such as SpCas9, SaCa9, StlCas9, and the like.

In some embodiments, the Cas9 is delivered to the cell as mRNA or protein.

In some embodiments, the gRNA is delivered simultaneously with the Cas9.

In some embodiments, wherein the delivering is by electroporation.

In some embodiments, CD7 gene expression in the regulated T cell is downregulated or knocked out compared with unregulated T cell.

In some embodiments, the method further includes modifying the specificity of the T cell by introducing a nucleic acid molecule encoding a CAR into the T cell.

In some embodiments, the nucleic acid molecule encoding CAR includes mRNA.

In some embodiments, the mRNA encodes anti-CD7 CAR.

In some embodiments, the CAR comprises an extracellular antigen-binding domain, and the extracellular antigen-binding domain comprises the amino acid sequence shown in any one of SEQ ID NO: 162 to SEQ ID NO: 174.

In some embodiments, the anti-CD7 CAR comprises the amino acid sequence shown in any one of SEQ ID NO: 180 to SEQ ID NO: 192.

In some embodiments, the regulated T cell has reduced CD7 surface expression compared with corresponding T cells and express anti-CD7 CAR.

In some embodiments, the gRNA described herein, the nucleic acid molecule described herein, the expression vector described herein, the gene editing system described herein and/or the nucleic acid molecule encoding CAR are introduced into T cells by any method selected from the following: ultrasonic treatment, electric pulse, electroporation, osmotic pressure shock, calcium phosphate precipitation, DEAE dextran transfection, lipid-mediated delivery and passive delivery.

On the other hand, the present application provides a method for treating a tumor, including administering an effective amount of the cell described herein or the pharmaceutical composition described herein to a subject in need thereof.

In some embodiments, the T-cell malignancy includes a CD7-positive hematological malignancy.

In some embodiments, the T-cell malignancy includes acute T-lymphocytic leukemia (T-ALL), acute myeloid leukemia, or NK/T-cell lymphoma.

On the other hand, the present application provides a method for treating a tumor, the method including administering an effective amount of CAR-T cells described in the present application to a subject in need thereof. Wherein the CAR-T cells target CD7, and CD7 is absent or suppressed. CAR-T cell therapy can also be accompanied by other therapies, including but not limited to immunotherapy, chemotherapy or radiotherapy.

CAR-T cells can be administered to a subject by an intravenous route (e.g., by intravenous infusion). CAR-T cells can be administered in a single dose or in multiple doses. CAR-T cells can be injected in a pharmaceutical composition suitable for intravenous administration. Suitable pharmaceutical compositions for IV administration are known in the art. The pharmaceutical composition of the present disclosure can also comprise additional components. For example, such components can be used to maintain the viability and/or activity of injected CAR-T cells.

CAR-T cells can be administered at an effective dose. An effective dose can be one dose or multiple doses and is sufficient to produce the desired therapeutic effect. A typical dose of CAR-T cells can range from about 1×10⁵ to 5×10⁷ cells/Kg of the body weight of the subject receiving therapy. The effective dose can be calculated based on the stage of the malignant tumor, the health status of the subject, and the type of malignant tumor. In the case of administering multiple doses, this dose and the interval between doses can be determined based on the subject's response to therapy.

On the other hand, the present application provides a method for killing malignant T cell, including contacting the malignant T cell with the CAR-T cell of the present application.

This application includes the following embodiments:

1. An isolated antigen-binding protein that specifically binds to a CD7 protein, wherein the isolated antigen-binding protein comprises a heavy chain variable region (VH), wherein the VH comprises HCDR1, HCDR2 and HCDR3, wherein HCDR1 comprises the amino acid sequence shown in SEQ ID NO: 1.

2. The isolated antigen-binding protein of embodiment 1, the HCDR1 comprises the amino acid sequence shown in any one of SEQ ID NO: 2 to SEQ ID NO: 9.

3. The isolated antigen-binding protein of any one of embodiments 1-2, wherein the HCDR2 comprises the amino acid sequence shown in any one of SEQ ID NO: 10 to SEQ ID NO: 17.

4. The isolated antigen-binding protein of any one of embodiments 1 to 3, wherein the HCDR3 comprises the amino acid sequence shown in SEQ ID NO: 18 or SEQ ID NO: 27.

5. The isolated antigen-binding protein of any one of embodiments 1 to 4, wherein the HCDR1 comprises the amino acid sequence shown in SEQ ID NO: 2, the HCDR2 comprises the amino acid sequence shown in SEQ ID NO: 10, and the HCDR3 comprises the amino acid sequence shown in SEQ ID NO: 18; or
The HCDR1 comprises the amino acid sequence shown in SEQ ID NO: 3, the HCDR2 comprises the amino acid sequence shown in SEQ ID NO: 11, and the HCDR3 comprises the amino acid sequence shown in SEQ ID NO: 19; or
The HCDR1 comprises the amino acid sequence shown in SEQ ID NO: 2, the HCDR2 comprises the amino acid sequence shown in SEQ ID NO: 10, and the HCDR3 comprises the amino acid sequence shown in SEQ ID NO: 20; or
The HCDR1 comprises the amino acid sequence shown in SEQ ID NO: 4, the HCDR2 comprises the amino acid sequence shown in SEQ ID NO: 12, and the HCDR3 comprises the amino acid sequence shown in SEQ ID NO: 21; or
The HCDR1 comprises the amino acid sequence shown in SEQ ID NO: 5, the HCDR2 comprises the amino acid sequence shown in SEQ ID NO: 13, and the HCDR3 comprises the amino acid sequence shown in SEQ ID NO: 22; or
The HCDR1 comprises the amino acid sequence shown in SEQ ID NO: 6, the HCDR2 comprises the amino acid sequence shown in SEQ ID NO: 14, and the HCDR3 comprises the amino acid sequence shown in SEQ ID NO: 23; or
The HCDR1 comprises the amino acid sequence shown in SEQ ID NO: 2, the HCDR2 comprises the amino acid sequence shown in SEQ ID NO: 10, and the HCDR3 comprises the amino acid sequence shown in SEQ ID NO: 24; or
The HCDR1 comprises the amino acid sequence shown in SEQ ID NO: 7, the HCDR2 comprises the amino acid sequence shown in SEQ ID NO: 15, and the HCDR3 comprises the amino acid sequence shown in SEQ ID NO: 25; or
The HCDR1 comprises the amino acid sequence shown in SEQ ID NO: 8, the HCDR2 comprises the amino acid sequence shown in SEQ ID NO: 16, and the HCDR3 comprises the amino acid sequence shown in SEQ ID NO: 26; or
The HCDR1 comprises the amino acid sequence shown in SEQ ID NO: 9, the HCDR2 comprises the amino acid sequence shown in SEQ ID NO: 17, and the HCDR3 comprises the amino acid sequence shown in SEQ ID NO: 27.

6. The isolated antigen-binding protein of any one of embodiments 1 to 5, wherein the VH includes framework regions HFR1, HFR2, HFR3 and HFR4, the C-terminus of the HFR1 is directly or indirectly connected to the N-terminus of the HCDR1, and the HFR1 comprises the amino acid sequence shown in any one of SEQ ID NO: 28 to SEQ ID NO: 38.

7. The isolated antigen-binding protein of any one of embodiments 1 to 6, wherein the HFR2 is located between the HCDR1 and the HCDR2, and the HFR2 comprises the amino acid sequence shown in any one of SEQ ID NO: 39 to SEQ ID NO: 47.

8. The isolated antigen-binding protein of any one of embodiments 1 to 7, wherein the HFR3 is located between the HCDR2 and the HCDR3, and the HFR3 comprises the amino acid sequence shown in any one of SEQ ID NO: 48 to SEQ ID NO: 56.

9. The isolated antigen-binding protein of any one of embodiments 1 to 8, wherein the N-terminus of the HFR4 is directly or indirectly connected to the C-terminus of the HCDR3, and the HFR4 comprises the amino acid sequence shown in any one of SEQ ID NO: 57 to SEQ ID NO: 60.

10. The isolated antigen-binding protein of embodiments 1 to 9 comprises a VH, wherein the VH includes framework regions HFR1, HFR2, HFR3 and HFR4, the C-terminus of the HFR1 is directly or indirectly connected to the N-terminus of the HCDR1, the HFR2 is located between the HCDR1 and the HCDR2, the HFR3 is located between the HCDR2 and the HCDR3, and the N-terminus of the HFR4 is directly or indirectly connected to the C-terminus of the HCDR3; wherein the HFR1 comprises the amino acid sequence shown in SEQ ID NO: 28, the HIFR2 comprises the amino acid sequence shown in SEQ ID NO: 39, the HIFR3 comprises the amino acid sequence shown in SEQ ID NO: 48, and the HIFR4 comprises the amino acid sequence shown in SEQ ID NO: 57; or
The HIFR1 comprises the amino acid sequence shown in SEQ ID NO: 29, the HIFR2 comprises the amino acid sequence shown in SEQ ID NO: 40, the HIFR3 comprises the amino acid sequence shown in SEQ ID NO: 49, and the HIFR4 comprises the amino acid sequence shown in SEQ ID NO: 58; or
The HIFR1 comprises the amino acid sequence shown in SEQ ID NO: 30, the HIFR2 comprises the amino acid sequence shown in SEQ ID NO: 39, the HIFR3 comprises the amino acid sequence shown in SEQ ID NO: 48, and the HIFR4 comprises the amino acid sequence shown in SEQ ID NO: 58; or
The amino acid sequence shown in SEQ ID NO: 31, the HIFR2 comprises the amino acid sequence shown in SEQ ID NO: 41, the HIFR3 comprises the amino acid sequence shown in SEQ ID NO: 50, and the HIFR4 comprises the amino acid sequence shown in SEQ ID NO: 59; or
The HIFR1 comprises the amino acid sequence shown in SEQ ID NO: 32, the HIFR2 comprises the amino acid sequence shown in SEQ ID NO: 42, the HIFR3 comprises the amino acid sequence shown in SEQ ID NO: 51, and the HIFR4 comprises the amino acid sequence shown in SEQ ID NO: 58; or
The HIFR1 comprises the amino acid sequence shown in SEQ ID NO: 33, the HIFR2 comprises the amino acid sequence shown in SEQ ID NO: 39, the HIFR3 comprises the amino acid sequence shown in SEQ ID NO: 48, and the HIFR4 comprises the amino acid sequence shown in SEQ ID NO: 58; or
The HIFR1 comprises the amino acid sequence shown in SEQ ID NO: 34, the HIFR2 comprises the amino acid sequence shown in SEQ ID NO: 39, the HIFR3 comprises the amino acid sequence shown in SEQ ID NO: 48, and the HIFR4 comprises the amino acid sequence shown in SEQ ID NO: 58; or
The HIFR1 comprises the amino acid sequence shown in SEQ ID NO: 35, the HFR2 comprises the amino acid sequence shown in SEQ ID NO: 43, the HFR3 comprises the amino acid sequence shown in SEQ ID NO: 52, and the HIFR4 comprises the amino acid sequence shown in SEQ ID NO: 58; or
The HIFR1 comprises the amino acid sequence shown in SEQ ID NO: 28, the HIFR2 comprises the amino acid sequence shown in SEQ ID NO: 39, the HIFR3 comprises the amino acid sequence shown in SEQ ID NO: 48, and the HIFR4 comprises the amino acid sequence shown in SEQ ID NO: 58; or
The HFR1 comprises the amino acid sequence shown in SEQ ID NO: 30, the HFR2 comprises the amino acid sequence shown in SEQ ID NO: 44, the HFR3 comprises the amino acid sequence shown in SEQ ID NO: 53, and the HFR4 comprises the amino acid sequence shown in SEQ ID NO: 58; or
The HFR1 comprises the amino acid sequence shown in SEQ ID NO: 36, the HFR2 comprises the amino acid sequence shown in SEQ ID NO: 45, the HFR3 comprises the amino acid sequence shown in SEQ ID NO: 54, and the HFR4 comprises the amino acid sequence shown in SEQ ID NO: 60; or
The HFR1 comprises the amino acid sequence shown in SEQ ID NO: 37, the HFR2 comprises the amino acid sequence shown in SEQ ID NO: 46, the HFR3 comprises the amino acid sequence shown in SEQ ID NO: 55, and the HFR4 comprises the amino acid sequence shown in SEQ ID NO: 60; or
The HFR1 comprises the amino acid sequence shown in SEQ ID NO: 38, the HFR2 comprises the amino acid sequence shown in SEQ ID NO: 47, the HFR3 comprises the amino acid sequence shown in SEQ ID NO: 56, and the HFR4 comprises the amino acid sequence shown in SEQ ID NO: 58.

11. The isolated antigen-binding protein of any one of embodiments 1 to 10, comprising a VH, wherein the VH comprises the amino acid sequence shown in SEQ ID NO: 61 to SEQ ID NO: 73.

12. The isolated antigen-binding protein of any one of embodiments 1 to 11, comprising a VL, wherein the VL comprises LCDR1, LCDR2, and LCDR3, and the LCDR1 comprises the amino acid sequence shown in any one of SEQ ID NO: 74 to SEQ ID NO: 86.

13. The isolated antigen-binding protein of any one of embodiments 1 to 12, comprising a VL, wherein the VL comprises LCDR1, LCDR2, and LCDR3, and the LCDR2 comprises the amino acid sequence shown in any one of SEQ ID NO: 87 to SEQ ID NO: 98.

14. The isolated antigen-binding protein of any one of embodiments 1 to 13, including a VL, wherein the VL comprises LCDR1, LCDR2, LCDR3, and the LCDR3 comprises the amino acid sequence shown in any one of SEQ ID NO: 99 to SEQ ID NO: 111.

15. The isolated antigen-binding protein of any one of embodiments 1 to 14, comprising a VL, wherein the VL comprises LCDR1, LCDR2 and LCDR3, the LCDR1 comprises the amino acid sequence shown in SEQ ID NO: 74, the LCDR2 comprises the amino acid sequence shown in SEQ ID NO: 87, and the LCDR3 comprises the amino acid sequence shown in SEQ ID NO: 99; or
The LCDR1 comprises the amino acid sequence shown in SEQ ID NO: 75, the LCDR2 comprises the amino acid sequence shown in SEQ ID NO: 88, and the LCDR3 comprises the amino acid sequence shown in SEQ ID NO: 100; or
The LCDR1 comprises the amino acid sequence shown in SEQ ID NO: 76, the LCDR2 comprises the amino acid sequence shown in SEQ ID NO: 89, and the LCDR3 comprises the amino acid sequence shown in SEQ ID NO: 101; or
The LCDR1 comprises the amino acid sequence shown in SEQ ID NO: 77, the LCDR2 comprises the amino acid sequence shown in SEQ ID NO: 90, and the LCDR3 comprises the amino acid sequence shown in SEQ ID NO: 102; or
The LCDR1 comprises the amino acid sequence shown in SEQ ID NO: 78, the LCDR2 comprises the amino acid sequence shown in SEQ ID NO: 91, and the LCDR3 comprises the amino acid sequence shown in SEQ ID NO: 103; or
The LCDR1 comprises the amino acid sequence shown in SEQ ID NO: 79, the LCDR2 comprises the amino acid sequence shown in SEQ ID NO: 92, and the LCDR3 comprises the amino acid sequence shown in SEQ ID NO: 104; or
The LCDR1 comprises the amino acid sequence shown in SEQ ID NO: 80, the LCDR2 comprises the amino acid sequence shown in SEQ ID NO: 93, and the LCDR3 comprises the amino acid sequence shown in SEQ ID NO: 105; or
The LCDR1 comprises the amino acid sequence shown in SEQ ID NO: 81, the LCDR2 comprises the amino acid sequence shown in SEQ ID NO: 94, and the LCDR3 comprises the amino acid sequence shown in SEQ ID NO: 106; or
The LCDR1 comprises the amino acid sequence shown in SEQ ID NO: 82, the LCDR2 comprises the amino acid sequence shown in SEQ ID NO: 95, and the LCDR3 comprises the amino acid sequence shown in SEQ ID NO: 107; or
The LCDR1 comprises the amino acid sequence shown in SEQ ID NO: 84, the LCDR2 comprises the amino acid sequence shown in SEQ ID NO: 97, and the LCDR3 comprises the amino acid sequence shown in SEQ ID NO: 109; or
The LCDR1 comprises the amino acid sequence shown in SEQ ID NO: 85, the LCDR2 comprises the amino acid sequence shown in SEQ ID NO: 98, and the LCDR3 comprises the amino acid sequence shown in SEQ ID NO: 110; or
The LCDR1 comprises the amino acid sequence shown in SEQ ID NO: 86, the LCDR2 comprises the amino acid sequence shown in SEQ ID NO: 98, and the LCDR3 comprises the amino acid sequence shown in SEQ ID NO: 111.

16. The isolated antigen-binding protein of any one of embodiments 1 to 15, comprising VH and VL, wherein the VH comprises HCDR1, HCDR2 and HCDR3, and the VL comprises LCDR1, LCDR2 and LCDR3; wherein the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 74, the LCDR2 comprises the amino acid sequence shown in SEQ ID NO: 87, and the LCDR3 comprises the amino acid sequence shown in SEQ ID NO: 99; or
The HCDR1 comprises the amino acid sequence shown in SEQ ID NO: 3, the HCDR2 comprises the amino acid sequence shown in SEQ ID NO: 11, the HCDR3 comprises the amino acid sequence shown in SEQ ID NO: 19, the LCDR1 comprises the amino acid sequence shown in SEQ ID NO: 75, the LCDR2 comprises the amino acid sequence shown in SEQ ID NO: 88, and the LCDR3 comprises the amino acid sequence shown in SEQ ID NO: 100; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 20, the LCDR1 comprises the amino acid sequence shown in SEQ ID NO: 76, the LCDR2 comprises the amino acid sequence shown in SEQ ID NO: 89, and the LCDR3 comprises the amino acid sequence shown in SEQ ID NO: 101; or
The HCDR1 comprises the amino acid sequence shown in SEQ ID NO: 4, the HCDR2 comprises the amino acid sequence shown in SEQ ID NO: 12, the HCDR3 comprises the amino acid sequence shown in SEQ ID NO: 21, the LCDR1 comprises the amino acid sequence shown in SEQ ID NO: 77, the LCDR2 comprises the amino acid sequence shown in SEQ ID NO: 90, and the LCDR3 comprises the amino acid sequence shown in SEQ ID NO: 102; or
The HCDR1 comprises the amino acid sequence shown in SEQ ID NO: 5, the HCDR2 comprises the amino acid sequence shown in SEQ ID NO: 13, the HCDR3 comprises the amino acid sequence shown in SEQ ID NO: 22, the LCDR1 comprises the amino acid sequence shown in SEQ ID NO: 91, the LCDR2 comprises the amino acid sequence shown in SEQ ID NO: 91, and the LCDR3 comprises the amino acid sequence shown in SEQ ID NO: 103; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 79, the LCDR2 comprises the amino acid sequence shown in SEQ ID NO: 92, and the LCDR3 comprises the amino acid sequence shown in SEQ ID NO: 104; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 80, the LCDR2 comprises the amino acid sequence shown in SEQ ID NO: 93, and the LCDR3 comprises the amino acid sequence shown in SEQ ID NO: 105; or
The HCDR1 comprises the amino acid sequence shown in SEQ ID NO: 6, the HCDR2 comprises the amino acid sequence shown in SEQ ID NO: 14, the HCDR3 comprises the amino acid sequence shown in SEQ ID NO: 23, the LCDR1 comprises the amino acid sequence shown in SEQ ID NO: 81, the LCDR2 comprises the amino acid sequence shown in SEQ ID NO: 94, and the LCDR3 comprises the amino acid sequence shown in SEQ ID NO: 106; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 82, the LCDR2 comprises the amino acid sequence shown in SEQ ID NO: 95, and the LCDR3 comprises the amino acid sequence shown in SEQ ID NO: 107; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 24, the LCDR1 comprises the amino acid sequence shown in SEQ ID NO: 83, the LCDR2 comprises the amino acid sequence shown in SEQ ID NO: 96, and the LCDR3 comprises the amino acid sequence shown in SEQ ID NO: 108; or
The HCDR1 comprises the amino acid sequence shown in SEQ ID NO: 7, the HCDR2 comprises the amino acid sequence shown in SEQ ID NO: 15, the HCDR3 comprises the amino acid sequence shown in SEQ ID NO: 25, the LCDR1 comprises the amino acid sequence shown in SEQ ID NO: 84, the LCDR2 comprises the amino acid sequence shown in SEQ ID NO: 97, and the LCDR3 comprises the amino acid sequence shown in SEQ ID NO: 109; or
The HCDR1 comprises the amino acid sequence shown in SEQ ID NO: 8, the HCDR2 comprises the amino acid sequence shown in SEQ ID NO: 16, the HCDR3 comprises the amino acid sequence shown in SEQ ID NO: 26, the LCDR1 comprises the amino acid sequence shown in SEQ ID NO: 85, the LCDR2 comprises the amino acid sequence shown in SEQ ID NO: 98, and the LCDR3 comprises the amino acid sequence shown in SEQ ID NO: 110; or
The HCDR1 comprises the amino acid sequence shown in SEQ ID NO: 9, the HCDR2 comprises the amino acid sequence shown in SEQ ID NO: 17, the HCDR3 comprises the amino acid sequence shown in SEQ ID NO: 27, the LCDR1 comprises the amino acid sequence shown in SEQ ID NO: 86, the LCDR2 comprises the amino acid sequence shown in SEQ ID NO: 98, and the LCDR3 comprises the amino acid sequence shown in SEQ ID NO: 111.

17. The isolated antigen-binding protein of any one of embodiments 1 to 16, comprising a VL, wherein the VL includes framework regions LFR1, LFR2, LFR3 and LFR4, wherein the C-terminus of the LFR1 is directly or indirectly connected to the N-terminus of the LCDR1, and the LFR1 comprises the amino acid sequence shown in any one of SEQ ID NO: 112 to SEQ ID NO: 123.

18. The isolated antigen-binding protein of any one of embodiments 1 to 17, wherein the LFR2 is located between the LCDR1 and the LCDR2, and the LFR2 comprises the amino acid sequence shown in any one of SEQ ID NO: 124 to SEQ ID NO: 130.

19. The isolated antigen-binding protein of any one of embodiments 1-18, comprising a VL, wherein the VL includes a framework region LFR3, the LFR3 is located between the LCDR2 and the LCDR3, and the LFR3 comprises the amino acid sequence shown in any one of SEQ ID NO: 131 to SEQ ID NO: 141.

20. The isolated antigen-binding protein of any one of embodiments 1 to 19, comprising a VL, wherein the VL includes a framework region LFR4, the N-terminus of the LFR4 is directly or indirectly connected to the C-terminus of the LCDR3, and the LFR4 comprises the amino acid sequence shown in any one of SEQ ID NO: 142 to SEQ ID NO: 147.

21. The isolated antigen-binding protein of any one of embodiments 1 to 20, comprising a VL, wherein the VL includes framework regions LFR1, LFR2, LFR3 and LFR4, wherein the C-terminus of the LFR1 is directly or indirectly connected to the N-terminus of the LCDR1, the LFR2 is located between the LCDR1 and the LCDR2, the LFR3 is located between the LCDR2 and the LCDR3, and the N-terminus of the LFR4 is directly or indirectly connected to the C-terminus of the LCDR3; wherein the LFR1 comprises the amino acid sequence shown in SEQ ID NO: 112, the LFR2 comprises the amino acid sequence shown in SEQ ID NO: 124, the LFR3 comprises the amino acid sequence shown in SEQ ID NO: 131, and the LFR4 comprises the amino acid sequence shown in SEQ ID NO: 142; or
The LFR1 comprises the amino acid sequence shown in SEQ ID NO: 113, the LFR2 comprises the amino acid sequence shown in SEQ ID NO: 125, the LFR3 comprises the amino acid sequence shown in SEQ ID NO: 132, and the LFR4 comprises the amino acid sequence shown in SEQ ID NO: 142; or
The LFR1 comprises the amino acid sequence shown in SEQ ID NO: 114, the LFR2 comprises the amino acid sequence shown in SEQ ID NO: 126, the LFR3 comprises the amino acid sequence shown in SEQ ID NO: 133, and the LFR4 comprises the amino acid sequence shown in SEQ ID NO: 142; or
The LFR1 comprises the amino acid sequence shown in SEQ ID NO: 115, the LFR2 comprises the amino acid sequence shown in SEQ ID NO: 127, the LFR3 comprises the amino acid sequence shown in SEQ ID NO: 134, and the LFR4 comprises the amino acid sequence shown in SEQ ID NO: 142; or
The LFR1 comprises the amino acid sequence shown in SEQ ID NO: 116, the LFR2 comprises the amino acid sequence shown in SEQ ID NO: 125, the LFR3 comprises the amino acid sequence shown in SEQ ID NO: 135, and the LFR4 comprises the amino acid sequence shown in SEQ ID NO: 143; or
The LFR1 comprises the amino acid sequence shown in SEQ ID NO: 117, the LFR2 comprises the amino acid sequence shown in SEQ ID NO: 126, the LFR3 comprises the amino acid sequence shown in SEQ ID NO: 136, and the LFR4 comprises the amino acid sequence shown in SEQ ID NO: 143; or
The LFR1 comprises the amino acid sequence shown in SEQ ID NO: 118, the LFR2 comprises the amino acid sequence shown in SEQ ID NO: 128, the LFR3 comprises the amino acid sequence shown in SEQ ID NO: 137, and the LFR4 comprises the amino acid sequence shown in SEQ ID NO: 142; or
The LFR1 comprises the amino acid sequence shown in SEQ ID NO: 119, the LFR2 comprises the amino acid sequence shown in SEQ ID NO: 125, the LFR3 comprises the amino acid sequence shown in SEQ ID NO: 138, and the LFR4 comprises the amino acid sequence shown in SEQ ID NO: 143; or
The LFR1 comprises the amino acid sequence shown in SEQ ID NO: 120, the LFR2 comprises the amino acid sequence shown in SEQ ID NO: 129, the LFR3 comprises the amino acid sequence shown in SEQ ID NO: 139, and the LFR4 comprises the amino acid sequence shown in SEQ ID NO: 142; or
The LFR1 comprises the amino acid sequence shown in SEQ ID NO: 114, the LFR2 comprises the amino acid sequence shown in SEQ ID NO: 126, the LFR3 comprises the amino acid sequence shown in SEQ ID NO: 140, and the LFR4 comprises the amino acid sequence shown in SEQ ID NO: 144; or
The LFR1 comprises the amino acid sequence shown in SEQ ID NO: 121, the LFR2 comprises the amino acid sequence shown in SEQ ID NO: 130, the LFR3 comprises the amino acid sequence shown in SEQ ID NO: 141, and the LFR4 comprises the amino acid sequence shown in SEQ ID NO: 145; or
The LFR1 comprises the amino acid sequence shown in SEQ ID NO: 122, the LFR2 comprises the amino acid sequence shown in SEQ ID NO: 130, the LFR3 comprises the amino acid sequence shown in SEQ ID NO: 141, and the LFR4 comprises the amino acid sequence shown in SEQ ID NO: 146; or
The LFR1 comprises the amino acid sequence shown in SEQ ID NO: 123, the LFR2 comprises the amino acid sequence shown in SEQ ID NO: 130, the LFR3 comprises the amino acid sequence shown in SEQ ID NO: 141, and the LFR4 comprises the amino acid sequence shown in SEQ ID NO: 147.

22. The isolated antigen-binding protein of any one of embodiments 1 to 21, comprising a VL, wherein the VL comprises the amino acid sequence shown in any one of SEQ ID NO: 148 to SEQ ID NO: 160.

23. The isolated antigen-binding protein of any one of embodiments 1 to 22, comprising VH and VL, wherein the VH comprises the amino acid sequence shown in SEQ ID NO: 61, and the VL comprises the amino acid sequence shown in SEQ ID NO: 148; or
The VH comprises the amino acid sequence shown in SEQ ID NO: 62, and the VL comprises the amino acid sequence shown in SEQ ID NO: 149; or
The VH comprises the amino acid sequence shown in SEQ ID NO: 63, and the VL comprises the amino acid sequence shown in SEQ ID NO: 150; or
The VH comprises the amino acid sequence shown in SEQ ID NO: 64, and the VL comprises the amino acid sequence shown in SEQ ID NO: 151; or
The VH comprises the amino acid sequence shown in SEQ ID NO: 65, and the VL comprises the amino acid sequence shown in SEQ ID NO: 152; or
The VH comprises the amino acid sequence shown in SEQ ID NO: 66, and the VL comprises the amino acid sequence shown in SEQ ID NO: 153; or
The VH comprises the amino acid sequence shown in SEQ ID NO: 67, and the VL comprises the amino acid sequence shown in SEQ ID NO: 154; or
The VH comprises the amino acid sequence shown in SEQ ID NO: 68, and the VL comprises the amino acid sequence shown in SEQ ID NO: 155; or
The VH comprises the amino acid sequence shown in SEQ ID NO: 69, and the VL comprises the amino acid sequence shown in SEQ ID NO: 156; or
The VH comprises the amino acid sequence shown in SEQ ID NO: 70, and the VL comprises the amino acid sequence shown in SEQ ID NO: 157; or
The VH comprises the amino acid sequence shown in SEQ ID NO: 71, and the VL comprises the amino acid sequence shown in SEQ ID NO: 158; or
The VH comprises the amino acid sequence shown in SEQ ID NO: 72, and the VL comprises the amino acid sequence shown in SEQ ID NO: 159; or
The VH comprises the amino acid sequence shown in SEQ ID NO: 73, and the VL comprises the amino acid sequence shown in SEQ ID NO: 160.
24. The isolated antigen-binding protein of any one of embodiments 1 to 23, including an antibody or an antigen-binding fragment thereof.

25. The isolated antigen-binding protein of any one of embodiments 1 to 24, the antibody includes a monoclonal antibody, a polyclonal antibody, a dimer, a multimer, a multispecific antibody, a full-length antibody, an antibody fragment, a human antibody, a humanized antibody or a chimeric antibody.

26. The isolated antigen-binding protein of any one of embodiments 1 to 25, wherein the antigen-binding fragment includes Fab, Fab′, Fv fragment, F(ab′)2, scFv, di-scFv and/or dAb.

27. The antigen-binding protein of embodiments 1-26, including an scFv.

28. The antigen-binding protein of embodiments 1-27, wherein the VL and VH are connected by a linker.

29. The antigen-binding protein of embodiment 28, wherein the linker includes a polypeptide linker.

30. The antigen-binding protein of embodiment 29, wherein the polypeptide linker comprises an amino acid sequence shown in (GGGGS)n, wherein n is any integer from 1 to 5.

31. The isolated antigen-binding protein of any one of embodiments 1 to 30, comprising the amino acid sequence shown in any one of SEQ ID NO: 162 to SEQ ID NO: 174.

32. An isolated polypeptide comprising the isolated antigen-binding protein of any one of embodiments 1-31.

33. An immunoconjugate comprising the antigen-binding protein of any one of embodiments 1-31.

34. A chimeric antigen receptor comprising at least one extracellular antigen-binding domain that comprises the antigen-binding protein of any one of embodiments 1 to 31.

35. The chimeric antigen receptor of embodiment 34, wherein the extracellular antigen-binding domain includes an scFv.

36. The chimeric antigen receptor of any one of embodiments 1 to 35, which includes a transmembrane domain, wherein the transmembrane domain comprises a transmembrane domain derived from one or more proteins selected from the following group: CD8, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64 and SLAM.

37. The chimeric antigen receptor of embodiment 36, wherein the transmembrane domain comprises a transmembrane domain derived from CD8.

38. The chimeric antigen receptor of any one of embodiments 34 to 37, wherein the transmembrane domain comprises the amino acid sequence shown in SEQ ID NO: 177.

39. The chimeric antigen receptor of any one of embodiments 34 to 38, the chimeric antigen receptor includes an intracellular co-stimulatory signaling domain, comprising an intracellular co-stimulatory signaling domain derived from one or more proteins selected from the group consisting of CD28, CD137, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40 and MyD88.

40. The chimeric antigen receptor of embodiment 39, wherein the intracellular co-stimulatory signaling domain is derived from the co-stimulatory signaling domain of 4-1BB.

41. The chimeric antigen receptor of any one of embodiments 39-40, wherein the intracellular co-stimulatory signaling domain comprises the amino acid sequence shown in any one of SEQ ID NO: 178.

42. The chimeric antigen receptor of any one of embodiments 34 to 41, includes an intracellular signaling domain, wherein the intracellular signaling domain comprises an intracellular signaling domain derived from one or more proteins selected from the following group: CD3ζ, CD3δ, CD3γ, CD3ε, CD79a, CD79b, FceRIγ, FceRIβ, FcγRIIa, bovine leukemia virus gp30, Epstein-Barr virus (EBV) LMP2A, simian immunodeficiency virus PBj14 Nef, DAP10, DAP-12 and a domain comprising at least one ITAM.

43. The chimeric antigen receptor of embodiment 42, wherein the intracellular signaling domain comprises a signaling domain derived from CD3ζ.

44. The chimeric antigen receptor of any one of embodiments 42 to 43, wherein the intracellular signaling domain comprises the amino acid sequence shown in SEQ ID NO: 179.

45. The chimeric antigen receptor of any one of embodiments 34 to 44, the chimeric antigen receptor includes a hinge region between the extracellular antigen-binding domain and the transmembrane domain, and the hinge region comprises a hinge region derived from one or more proteins selected from the following group: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, TIM1, SLAM, CD30 and LIGHT.

46. The chimeric antigen receptor of embodiment 45, wherein the hinge region comprises a hinge region derived from CD8.

47. The chimeric antigen receptor of any one of embodiments 45 to 46, the hinge region comprises the amino acid sequence shown in SEQ ID NO: 176.

48. The chimeric antigen receptor of any one of embodiments 34 to 47, the non-targeting portion of the chimeric antigen receptor comprises a transmembrane domain, a hinge region, an intracellular co-stimulatory signaling domain, and an intracellular signaling domain.

49. The chimeric antigen receptor of any one of embodiments 34 to 48, the non-targeting portion of the chimeric antigen receptor comprises the transmembrane domain of the CD8 molecule, the hinge region of CD8, the intracellular co-stimulatory signaling domain of 4-1BB and the intracellular signaling domain of CD3ζ.

50. The chimeric antigen receptor of any one of embodiments 34 to 49, further comprises a signal peptide fragment, wherein the C-terminus of the signal peptide fragment is connected to the N-terminus of the extracellular antigen-binding domain.

51. The chimeric antigen receptor of embodiment 50, wherein the signal peptide fragment includes a CD8 signal peptide fragment.

52. The chimeric antigen receptor of any one of embodiments 50 to 51, wherein the signal peptide fragment comprises the amino acid sequence shown in SEQ ID NO: 175.

53. The chimeric antigen receptor of any one of embodiments 34 to 52, comprising the amino acid sequence shown in any one of SEQ ID NO: 180 to SEQ ID NO: 192.

54. An isolated nucleic acid molecule encoding the isolated antigen-binding protein of any one of embodiments 1 to 31, the polypeptide of embodiment 32, or the chimeric antigen receptor of any one of embodiments 34 to 53.

55. An expression vector comprising the nucleic acid molecule of embodiment 54.

56. A cell comprising a nucleic acid molecule of embodiment 54 or an expression vector of embodiment 55, and/or ii) the cell expresses an antigen-binding protein of any one of embodiments 1 to 31, a polypeptide of embodiment 32, or a chimeric antigen receptor of any one of embodiments 34 to 53.

57. A method of preparing the isolated antigen-binding protein of any one of embodiments 1 to 31, the method including culturing the cell of embodiment 56 under conditions such that the isolated antigen-binding protein of any one of embodiments 1 to 31 is expressed.

58. An engineered cell comprising the nucleic acid molecule of any one of embodiments 1 to 31 or the vector of any one of embodiments 55, and/or expressing the chimeric antigen receptor of any one of embodiments 34 to 53.

59. The engineered cell of embodiment 58, the cell includes immune effector cell.

60. The engineered cell of embodiment 59, the immune effector cell includes a human cell.

61. The engineered cell of any one of embodiments 59 to 60, the immune effector cell includes a T cell, a B cell, a natural killer cell (NK cell), a macrophage, a NKT cell, a monocyte, a dendritic cell, a granulocyte, a lymphocyte, a leukocyte and/or a peripheral blood mononuclear cell.

62. The engineered cell of any one of embodiments 59 to 61, the immune effector cell includes an autologous or allogeneic immune effector cell.

63. The engineered cell of any one of embodiments 59 to 62, wherein the immune effector cell includes an allogeneic T cell or autologous T cell.

64. The engineered cell of any one of embodiments 59 to 63, the immune effector cell includes modified immune effector cell.

65. The engineered cell of embodiment 64, wherein the expression of CD7 of the modified immune effector cell is absent or suppressed.

66. The engineered cell of any one of embodiments 64 to 65, wherein the modified immune effector cell has reduced CD7 surface expression compared with the corresponding immune cell and expresses anti-CD7 CAR.

67. The engineered cells of any one of embodiments 69 to 66, the immune effector cell includes a CAR-T cell.

68. The engineered cell of embodiment 67, the CAR-T cell does not induce fratricide.

69. A method of preparing a population of chimeric antigen receptor T (CAR-T) cells, wherein the CAR targets CD7, including the following steps:
a. Modifying the CD7 gene in the population of T cells;
b. Activating the population of T cells;
c. Transducing the T cells with a chimeric antigen receptor of any one of embodiments 34 to 53; and
d. Expanding the chimeric antigen receptor T cells.

70. The method of embodiment 69, CD7 is absent or suppressed in the population of modified T cells compared with the corresponding unmodified cells.

71. The method of any of embodiments 69 to 70, wherein the modification includes administering to the T cell population one or more substances selected from the group consisting of antisense RNA, siRNA, shRNA, transcription activator-like effector nuclease (TALEN), zinc finger nuclease (ZFN) and CRISPR/Cas system.

72. The method of any of embodiments 69 to 71, wherein the modification includes administering a CRISPR/Cas system to the population of T cells.

73. The method of any of embodiments 68 to 72, wherein the modification includes administering a CRISPR/Cas9 system to the population of T cells.

74. The method of any one of embodiments 68 to 73, wherein the modification includes administering Cas9 and a gRNA targeting the CD7 gene to the population of T cells.

75. The method of embodiment 74, wherein the gRNA targeting the CD7 gene comprises a nucleotide sequence shown in any one of SEQ ID NO: 211 to SEQ ID NO: 218.

76. The method of embodiment 74, wherein the Cas9 is delivered to the cell as mRNA or protein.

77. The method of embodiment 74, wherein the gRNA is delivered simultaneously with the Cas9.

78. The method of embodiment 77, wherein the delivering is performed by electroporation.

79. A pharmaceutical composition comprising the isolated antigen-binding protein of any one of embodiments 1 to 31, the polypeptide of embodiment 31, the immunoconjugate of embodiment 33, the nucleic acid molecule of embodiment 54, the expression vector of embodiment 55, the cell of embodiment 56, the chimeric antigen receptor of any one of embodiments 34 to 53 and/or the engineered cell of any one of embodiments 58 to 68, and optionally a pharmaceutically acceptable carrier.

80. A kit comprising the isolated antigen-binding protein of any one of embodiments 1-31, the polypeptide of embodiment 31, the immunoconjugate of embodiment 33, the nucleic acid molecule of embodiment 54, the expression vector of embodiment 55, the cell of embodiment 56, the chimeric antigen receptor of any one of embodiments 34 to 53, the engineered cell of any one of embodiments 58 to 68, or the pharmaceutical composition of embodiment 79.

81. Use of the isolated antigen-binding protein of any one of embodiments 1 to 31, the polypeptide described in embodiment 31, the immunoconjugate described in embodiment 33, the nucleic acid molecule described in embodiment 54, the expression vector described in embodiment 55, the cell described in embodiment 56, the chimeric antigen receptor described in any of embodiments 34 to 53, the engineered cell described in any of embodiments 58 to 68, or the pharmaceutical composition described in embodiment 79 in the preparation of a medicament for preventing and/or treating a CD7-related disease or condition.

82. The use of embodiment 81, wherein the CD7-related disease or condition includes a tumor.

83. The use of embodiment 82, wherein the tumor includes a tumor expressing CD7.

84. The use of any one of embodiments 82 to 83, wherein the tumor includes a hematological tumor.

85. The use of any one of embodiments 82 to 84, wherein the tumor includes a CD7-positive hematological malignancy.

86. The use of any one of embodiments 82 to 85, wherein the tumor includes a T cell malignancy.

87. A method for treating a tumor, the method including administering to a subject in need thereof an effective amount of the isolated antigen-binding protein of any one of embodiments 1-31, the polypeptide of embodiment 31, the immunoconjugate of embodiment 33, the nucleic acid molecule of embodiment 54, the expression vector of embodiment 55, the cell of embodiment 56, the chimeric antigen receptor of any one of embodiments 34-53, the engineered cell of any one of embodiments 58-68, or the pharmaceutical composition of embodiment 79.

88. The method of embodiment 87, wherein the tumor includes a tumor expressing CD7.

89. The method of embodiment 87, wherein the tumor includes a hematological tumor.

90. The method of embodiment 87, wherein the tumor includes a CD7-positive hematological malignancy.

91. The method of embodiment 87, wherein the tumor includes a T cell malignancy.

92. The method of embodiment 91, wherein the T cell malignancy includes acute T lymphocytic leukemia (T-ALL), acute myeloid leukemia or NK/T cell lymphoma.

93. A method for killing malignant T cells, the method including contacting the malignant T cells with the engineered cells of any one of embodiments 58-68.

The present application also provides the following implementation modes:
1. A gRNA targeting the CD7 gene, comprising a nucleotide sequence shown in any one of SEQ ID NO: 212 to SEQ ID NO: 218.

2. An isolated nucleic acid molecule comprising the gRNA of embodiment 1 or a DNA molecule encoding the gRNA.

3. An expression vector comprising the gRNA of embodiment 1 or the nucleic acid molecule of embodiment 2.

4. A gene editing system, comprising the gRNA of embodiment 1, the nucleic acid molecule of embodiment 2, or the expression vector of embodiment 3.

5. The gene editing system of embodiment 4, including a CRISPR/Cas gene editing system.

6. The gene editing system of embodiment 4, including a CRISPR/Cas9 gene editing system.

7. The gene editing system of embodiment 4, further comprising DNA encoding Cas9, mRNA encoding Cas9 or Cas9 protein molecule.

8. The gene editing system of embodiment 4, comprising an expression vector encoding the gRNA and Cas9 targeting the CD7 gene of embodiment 1.

9. A cell comprising the gRNA of embodiment 1, the nucleic acid molecule of embodiment 2, the expression vector of embodiment 3, or the gene editing system of any one of embodiment 4.

10. The cell of embodiment 9, includes a cell expressing CD7.

11. The cell of embodiment 9 includes an immune effector cell.

12. The cell of embodiment 11, wherein the immune effector cell includes a T cell, a B cell, a natural killer (NK) cell, a mast cell or a phagocyte.

13. The cell of any one of embodiments 11 to 12, wherein the immune effector cell includes an engineered immune effector cell.

14. The cell of embodiment 13, wherein the engineered immune effector cell targets CD7.

15. The cell of any one of embodiments 13 to 14, wherein the engineered immune effector cell includes a CAR-T cell.

16. The cell of any one of embodiments 13 to 15, wherein the engineered immune effector cell includes anti-CD7 CAR-T cell.

17. The cell of any one of embodiments 15 to 16, wherein the CAR comprises an extracellular antigen-binding domain, and the extracellular antigen-binding domain comprises the amino acid sequence shown in any one of SEQ ID NO: 162 to SEQ ID NO: 174.

18. The cell of any one of embodiments 15 to 17, wherein the CAR comprises the amino acid sequence shown in any one of SEQ ID NO: 180 to SEQ ID NO: 192.

19. Use of the gRNA of embodiment 1, the nucleic acid molecule of embodiment 2, the expression vector of embodiment 3, the gene editing system of embodiments 4 to 8 or the cell of any one of embodiments 9 to 18 in the preparation of a drug for treating tumors.

20. The use of embodiment 19, wherein the tumor includes a solid tumor or a hematological tumor.

21. The use of any one of embodiments 19 to 20, wherein the tumor includes a CD7-positive tumor.

22. The use of any one of embodiments 19 to 21, wherein the drug includes CAR-T cells.

23. The use of any one of embodiments 19 to 22, wherein the drug includes CD7 targeting CAR-T cells.

24. A method for gene editing of the CD7 gene in a cell, including using the gRNA of embodiment 1 to mediate Cas9 gene editing of the CD7 gene.

25. The method of embodiment 24, wherein the gene editing includes gene knockout.

26. A method for regulating T cell function, the method including introducing the gRNA of embodiment 1, the nucleic acid molecule of embodiment 2, the expression vector of embodiment 3, or the gene editing system of any one of embodiments 4 to 8 into T cell.

27. The method of embodiment 26, further including administering a Cas enzyme to the cell.

28. The method of embodiment 27, wherein the Cas enzyme includes a Cas9 protein.

29. The method of embodiment 28, wherein the Cas9 is delivered to the cell as mRNA or protein.

30. The method of embodiment 29, wherein the gRNA is delivered simultaneously with the Cas9.

31. The method of embodiment 30, wherein the delivering is performed by electroporation.

32. The method of any one of embodiments 26 to 31, the CD7 gene expression of the regulated T cells is downregulated or knocked out compared with unregulated T cells.

33. The method of any one of embodiments 26 to 32, the method also includes modifying the specificity of the T cell by introducing a nucleic acid molecule encoding CAR into the T cell.

34. The method of embodiment 33, wherein the nucleic acid molecule encoding CAR includes mRNA.

35. The method of embodiment 34, wherein the mRNA encodes anti-CD7 CAR.

36. The method of embodiment 35, wherein the CAR comprises an extracellular antigen-binding domain, and the extracellular antigen-binding domain comprises the amino acid sequence shown in any one of SEQ ID NO: 162 to SEQ ID NO: 174.

37. The method of embodiment 36, wherein the anti-CD7 CAR comprises an amino acid sequence shown in any one of SEQ ID NO: 180 to SEQ ID NO: 192.

38. The method of any one of embodiments 26 to 37, the regulated T cell has reduced CD7 surface expression compared with corresponding T cell and expresses anti-CD7 CAR.

39. The method of any one of embodiments 26 to 38, the gRNA of embodiment 1, the nucleic acid molecule of embodiment 2, the expression vector of embodiment 3, the gene editing system of any one of embodiments 4 to 8, and/or the nucleic acid molecule encoding CAR are introduced into T cells by any method selected from the following: ultrasonic treatment, electric pulse, electroporation, osmotic pressure shock, calcium phosphate precipitation, DEAE dextran transfection, lipid-mediated delivery, and passive delivery.

The present application also provides the following embodiments:

1. A modified immune effector cell, comprising a nucleic acid molecule encoding a fusion protein, wherein the fusion protein comprises a first domain that activates an antigen presenting cell (APC) and a second domain that activates an immune effector cell, wherein (i) the first domain includes (a) an activating receptor ligand or a receptor binding fragment thereof that binds to the APC, or (b) an activating receptor antibody or an antigen-binding fragment thereof that binds to the APC; and (ii) the second domain includes (a) a co-stimulatory ligand or a receptor binding fragment thereof of the immune effector cell, (b) an antibody or an antigen-binding fragment thereof that binds to a co-stimulatory receptor of the immune effector cell, or (c) a co-stimulatory receptor or a functional fragment thereof of the immune effector cell.

2. The modified immune effector cell of embodiment 1, wherein the N-terminus of the first domain is connected to the C-terminus of the second domain.

3. The modified immune effector cell of embodiment 1, wherein the C-terminus of the first domain is connected to the N-terminus of the second domain.

4. The modified immune effector cell of any one of embodiments 1 to 3, wherein the first domain is directly or indirectly connected to the second domain.

5. The modified immune effector cell of any one of embodiments 1 to 4, wherein the first domain is connected to the second domain by a linker.

6. The modified immune effector cell of embodiment 5, wherein the linker includes a peptide linker.

7. The modified immune effector cell of any one of embodiments 1 to 6, wherein the antibody or antigen-binding fragment thereof is an scFv.

8. The modified immune effector cell of any one of embodiments 1 to 7, wherein the APC is selected from the group consisting of a dendritic cell, a macrophage, a myeloid-derived suppressor cell, a monocyte, a B cell, a T cell, and a Langerhans cell.

9. The modified immune effector cell of any one of embodiments 1 to 8, wherein the activation receptor of the APC is selected from CD40, CD80, CD86, CD91, DEC-205 and DC-SIGN.

10. The modified immune effector cell of any one of embodiments 1 to 9, wherein the first domain comprises a ligand that binds CD40, CD80, CD86, CD91, DEC-205, DC-SIGN, or a receptor binding fragment thereof.

1 The modified immune effector cell of any one of embodiments 1 to 10, wherein the first domain comprises a receptor binding fragment of CD40 ligand (CD40L).

12. The modified immune effector cell of any one of embodiments 1 to 11, wherein the first domain comprises an antibody or antigen-binding fragment thereof that binds to an activating receptor of the APC.

13. The modified immune effector cell of any one of embodiments 1 to 12, wherein the first domain is an anti-CD40 antibody or an antigen-binding fragment thereof.

14. The modified immune effector cell of any one of embodiments 1 to 13, wherein the immune effector cell is selected from the group consisting of a T cell, a NK cell, an NKT cell, a macrophage, a neutrophil and a granulocyte.

15. The modified immune effector cell of any one of embodiments 1 to 14, wherein the second domain comprises an intracellular domain of a co-stimulatory receptor.

16. The modified immune effector cell of any one of embodiments 1 to 15, wherein the co-stimulatory receptor is selected from CD28, 4-1BB, ICOS, CD27, OX40, DAP10, 2B4, CD30, CD2, LIGHT, GITR, TLR, DR3 and CD43.

17. The modified immune effector cell of any one of embodiments 1 to 16, wherein the co-stimulatory receptor is CD28.

18. The modified immune effector cell of any one of embodiments 1 to 17, wherein the second domain is a co-stimulatory ligand or a receptor binding fragment thereof of the immune effector cell.

19. The modified immune effector cell of any one of embodiments 1 to 18, wherein the co-stimulatory ligand is selected from CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R and CD44.

20. The modified immune effector cell of any one of embodiments 1 to 19, wherein the second domain is an antibody or antigen-binding fragment thereof that binds a co-stimulatory receptor.

21. The modified immune effector cell of any one of embodiments 1 to 21, wherein the co-stimulatory receptor is selected from CD28, 4-1BB, ICOS, CD27, OX40, DAP10, 2B4, CD30, CD2, LIGHT, GITR, TLR, DR3 and CD43.

22. The modified immune effector cell of embodiment 22, wherein the co-stimulatory receptor is CD28 or 4-1BB.

23. The modified immune effector cell of any one of embodiments 1 to 22, wherein the first domain and the second domain are selected from the following combinations:
i) the first domain comprises CD40L or a receptor binding fragment thereof, and the second domain comprises a CD28 or 4-1BB intracellular domain;
ii) the first domain comprises CD40L or a receptor binding fragment thereof, and the second domain comprises an anti-CD28 antibody or an antigen-binding fragment thereof;
ii) the first domain comprises an anti-CD40 antibody or an antigen-binding fragment thereof, and the second domain comprises a CD28 intracellular domain;
iv) the first domain comprises an anti-CD40 antibody or an antigen-binding fragment thereof, and the second domain comprises an anti-CD28 antibody or an antigen-binding fragment thereof;
v) the first domain comprises CD40L or a receptor binding fragment thereof, and the second domain comprises a CD28 co-stimulatory ligand or a receptor binding fragment thereof; and
vi) the first domain comprises an anti-CD40 antibody or an antigen-binding fragment thereof, and the second domain comprises a CD28 co-stimulatory ligand or a receptor-binding fragment thereof.

24. The modified immune effector cell of any one of embodiments 1 to 23, wherein the fusion protein comprises the amino acid sequence shown in any one of SEQ ID NO: 83 to SEQ ID NO: 99.

25. The modified immune effector cell of any one of embodiments 1 to 24, wherein the immune effector cell includes an engineered immune effector cell.

26. The modified immune effector cell of embodiment 25, wherein the engineered immune effector cell includes a CAR-T cell, a CAR-NK cell or a TCR-T cell.

27. The modified immune effector cell of any one of embodiments 1 to 26, wherein the modified immune effector cell has reduced CD7 surface expression and expresses anti-CD7 CAR compared with the corresponding immune cell.

28. The modified immune effector cell of embodiment 27, wherein the engineered immune effector cell includes anti-CD7 CAR-T cell.

29. The modified immune effector cell of embodiment 28, wherein the CAR comprises an extracellular antigen-binding domain, and the extracellular antigen-binding domain comprises the amino acid sequence shown in any one of SEQ ID NO: 28 to SEQ ID NO: 40.

30. The modified immune effector cell of embodiment 29, wherein the CAR comprises the amino acid sequence shown in any one of SEQ ID NO: 46 to SEQ ID NO: 58.

31. The modified immune effector cell of any one of embodiments 1 to 30, wherein the expression of CD7 of the modified immune effector cell is absent or suppressed.

32. The modified immune effector cell of any one of embodiments 1 to 31, the modified immune effector cell does not induce fratricide.

33. A chimeric antigen receptor T (CAR-T) cell, which expresses anti-CD7 CAR and comprises a nucleic acid molecule encoding a fusion protein, wherein the fusion protein comprises a first domain that activates an antigen presenting cell (APC) and a second domain that activates an immune effector cell, wherein (i) the first domain includes (a) an activating receptor ligand or a receptor binding fragment thereof that binds to an APC, or (b) an activating receptor antibody or an antigen-binding fragment thereof that binds to an APC; and (ii) the second domain includes (a) a co-stimulatory ligand or a receptor binding fragment thereof of an immune effector cell, (b) an antibody or an antigen-binding fragment thereof that binds to a co-stimulatory receptor of an immune effector cell, or (c) a co-stimulatory receptor or a functional fragment thereof of an immune effector cell.

34. The CAR-T cell of embodiment 33, wherein the expression of CD7 of the CAR-T cell is absent or suppressed.

35. The CAR-T cell of any one of embodiments 33 to 34, the CAR-T cell does not induce fratricide.

36. A drug combination comprising an immune effector cell and a fusion protein, wherein the fusion protein comprises a first domain that activates an antigen presenting cell (APC) and a second domain that activates an immune effector cell, wherein (i) the first domain includes (a) an activating receptor ligand or a receptor binding fragment thereof that binds to the APC, or (b) an activating receptor antibody or an antigen-binding fragment thereof that binds to the APC; and (ii) the second domain includes (a) a co-stimulatory ligand or a receptor binding fragment thereof of the immune effector cell, or (b) an antibody or an antigen-binding fragment thereof that binds to a co-stimulatory receptor of the immune effector cell.

37. The drug combination of embodiment 36, the N-terminus of the first domain is connected to the C-terminus of the second domain.

38. The drug combination of embodiment 36, the C-terminus of the first domain is connected to the N-terminus of the second domain.

39. The drug combination of any one of embodiments 36 to 38, wherein the first domain is directly or indirectly connected to the second domain.

40. The drug combination of any one of embodiments 36 to 39, wherein the first domain and the second domain are connected by a linker.

41. The drug combination of embodiment 40, the linker includes a peptide linker.

42. The drug combination of any one of embodiments 36 to 41, wherein the antibody or antigen-binding fragment thereof is an scFv.

43. The drug combination of any one of embodiments 36 to 42, wherein the APC is selected from the group consisting of a dendritic cell, a macrophage, a myeloid-derived suppressor cell, a monocyte, a B cell, a T cell and a Langerhans cell.

44. The drug combination of any one of embodiments 36 to 43, wherein the activation receptor of the APC is selected from CD40, CD80, CD86, CD91, DEC-205 and DC-SIGN.

45. The drug combination of any one of embodiments 36 to 44, wherein the first domain comprises a ligand that binds to CD40, CD80, CD86, CD91, DEC-205, DC-SIGN, or a receptor binding fragment thereof.

46. The drug combination of any one of embodiments 36 to 45, wherein the first domain comprises a receptor binding fragment of CD40 ligand (CD40L).

47. The drug combination of any one of embodiments 36 to 46, wherein the first domain comprises an antibody or an antigen-binding fragment thereof that binds to an activating receptor of the APC.

48. The drug combination of any one of embodiments 36 to 47, wherein the first domain is an anti-CD40 antibody or an antigen-binding fragment thereof.

49. The drug combination of any one of embodiments 36 to 48, wherein the immune effector cells are selected from the group consisting of a T cell, a NK cell, an NKT cell, a macrophage, a neutrophil and a granulocyte.

50. The drug combination of any one of embodiments 36 to 49, wherein the second domain comprises an intracellular domain of a co-stimulatory receptor.

51. The drug combination of any one of embodiments 36 to 50, wherein the co-stimulatory receptor is selected from CD28, 4-1BB, ICOS, CD27, OX40, DAP10, 2B4, CD30, CD2, LIGHT, GITR, TLR, DR3 and CD43.

52. The drug combination of any one of embodiments 36 to 51, wherein the co-stimulatory receptor is CD28 or 4-1BB.

53. The drug combination of any one of embodiments 36 to 52, wherein the second domain is a co-stimulatory ligand of an immune effector cell or a receptor binding fragment thereof.

54. The drug combination of any one of embodiments 36 to 53, wherein the co-stimulatory ligand is selected from CD58, CD70, CD83, CD80, CD86, CD137L, CD252, CD275, CD54, CD49a, CD112, CD150, CD155, CD265, CD270, TL1A, CD127, IL-4R, GITR-L, TIM-4, CD153, CD48, CD160, CD200R and CD44.

55. The drug combination of any one of embodiments 36 to 54, the second domain is an antibody or an antigen-binding fragment thereof that binds to a co-stimulatory receptor.

56. The drug combination of any one of embodiments 36 to 55, wherein the co-stimulatory receptor is selected from CD28, 4-1BB, ICOS, CD27, OX40, DAP10, 2B4, CD30, CD2, LIGHT, GITR, TLR, DR3 and CD43.

57. The drug combination of any one of embodiments 36 to 56, wherein the co-stimulatory receptor is CD28 or 4-1BB.

58. The drug combination of any one of embodiments 36-57, wherein the first domain and the second domain are selected from the following combinations:
i) the first domain comprises CD40L or a receptor binding fragment thereof, and the second domain comprises a CD28 co-stimulatory ligand or a receptor binding fragment thereof;
ii) the first domain comprises CD40L or a receptor binding fragment thereof, and the second domain comprises an anti-CD28 antibody or an antigen-binding fragment thereof;
iii) the first domain comprises an anti-CD40 antibody or an antigen-binding fragment thereof, and the second domain comprises a CD28 co-stimulatory ligand or a receptor-binding fragment thereof; and
iv) the first domain comprises an anti-CD40 antibody or an antigen-binding fragment thereof, and the second domain comprises an anti-CD28 antibody or an antigen-binding fragment thereof.

59. The drug combination of any one of embodiments 36 to 58, wherein the fusion protein comprises the amino acid sequence shown in any one of SEQ ID NO: 86 to SEQ ID NO: 90.

60. The drug combination of any one of embodiments 36 to 59, wherein the immune effector cell includes an engineered immune effector cell.

61. The drug combination of any one of embodiments 36 to 60, wherein the engineered immune effector cell includes a CAR-T cell, a CAR-NK or a TCR-T cell.

62. The drug combination of any one of embodiments 36 to 61, wherein the drug combination has reduced CD7 surface expression compared with corresponding immune cells and expresses anti-CD7 CAR.

63. The drug combination of any one of embodiments 36 to 62, wherein the engineered immune effector cell includes an anti-CD7 CAR-T cell.

64. The drug combination of any one of embodiments 36 to 63, wherein the CAR comprises an extracellular antigen-binding domain, and the extracellular antigen-binding domain comprises the amino acid sequence shown in any one of SEQ ID NO: 28 to SEQ ID NO: 40.

65. The drug combination of any one of embodiments 36 to 64, wherein the CAR comprises an amino acid sequence shown in any one of SEQ ID NO: 46 to SEQ ID NO: 58.

66. The drug combination of any one of embodiments 36 to 65, the immune effector cell does not induce fratricide.

67. A pharmaceutical composition, comprising the modified immune effector cell of any one of embodiments 1 to 32, the CAR-T cell of any one of embodiments 33 to 35, or the drug combination of any one of embodiments 36 to 66, and a pharmaceutically acceptable carrier.

68. Use of the modified immune effector cell of any one of embodiments 1 to 32, the CAR-T cell of any one of embodiments 33 to 35, the drug combination of any one of embodiments 36 to 66, or the pharmaceutical composition of embodiment 67 in the preparation of a drug for treating a tumor.

69. The use of embodiment 68, wherein the tumor includes hematological tumors and solid tumors.

70. The use of any one of embodiments 68 to 69, wherein the tumor includes a tumor expressing CD7.

71. The use of any one of embodiments 68 to 70, wherein the tumor includes a CD7-positive hematological malignancy.

72. The use of any one of embodiments 68 to 71, wherein the tumor includes a T cell malignancy.

73. The use of any one of embodiments 68 to 72, wherein the T-cell malignancy includes acute T-lymphocytic leukemia (T-ALL), acute myeloid leukemia, or NK/T-cell lymphoma.

74. A method for treating a tumor, the method including administering to a subject in need thereof an effective amount of the modified immune effector cell of any one of embodiments 1 to 32, the CAR-T cell of any one of embodiments 33 to 35, the drug combination of any one of embodiments 36 to 66, or the pharmaceutical composition of embodiment 67.

75. A method for killing malignant T cells, the method including contacting the malignant T cells with the modified immune effector cells of any one of embodiments 1 to 32, the CAR-T cells of any one of embodiments 33 to 35, the drug combination of any one of embodiments 36 to 66, or the pharmaceutical composition of embodiment 67.

76. A method for preparing a chimeric antigen receptor T (CAR-T) cell population, wherein the CAR targets CD7, including the following steps:
a. Modifying the CD7 gene in the population of T cells;
b. Activating the population of T cells;
c. Transducing the population of T cells with anti-CD7 CAR and fusion protein; and
d. Expanding the population of chimeric antigen receptor T cells.

77. The method of embodiment 76, wherein the CD7 is absent or suppressed in the population of modified T cells compared with the corresponding unmodified cells.

78. The method of any one of embodiments 76 to 77, wherein the modification includes administering to the T cell population one or more substances selected from the group consisting of antisense RNA, siRNA, shRNA, transcription activator-like effector nuclease (TALEN), zinc finger nuclease (ZFN) and CRISPR/Cas system.

79. The method of any one of embodiments 76 to 78, wherein the modification includes administering a CRISPR/Cas system to the T cell population.

80. The method of any one of embodiments 76 to 79, wherein the modification includes administering a CRISPR/Cas9 system to the T cell population.

81. The method of embodiment 80, wherein the modification includes administering Cas9 and a gRNA targeting the CD7 gene to the T cell population.

82. The modified method of embodiment 81, wherein the gRNA targeting the CD7 gene comprises a nucleotide sequence shown in any one of SEQ ID NO: 64 to SEQ ID NO: 71.

83. The method of embodiment 82, wherein the Cas9 is delivered to the cell as mRNA or protein.

84. The method of embodiment 83, wherein the gRNA is delivered simultaneously with the Cas9.

85. The method of embodiment 84, wherein the delivering is performed by electroporation.

86. The method of any one of embodiments 76 to 85, transducing the anti-CD7 CAR and the fusion protein into the T cells includes introducing a nucleic acid molecule encoding the anti-CD7 CAR and a nucleic acid molecule encoding the fusion protein into the T cells.

87. A population of cells comprising the modified immune effector cell of any one of embodiments 1 to 32 or the CAR-T cell of any one of embodiments 33 to 35, wherein the population of cells is derived from peripheral blood mononuclear cells (PBMC), peripheral blood lymphocytes (PBL), tumor-infiltrating lymphocytes (TIL), cytokine-induced killer cells (CIK), lymphokine-activated killer cells (LAK), or marrow-infiltrating lymphocytes (MIL).

### CircRNA

The present application provides a circRNA, comprising, in the following order, an internal ribosome entry site (IRES) element, a protein coding sequence targeting CD7, and polyadenylic acid (polyA).

In the present application, the length of the poly A can be at least 45 nucleotides. For example, the length of the poly A can be at least 50 nucleotides, at least 55 nucleotides, at least 60 nucleotides, at least 65 nucleotides, at least 70 nucleotides, at least 75 nucleotides, at least 80 nucleotides or more.

In the present application, the protein targeting CD7 can include an antibody or an antigen-binding fragment thereof, a chimeric antigen receptor (CAR) and/or a T cell receptor (TCR) targeting CD7.

In the present application, the antibody can include a monoclonal antibody, a polyclonal antibody, a dimer, a multimer, a multispecific antibody, a full-length antibody, an antibody fragment, a human antibody, a humanized antibody or a chimeric antibody.

In the present application, the antigen-binding fragment can include Fab, Fab′, Fv fragment, F(ab′)2, scFv, di-scFv and/or dAb.

In the present application, the protein targeting CD7 can be a chimeric antigen receptor (CAR) targeting CD7. The CAR can comprise an extracellular domain of a chimeric antigen receptor that specifically binds to CD7. The extracellular domain can also be referred to as a binding domain. For example, the binding domain can include a CD7 scFv.

In the present application, the antibody (e.g., the binding domain of the antibody or the CAR, such as the CD7 scFV) can include a light chain variable region comprising LCDR1, LCDR2 and LCDR3 and a heavy chain variable region comprising HCDR1, HCDR2 and HCDR3, wherein: the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 74, the LCDR2 comprises the amino acid sequence shown in SEQ ID NO: 87, and the LCDR3 comprises the amino acid sequence shown in SEQ ID NO: 99; or the HCDR1 comprises the amino acid sequence shown in SEQ ID NO: 3, the HCDR2 comprises the amino acid sequence shown in SEQ ID NO: 11, the HCDR3 comprises the amino acid sequence shown in SEQ ID NO: 19, the LCDR1 comprises the amino acid sequence shown in SEQ ID NO: 75, the LCDR2 comprises the amino acid sequence shown in SEQ ID NO: 88, and the LCDR3 comprises the amino acid sequence shown in SEQ ID NO: 100; or the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 20, the LCDR1 comprises the amino acid sequence shown in SEQ ID NO: 76, the LCDR2 comprises the amino acid sequence shown in SEQ ID NO: 89, and the LCDR3 comprises the amino acid sequence shown in SEQ ID NO: 101; or the HCDR1 comprises the amino acid sequence shown in SEQ ID NO: 4, the HCDR2 comprises the amino acid sequence shown in SEQ ID NO: 12, the HCDR3 comprises the amino acid sequence shown in SEQ ID NO: 21, the LCDR1 comprises the amino acid sequence shown in SEQ ID NO: 77, the LCDR2 comprises the amino acid sequence shown in SEQ ID NO: 90, and the LCDR3 comprises the amino acid sequence shown in SEQ ID NO: 102; or the HCDR1 comprises the amino acid sequence shown in SEQ ID NO: 5, the HCDR2 comprises the amino acid sequence shown in SEQ ID NO: 13, the HCDR3 comprises the amino acid sequence shown in SEQ ID NO: 22, and the LCDR1 comprises the amino acid sequence shown in SEQ ID NO: wherein the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 79, the LCDR2 comprises the amino acid sequence shown in SEQ ID NO: 92, and the LCDR3 comprises the amino acid sequence shown in SEQ ID NO: 104; or the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 80, the LCDR2 comprises the amino acid sequence shown in SEQ ID NO: 93, and the LCDR3 comprises the amino acid sequence shown in SEQ ID NO: 105; or the HCDR1 comprises the amino acid sequence shown in SEQ ID NO: 6, the HCDR2 comprises the amino acid sequence shown in SEQ ID NO: 14, the HCDR3 comprises the amino acid sequence shown in SEQ ID NO: 23, the LCDR1 comprises the amino acid sequence shown in SEQ ID NO: 81, the LCDR2 comprises the amino acid sequence shown in SEQ ID NO: 94, and the LCDR3 comprises the amino acid sequence shown in SEQ ID NO: 106; or the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 82, the LCDR2 comprises the amino acid sequence shown in SEQ ID NO: 95, and the LCDR3 comprises the amino acid sequence shown in SEQ ID NO: 107; or the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 24, and the LCDR1 comprises the amino acid sequence shown in SEQ ID NO: wherein the HCDR1 comprises the amino acid sequence shown in SEQ ID NO: 83, the LCDR2 comprises the amino acid sequence shown in SEQ ID NO: 96, and the LCDR3 comprises the amino acid sequence shown in SEQ ID NO: 108; or the HCDR1 comprises the amino acid sequence shown in SEQ ID NO: 7, the HCDR2 comprises the amino acid sequence shown in SEQ ID NO: 15, and the HCDR3 comprises the amino acid sequence shown in SEQ ID NO: 25, the LCDR1 comprises the amino acid sequence shown in SEQ ID NO: 84, the LCDR2 comprises the amino acid sequence shown in SEQ ID NO: 97, and the LCDR3 comprises the amino acid sequence shown in SEQ ID NO: 109; or the HCDR1 comprises the amino acid sequence shown in SEQ ID NO: 8, the HCDR2 comprises the amino acid sequence shown in SEQ ID NO: 16, and the HCDR3 comprises the amino acid sequence shown in SEQ ID NO: 26, the LCDR1 comprises the amino acid sequence shown in SEQ ID NO: 85, the LCDR2 comprises the amino acid sequence shown in SEQ ID NO: 98, and the LCDR3 comprises the amino acid sequence shown in SEQ ID NO: 110; or the HCDR1 comprises SEQ ID NO: The present invention relates to a novel amino acid sequence comprising the amino acid sequence shown in SEQ ID NO: 9, the HCDR2 comprises the amino acid sequence shown in SEQ ID NO: 17, the HCDR3 comprises the amino acid sequence shown in SEQ ID NO: 27, the LCDR1 comprises the amino acid sequence shown in SEQ ID NO: 86, the LCDR2 comprises the amino acid sequence shown in SEQ ID NO: 98, and the LCDR3 comprises the amino acid sequence shown in SEQ ID NO: 111.

In the present application, the antibody (e.g., the binding domain of the antibody or the CAR, such as the CD7 scFV) can include VH and VL, wherein the VH comprises the amino acid sequence shown in SEQ ID NO: 61, and the VL comprises the amino acid sequence shown in SEQ ID NO: 148; or the VH comprises the amino acid sequence shown in SEQ ID NO: 62, and the VL comprises the amino acid sequence shown in SEQ ID NO: 149; or the VH comprises the amino acid sequence shown in SEQ ID NO: 63, and the VL comprises the amino acid sequence shown in SEQ ID NO: 150; or the VH comprises the amino acid sequence shown in SEQ ID NO: 64, and the VL comprises the amino acid sequence shown in SEQ ID NO: 151; or the VH comprises the amino acid sequence shown in SEQ ID NO: 65, and the VL comprises the amino acid sequence shown in SEQ ID NO: 152; or the VH comprises the amino acid sequence shown in SEQ ID NO: 66, and the VL comprises the amino acid sequence shown in SEQ ID NO: 153; or the VH comprises the amino acid sequence shown in SEQ ID NO: 67, and the VL comprises the amino acid sequence shown in SEQ ID NO: 154; or the VH comprises SEQ ID NO: or the VH comprises the amino acid sequence shown in SEQ ID NO: 71, and the VL comprises the amino acid sequence shown in SEQ ID NO: 158; or the VH comprises the amino acid sequence shown in SEQ ID NO: 72, and the VL comprises the amino acid sequence shown in SEQ ID NO: 159; or the VH comprises the amino acid sequence shown in SEQ ID NO: 73, and the VL comprises the amino acid sequence shown in SEQ ID NO: 160.

In the present application, the circRNA can comprise a nucleotide sequence shown in any one of SEQ ID NOs: 256 to 257.

The present application also provides an agent for regulating CD7 expression level and/or activity, which can comprise the circRNA described in the present application. The present application also provides the use of the circRNA described in the present application in regulating CD7 expression level and/or activity.

The present application also provides a method for regulating CD7 expression level and/or activity, which can include the following steps: administering the circRNA described in the present application.

In the present application, the reagent can be provided in the form of a kit. The kit can also include other reagents and/or instruments that can regulate CD7 expression level and/or activity.

In the present application, the expression level can include the expression level of nucleic acid (DNA and/or mRNA) encoding CD7 in the detection sample; the expression level can include the expression level of CD7 protein in the detection sample. The regulation can be performed in vivo, in vitro and/or ex vivo.

The regulation can include upregulation and/or downregulation. For example, the downregulation may be at least 10%, at least 20%, at least 30%, at least 40%, at least 50%, at least 60%, at least 70%, at least 80%, at least 90%, at least 100%, or more of the CD7 expression level and/or activity compared to without the administration of the circRNA.

The administration may include delivery, for example, the circRNA described herein may be delivered by electroporation.

In the present application, the circRNA, which can further comprise a nucleic acid molecule encoding a fusion protein, wherein the fusion protein comprises a first domain that activates an antigen presenting cell (APC) and a second domain that activates an immune effector cell, wherein (i) the first domain comprises (a) an activating receptor ligand or a receptor binding fragment thereof that binds to the APC, or (b) an activating receptor antibody or an antigen-binding fragment thereof that binds to the APC; and (ii) the second domain comprises (a) a co-stimulatory ligand or a receptor binding fragment thereof of an immune effector cell, (b) an antibody or an antigen-binding fragment thereof that binds to a co-stimulatory receptor of an immune effector cell, or (c) a co-stimulatory receptor or a functional fragment thereof of an immune effector cell.

In the present application, the first domain can be connected to the C-terminus or N-terminus of the second domain.

In the present application, the linker can include a peptide linker.

In the present application, the first domain can comprise CD40L or a receptor binding fragment thereof, an anti-CD40 antibody or an antigen-binding fragment thereof, and the second domain comprises a CD28 intracellular domain or an anti-CD28 antibody or an antigen-binding fragment thereof.

In the present application, the first domain can comprise an anti-CD40 antibody or an antigen-binding fragment thereof, and the second domain comprises a CD28 intracellular domain.

In the present application, the fusion protein can comprise the amino acid sequence shown in any one of SEQ ID NO: 234 to SEQ ID NO: 250.

In the present application, the circRNA can comprise a precursor RNA. The precursor RNA can include a circularizing element, an internal ribosome entry site (IRES) element, a protein coding sequence targeting CD7, and polyadenylic acid (polyA).

The circularizing element can comprise a first intron sequence located 5′ to an internal ribosome entry site (IRES) element and a second intron sequence located 3′ to the polyadenylation. The first intron sequence and the second intron sequence can be derived from a group I or group II intron self-splicing sequence.

The first intron sequence can comprise a group I intron fragment containing 3′ splice site nucleotides, and the second intron sequence can comprise a group I intron fragment containing a 5′ splice.

The precursor RNA can further comprise a 5′ spacer sequence between the first intron sequence and the IRES, and a 3′ spacer sequence between the first intron sequence and the IRES.

The precursor RNA can further comprise a 5′ homology arm outside the first intron sequence and a 3′ homology arm outside the second intron sequence.

The present application further provides a vector comprising the precursor RNA described herein. The vector can comprise DNA encoding the precursor RNA.

The present application further provides a method for producing the circRNA described in the present application. For example, it can comprise the following steps:
transcribing the vector comprising the precursor RNA described in the present application. The transcription can be performed in cells and/or under cell-free condition. In the present application, the method can further include the step of purifying the circRNA of the present application. For example, purification is performed by capture based of oligo dT.

The present application also provides cell and/or the population of cells comprising the circRNA described in the present application.

The present application also provides the use of the circRNA, the above-mentioned precursor RNA, the vector, the cell and/or the population of cells described in the present application in the preparation of a drug for treating a disease, wherein the drug is used to treat a tumor. The tumor can include a blood tumor and a solid tumor.

The tumor can include a tumor expressing CD7. The tumor can include a CD7-positive hematological malignancy. The tumor can include a T-cell malignancy. The T-cell malignancy can include acute T-lymphocytic leukemia (T-ALL), acute myeloid leukemia, or NK/T-cell lymphoma.

Without intending to be bound by any theory, the following examples are merely intended to illustrate the cells, preparation methods and uses of the present application, and are not intended to limit the scope of the invention of the present application.

### EXAMPLE

### Example 1 Screening of Anti-CD7 Antibodies from a Fully Human Antibody Phage Display Library

1. Infect logarithmic phase TG1 library culture at a multiplicity of infection of 20:1 (phage-to-cell ratio) by adding freshly thawed M13K07 helper phage and induce expression of phage display library with IPTG overnight.
2. Purify the phage library by PEG/NaCl precipitation and determine the phage titer. Store the phage at 4° C. and proceed to scFv selection as soon as possible.
3. Selection of CD7-specific scFv-phages. For the first round of selection, plate Maxisorp plates with 20 μg/ml CD7-6His protein dissolved in 1×PBS and incubate overnight at 4° C. (For subsequent rounds, reduce the protein concentration for more stringent selection, 2 μg/ml for the second round of bio-panning and 0.5 ug/ml for the third round of bio-panning).
4. Wash three times with PBS, add blocking buffer (5% milk+1% BSA in 1×PBS) to each well and incubate at RT for 2 h.
5. Discard the blocking buffer, add the phage solution, seal with sealing film, and incubate with gentle shaking for 2 hours.
6. In the first round of screening, wash 10 times with PBST. In the following rounds, increase the stringency of washing by increasing the number of washing cycles (20 times in the second round and 30 times in the third round).
7. Elute the antigen-bound scFv phage. Add 1 ml of acidic elution buffer (pH 2.2), incubate for about 8 minutes, and then use a pipette to aspirate the eluted phage to about 100 μl neutralization buffer and placed in a new PP tube.
8. Inoculate 15 mL of the logarithmic phase TG1 culture (OD600=0.5) with the eluted phage solution in a new 50 mL test tube. Incubate at 37° C., let stand for 30 minutes, shake for 30 minutes. Then culture on a 15 cm plate containing 2×YT-GA agar.
9. Incubate the plates at 30° C. overnight and harvest the bacteria for subsequent screening.
10. After three rounds of selection, select hundreds of positive colonies for monoclonal phage ELISA (mpELISA) screening.
11. mpELISA screening. Incubate phage supernatants produced by a single bacterium with pre-blocked Maxisorp plates coated with 2 μg/ml CD7-6His protein. After washing three times, add 100 μl/well of HRP-conjugated anti-M13 antibody diluted 1:5000 in blocking buffer (5% milk+1% BSA in 1×PBS) and incubate for 60 minutes at RT. After washing the plate 5 times with PBST, add 100 μl/well of TMB substrate solution and incubate for 10-30 minutes until blue color appears. Add 50 μl/well of stop solution (2N H2SO4) to stop the reaction. Read the absorbance at 450 nm in a microplate reader.
12. Positive clones were selected based on the ELISA results and used as templates for PCR cloning of scFv sequences (forward primer sequence: tgcagctggcacgacaggtttc (SEQ ID NO: 219), reverse primer sequence: cgtcagactgtagcacgtt (SEQ ID NO: 220)). The PCR products were then sequenced using the Sanger sequencing method (forward primer sequence: aacaattgaattcaggagga (SEQ ID NO: 221), reverse primer sequence: cctcctaagaagcgtagtc (SEQ ID NO: 222)).
13. The CDR region of scFv was analyzed using the abysis website (http://abysis.org/).

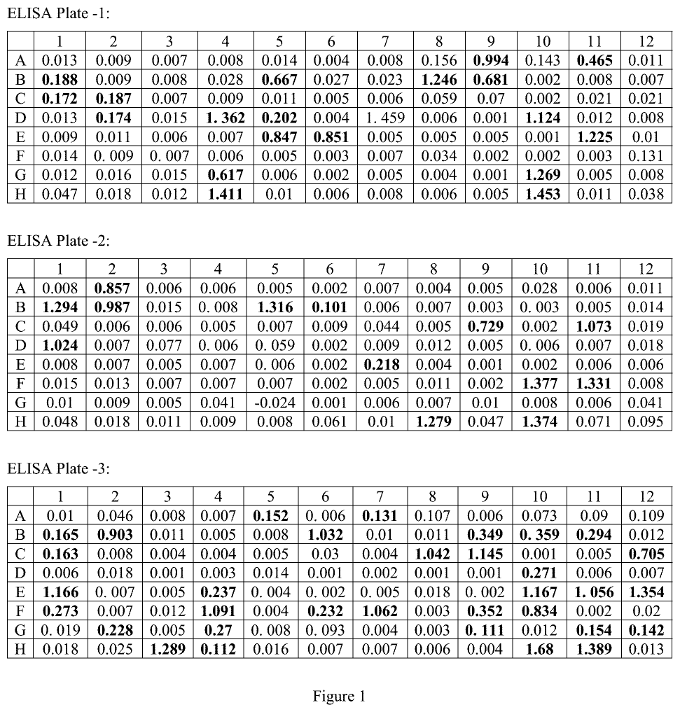

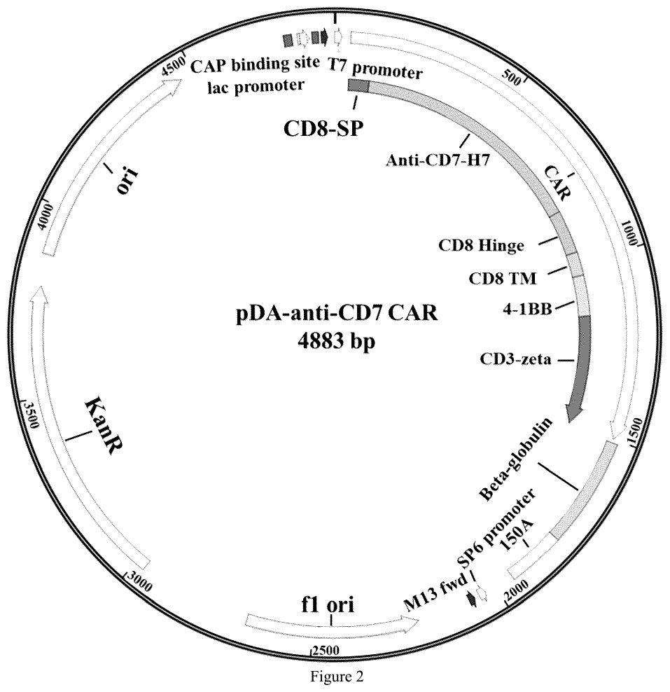

The results are shown in FIG. 1. After three rounds of bio-panning, 288 colonies were selected for culture and phage supernatant was produced to test its binding to CD7-Fc protein. The positive clones are highlighted in gray. These figures show that many anti-CD7 antibodies were successfully screened in the phage display screening experiment. In the scFv screening part, 18 anti-CD7 antibodies were successfully screened from the phage display library: anti-CD7 scFv-H1 to anti-CD7 scFv-H18.

### Example 2 Construction of Anti-CD7 CAR Vector for Generating mRNA

1. Digest the pDA vector with Xba1 and Sal1 enzymes and purify by gel purification.
2. Amplify the scFv sequence and CAR fragment (from the hinge domain to the CD3-zeta domain) by PCR and purify by gel purification.
3. Use the Gibson assembly method to connect the scFv fragment, CAR fragment (from the hinge domain to the CD3-zeta domain) and pDA vector (as shown in FIG. 2) and transform competent cells.
4. Confirm suitable colonies by Sanger sequencing and select for further experiments.

### Example 3 In Vitro Transcription (IVT) of CAR mRNA

1. Digest the pDA-CAR plasmid with Spe1 enzyme to linearize it.
2. Purify the linearized vector using a PCR cleanup kit and elute with RNase-free water.
3. Measure the concentration of DNA using a nanodrop and detect it using agarose DNA gel.
4. Perform IVT according to the manufacturer's protocol (Thermofisher, Cat No: AMB13455). Specifically, add 1 ug template DNA, NTP/ARCA buffer, T7 buffer, GTP, T7 enzyme, and RNase-free H2O in a 20 ul volume into a 0.2 ml PCR tube and incubate at 37° C. for 3 hours.
5. After 3 hours, add 2 μl of DNase to each reaction and incubate at 37° C. for 15 minutes.
6. Then follow the trailing procedure according to the manufacturer's recommendations.
7. Purify IVT mRNA using the RNasy kit (Qiagen).
8. Measure the concentration of RNA using a nanodrop and check by running a PAGE gel.

### Example 4 In Vitro Transcription (IVT) of CD7 gRNA

1. Digest the pUC57-CD7-gRNA plasmid with EcoR1 enzyme to linearize it.
2. Purify the linearized vector using a PCR cleanup kit and elute with RNase-free water.
3. Measure the concentration of DNA using a nanodrop and detect it using agarose DNA gel.
4. Perform IVT according to the manufacturer's protocol (Thermofisher, Cat No: AMB13345). Briefly, add 1 ug template DNA, NTP buffer, T7 buffer, T7 enzyme, and RNase-free H2O in a volume of 20 ul into a 0.2 ml PCR tube and incubate at 37° C. for 3-4 hours.
5. After 3-4 hours, add 1 μl of DNase to each reaction and incubate at 37° C. for 30 minutes.
6. Purify IVT gRNA using RNasy kit (Qiagen).
7. Measure the concentration of gRNA using a nanodrop and check by running a PAGE gel.

### Example 5 Tumor Cell Lines and Primary Human Lymphocytes

All tumor cell lines, including H226, OVCAR3, SKOV3, Caski, U87, U251, ASPC1, A549, Raji, Nalm6, MOLM14, SupT1, DOHH2, RPMI8226, Jurkat, and CEM, were cultured in RPMI-1640 medium supplemented with 10% FCS. Primary lymphocytes from normal donors were stimulated with anti-CD3/CD28 Dynabeads (Life Technologies) and cultured in R10 medium (RPMI-1640 supplemented with 10% FCS, penicillin-streptomycin (100×), HEPES (100×), sodium pyruvate (100×), Glutamax (100×), NEAA (100×)). T cells were cryopreserved in a solution of 90% FCS and 10% DMSO at day 12 after stimulation at 1×10e8 cells per vial.

### Example 6 Electroporation of mRNA into A549 and T Cells

1. Collect A549 tumor cells and T cells and wash them three times with Opti-MEM medium.
2. Resuspend the cell pellet in Opti-MEM medium. The cell concentration should be 1×10e7/ml.
3. Aliquot 10 ug RNA into 1.5 ml EP tubes, then add 100 μl T cells or A549 cells and mix well.
4. Set parameters on the BTX machine:
a) For T cells: 500V, 0.7 ms;
b) For A549 tumor cells: 300 V, 0.5 ms.
5. Add 100 ul of cells mixed with RNA into the BTX electroporation cup and tap gently to avoid bubbles.
6. Perform electroporation, then transfer cells to pre-warmed culture medium and culture at 37° C.

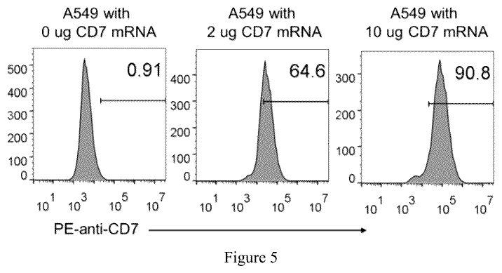

A549 cells electroporated with different amounts of CD7 mRNA were subjected to FACS staining using isotype and anti-CD7 antibodies. FIG. 5 shows that the ectopic expression level of CD7 is related to the amount of CD7 mRNA electroporated into A549 cells.

### Example 7 Preparation of CD7 KO CAR-T Cells and CD7 KO CAR+ LACO T Cells

1. On day 0, CD3+ T cells were activated with CD3/CD28 Dynabeads (CTS Dynabeads CD3/CD28: Gibco; Cat #402031; 4E8 beads/mL) at a ratio of CD3/CD28 beads:T cells=3:1.
2. On day 4, remove CD3/CD28 Dynabeads from T cells. Transfer T cells to a 15 ml French tube and place in a magnetic tube for 3-5 minutes, and transfer the supernatant to a new 15 ml French tube.
3. Count the number of T cells, wash three times with OPTI-MEM, and resuspend the T cells to 50E6/mL.
4. Electroporate T cells with Cas9 recombinant protein and CD7 gRNA. After washing T cells three times with OPTI-MEM, mix 15 ug Cas9 protein and 7.5 ug CD7 gRNA at room temperature for 10 minutes. Then add the Cas9 protein/gRNA mixture to T cells and mix gently. Perform electroporation with BTX ECM 830, with the condition parameters of 360V, 1 ms. Continue to culture the electroporated T cells.
5. On day 9, the efficiency of CD7 gene knockout was tested by staining with anti-CD7 antibody and analyzed by flow cytometry.

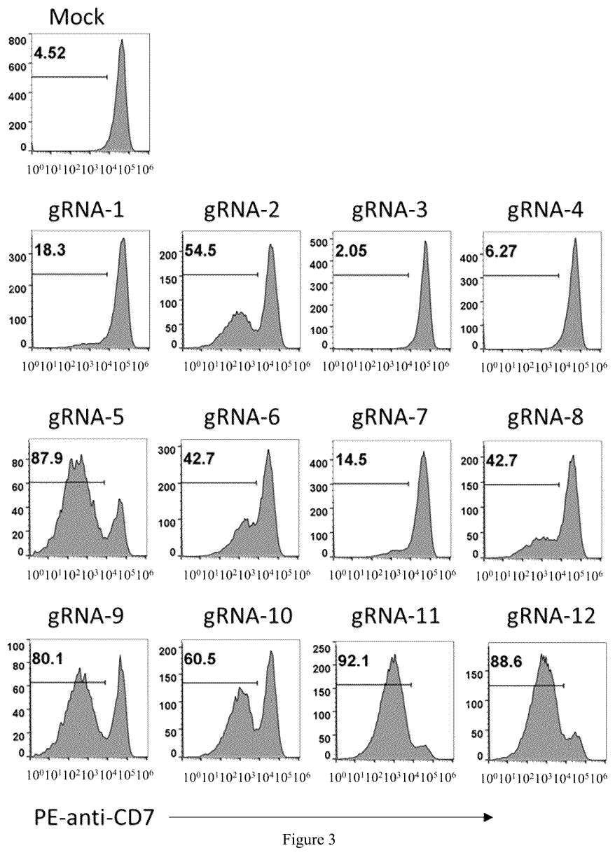

The FACS staining results (FIG. 3) showed the knockout efficiency of different CD7 gRNAs by CRISPR-Cas9 gene editing technology: the knockout efficiencies of CD7 gRNA-2, gRNA-5, gRNA-6, gRNA-8, gRNA-9, gRNA-10, gRNA-11, and gRNA-12 were 54.5%, 87.9%, 42.7%, 42.7%, 80.1%, 60.5%, 92%, and 88.6%, respectively, while CD7 gRNA-1, gRNA-3, gRNA-4, and gRNA-7 had almost no knockout effect.
6. On day 12, electroporation of anti-CD7 CAR mRNA to generate CD7 KO anti-CD7 CAR-T cells; or
CD7 CAR mRNA and LACO mRNA (the LACO molecule used in this experiment is A40C.CD28 (A40C28)) were electroporated to generate CD7 KO anti-CD7 CAR+A40C28 T cells.

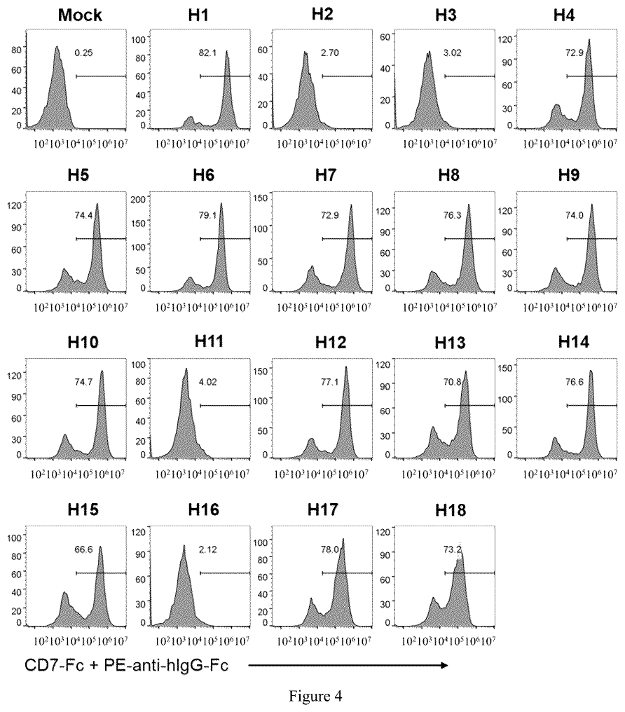

The binding ability of anti-CD7 scFv expressed in CAR-T cells to CD7-Fc protein was detected, where Mock was a control T cell without CAR molecules. FACS staining results (FIG. 4) showed that anti-CD7 scFv-H1, -H4, -H5, -H6, -H7, -H8, -H9, -H10, -H12, -H13, -H14, -H15, -H17 and -H18 had binding ability to CD7-Fc recombinant protein, while anti-CD7 scFv-H2, -H3, -H11 and -H16 had no binding ability or weak binding ability to CD7-Fc recombinant protein.

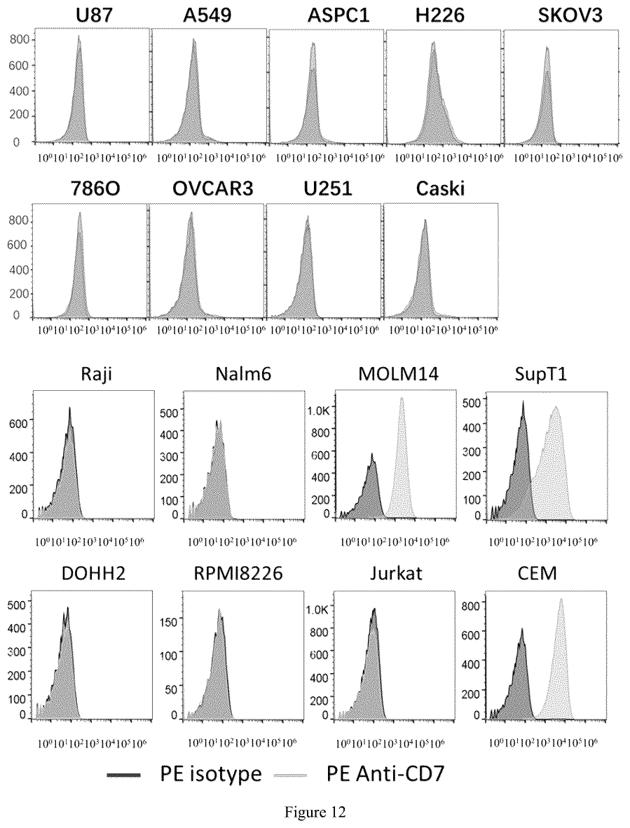

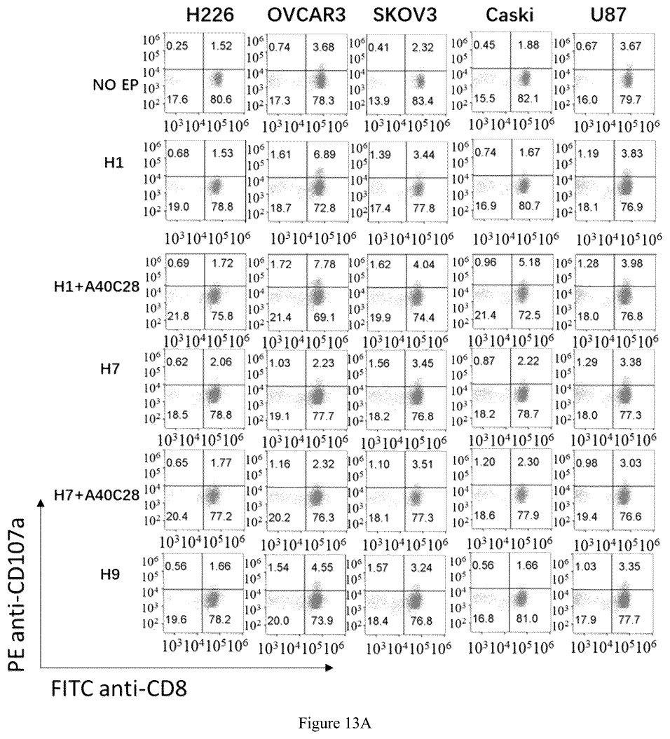

FACS staining of 17 different types of tumor cell lines was performed using isotype control and anti-CD7 mAb. FIG. 12 showed that MOLM14, SupT1, and CEM tumor cells expressed CD7 at high levels, while U87, A549, ASPC1, H226, SKOV3, 7860, OVCAR3, U251, Caski, Raji, Nalm6, DOHH2, RPMI8226, and Jurkat did not express CD7 or expressed CD7 at such low levels that it could not be detected.

### Example 8 In Vitro Cytotoxicity Test of Anti-CD7 CAR-T Cells

1. Inoculate EGFP-expressing tumor cell lines or electroporated EGFP-A549 cells into a flat-bottom 96-well plate at 3,000 cells/100 μl/well.
2. Dilute the CAR-T cells to an appropriate concentration and inoculate 100 ul/well of cells onto tumor cells with different E/T ratios, such as 10:1, 3:1, and 1:1.
3. Place the co-culture plate into the IncuCyte S3 machine and set the scanning parameters.
4. After 3 days of scanning, analyze the total integrated intensity of green objects (GCU×μm²/Well) and calculate the killing efficiency.

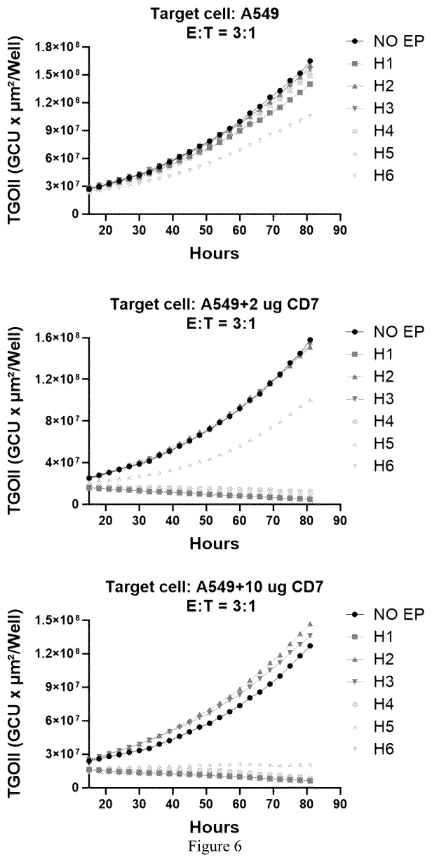

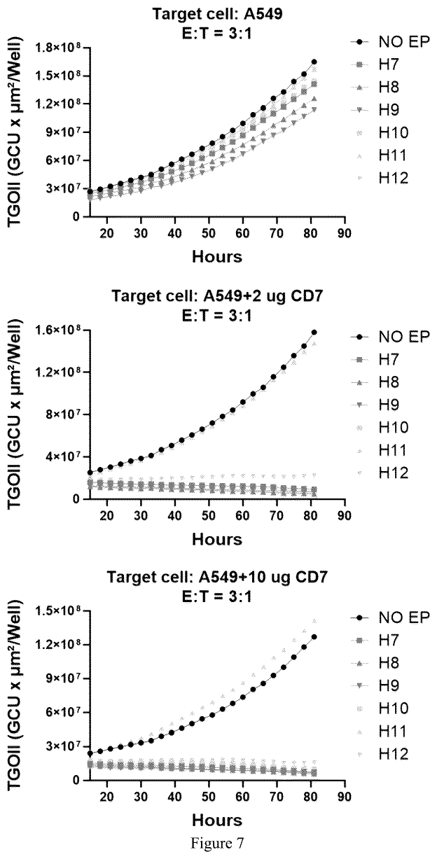

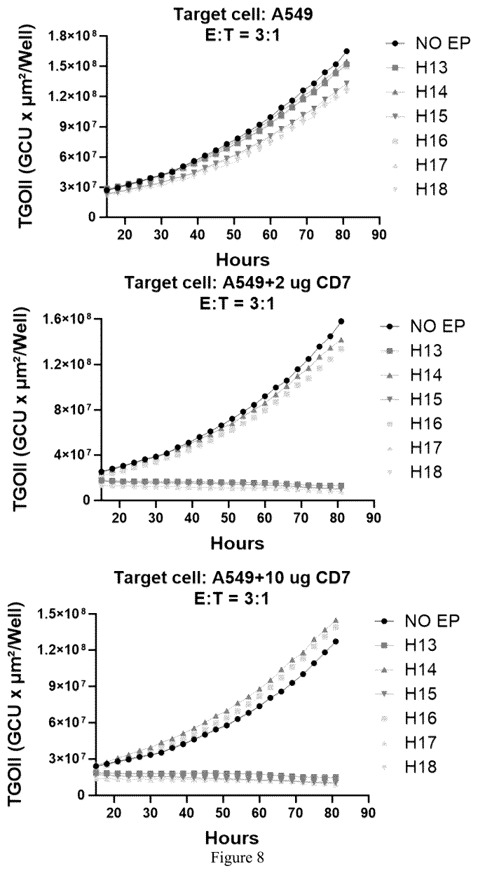

The results are shown in FIGS. 6-8. Except for anti-CD7 scFv-H2 CAR-T, anti-CD7 scFv-H3 CAR-T, anti-CD7 scFv-H11 CAR-T, anti-CD7 scFv-H14 CAR-T and anti-CD7 scFv-H16 CAR-T cells, the remaining anti-CD7 scFv CAR-T cells were cytotoxic to A549 tumor cells expressing CD7.

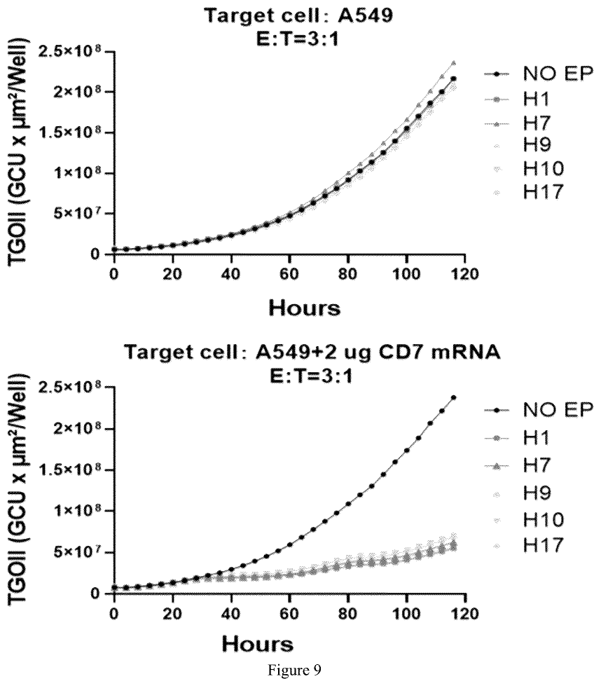

Among them, FIG. 9 shows that anti-CD7 scFv-H1, -H7, -H9, -H10 and -H17 CAR-T cells have strong killing effects on A549 tumor cells that overexpress CD7.

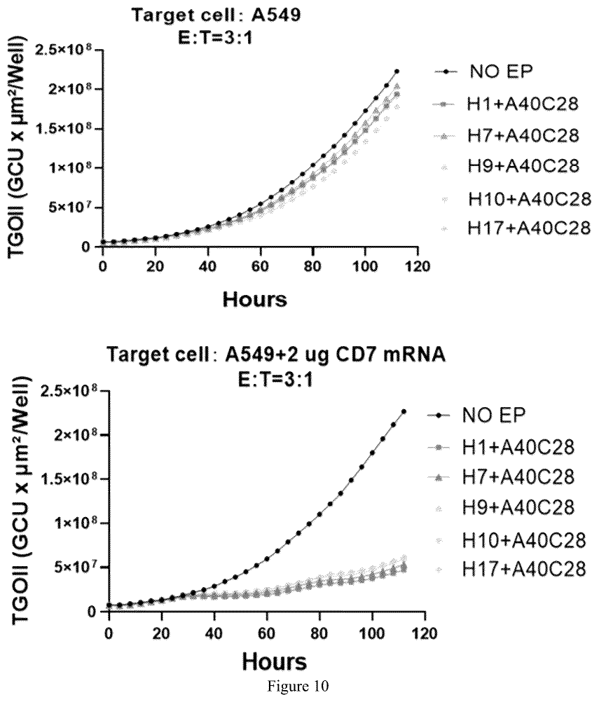

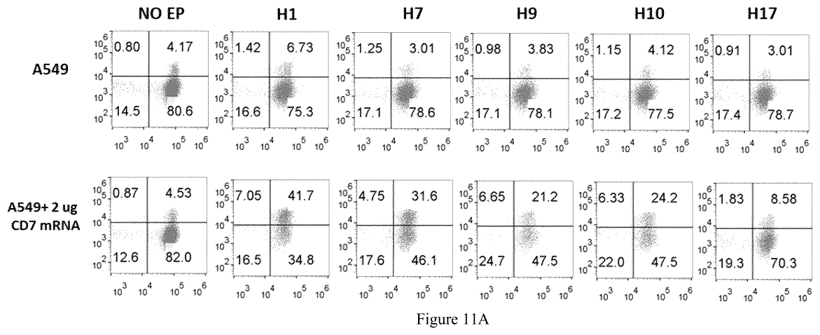

FIG. 10 shows that anti-CD7 CAR (H1, H7, H9, H10, H17)+A40C28 T cells have a strong killing effect on A549 tumor cells that overexpress CD7.

### Example 9 CD107a Staining of CAR-T Cells

1. Add 20 ul PE-CD107a mAb to each well of a 96-well plate.
2. Dilute tumor cells to 2×10e6/ml and seed the cells at 100 ul/well/96-well round plate.
3. Dilute CAR-T cells to 1×10e6/ml and seed the cells at 100 ul/well/96-well round plate.
4. Centrifuge at 500 rpm for 5 minutes to allow the cells to adhere.
5. Incubate the cells at 37° C. for 1 hour.
6. Dilute the Golgi hemostatic agent 1500 times with culture medium and add 20 ul of the diluted Golgi hemostatic agent to each well.
7. Incubate the cells at 37° C. for an additional 2.5 hours.
8. Stain with anti-CD3-APC and anti-CD8-FITC antibodies at 37° C. for 30 minutes.
9. Wash and analyze by flow cytometry.

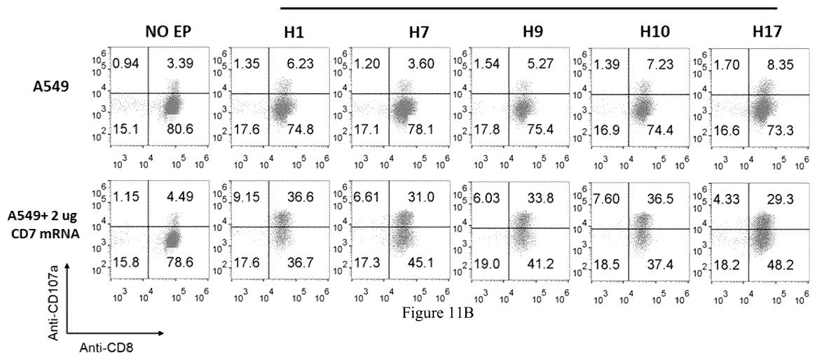

CD107a is an early activation marker. FIG. 11 shows that anti-CD7 CAR (H1, -H7, -H9, -H10, and -H17) T cells can be specifically activated by the CD7 antigen.

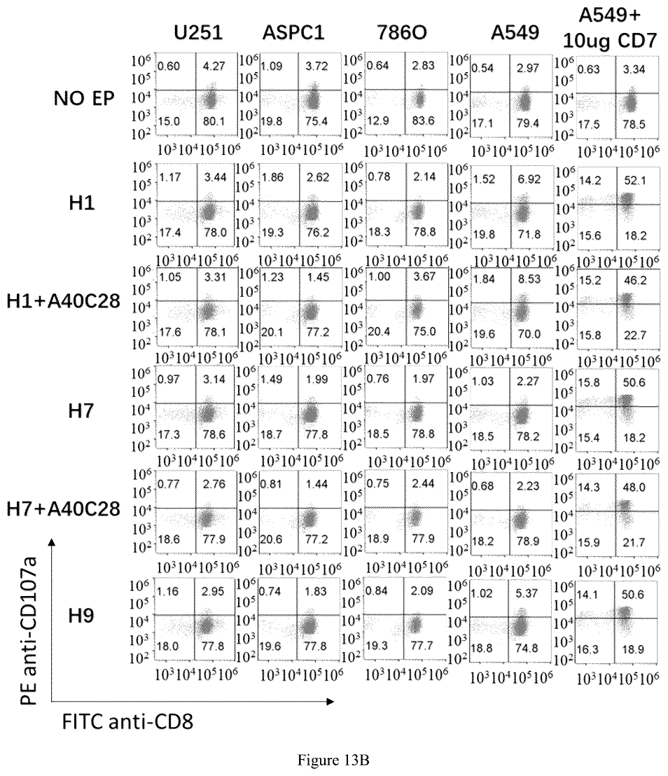

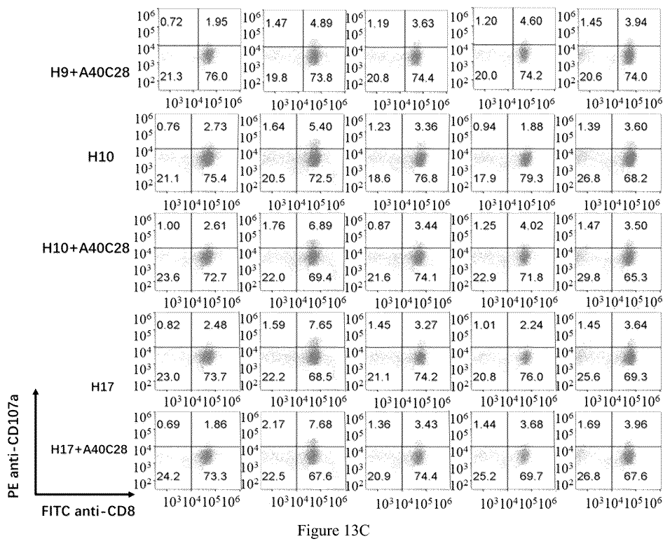

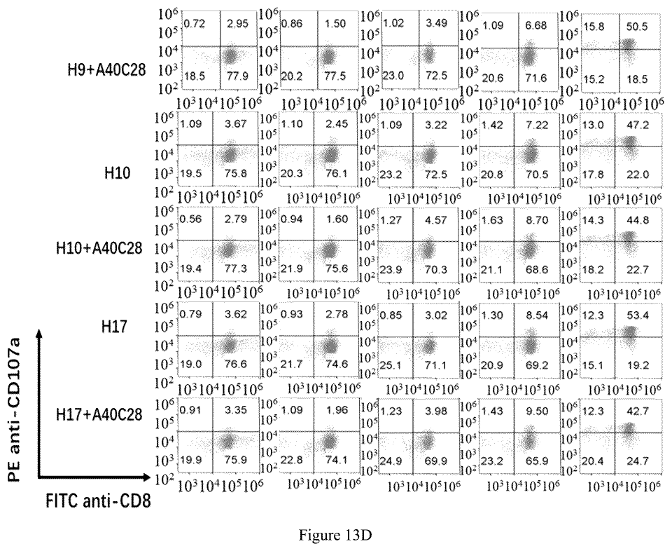

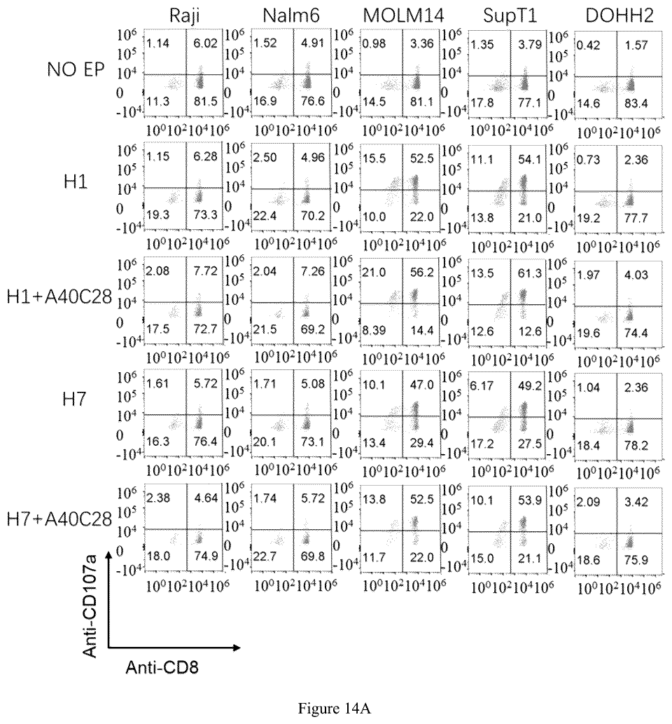

In co-culture and cytotoxicity assays with H226, OVCAR3, SKOV3, Caski, U87, U251, ASPC1, A549, and A549+10 μg CD7 mRNA, the CD107a staining of anti-CD7 CAR-T cells with different mRNAs was analyzed, including mock T cells (NO EP) and T cells electroporated with anti-CD7 CAR-H1, -H7, -H9, -H10, and -H17. FIGS. 13-14 show that all five anti-CD7 CAR (H1, H7, H9, H10, H17) T cells were specifically activated by cells expressing CD7 antigen, but not CD7-negative tumor cell lines.

In the gRNA screening part, among the 12 candidate gRNAs, the CD7 knockout efficiencies of CD7 gRNA-2, gRNA-5, gRNA-6, gRNA-8, gRNA-9, gRNA-10, gRNA-11 and gRNA-12 were 54.5%, 87.9%, 42.7%, 42.7%, 80.1%, 60.5%, 92% and 88.6%, respectively, while CD7 gRNA-1, gRNA-3, gRNA-4 and gRNA-7 had almost no knockout effect.

Among the 18 anti-CD7 scFv sequences, anti-CD7 scFv-H1, -H4, -H5, -H6, -H7, -H8, -H9, -H10, -H12, -H13, -H14, -H15, -H17 and -H18 have the ability to bind to CD7-Fc recombinant protein, while anti-CD7 scFv-H2, -H3, -H11 and -H16 have no or weak binding ability to CD7-Fc recombinant protein. Further results showed that in tumor killing and CD107a staining experiments, five anti-CD7 CAR (H1, H7, H9, H10, H17) T cells were specifically activated by cells expressing CD7 antigen, but not CD7 negative tumor cell lines.

### Example 10 Construction of Lymphocyte-APCs Co-Stimulatory Molecules

Four different forms of fusion proteins targeting both CD28 and CD40 (lymphocyte-APC co-stimulatory molecule (LACO-Stim)) were designed and constructed (FIG. 15).
1) A fusion protein consisting of extracellular CD40 ligand (CD40L) and intracellular CD28.
2) A fusion protein consisting of anti-CD28 scFv and the extracellular region of CD40L (Ab-based soluble protein).
3) Bispecific antibodies composed of scFv against CD28 and CD40.
4) Chimeric proteins consisting of anti-CD40 scFv as the extracellular region and CD28 (or 4-1BB) intracellular region (Ab-based membrane fusion protein).

### Example 11 Construction of Co-Expression of LACO-Stim and CAR-T Cells or TCR-T Cells

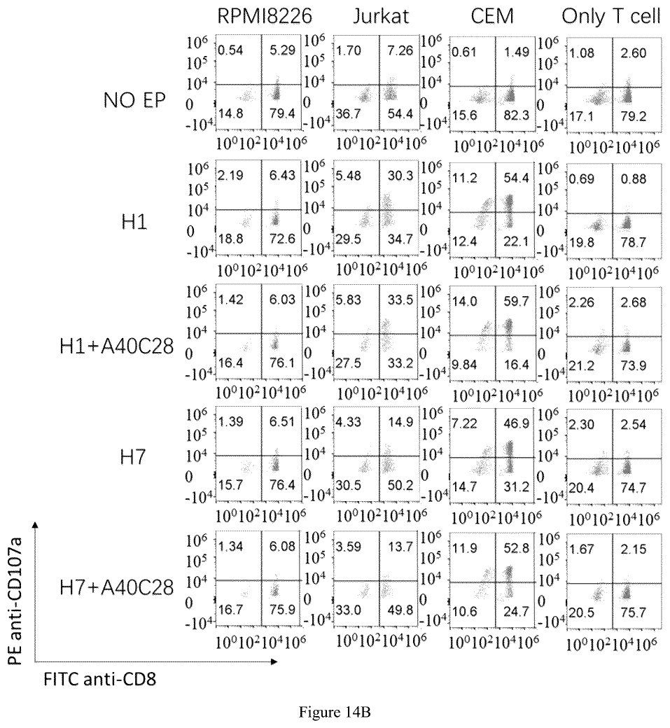

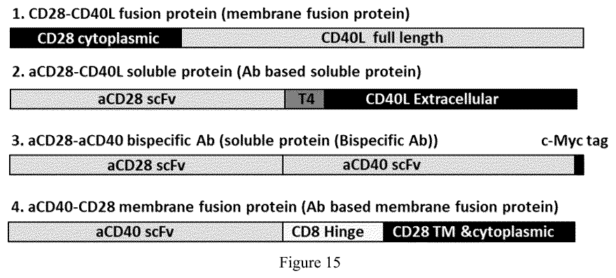

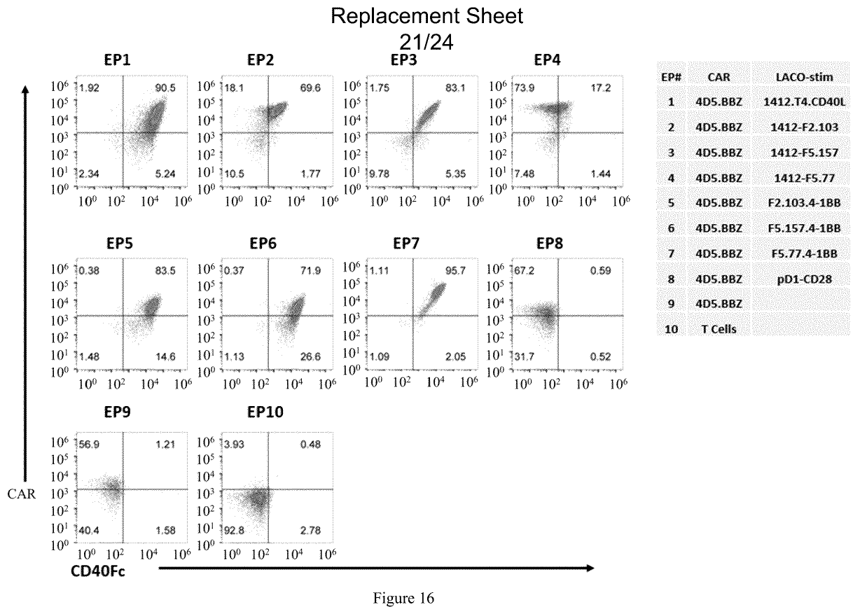

TCR and LACO-stim in Table 3 were co-transferred to T cells by RNA electroporation, and the expression of CAR/LACO-stim was examined by flow cytometry. All T cells co-transfected with CAR and LACO-stim were detected to have CAR staining and CD40-Fc protein binding (FIG. 16)

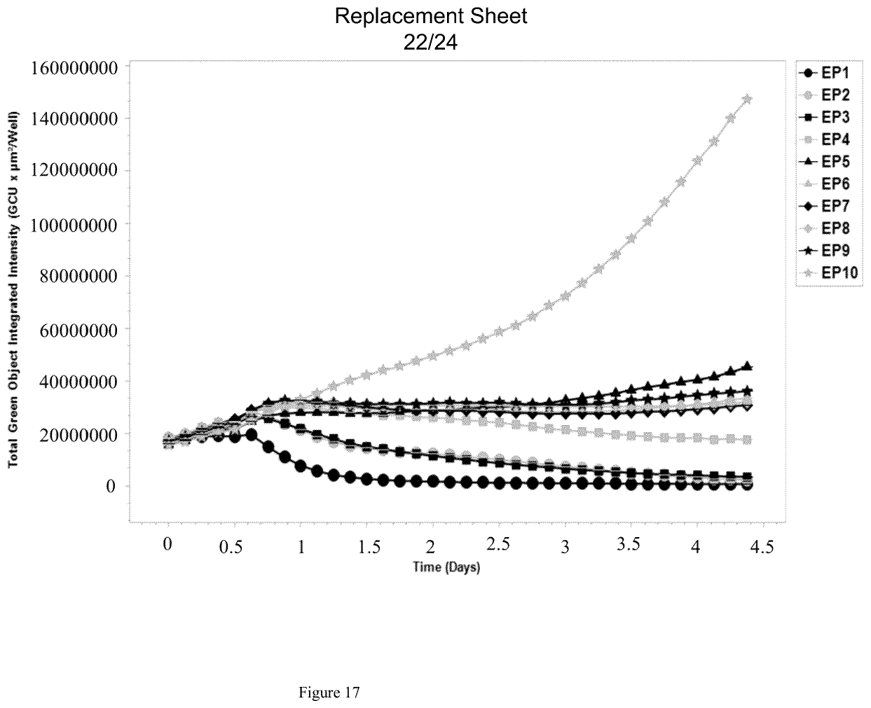

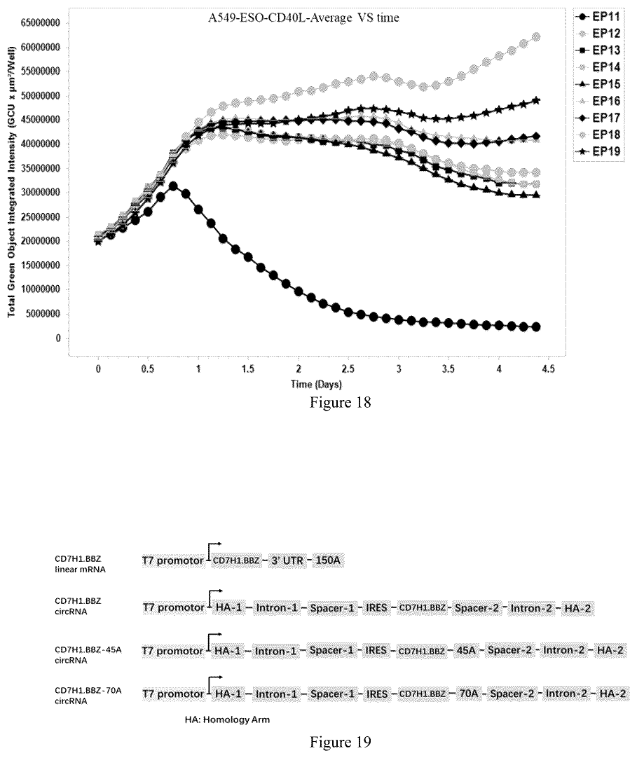

In the Incucyte Live Cell Analysis System, a real-time quantitative live cell imaging and analysis platform that enables visualization and quantification of cell behavior. Using the Her2-positive cancer line A549 transduced with HLA-A2 and NY-ESO-1 (A549-ESO) as the targeted tumor cell line, it was found that T cells co-expressing CAR and 1412.T4.CD4L, or 1412-F2.103, or 1412-F5.157 or 1412-F5.77 showed an improved ability to control tumor growth compared with CAR alone or CAR co-expressed with the switching molecule PD1-CD28, indicating that the soluble form of LACO-stim provides T cells with additional CD28 signals to further activate T cells, proliferation and survival, resulting in an improved ability to control tumor growth (FIG. 17). When LACO-stim was co-introduced with NY-ESO-1 TCR, it was found that all LACO-stim molecules helped TCR T cells to further control tumor growth compared with TCR alone or TCR co-transferred with PD1-CD28 (FIG. 18).

TABLE 3

| EP# | CAR/TCR | LACO-stim |
|---|---|---|
| 1 | 4D5.BBZ | 1412.T4.CD40L |
| 2 | 4D5.BBZ | 1412-F2.103 |
| 3 | 4D5.BBZ | 1412-F5.157 |
| 4 | 4D5.BBZ | 1412-F5.77 |
| 5 | 4D5.BBZ | F2.103.4-1BB |
| 6 | 4D5.BBZ | F5.157.4-1BB |
| 7 | 4D5.BBZ | F5.77.4-1BB |
| 8 | 4D5.BBZ | PD1-CD28 |
| 9 | 4D5.BBZ | |
| 10 | NO EP | |
| 11 | TCR | 1412.T4.CD40L |
| 12 | TCR | 1412-F2.103 |
| 13 | TCR | 1412-F5.157 |
| 14 | TCR | 1412-F5.77 |
| 15 | TCR | F2.103.4-1BB |
| 16 | TCR | F5.157.BB |
| 17 | TCR | F5.77.4-1BB |
| 18 | TCR | PD1-CD28 |
| 19 | TCR | |
| 20 | NO EP | |

### Example 12 Preparation and Characterization of CirRNA Electroporated Cells

1. In Vitro Transcription (IVT) of CD7 H1.BBZ CAR Linear mRNA
1.1 Linearize the pDA-CD7 H1.BBZ CAR (i.e., CAR-H1) plasmid by digestion with BspQ1 enzyme. The amino acid sequence of CAR-H1 is shown in SEQ ID NO: 180.
1.2 Purify the linearized vector using PCR Cleanup kit (Qiagen) and elute with RNase-free water.
1.3 DNA concentration was determined by nanodrop and checked by agarose DNA gel electrophoresis.
1.4 IVT was performed according to the manufacturer's instructions (Thermofisher, catalog number: AMB13455). Briefly, 1 μg of template DNA, NTP/ARCA buffer, T7 buffer, GTP, T7 enzyme and RNase free H₂O were added to a 0.2 ml PCR tube in a volume of 20 μl and incubated at 37° C. for 4 h.
1.5 After 4 hours, add 2 μl of DNase I to each reaction and incubate at 37° C. for 15 minutes.
1.6 Then carry out the tailing procedure.
1.7 IVT mRNA was purified using the RNasy kit (Qiagen).
1.8 RNA concentration was determined by nanodrop method and detected by PAGE gel electrophoresis.
   2. In Vitro Transcription (IVT) of CD7 H1.BBZ CAR circRNA
2.1 Linearize the pCA-CD7 H1.BBZ, pCA45-CD7 H1.BBZ, pCA70-CD7 H1.BBZ CAR plasmids by digestion with BspQ1 enzyme.
2.2 Purify the linearized vector using PCR Cleanup kit (Qiagen) and elute with RNase-free water.
2.3 DNA concentration was determined by nanodrop and checked by agarose DNA gel electrophoresis.
2.4 IVT was performed according to the manufacturer's instructions (Thermofisher, catalog number: AMB13345). Briefly, 1 μg template DNA, NTP mixture, T7 buffer, GTP, T7 enzyme and RNase free H₂O were added to a 0.2 ml PCR tube in a volume of 20 μl and incubated at 37° C. for 4 h.
2.5 After 4 hours, add 2 μl DNase I to each reaction and incubate at 37° C. for 15 minutes.
2.6 After DNase treatment, add additional GTP to a final concentration of 2 mM and heat the reaction at 55° C. for 15 min.
2.7 circRNA was purified by column or AKTA system.
2.8 RNA concentration was determined by nanodrop method and detected by PAGE gel electrophoresis.

The structures of CD7 H1.BBZ CAR linear mRNA and CD7 H1.BBZ CAR circRNA are shown in FIG. 19. The nucleotide sequence of the T7 promoter is shown in SEQ ID NO: 258; the nucleotide sequence of homology arm 1 is shown in SEQ ID NO: 259; the nucleotide sequence of intron 1 is shown in SEQ ID NO: 260; the nucleotide sequence of spacer 1 is shown in SEQ ID NO: 261; The nucleotide sequence of CVB 3 IRES is shown in SEQ ID NO: 262; the nucleotide sequence of spacer 2 is shown in SEQ ID NO: 263; the nucleotide sequence of intron 2 is shown in SEQ ID NO: 264; the nucleotide sequence of homology arm 2 is shown in SEQ ID NO: 265; the nucleotide sequence of 70A is shown in SEQ ID NO: 266.

CD7 H1.BBZ CAR linear mRNA (CD7 CAR-H1 linear mRNA) is shown in SEQ ID NO: 255; the nucleotide sequence of CD7 H1.BBZ CAR circRNA (CD7 CAR-H1 circRNA) is shown in SEQ ID NO: 256; the nucleotide sequence of CD7 H1.BBZ CAR circRNA (CD7 CAR-H1 circRNA-70A) is shown in SEQ ID NO: 257.

3. Purification of circRNA Using CIMmultus Oligo dT Columns
3.1 Buffer preparation: Buffer A, equilibration/wash buffer: 50 mM sodium phosphate, 250 mM sodium chloride, 5 mM EDTA, pH 7.0; Buffer B, second wash buffer: 50 mM sodium phosphate, 5 mM EDTA, pH 7.0; Buffer C, elution buffer: 10 mM Tris, pH 8.0;
Buffer D, cleaning buffer: 3 M guanidine hydrochloride, 5 mM EDTA, pH 7.0.
3.2 Equilibrate column with buffer A: Pump equilibration buffer (A) through the CIMmultus Oligo dT column until the output pH and conductivity are the same as the input buffer. Flow rate: 5 column volumes per minute (CV/min).
3.3 Inject the sample. Inject the sample according to the instructions.
3.4 Perform the first elution with buffer A (Wash 1): 10 CV of equilibration buffer.
3.5 Perform a second elution with buffer B (Wash 2): 10 CV of the second wash buffer.
3.6 Dilute with buffer C: Elute and collect circRNA samples until the UV absorbance returns to baseline.
3.7 Wash with Buffer D. Wash the column system with at least 10 CV of wash buffer.
   4. Electroporation of RNA into A549-GFP and T Cells
4.1 Collect A549-GFP tumor cells and T cells and wash them three times with Opti-MEM medium.
4.2 Resuspend the cell pellet in Opti-MEM medium and adjust the cell concentration to 1×10e7/ml.
4.3 Aliquot 5 μg MSLN mRNA into 1.5 ml EP tubes, add 100 μl A549 cells and mix. For T cell electroporation, aliquot 10 μg CD7 H1.BBZ mRNA or circRNA into 1.5 ml EP tubes, then add 100 μl A549 cells and mix.
4.4 Set BTX machine parameters:
a) T cells: 500 V, 0.7 ms
b) A549 tumor cells: 300 V, 0.5 ms;
4.5 Add 100 μl of cells mixed with RNA into the BTX electroporation cuvette and tap gently to avoid bubbles.
4.6 Perform electroporation, then transfer the cells to pre-warmed culture medium and culture at 37° C.

### 5. In Vitro Cytotoxicity Assay of CD7 H1.BBZ CAR-T Cells

5.1 12 hours before co-culture, A549-EGFP cells electroporated with 5 μg CD7 mRNA were seeded at 3000 cells/100 μl/well in a flat-bottom 96-well plate.
5.2 About 12-24 hours after electroporation, count and dilute the CAR-T cells to an appropriate concentration, and add 100 μl/well of seed cells to tumor cells with different E/T ratios such as 3:1 and 1:1.
5.3 Place the co-culture plate into the IncuCyte S3 machine and set the scanning parameters.
5.4 4 days after scanning, analyze the total green object integrated intensity (GCU×μm²/Well) and calculate the killing efficiency.

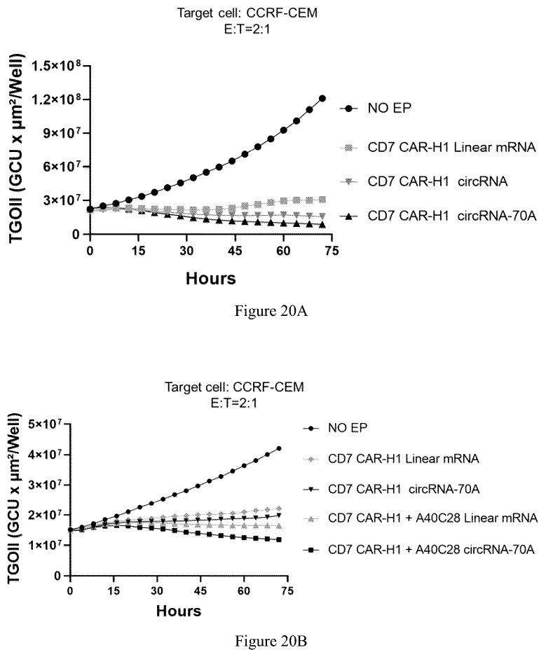

The results are shown in FIGS. 20A-20B, which respectively show that CD7 H1.BBZ CAR linear mRNA and CD7 H1.BBZ CAR circRNA have specific killing effects on tumor cells. Among them, A40C28 in FIG. 20B refers to T cells of the simultaneous electroporation of CD7 H1.BBZ CAR linear mRNA and A40C28 linear mRNA; or, T cells electroporated CD7 H1.BBZ CAR circRNA and A40C28 cirRNA. FIG. 20A showed that the cirRNA group was better than the linear mRNA group. The cirRNA ployA group had a better effect than the cirRNA group; FIG. 20B showed that in cells of co-expressing A40C28, The effect of the cirRNA ployA group was better than that of the linear mRNA group.

### REFERENCES

1. Morgan R A, Dudley M E, Wunderlich J R, Hughes M S, Yang J C, Sherry R M, Royal R E, Topalian S L, Kammula U S, Restifo N P, Zheng Z et al: Cancer regression in patients after transfer of genetically engineered lymphocytes. Science (2006) 314(5796):126-129.
2. Robbins P F, Morgan R A, Feldman S A, Yang J C, Sherry R M, Dudley M E, Wunderlich J R, Nahvi A V, Helman L J, Mackall C L, Kammula U S et al: Tumor regression in patients with metastatic synovial cell sarcoma and melanoma using genetically engineered lymphocytes reactive with ny-eso-1. J Clin Oncol (2011) 29(7):917-924.
3. Rapoport A P, Stadtmauer E A, Binder-Scholl G K, Goloubeva O, Vogl D T, Lacey S F, Badros A Z, Garfall A, Weiss B, Finklestein J, Kulikovskaya I et al: Ny-eso-1-specific tcr-engineered t cells mediate sustained antigen-specific antitumor effects in myeloma. Nature medicine (2015) 21(8):914-921.
4. Kochenderfer J N, Wilson W H, Janik J E, Dudley M E, Stetler-Stevenson M, Feldman S A, Maric I, Raffeld M, Nathan D A, Lanier B J, Morgan R A et al: Eradication of b-lineage cells and regression of lymphoma in a patient treated with autologous t cells genetically engineered to recognize cd19. Blood (2010) 116(20):4099-4102.
5. Kalos M, Levine B L, Porter D L, Katz S, Grupp S A, Bagg A, June C H: T cells with chimeric antigen receptors have potent antitumor effects and can establish memory in patients with advanced leukemia. Science translational medicine (2011) 3(95):95ra73.
6. Anderson K G, Stromnes I M, Greenberg P D: Obstacles posed by the tumor microenvironment to t cell activity: A case for synergistic therapies. Cancer Cell (2017) 31(3):311-325.
7. Boutros C, Tarhini A, Routier E, Lambotte O, Ladurie F L, Carbonnel F, Izzeddine H, Marabelle A, Champiat S, Berdelou A, Lanoy E et al: Safety profiles of anti-ctla-4 and anti-pd-1 antibodies alone and in combination. Nature reviews Clinical oncology (2016) 13(8):473-486.
8. Stephan M T, Ponomarev V, Brentjens R J, Chang A H, Dobrenkov K V, Heller G, Sadelain M: T cell-encoded cd80 and 4-1bbl induce auto- and transco-stimulation, resulting in potent tumor rejection. Nat Med (2007) 13(12):1440-1449.
9. Topp M S, Riddell S R, Akatsuka Y, Jensen M C, Blattman J N, Greenberg P D: Restoration of cd28 expression in cd28− cd8+ memory effector t cells reconstitutes antigen-induced il-2 production. J Exp Med (2003) 198(6):947-955.
10. Daniel-Meshulam I, Horovitz-Fried M, Cohen C J: Enhanced antitumor activity mediated by human 4-1bb-engineered t cells. International journal of cancer (2013) 133(12):2903-2913.
11. Ankri C, Cohen C J: Out of the bitter came forth sweet: Activating cd28-dependent co-stimulation via pd-1 ligands. Oncoimmunology (2014) 3(1):e27399.
12. Prosser M E, Brown C E, Shami A F, Forman S J, Jensen M C: Tumor pd-li co-stimulates primary human cd8(+) cytotoxic t cells modified to express a pd1:Cd28 chimeric receptor. Molecular immunology (2012) 51(3-4):263-272.
13. Ankri C, Shamalov K, Horovitz-Fried M, Mauer S, Cohen C J: Human t cells engineered to express a programmed death 1/28 co-stimulatory retargeting molecule display enhanced antitumor activity. J Immunol (2013) 191(8):4121-4129.
14. Kobold S, Grassmann S, Chaloupka M, Lampert C, Wenk S, Kraus F, Rapp M, Duwell P, Zeng Y, Schmollinger J C, Schnurr M et al: Impact of a new fusion receptor on pd-1-mediated immunosuppression in adoptive t cell therapy. J Natl Cancer Inst (2015) 107(8).
15. Liu X, Ranganathan R, Jiang S, Fang C, Sun J, Kim S, Newick K, Lo A, June C H, Zhao Y, Moon E K: A chimeric switch-receptor targeting pd1 augments the efficacy of second-generation CAR T cells in advanced solid tumors. Cancer research (2016) 76(6):1578-1590.
16. Schlenker R, Olguin-Contreras L F, Leisegang M, Schnappinger J, Disovic A, Ruhland S, Nelson P J, Leonhardt H, Harz H, Wilde S, Schendel D J et al: Chimeric pd-1:28 receptor upgrades low-avidity t cells and restores effector function of tumor-infiltrating lymphocytes for adoptive cell therapy. Cancer research (2017) 77(13):3577-3590.
17. Li S, Siriwon N, Zhang X, Yang S, Jin T, He F, Kim Y J, Mac J, Lu Z, Wang S, Han X et al: Enhanced cancer immunotherapy by chimeric antigen receptor-modified t cells engineered to secrete checkpoint inhibitors. Clinical cancer research: an official journal of the American Association for Cancer Research (2017) 23(22):6982-6992.
18. Rafiq S, Yeku O O, Jackson H J, Purdon T J, van Leeuwen D G, Drakes D J, Song M, Miele M M, Li Z, Wang P, Yan S et al: Targeted delivery of a pd-1-blocking scfv by car-t cells enhances anti-tumor efficacy in vivo. Nature biotechnology (2018) 36(9):847-856.
19. Liu X, Jiang S, Fang C, Yang S, Olalere D, Pequignot E C, Cogdill A P, Li N, Ramones M, Granda B, Zhou L et al: Affinity-tuned erbb2 or egfr chimeric antigen receptor t cells exhibit an increased therapeutic index against tumors in mice. Cancer Res (2015) 75(17):3596-3607.
20. Zhao Y, Zheng Z, Robbins P F, Khong H T, Rosenberg S A, Morgan R A: Primary human lymphocytes transduced with ny-eso-1 antigen-specific tcr genes recognize and kill diverse human tumor cell lines. J Immunol (2005) 174(7):4415-4423.
21. Zhao Y, Moon E, Carpenito C, Paulos C M, Liu X, Brennan A L, Chew A, Carroll R G, Scholler J, Levine B L, Albelda S M et al: Multiple injections of electroporated autologous t cells expressing a chimeric antigen receptor mediate regression of human disseminated tumor. Cancer research (2010) 70(22):9053-9061.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. An isolated antigen-binding protein that specifically binds to CD7 protein, wherein the isolated antigen-binding protein comprises a VH and a VL, wherein the VH comprises HCDR1, HCDR2 and HCDR3, and the VL comprises LCDR1, LCDR2 and LCDR3; wherein the HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 74, the LCDR2 comprises the amino acid sequence shown in SEQ ID NO: 87, and the LCDR3 comprises the amino acid sequence shown in SEQ ID NO: 99; or
The HCDR1 comprises the amino acid sequence shown in SEQ ID NO: 3, the HCDR2 comprises the amino acid sequence shown in SEQ ID NO: 11, the HCDR3 comprises the amino acid sequence shown in SEQ ID NO: 19, the LCDR1 comprises the amino acid sequence shown in SEQ ID NO: 75, the LCDR2 comprises the amino acid sequence shown in SEQ ID NO: 88, and the LCDR3 comprises the amino acid sequence shown in SEQ ID NO: 100; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 20, the LCDR1 comprises the amino acid sequence shown in SEQ ID NO: 76, the LCDR2 comprises the amino acid sequence shown in SEQ ID NO: 89, and the LCDR3 comprises the amino acid sequence shown in SEQ ID NO: 101; or
The HCDR1 comprises the amino acid sequence shown in SEQ ID NO: 4, the HCDR2 comprises the amino acid sequence shown in SEQ ID NO: 12, the HCDR3 comprises the amino acid sequence shown in SEQ ID NO: 21, the LCDR1 comprises the amino acid sequence shown in SEQ ID NO: 77, the LCDR2 comprises the amino acid sequence shown in SEQ ID NO: 90, and the LCDR3 comprises the amino acid sequence shown in SEQ ID NO: 102; or
The HCDR1 comprises the amino acid sequence shown in SEQ ID NO: 5, the HCDR2 comprises the amino acid sequence shown in SEQ ID NO: 13, the HCDR3 comprises the amino acid sequence shown in SEQ ID NO: 22, the LCDR1 comprises the amino acid sequence shown in SEQ ID NO: 91, the LCDR2 comprises the amino acid sequence shown in SEQ ID NO: 91, and the LCDR3 comprises the amino acid sequence shown in SEQ ID NO: 103; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 79, the LCDR2 comprises the amino acid sequence shown in SEQ ID NO: 92, and the LCDR3 comprises the amino acid sequence shown in SEQ ID NO: 104; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 80, the LCDR2 comprises the amino acid sequence shown in SEQ ID NO: 93, and the LCDR3 comprises the amino acid sequence shown in SEQ ID NO: 105; or
The HCDR1 comprises the amino acid sequence shown in SEQ ID NO: 6, the HCDR2 comprises the amino acid sequence shown in SEQ ID NO: 14, the HCDR3 comprises the amino acid sequence shown in SEQ ID NO: 23, the LCDR1 comprises the amino acid sequence shown in SEQ ID NO: 81, the LCDR2 comprises the amino acid sequence shown in SEQ ID NO: 94, and the LCDR3 comprises the amino acid sequence shown in SEQ ID NO: 106; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 18, the LCDR1 comprises the amino acid sequence shown in SEQ ID NO: 82, the LCDR2 comprises the amino acid sequence shown in SEQ ID NO: 95, and the LCDR3 comprises the amino acid sequence shown in SEQ ID NO: 107; or
The HCDR1 comprises the amino acid sequence shown in SEQ ID NO: 2, the HCDR2 comprises the amino acid sequence shown in SEQ ID NO: 10, the HCDR3 comprises the amino acid sequence shown in SEQ ID NO: 24, the LCDR1 comprises the amino acid sequence shown in SEQ ID NO: 83, the LCDR2 comprises the amino acid sequence shown in SEQ ID NO: 96, and the LCDR3 comprises the amino acid sequence shown in SEQ ID NO: 108; or
The HCDR1 comprises the amino acid sequence shown in SEQ ID NO: 7, the HCDR2 comprises the amino acid sequence shown in SEQ ID NO: 15, the HCDR3 comprises the amino acid sequence shown in SEQ ID NO: 25, the LCDR1 comprises the amino acid sequence shown in SEQ ID NO: 84, the LCDR2 comprises the amino acid sequence shown in SEQ ID NO: 97, and the LCDR3 comprises the amino acid sequence shown in SEQ ID NO: 109; or
The HCDR1 comprises the amino acid sequence shown in SEQ ID NO: 8, the HCDR2 comprises the amino acid sequence shown in SEQ ID NO: 16, the HCDR3 comprises the amino acid sequence shown in SEQ ID NO: 26, the LCDR1 comprises the amino acid sequence shown in SEQ ID NO: 85, the LCDR2 comprises the amino acid sequence shown in SEQ ID NO: 98, and the LCDR3 comprises the amino acid sequence shown in SEQ ID NO: 110; or
The HCDR1 comprises the amino acid sequence shown in SEQ ID NO: 9, the HCDR2 comprises the amino acid sequence shown in SEQ ID NO: 17, the HCDR3 comprises the amino acid sequence shown in SEQ ID NO: 27, the LCDR1 comprises the amino acid sequence shown in SEQ ID NO: 86, the LCDR2 comprises the amino acid sequence shown in SEQ ID NO: 98, and the LCDR3 comprises the amino acid sequence shown in SEQ ID NO: 111.

17. (canceled)

18. (canceled)

19. (canceled)

20. (canceled)

21. (canceled)

22. (canceled)

23. The isolated antigen-binding protein of claim 16, wherein the VH comprises the amino acid sequence shown in SEQ ID NO: 61, and the VL comprises the amino acid sequence shown in SEQ ID NO: 148; or
The VH comprises the amino acid sequence shown in SEQ ID NO: 62, and the VL comprises the amino acid sequence shown in SEQ ID NO: 149; or
The VH comprises the amino acid sequence shown in SEQ ID NO: 63, and the VL comprises the amino acid sequence shown in SEQ ID NO: 150; or
The VH comprises the amino acid sequence shown in SEQ ID NO: 64, and the VL comprises the amino acid sequence shown in SEQ ID NO: 151; or
The VH comprises the amino acid sequence shown in SEQ ID NO: 65, and the VL comprises the amino acid sequence shown in SEQ ID NO: 152; or
The VH comprises the amino acid sequence shown in SEQ ID NO: 66, and the VL comprises the amino acid sequence shown in SEQ ID NO: 153; or
The VH comprises the amino acid sequence shown in SEQ ID NO: 67, and the VL comprises the amino acid sequence shown in SEQ ID NO: 154; or
The VH comprises the amino acid sequence shown in SEQ ID NO: 68, and the VL comprises the amino acid sequence shown in SEQ ID NO: 155; or
The VH comprises the amino acid sequence shown in SEQ ID NO: 69, and the VL comprises the amino acid sequence shown in SEQ ID NO: 156; or
The VH comprises the amino acid sequence shown in SEQ ID NO: 70, and the VL comprises the amino acid sequence shown in SEQ ID NO: 157; or
The VH comprises the amino acid sequence shown in SEQ ID NO: 71, and the VL comprises the amino acid sequence shown in SEQ ID NO: 158; or
The VH comprises the amino acid sequence shown in SEQ ID NO: 72, and the VL comprises the amino acid sequence shown in SEQ ID NO: 159; or
The VH comprises the amino acid sequence shown in SEQ ID NO: 73, and the VL comprises the amino acid sequence shown in SEQ ID NO: 160;
preferably, the isolated antigen-binding protein includes an antibody or an antigen-binding fragment thereof;
more preferably, the antibody preferably includes a monoclonal antibody, a polyclonal antibody, a dimer, a multimer, a multispecific antibody, a full-length antibody, an antibody fragment, a human antibody, a humanized antibody or a chimeric antibody; the antigen-binding fragment preferably includes Fab, Fab′, Fv fragment, F(ab′)₂, scFv, di-scFv and/or dAb;
further more preferably, the isolated antigen-binding protein comprises the amino acid sequence shown in any one of SEQ ID NO: 162 to SEQ ID NO: 174.

24. (canceled)

25. (canceled)

26. (canceled)

27. (canceled)

28. (canceled)

29. (canceled)

30. (canceled)

31. (canceled)

32. An isolated polypeptide comprising the isolated antigen-binding protein of claim 16.

33. An immunoconjugate comprising the antigen-binding protein of claim 16.

34. A chimeric antigen receptor comprising at least one extracellular antigen-binding domain comprising the antigen-binding protein of claim 16;
preferably, the extracellular antigen-binding domain includes an scFv;
more preferably, the chimeric antigen receptor comprises the amino acid sequence shown in any one of SEQ ID NO: 180 to SEQ ID NO: 192.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. An isolated nucleic acid molecule encoding the isolated antigen-binding protein of claim 16.

55. An expression vector comprising the nucleic acid molecule of claim 54.

56. A cell expressing the antigen-binding protein of claim 16.

57. A method for preparing an isolated antigen-binding protein, including culturing the cell of claim 56 under conditions that allow for the expression of the isolated antigen-binding protein.

58. An engineered cell comprising the antigen-binding protein of claim 16;
preferably, the cell includes an immune effector cell, the immune effector cell preferably includes a human cell;
more preferably, the immune effector cell includes a T cell, a B cell, a natural killer cell (NK cell), a macrophages, an NKT cell, a monocyte, a dendritic cell, a granulocyte, a lymphocyte, a leukocyte and/or a peripheral blood mononuclear cell.

59. (canceled)

60. (canceled)

61. (canceled)

62. (canceled)

63. (canceled)

64. The engineered cell of claim 58, wherein the immune effector cell includes a modified immune effector cell;
preferably, the expression of CD7 of the modified immune effector cell is absent or suppressed;
more preferably, the immune effector cell includes CAR-T cell.

65. (canceled)

66. (canceled)

67. (canceled)

68. (canceled)

69. A method for preparing a population of chimeric antigen receptor T (CAR-T) cells, wherein the CAR targets CD7, the method including the following steps:
a. Modifying the CD7 gene in the population of T cells;
b. Activating the population of T cells;
c. Transducing the T cell with the chimeric antigen receptor of claim 34; and
d. Amplify the chimeric antigen receptor T cell.

70. The method of claim 69, wherein CD7 is absent or suppressed in the modified population of T cells compared with the unmodified corresponding cell;
preferably, the modification includes administering to the population of T cells one or more substances selected from the group consisting of antisense RNA, siRNA, shRNA, transcription activator-like effector nuclease (TALEN), zinc finger nuclease (ZFN) and CRISPR/Cas system.

71. (canceled)

72. The method of claim 69, wherein the modification includes administering a CRISPR/Cas system to the population of T cells;
preferably, the modification includes administering a CRISPR/Cas9 system to the population of T cells;
more preferably, the modification includes administering Cas9 and a gRNA targeting a CD7 gene to the population of T cells; the gRNA targeting the CD7 gene preferably comprises a nucleotide sequence shown in any one of SEQ ID NO: 211 to SEQ ID NO: 218; the Cas9 is preferably delivered to the cell as mRNA or protein; the gRNA is preferably delivered simultaneously with the Cas9;
further more preferably, the delivering is performed by electroporation.

73. (canceled)

74. (canceled)

75. (canceled)

76. (canceled)

77. (canceled)

78. (canceled)

79. The method of claim 69, wherein the transduction of the chimeric antigen receptor includes transduction of a circRNA chimeric antigen receptor;
preferably, the circRNA chimeric antigen receptor comprises, in the following order, an internal ribosome entry site (IRES) element, a protein coding sequence targeting CD7, and polyadenylic acid (polyA).

80. (canceled)

81. A pharmaceutical composition comprising the isolated antigen-binding protein of claim 16, and optionally a pharmaceutically acceptable carrier.

82. A kit comprising the isolated antigen-binding protein of claim 16.

83. (canceled)

84. (canceled)

85. (canceled)

86. (canceled)

87. (canceled)

88. (canceled)

89. A method for treating tumor, the method including administering to a subject in need thereof an effective amount of the isolated antigen-binding protein of claim 16;
preferably, the tumor includes a tumor expressing CD7; or the tumor includes a hematological tumor;
more preferably, the tumor includes a CD7-positive hematological malignancy.

90. (canceled)

91. (canceled)

92. (canceled)

93. The method of claim 89, wherein the tumor includes a T cell malignancy;
preferably, the T-cell malignancy includes acute T-lymphocytic leukemia (T-ALL), acute myeloid leukemia, or NK/T-cell lymphoma.

94. (canceled)

95. A method for killing a malignant T cell, including contacting the malignant T cell with the engineered cell of claim 58.

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)
